(19) United States
(12) Patent Application Publication
(10) Pub. No.: US 2004/0197799 A1
(43) Pub. Date: Oct. 7, 2004

(54) Determination of a genetic predisposition for behavioral disorders

(76) Inventors: Robert Williamson, Parkville (AU); Hans-Henrik Marstrand Dahl, Princes Hill (AU); Susan Mary Forrest, Kew (AU); Stephen Andrew Wilcox, Plenty (AU); Michelle Gina De Silva, Abbotsford (AU); Katherine Seymour Elliott, Elwood (AU); Michael Lynch, Sandringham (AU); Martin Delatycki, East Hawthorn (AU)

Correspondence Address: BAKER & BOTTS, 30 ROCKEFELLER PLAZA, NEW YORK, NY, 10112

(21) Appl. No.: 10/699,156

(22) Filed: Oct. 31, 2003

Related U.S. Application Data
(63) Continuation of application No. PCT/AU02/00556, filed on May 3, 2002.
(60) Provisional application No. 60/295,811, filed on Jun. 4, 2001.

(30) Foreign Application Priority Data
May 3, 2001 (AU) PR4756/01
Jun. 4, 2001 (AU) PR5426/01

(51) Int. Cl. C12Q 1/68

(52) U.S. Cl. 435/6

(57) ABSTRACT

The present invention relates generally to a molecular marker of a behavioral disorder such as but not limited to Attention Deficit Hyperactivity Disorder (ADHD) and to its use in the diagnosis of a behavioral disorder or an assessment of a likelihood that a subject may develop the behavioral disorder. A behavioral disorder also includes an intellectual disability. The molecular marker in essence determines the presence of genetic predisposition for development of the behavioral disorder, the development of which, or its degree of severity, may be further determined or exacerbated by environmental or social conditions. The molecular marker of the present invention may be in the form of a proteinaceous molecule or a genetic sequence. The ability to identify “at risk” individuals permits the implementation of medicinal, behavioral and/or personal management protocols to reduce the likelihood of development of, or to ameliorate one or more of the symptoms of, a behavioral disorder. The present invention contemplates, therefore, diagnostic assays and therapeutic agents in the prophylaxis and/or treatment of a behavioral disorder. The present invention further contemplates screening in utero as well as screening parents, or potential parents, for a likelihood of passing on a genetic predisposition to a behavioral disorder. The latter individuals or subjects identified as having a predisposition to the development of a behavioral disorder can then undergo behavioral modification protocols to control any development of a behavioral disorder.

The present application is a continuation of International Patent Application No. PCT/AU02/00556, filed May 3, 2002, published in English as International Publication No, WO02/090541 on Nov. 14, 2002, and claims priority to Australian Patent Application No. PR4756/01, filed May 3, 2001, Australian Patent Application No. PR5426/01, filed Jun. 4, 2001, and U.S. Provisional Application Serial No. 60/295,811, filed Jun. 4, 2001, all of which are incorporated herein by reference in their entirety.

### FIELD OF THE INVENTION

The present invention relates generally to a molecular marker of a behavioral disorder such as but not limited to Attention Deficit Hyperactivity Disorder (ADHD) and to its use in the diagnosis of a behavioral disorder or an assessment of a likelihood that a subject may develop the behavioral disorder. A behavioral disorder also includes an intellectual disability. The molecular marker in essence determines the presence of genetic predisposition for development of the behavioral disorder, the development of which, or its degree of severity, may be further determined or exacerbated by environmental or social conditions. The molecular marker of the present invention may be in the form of a proteinaceous molecule or a genetic sequence. The ability to identify “at risk” individuals permits the implementation of medicinal, behavioral and/or personal management protocols to reduce the likelihood of development of, or to ameliorate one or more of the symptoms of, a behavioral disorder. The present invention contemplates, therefore, diagnostic assays and therapeutic agents in the prophylaxis and/or treatment of a behavioral disorder. The present invention further contemplates screening in utero as well as screening parents, or potential parents, for a likelihood of passing on a genetic predisposition to a behavioral disorder. The latter individuals or subjects identified as having a predisposition to the development of a behavioral disorder can then undergo behavioral modification protocols to control any development of a behavioral disorder.

### BACKGROUND OF THE INVENTION

Reference to any prior art in this specification is not, and should not be taken as, an acknowledgment or any form of suggestion that this prior art forms part of the common general knowledge in any country.

The rapidly increasing sophistication of recombinant DNA technology is greatly facilitating research and development in the medical and allied health field. A particularly important area involves human genetics and the elucidation of the mechanisms underpinning inherited disorders or disorders having a genetic component. Behavioral disorders are particularly challenging and a prevalent example of this is Attention Deficit Hyperactivity Disorder (ADHD). Genetic association and linkage studies have previously failed to contribute significantly to understanding the genetic basis of behavioral or neuropsychiatric disorders. Although some positive findings have been reported, these are often closely followed by reports that refute the finding. This has certainly been the case for ADHD.

ADHD is one of the most commonly diagnosed behavioral disorders and is a significant burden on health systems. The prevalence of ADHD in the child population in the USA is 3-6% (Ballard et al., Adolescence 32(128): 855-862, 1997) and it is estimated that about 40% of children referred to mental health clinics show symptoms of ADHD. Progress in understanding the basis of ADHD has been hampered by a lack of consensus regarding its diagnosis, cause, prevalence, and treatment (Levy, BMJ 315(7113): 894-895, 1997).

Over the past two decades there have been 19 community-based studies in the USA offering estimates of prevalence ranging from 2% to 17%. The dramatic difference between these estimates is due to the choice of informant, methods of sampling and data collection and the diagnostic definition.

Treatment often requires trialing a number of stimulant medications until symptoms are alleviated.

There are currently no molecular markers of behavioral disorders such as, for example, ADHD.

In the work leading up to the present invention, the instant inventors investigated the molecular basis for behavioral disorders and in particular ADHD and, in accordance with the present invention, have identified molecular markers suitable for use in the diagnosis of a behavioral disorder or at least in assessing the risk of developing same. The “risk” constitutes the identification of a genetic predisposition of development of a behavioral disorder. The identification of the instant markers further facilitates the development of therapeutic protocols for treatment of such disorders.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

The gene “NHE7” which is referred to in the priority applications upon which the subject specification is based is now called “NHE”. The new nomenclature is adopted in the instant specification.

The present invention is predicated in part on the identification of a mutation on chromosome 3 which is associated with or co-incident to or which otherwise facilitates the development or progression, either alone or in association with environmental or other genetic factors, of a behavioral disorder such as but not limited to ADHD.

The present invention provides, therefore, a molecular marker of a behavioral disorder such as ADHD comprising, in a genetic form, a location on chromosome 3 or an equivalent location on another chromosome wherein a mutation at this location alone or in combination with environmental or other genetic factors is proposed to be associated with or otherwise facilitates the development or progression of the behavioral disorder. The location on chromosome 3 corresponds to two genes: the DOCK 3 (KIAA0299) gene and the NHE gene (also known as Homo sapiens solute carrier family 9 (sodium/hydrogen exchanger) isoform 8 (SLC9A8)) and formerly referred to as NHE7 in the priority applications of the subject specification. It is proposed that a mutation at DOCK 3 and/or NHE alone or in combination with environmental or other genetic factors is associated with or otherwise facilitates the development or progression of the behavioral disorder, such as but not limited to, ADHD.

The location on chromosome 3 is also in genetic proximity to three other genes: KIAA0800, HUMAGCGB and ARP (also known as ARMET). In addition to any disruption to DOCK 3 and/or NHE, the mutation at chromosome 3 may also cause some disruption to the function of these genes. Any disruption to DOCK 3 and/or NHE together with a disruption to the expression of KIAA0800, HUMAGCGB and/or ARP is further proposed to indicate a propensity for a subject to develop a behavioral disorder such as ADHD.

In yet a further embodiment, any of the aforementioned genes may represent a member of a genetic pathway or network or may encode products involved in a metabolic, physiological or neurological pathway. Accordingly, analysis of a possible disruption of these pathways at other genetic or gene-product locations is also proposed to provide an indication of a propensity for development of a behavioral disorder.

It is proposed that a mutation in any of the genes associated with the marker or in a gene or genetic sequence encoding a product or otherwise associated with a genetic, metabolic, neurological or physiological pathway involving DOCK 3 and/or NHE and optionally KIAA0800, HUMAGCGB and/or ARP is indicative of a genetic predisposition to the behavioral disorder, such a ADHD. A mutation in this context includes an insertion, substitution and/or deletion or an inversion and may result in no gene product, reduced amounts of a gene product, excess amounts of a gene product or a mutated gene product being produced.

Another aspect of the present invention provides a nucleic acid probe useful in a genetic assay for an aberration in DOCK 3 or NHE or other associated gene or genetic sequence.

Furthermore, the present invention contemplates an antibody for use in the diagnosis and/or treatment of a behavioral phenotype such as a disorder including but not limited to ADHD which specifically recognizes an epitope determined by a proteinaceous form of the subject molecular markers. The nucleic acid probe and/or the antibody are conveniently included in a kit for determining the presence of a genetic predisposition to a behavioral disorder.

A further aspect of the invention pertains to a composition for the treatment and/or prophylaxis of a behavioral phenotype such as but not limited to ADHD, the composition containing a nucleic acid molecule comprising a sequence of nucleotides which hybridizes under conditions of low stringency to a sequence of nucleotides comprising SEQ ID NO:1 and/or SEQ ID NO:2 and/or SEQ ID NO:12 and/or SEQ ID NO:14 and/or SEQ ID NO:20 and/or SEQ ID NO:22 or contained within SEQ ID NO:1 and/or SEQ ID NO:2 and/or SEQ ID NO:12 and/or SEQ ID NO:14 and/or SEQ ID NO:20 and/or SEQ ID NO:22 or a complementary or derivative from thereof and one or more acceptable carriers and/or excipients.

Yet another aspect of the invention pertains to a composition for the treatment and/or prophylaxis of a behavioral phenotype such as but not limited to ADHD, the composition containing a polypeptide or protein or peptide comprising a contiguous sequence of amino acids as set forth in SEQ ID NO:21 and/or SEQ ID NO:23 and/or SEQ ID NO:16 or SEQ ID NO:17 or SEQ ID NO:18 or an amino acid sequence having at least 70% similarity to any one of the above-mentioned sequences or a derivative thereof and one or more acceptable carriers and/or excipients.

The present invention further contemplates a method for diagnosing a behavioral phenotype or assessing the likelihood that a subject may develop a behavioral phenotype or otherwise has a genetic predisposition for development of a behavioral disorder such as but not limited to ADHD, the method comprising contacting a biological sample derived from said subject with a genetic form of a molecular marker comprising at least one of any one of SEQ ID NO:1 to SEQ ID NO:15 or SEQ ID NO:19 or SEQ ID NO:20 or SEQ ID NO:22 or a derivative thereof under conditions suitable for selective binding to occur.

In an alternative embodiment, the diagnosis is made by contacting a biological sample of a subject with a proteinaceous form of a molecular marker according to the present invention or antibodies thereto under conditions suitable for selective binding to occur.

In addition, another aspect of the present invention is directed to a method for screening a compound for an ability to ameliorate one or more symptoms of a behavioral phenotype in a human subject comprising administering one or more of said compounds to a genetically modified animal model of said behavioral phenotype and assessing the animal for changes consistent with the amelioration of one or more symptoms of said disorder.

The identification of DOCK 3 and NHE as candidate markers for a genetic predisposition for a behavioral disorder, such as ADHD, includes, as indicated above, the identification of a particular genetic or metabolic, physiological or neurological pathway wherein a disruption anywhere in this pathway may be indicative of a potential for development of a behavioral disorder. Once identified, appropriate behavioral modification protocols and/or therapeutic intervention can take place to reduce the likelihood of development of the behavioral disorder.

### BRIEF DESCRIPTION OF THE DRAWINGS

FIG. 1 is a schematic representation showing the pedigree of the family under investigation. The arrow indicates the index case. Carriers of the chromosome inversion are indicated by a shaded square in the top right hand corner of the symbol. Individuals marked with “N” had normal karyotypes and those with a query had not been karyotyped. Individuals with a confirmed clinical behavioral/developmental phenotype have a black box at the bottom left hand corner of their symbol. A grey box in the bottom left hand corner indicates a suspected behavioral/developmental phenotype not confirmed by clinical evaluation. Individuals indicated with an asterisk went on to further clinical assessment.

FIG. 2 is an ideogram of human chromosome 3. The diagram on the left represents a normal copy of human chromosome 3 and the diagram on the right depicts the inverted chromosome seen in the family of the study. The shaded area shows the extent of the inverted material.

FIG. 3 is a diagrammatic representation of the 3p breakpoint region. The two BAC clones that cross the breakpoint are shaded. The bars represent exons of the four genes mapping close to the breakpoint, i.e. the KIAA0299 gene, the ARP gene, the HUMAGCGB gene and the KIAA0800 gene.

FIG. 4 is a reproduction of a Southern blot of agarose gel separated restriction digest fragments of genomic DNA from two control individuals and the patient having the inverted chromosome. The normal sized band is seen in the patient lanes along with the altered band from the inverted chromosome. The difference in migration of the band in the middle lane of each triad is due to overloading of DNA for this control.

FIG. 5 is a diagrammatic representation of the KIAA0299 (DOCK 3) gene in the region of the 3p breakpoint. The top panel depicts the configuration of the breakpoint of the normal chromosome 3 and the bottom panel shows the scenario for the inverted chromosome. The grey bars represent exons of the genes beginning the numbering at 2, although this is not the true second exon of the gene. The arrow indicates the site of the breakpoint proper. The sequence to the right of the arrow is excised in the mutant chromosome, flipped around re-inserted next to 3q material as indicated by a horizontal line in the lower panel. The letter “E” stands for the restriction enzyme site EcoRI, “B” represents BamHI sites and “H” delineates HindIII sites. Sizes of the fragments seen on Southern analysis are indicated. The small horizontal bar represents the probe used on genomic Southern blots.

FIG. 6 is a schematic diagram of the inverse PCR method employed to identify unknown sequences of 3q origin from the inverted chromosome. The outward priming oligomers are indicated. Sequencing from primers directed towards the red line revealed the 3q sequence at the breakpoint.

FIG. 7 is a schematic diagram of the BAC/PAC contig across the 3q critical region with the breakpoint indicated by a star in the overlap between the clones RP11-56b20 (Bacpac Resources http://www.chori.org/bacpac/home.htm) and RP11-89n15. Sequence markers and known genes are indicated along the bottom of the horizontal axis.

FIG. 8 is a representation of BAC clone sequences across 3p breakpoint region (clones RP11-804h8, 646d13, 73i17. Sequences highlighted in grey are exons of the DOCK 3 gene. The 5′ end of the gene is towards the top of the page. The ATG start site is numbered as nucleotide one as the true transcription initiation site is unknown. The position of the breakpoint is indicated in bold, underlined type. Runs of “n” represent nucleotide sequences missing from the database. The size of the gaps in the sequence is unknown. The gene spans approximately 465 kb of genomic DNA. Exons from the ARP gene, the HUMAGCGB gene and some 3′ exons from the KIAA0800 gene are highlighted.

FIG. 9 is a representation of BAC clone sequences across 3q breakpoint region (clones RP11-20o3, 56b20, 89n15, PAC RP1-114b20, RP11-16p13, 165 m11 and 8j23). Sequences highlighted are exons of the NHE gene. The 5′ end of the gene is towards the bottom of the page. The ATG start site is in bold type and underlined. The position of the breakpoint is indicated in bold, underlined type. The gene spans approximately 470 kb of genomic DNA.

FIG. 10 is schematic representation of the p and q arm breakpoint sequences of the normal chromosome (SEQ ID NO:4 and SEQ ID NO:5 and SEQ ID NO:8 and SEQ ID NO:9 and SEQ ID NO:12 and SEQ ID NO:13) and an inverted chromosome (SEQ ID NO:6 and SEQ ID NO:7 and SEQ ID NO:10 and SEQ ID NO:11 and SEQ ID NO:14 and SEQ ID NO:15, respectively.

FIG. 11 is a representation of the amino acid sequences of KIAA0800 gene product (SEQ ID NO:16); HUMAGCGB gene product (SEQ ID NO:17) and ARP gene product (SEQ ID NO:1).

FIG. 12 is a representation of the nucleotide sequence of the probe used in to distinguish between normal and inverted chromosomes in Southern blot shown in FIG. 4.

FIG. 13 is a representation of the DOCK 3 gene nucleotide sequence (SEQ ID NO:20) from the 5′→3′ direction. There are 53 exons in total. The breakpoint lies between exons 19 and 20 and is marked with a vertical bar.

FIG. 14 is a representation of the NHE gene nucleotide sequence (SEQ ID NO:22) from the 5′→3′ direction. There are 16 exons in total. The breakpoint lines between exons 13 and 14 and is marked with a bar.

FIG. 15 is a photographic representation showing Northern blot analysis of a panel of tissues from adult human brain. A single message of 8.4 kb is seen for the DOCK 3 gene in all tissues examined except the spinal cord. The NHE probe hybridized to a single 3.5 kb species in all brain tissues examined. β-actin was used as a loading control. (1) Cerebellum; (2) cerebral cortex; (3) medulla; (4) spinal cord; (5) occipital lobe; (6) frontal lobe; (7) temporal lobe; and (8) putamen.

FIG. 16 is a photographic representation showing Northern blot analysis of a panel of adult human tissues. A single message of 8.4 kb is seen for the DOCK 3 gene and was detected only in the brain. Although more widely expressed, the NHE gene is also expressed in the brain. The NHE probe hybridized to a 3.5 kb species in all tissues examined except colon. In addition, the NHE probe hybridized to a larger, 7.5 kb band in skeletal muscle. β-actin was used as a loading control. (1) Brain [whole]; (2) heart; (3) skeletal muscle, (4) colon; (5) thymus; (6) spleen; (7) kidney; (8) liver; (9) small intestine; (10 placenta; (11) lung; and (12) peripheral blood leukocyte.

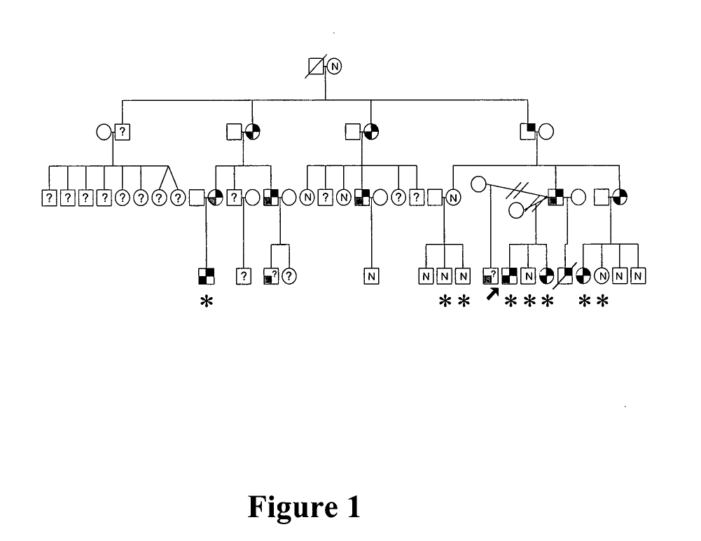

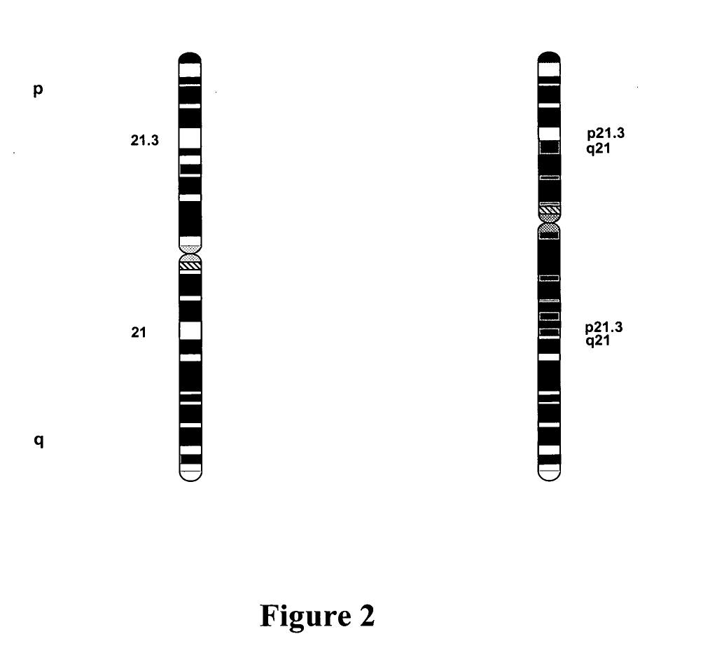

A summary of sequence identifiers used throughout the subject specification is provided in Table 1.

TABLE 1

| SE- | | |
|---|---|---|
| QUENCE | FIG- | |
| ID NO: | URE | DESCRIPTION |
| 1 | 8 | Nucleotide sequence of 3p breakpoint on normal |
| | | human chromosome 3 from clones BAC RP11-804h8 |
| 2 | 8 | Nucleotide sequence of 3p breakpoint on normal |
| | | human chromosome 3 from clones BAC RP11-804h8, |
| | | 646d'13, 73i17, 3f4 and 151d23 |
| 3 | 9 | Nucleotide sequence of 3q breakpoint region of |
| | | normal human chromosome 3 from BAC RP11-20o3, |
| | | 56b20, 89n15, BAC RP1-114b20, RP11-16p13, |
| | | 165mll and 8j23 |
| 4 | 10 | Nucleotide sequence of p-arm breakpoint on human |
| | | chromosome 3 |
| 5 | 10 | Complementary sequence to SEQ ID NO: 4 |
| 6 | 10 | Nucleotide sequence of q-arm breakpoint on human |
| | | chromosome 3 |
| 7 | 10 | Complementary sequence to SEQ ID NO: 6 |
| 8 | 10 | Nucleotide sequence of inverted p-arm on human |
| | | chromosome 3 |
| 9 | 10 | Complementary sequence to SEQ ID NO: 8 |
| 10 | 10 | Nucleotide sequence of inverted q-arm on human |
| | | chromosome 3 |
| 11 | 10 | Complementary sequence to SEQ ID NO: 10 |
| 12 | 10 | Nucleotide sequence of normal p-arm on human |
| | | chromosome 3 |
| 13 | 10 | Nucleotide sequence of inverted p-arm on human |
| | | chromosome 3 |
| 14 | 10 | Nucleotide sequence of normal q-arm on human |
| | | chromosome 3 |
| 15 | 10 | Nucleotide sequence of inverted q-arm on human |
| | | chromosome 3 |
| 16 | 11 | Amino acid sequence of KIAA0800 gene product |
| 17 | 11 | Amino acid sequence of HUMAGCGB gene product |
| 18 | 11 | Amino acid sequence of ARP gene product |
| 19 | 12 | Nucleotide sequence of probe used in Southern blot |
| | | hybridization of BAC 3f4 genetic region |
| 20 | 13 | Nucleotide sequence of human DOCK 3 gene |
| 21 | 13 | Amino acid sequence of DOCK 3 gene product |
| 22 | 14 | Nucleotide sequence of human NHE |
| 23 | 14 | Amino acid sequence of NHE gene product |

### DETAILED DESCRIPTION OF THE INVENTION

The present invention provides a molecular marker for a behavioral phenotype. The presence of one form of the marker is indicative of a low likelihood of the development of a behavioral disorder whereas another form is indicative of an increased risk of developing a behavioral disorder such as but not limited to Attention Deficit Hyperactivity Disorder (ADHD). A “behavioral phenotype” may be “normal” or classified as a “disorder”. The behavioral disorder may also include an environmental component in addition to a modified form of a molecular marker. The behavioral disorder in one form, is characterized by one or more behaviors such as but not limited to inattention, hyperactivity, impulsivity and/or intellectual disability. The latter includes subjects exhibiting a lower than average IQ. Consequently, in one form, the marker represents an altered nucleotide sequence leaving a subject predisposed to the development of a behavioral disorder depending on environmental, social and/or medical factors. Once a genetic predisposition is identified, it is proposed that behavioral modifying protocols and/or drugs may be used to facilitate socially acceptable behavior and/or to compensate for any intellectual disability. The former (socially acceptable behavior) constitutes behavioral patterns consistent with community standards in any given location regardless of the resistance of the particular individual concerned. The latter (intellectual disability) encompasses lower IQ values relative to well established standards.

Accordingly, as used herein, the term “a behavioral disorder”, such as but not limited to ADHD, broadly means a disorder which is associated or co-incident with or which is facilitated by the presence of one or more mutant forms of the present molecular markers notwithstanding that environmental conditions may influence the type and extent of the behavioral disorder. The term also includes a predisposition in a subject either to the development of a behavioral disorder such as but not limited to ADHD or to transmit the disorder, susceptibility to the disorder or carrier status for the disorder to an offspring. The markers, therefore, identify a genetic or metabolic, physiological and/or neurological predisposition towards development of a behavioral disorder. The term “behavioral disorder” as used herein includes intellectual disability, as well as inattention, hyperactivity and/or impulsivity.

The molecular marker may be in a genetic or proteinaceous form. Reference to genetic or proteinaceous forms includes the primary amino acid or nucleic acid sequence and a secondary structure, tertiary or later stage structure. Furthermore, the genetic marker may be a member of a genetic network or a proteinaceous form may be a member of a metabolic, physiological and/or neurological pathway. Consequently, another indication of a presence of or a predisposition for development of a behavioral disorder may be obtained by analyzing other members of the genetic or metabolic, physiological and/or neurological pathways. This can provide data which give an overall likelihood of a behavioral disorder or a likelihood of development of same.

The term “predisposition” is not to be construed as a subject not already having developed a behavioral disorder.

Accordingly, one aspect of the present invention provides a molecular marker of a behavioral disorder, said molecular marker, in a genetic form, comprises a genetic location on chromosome 3 or an equivalent location on another chromosome wherein a mutation at said location alone or in combination with environmental or other genetic factors is associated with or otherwise facilitates the development or progression of said behavioral disorder.

The molecular marker of the present invention may represent a “normal” polynucleotide sequence wherein an individual with such a sequence has a low risk of developing a behavioral disorder or a “mutant” polynucleotide sequence such as in an individual who has developed a behavioral disorder or has a high likelihood of developing a behavioral disorder. The normal and mutant polynucleotide sequences may include coding and intronic regions and intergenic regions (i.e. 3′ or 5′ regions) relative to a gene as well as nucleotide sequences which facilitate function or expression of a particular gene.

In a preferred embodiment, the genetic location is on chromosome 3 and is particularly associated with or comprises, or is otherwise genetically proximal to, a DOCK 3 gene or an NHE gene or both. A genetic location associated with DOCK 3 or NHE or both includes a genetic location which controls function or expression of a gene. A genetic location associated with DOCK 3 or NHE is considered to include genes in genetic proximity to DOCK 3 and/or NHE. Such genes include KIAA0800, ARP and HUMAGCGB. It is proposed that a mutation at or near DOCK 3 or NHE or involving both genes may also have an effect on one or more of KIAA0800, ARP and/or HUMAGCGB. Analysis of a disruption in DOCK 3 or NHE or both may, therefore, optionally further involve analysis of a disruption in KIAA0800, ARP and/or HUMAGCGB or other members of a genetic network or metabolic, physiological and/or neurological pathway in which any of the above genes are involved. Such analysis can result in an overall determination of the likelihood of the presence or predisposition of development of a behavioral disorder.

Reference herein to “genetically proximal” includes reference to a close physical distance such that the genetic sequence can function as a marker.

Accordingly, another aspect of the present invention contemplates a molecular marker of a behavioral disorder such as ADHD wherein said molecular marker, in genetic form, comprises a genetic location associated with a DOCK 3 gene or an NHE gene wherein a mutation at DOCK 3 or NHE alone or in combination with environmental or other genetic factors is associated with or otherwise facilitates the development or progression of said behavioral disorder.

In another embodiment, the present invention contemplates a molecular marker of a behavioral disorder, such as ADHD wherein said molecular marker, in genetic form, comprises a genetic location associated with a DOCK 3 gene or an NHE gene wherein a mutation at DOCK 3 or NHE alone or in combination with environmental or other genetic factors, such as a disruption in one or more of KIAA0800, ARP and/or HUMAGCGB is associated with or otherwise facilitates the development or progression of said behavioral disorder.

Where one form of the molecular marker constitutes a polynucleotide sequence, a mutant polynucleotide may comprise a single or multiple nucleotide substitution, addition and/or deletion or inversion and includes a polymorphism or natural variant. Another form would constitute a proteinaceous molecule or other gene product encoded by the genetic forms. The term “gene product” is used broadly to include RNA, mRNA, introns and exons. The present mutation may be dominant or recessive, somatic or germinal and may lead to loss of function or gain of function of a protein or other gene product encoded thereby. Furthermore, the mutation may be conditional and only cause the mutant phenotype under certain environmental conditions.

A particularly important mutation is a chromosome 3 inversion which, in accordance with the present invention, is associated or co-incident with or otherwise facilitates the development or progression of a behavioral disorder such as but not limited to ADHD. The inversion has been mapped and the p-arm and q-arm breakpoints have been sequenced. Polynucleotide molecular markers which map to the breakpoints on the inverted chromosome 3 and on the normal chromosome 3 have been identified and are described herein. These markers can now be used to assess or develop protocols for the assessment of a risk of a subject having or developing or transmitting a behavioral disorder.

The ability to identify at risk individuals permits the implementation of medicinal and/or behavioral protocols to reduce the likelihood of development of or to ameliorate one or more of the symptoms of a behavioral disorder.

In a particularly preferred embodiment, the mutation on chromosome 3 which is associated or co-incident with or which otherwise facilitates the development or progression of a behavioral disorder and particularly ADHD modulates the expression of one or more genes such as but not limited to DOCK 3 or NHE and optionally one or more of KIAA0800, ARP and/or HUMAGCGB.

The term “gene” is used in its broadest sense and includes cDNA corresponding to the exons of a gene. Accordingly, reference herein to a “gene” is to be taken to include:—

(i) a classical genomic gene consisting of transcriptional and/or translational regulatory sequences and/or a coding region and/or non-translated sequences (i.e. introns, 5′- and 3′-untranslated sequences); or

(ii) mRNA or cDNA corresponding to the coding regions (i.e. exons) and 5′- and 3′-untranslated sequences of the gene.

The term “gene” is also used to describe synthetic or fusion molecules encoding all or part of an expression product. In particular embodiments, the term “nucleic acid molecule” and “gene” may be used interchangeably.

The term “gene” is also used to describe synthetic, hybrid or fusion molecules encoding all or part of an expression product. In particular embodiments, the term “polynucleotide”, “nucleic acid molecule” and “gene” may be used interchangeably.

The term “modulates the expression of one or more genes” encompasses quantitative and qualitative changes in the expression product as well as up-regulation or down-regulation of transcription and/or translation. As known in the art, gene regulation may be upset by proximity to different genes and accordingly one or more genes in the vicinity of a mutation may be affected.

The molecular marker of the present invention is derived from chromosome 3 in humans or its functional equivalent on another chromosome or in another animal is within the scope of the present invention. A syntenic region is contemplated, therefore, from other organisms such as from primates, laboratory animals, livestock animals, companion animals and captured wild animals. In particular, a syntenic region from murine species may be conveniently predicted from available databases.

As used herein, the term “derived from” shall be taken to indicate that a particular integer or group of integers has originated from the source specified, but has not necessarily been obtained directly from the specified source.

A “mutation” or “modified” form of a genetic sequence includes a single or multiple nucleotide substitution, addition and/or deletion or inversion. An inversion is a particularly useful form of modification in terms of its use as a diagnostic indicator.

The inversion breakpoints on chromosome 3 of an individual (hereinafter referred to the as the “inverted chromosome”) have in accordance with the present invention been mapped to band p21.3 and band q21. Fluorescent In Situ Hybridization (FISH) analysis is conveniently used to identify BAC and/or YAC clones spanning these p-arm and q-arm breakpoints on normal chromosome 3. Clearly, however, other forms of analysis may be conducted.

In one embodiment, the instant inventors constructed a phage library from YAC clones spanning the p-arm breakpoint. PCR-based methods were used to screen the library for EST markers of genes in the region of the p-arm breakpoint. Although genes ARP, HUMAGCGB and KIAA0800 were identified in this region, the genes which were directly disrupted by the inversion were DOCK 3 and NHE. Three overlapping BAC clones identified as: BAC RP11-151d23, BAC RP11-3f4 and BAC RP11-89f17 (NCBI, Bethesda, Md.; http://www.ncbi.nlm.nih.gov/) were obtained encompassing this region.

Further FISH and Southern blot analyses demonstrated that the breakpoint on the p-arm was located within the DOCK3 gene. The region flanking the p-arm breakpoint has been sequenced.

Example 6 describe the expression of DOCK 3 and NHE in the brain. Without limitation to any particular mode of operation, it is contemplated that mutation in the DOCK 3 and/or NHE gene leads to modified expression of DOCK 3 and/or NHE or a modified expression product at least in the brain, which is associated with or otherwise facilitates the development or progression of a behavioral disorder or the risk of developing same.

Reference to a modified expression product includes loss of any product. An expression product may be inter alia a protein, RNA, mRNA, intron or exon.

Not wishing to limit the present invention to any one method or manner of performance, in one embodiment, an inverse PCR strategy was performed to identify the unknown sequences of the 3q-arm breakpoint based on the new sequence information from the 3p breakpoint. Pursuant to this strategy, it was determined that the 3q breakpoint was flanked by BAC clones RP11-89n15 and RP11-56b20. Within this region, an EST with homology to a Sodium/Hydrogen Ion-Exchanger family was identified. In accordance with the present invention, the gene has been termed NHE. The region flanking the q-arm breakpoint has been sequenced.

Contigs flanking the p-arm and q-arm breakpoints on chromosome 3 and genes in the region have been developed. The p-arm contig is exemplified in SEQ ID NO:1 and SEQ ID NO:2 and the q-arm contig is exemplified in SEQ ID NO:3. The sequences SEQ ID NO:1 and SEQ ID NO:2 are overlapping sequences.

Accordingly, yet another aspect of the present invention provides a molecular marker of a behavioral phenotype, said molecular marker comprising, in a genetic form, a nucleotide sequence or modified form thereof or an expression product encoded thereby, said nucleotide sequence derived from the p-arm of chromosome 3 and comprising or contained within a sequence of nucleotides which hybridizes under conditions of low stringency to a sequence of nucleotides exemplified in SEQ ID NO:1 or SEQ ID NO:2 or a complementary form thereof or having at least 60% similarity to either SEQ ID NO:1 or SEQ ID NO:2 wherein the presence of a modified form of said molecular marker is indicative of a behavioral disorder or the likelihood that a subject may develop a behavioral disorder.

Still another aspect of the present invention provides a molecular marker of a behavioral phenotype, said molecular marker, in a genetic form, comprising a genetic sequence or modified form thereof or an expression product encoded thereby, said nucleotide sequence derived from the q-arm of chromosome 3 and comprising or contained within a sequence of nucleotides which hybridizes under conditions of low stringency to a sequence of nucleotides exemplified in SEQ ID NO:3 or a complementary form thereof or having at least 60% similarity thereto wherein the presence of a modified form of said molecular marker is indicative of a behavioral disorder or the likelihood that a subject may develop a behavioral disorder.

The present invention further contemplates a molecular marker comprising nucleotide sequence derived from the inverted chromosome. The nucleotide sequence of the q-arm of an inverted chromosome are shown in SEQ ID NO:10 and SEQ ID NO:11 and SEQ ID NO:15. The nucleotide sequence on the p-arm are shown in SEQ ID NO:8 and SEQ ID NO:9 and SEQ ID NO:14.

Accordingly, yet another aspect of the present invention provides a molecular marker of a behavioral phenotype, said molecular marker, in a genetic form, comprising nucleotide sequence derived from the q-arm of chromosome 3 and comprising or contained within a sequence of nucleotides set forth in SEQ ID NO:10 and SEQ ID NO:11 and/or SEQ ID NO:15 or which hybridizes under conditions of low stringency to SEQ ID NO:10 or SEQ ID NO:11 or SEQ ID NO:15 or a complementary form thereof or having at least 60% similarity thereto wherein the presence of this sequence is indicative of a subject having a behavioral disorder or having a predisposition for development of a behavioral disorder.

In another embodiment, the present invention provides a molecular marker of a behavioral phenotype, said molecular marker, in a genetic form, comprising nucleotide sequence derived from the p-arm of chromosome 3 and comprising or contained within a sequence of nucleotides set forth in SEQ ID NO:8 and SEQ ID NO:9 and/or SEQ ID NO:14 or which hybridizes under conditions of low stringency to SEQ ID NO:8 or SEQ ID NO:9 or SEQ ID NO:14 or a complementary form thereof or having at least 60% similarity thereto wherein the presence of this sequence is indicative of a subject having a behavioral disorder or having a predisposition for development of a behavioral disorder.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, j. Mol. Biol. 5: 109, 1962). However, the Tₘ of a duplex DNA decreases by 1° C. with every increase of 1% in the number of mismatch base pairs (Bonner and Laskey, Eur. J. Biochem. 46(1). 83-88, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucleic Acids Res. 25(17): 3389-3402, 1997). A detailed discussion of sequence analysis can be found in Unit 19.3 of Ausubel et al. (“Current Protocols in Molecular Biology”, John Wiley & Sons, Inc., Chapter 15, 1994-1998).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The presence of genetic forms of molecular markers may be identified using any suitable protocol such as heteroduplex analysis, polymerase chain reaction (PCR), ligase chain reaction (LCR), sequence specific hybridization probes (SSO), single-stranded conformational polymorphism (SSCP), sequencing, mass spectrometry, enzyme cleavage and combinations of these.

Methods for assessing whether or not an association or significant correlation exists between a mutation in a gene and a particular phenotype are well known to those skilled in the art. Thus, another aspect of the present invention contemplates a method for screening for a mutation which is associated or co-incident with or which otherwise facilitates the development or progression of a behavioral disorder such as ADHD, said method comprising screening for a mutation in genetic sequences encoding a DOCK 3 gene or NHE gene wherein said mutation is shared by subjects having said behavioral disorder or which have a propensity to develop said disorder.

Reference to “contained within a sequence of nucleotides” encompasses fragments of the exemplified polynucleotide sequences. When genomic sequences are exemplified, reference to fragments includes reference to the full length mRNA encoded by the genomic sequences. The term fragments is used in a broad sense and includes reference to relatively short contiguous portions of the sequence for use as PCR primers or probes or the like as well as reference to full length genes. Fragments may range from about 10 bp to about 400 kb, from about 100 bp to about 1 kb, from about 500 bp to 5 kb, from about 900 bp to 100 kb, from about 80 kb to about 200 kb.

Derivatives of the genetic forms of the isolated molecular markers are clearly contemplated by the present invention. Generally, functional derivatives are preferred and derivatives with enhanced function. For example, derivative oligonucleotides having one or more nucleotide mutations or modifications which improve stability, hybridization and/or detection are contemplated. Derivatives also includes homologues, analogues, mimetics and variants.

Complementary sequences to the coding strand are provided for use as probes or primers or for modulating the level of expression of an endogenous gene. For example, a gain of function mutation may be suppressed by antisense or ribozyme molecules based on technology known in the art.

The present invention therefore provides nucleic acid molecules comprising breakpoint sequences for use in diagnosis for a behavioral disorder or at least of assessing the risk of developing same. For example, the detection of a normal breakpoint sequence is indicative of a low likelihood of the development of a behavioral disorder such as but not limited to ADHD while, the presence of the inverted chromosome breakpoint sequences is indicative of an increased risk of developing such a phenotype. A diagnostic test based on these sequences could take any one or more of the diverse forms known in the art including a PCR or affinity-based test or a cytogenetic test. Diagnosis may take place at any age including in utero. Genetic testing to determine the risk of a subject or family member developing certain disorders is becoming more commonplace and is explicitly contemplated.

Genetic sequences encoding genes which are affected by the mutant forms of the molecular markers described herein are preferred embodiments of the present invention. And, in a related embodiment, the proteins encoded by the normal or mutant polynucleotides are provided.

Gene replacement and genetic constructs designed to add, delete, supplement and/or manipulate genes affected by the present mutant polynucleotides is also contemplated. In one embodiment the therapeutic gene is delivered to the brain.

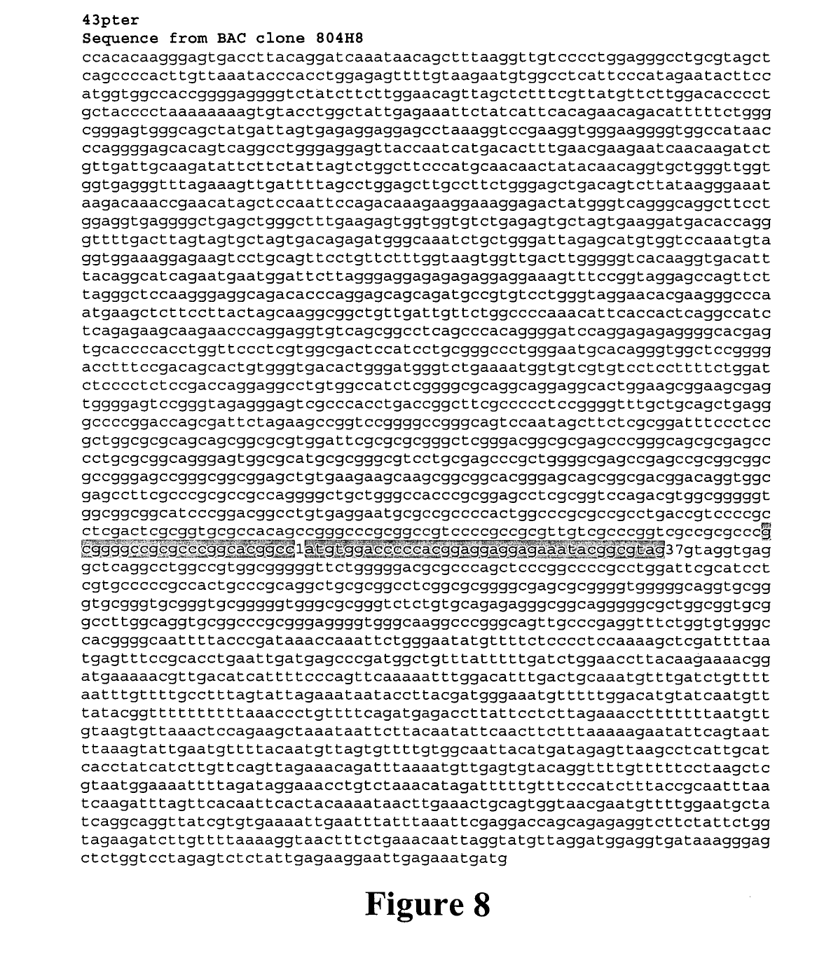

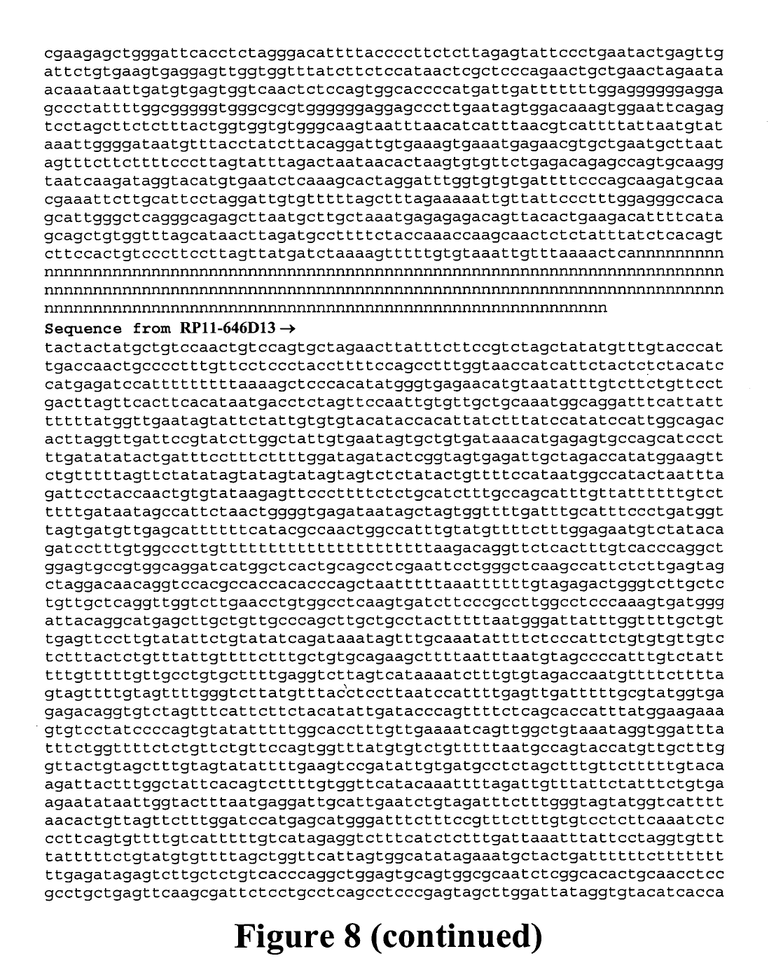

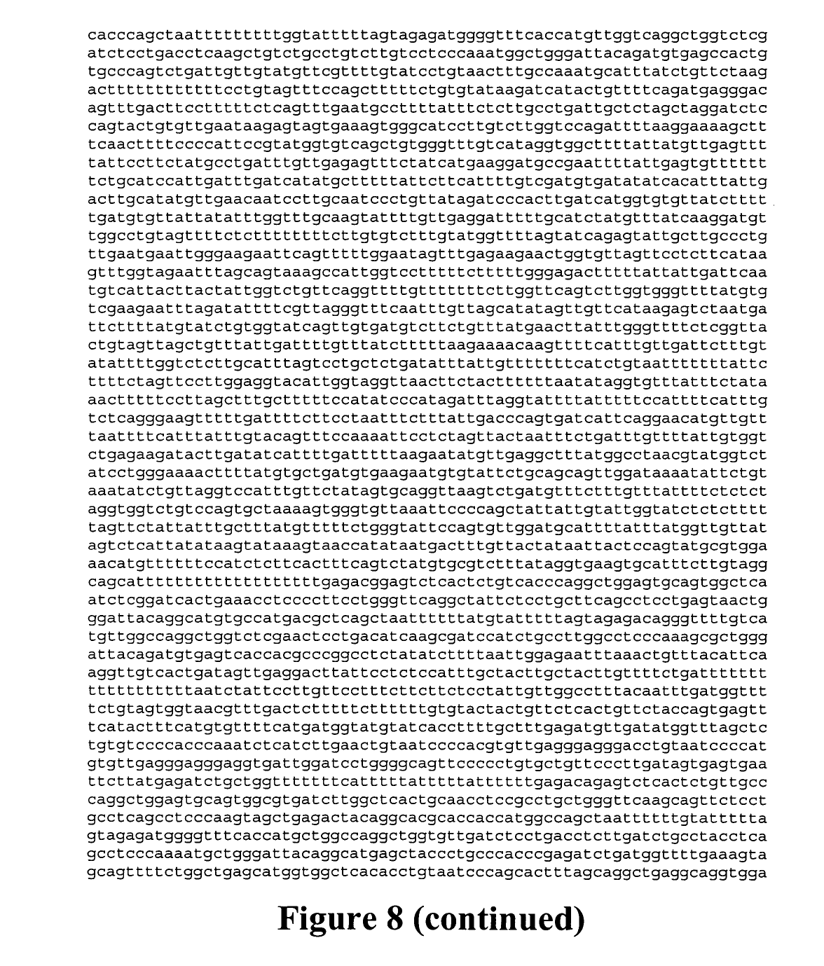

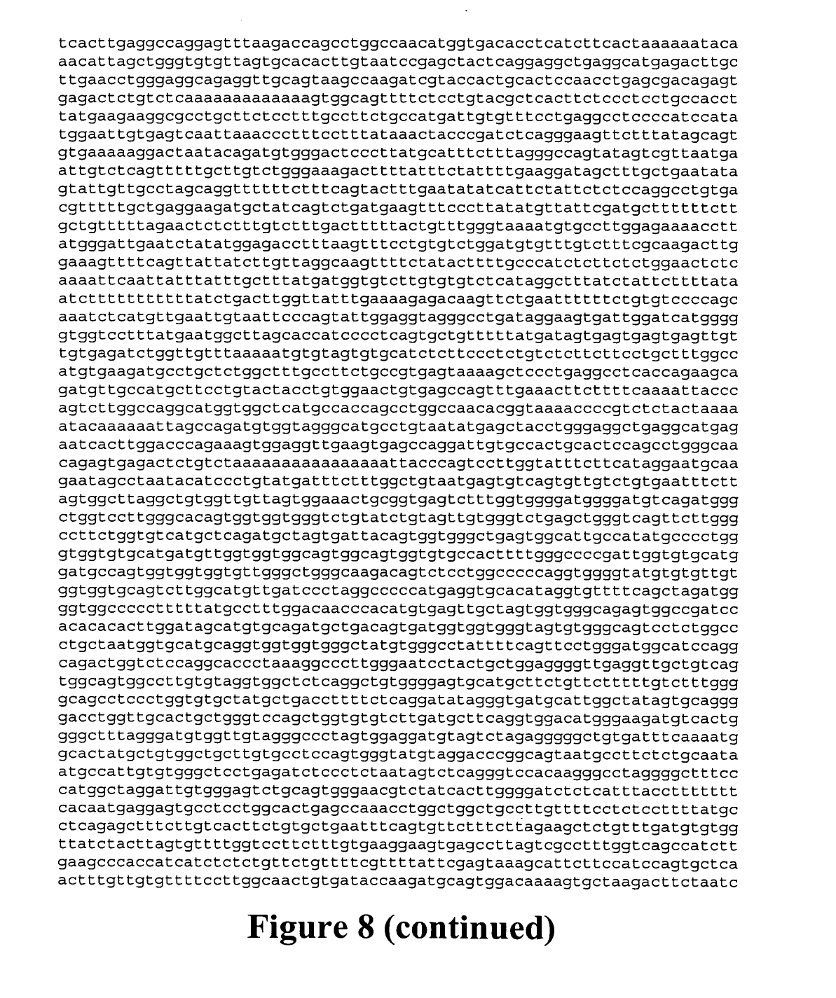

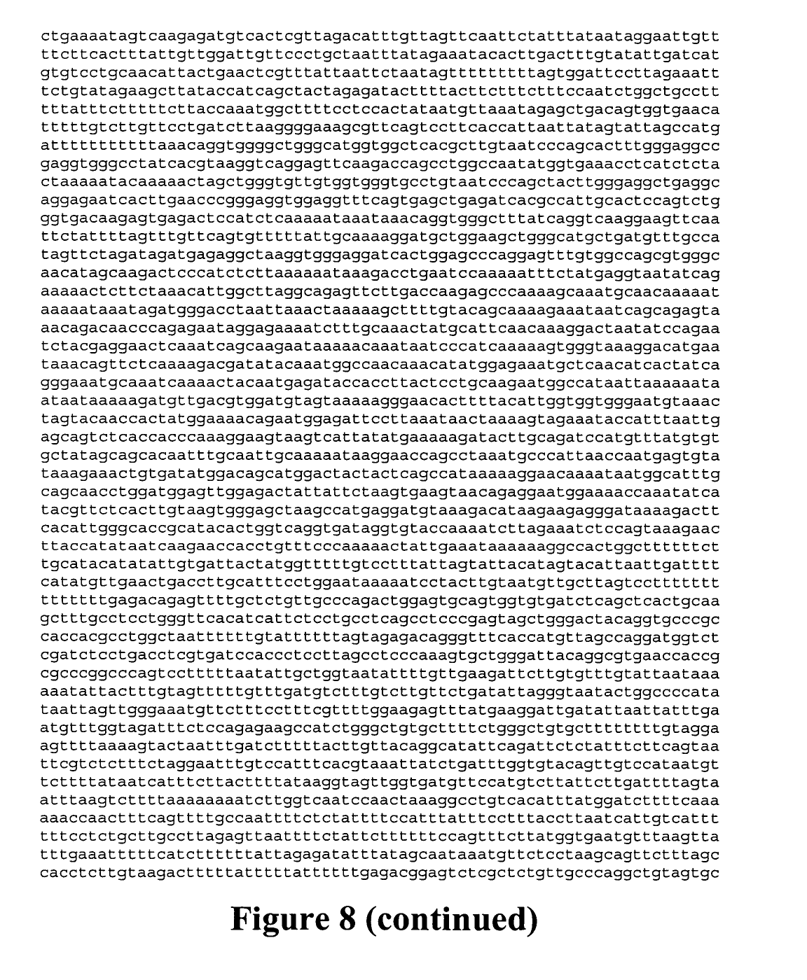

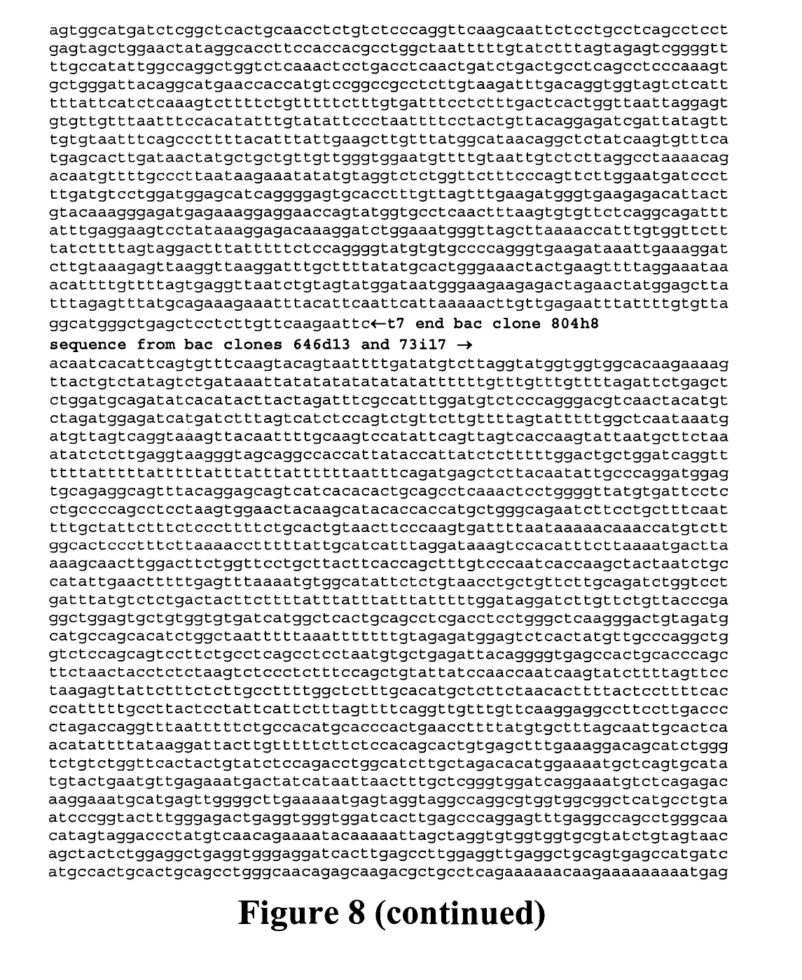

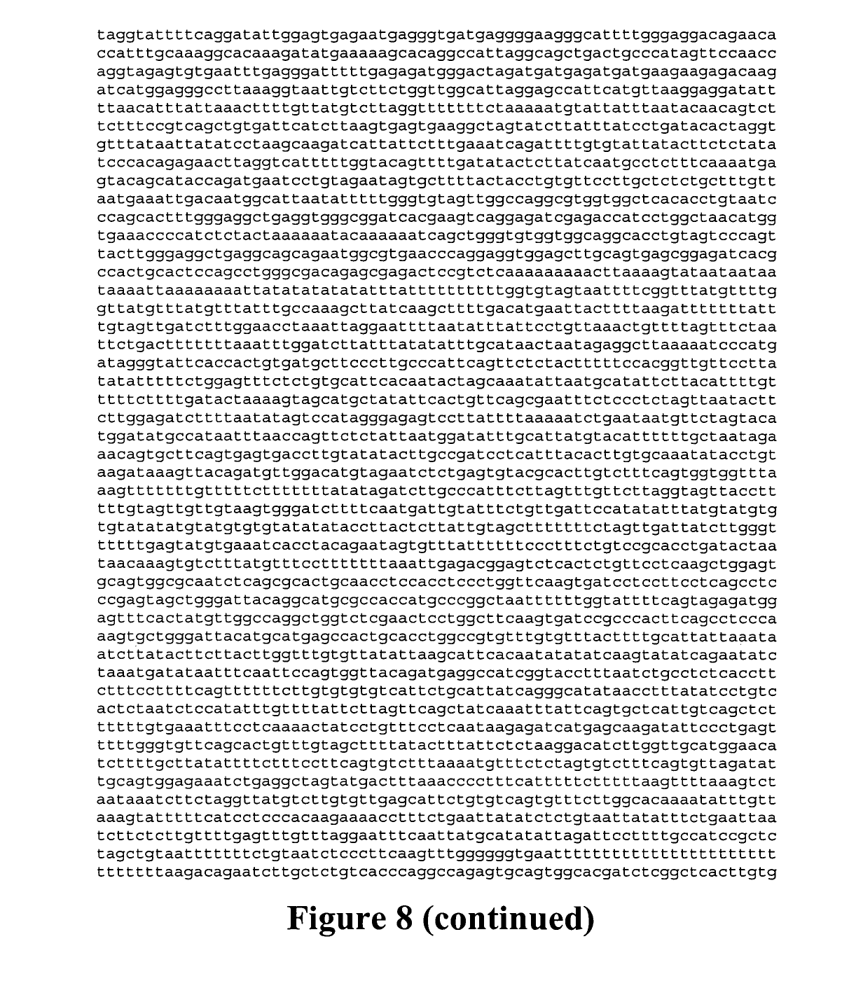

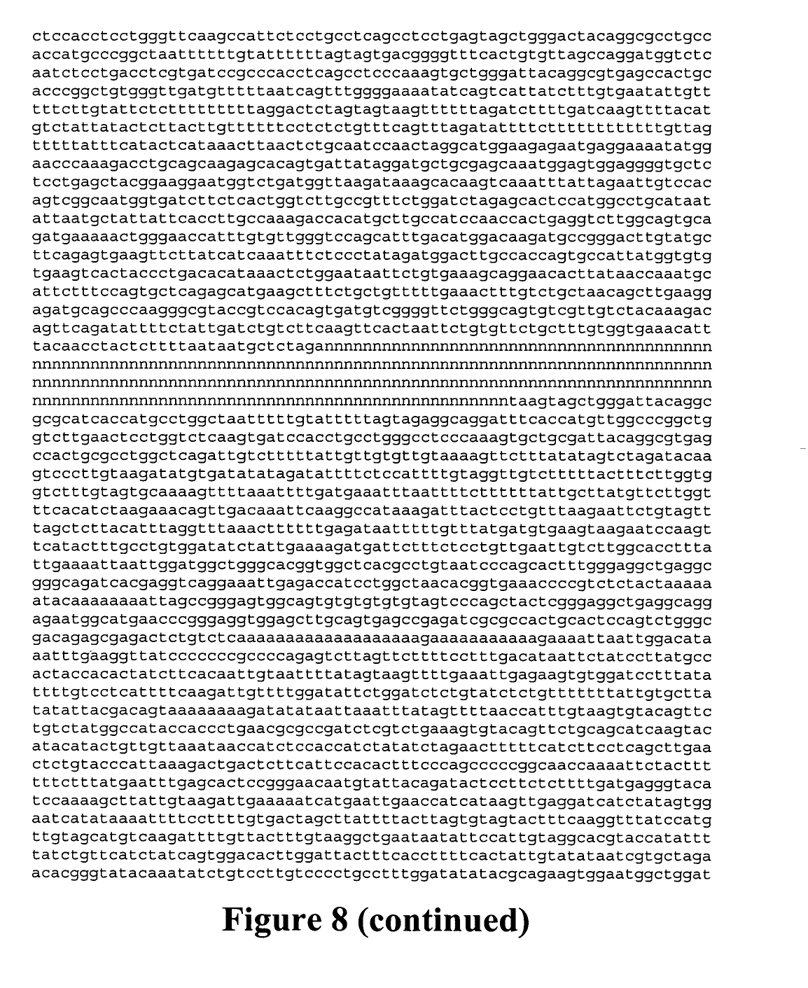

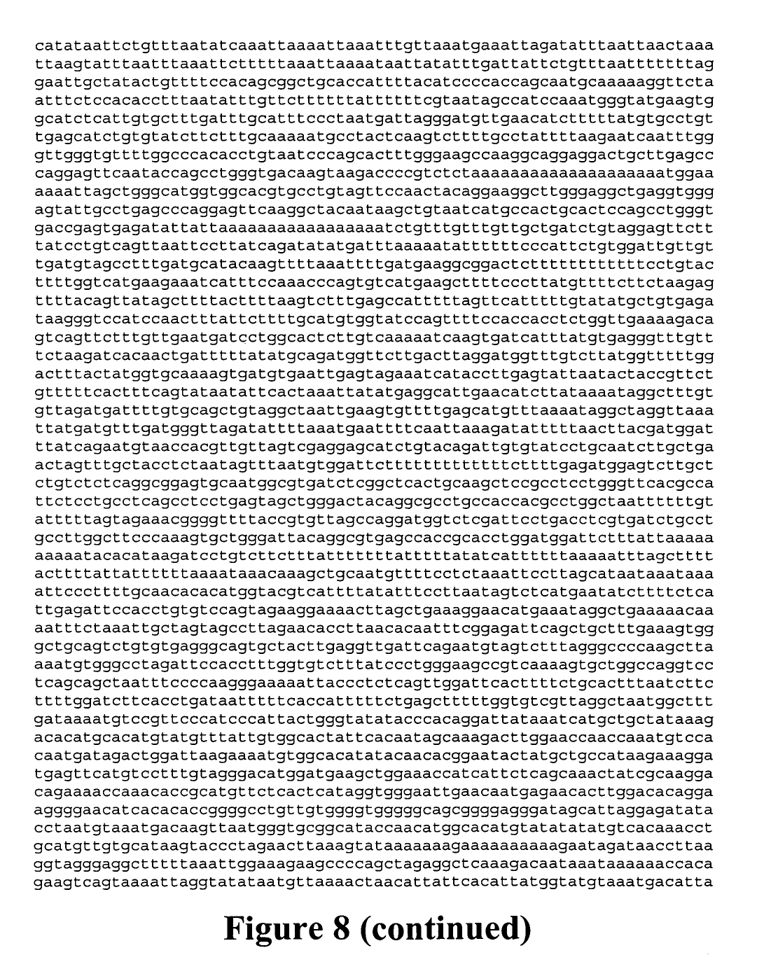

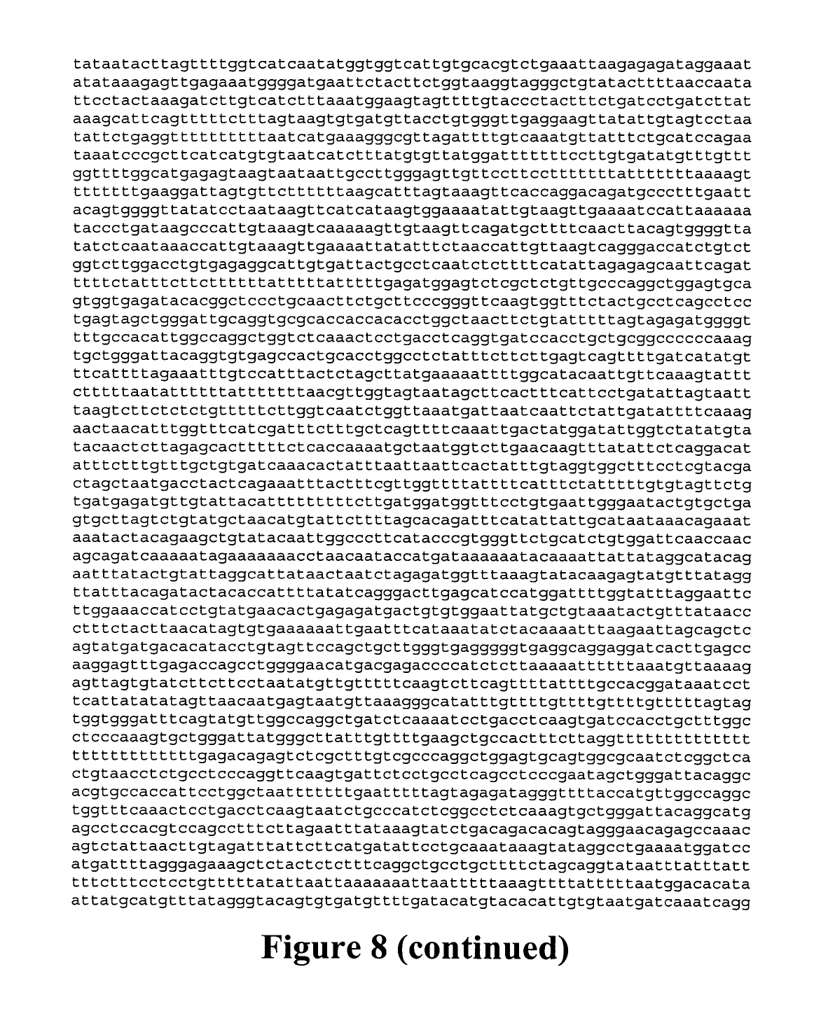

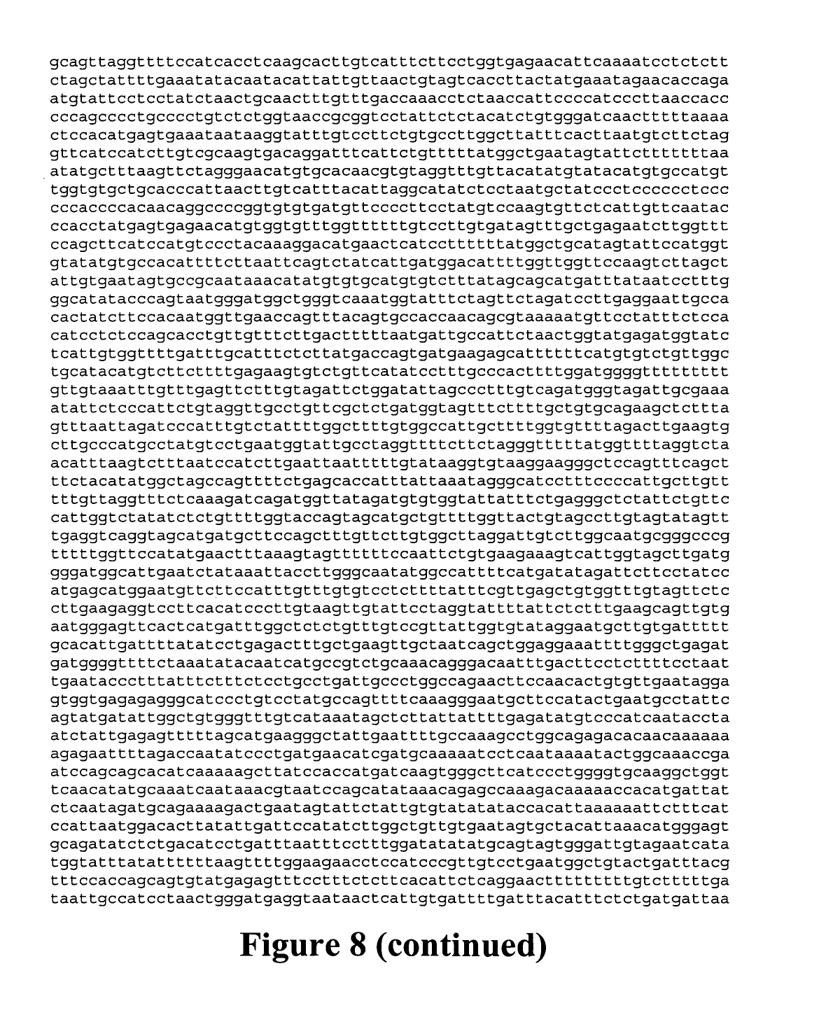

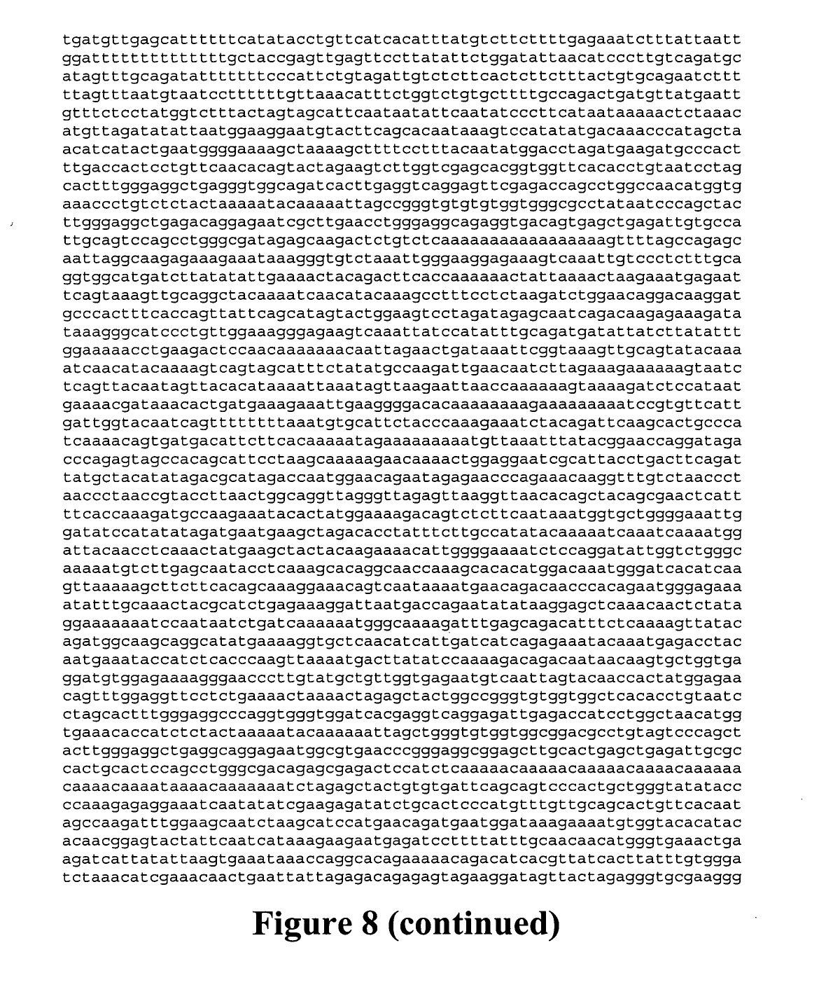

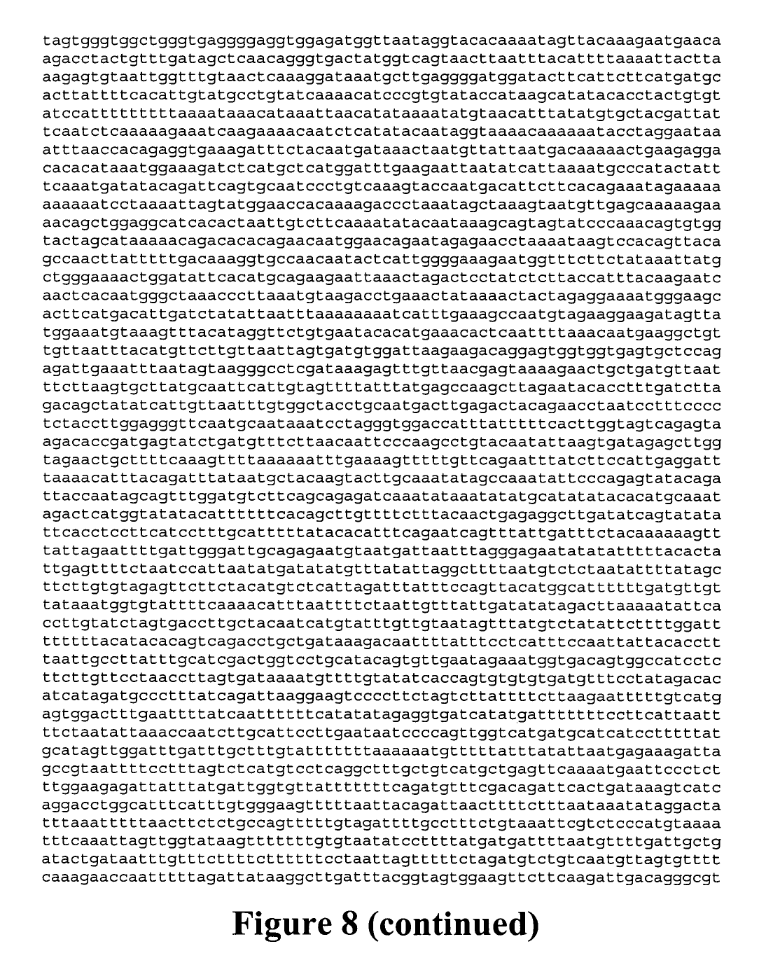

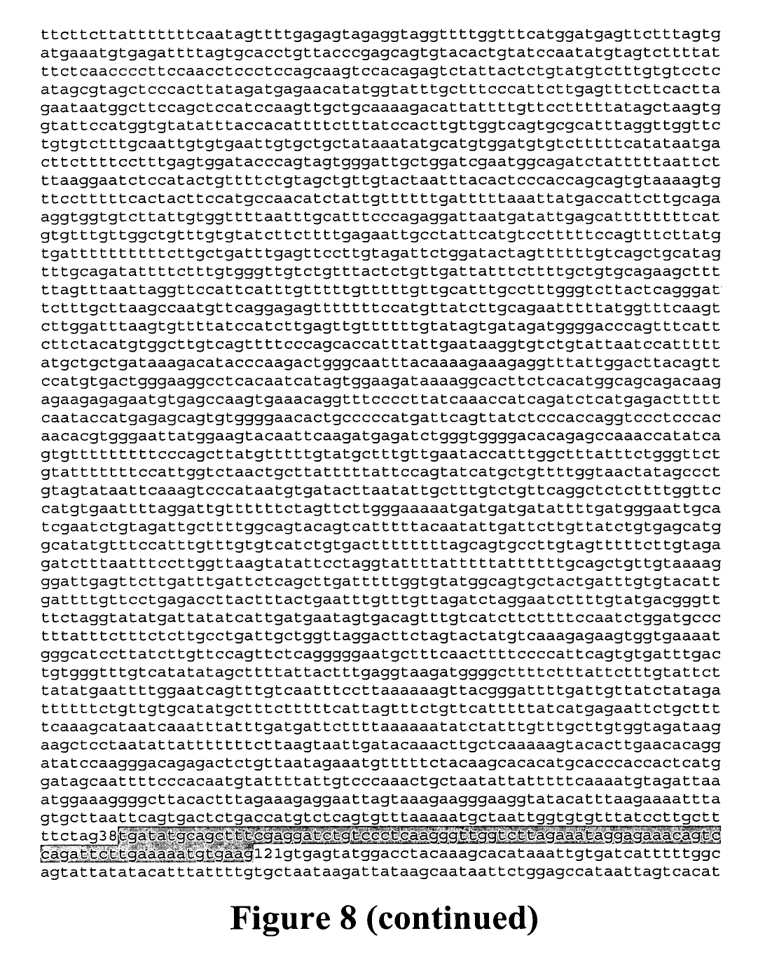

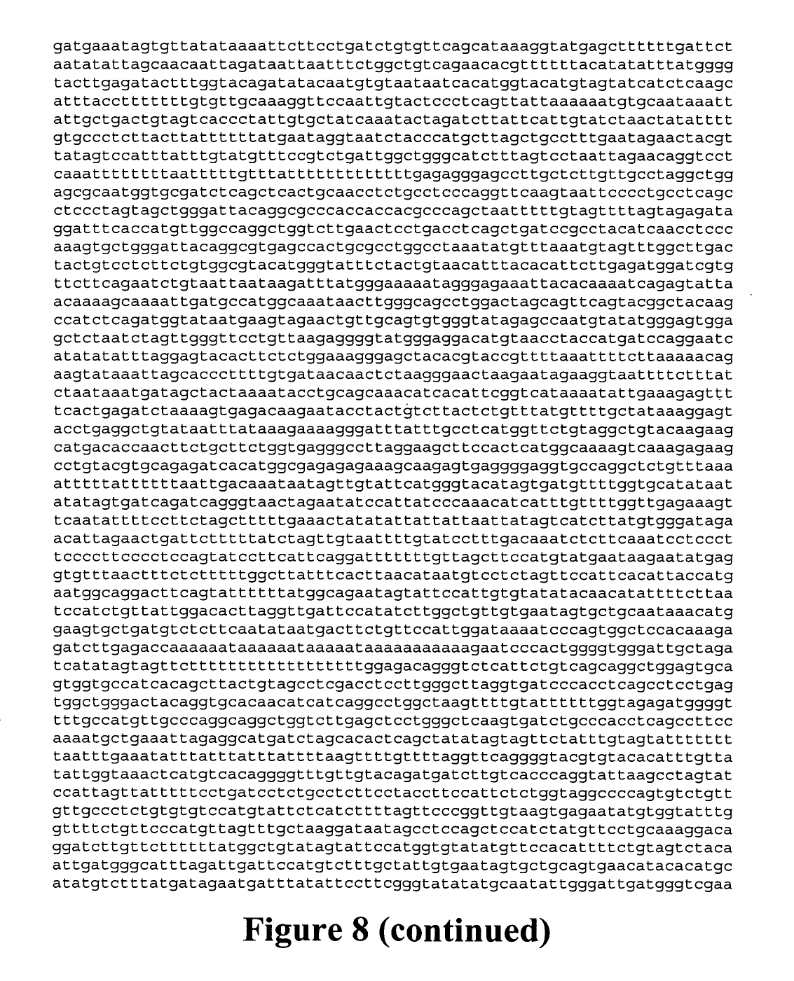

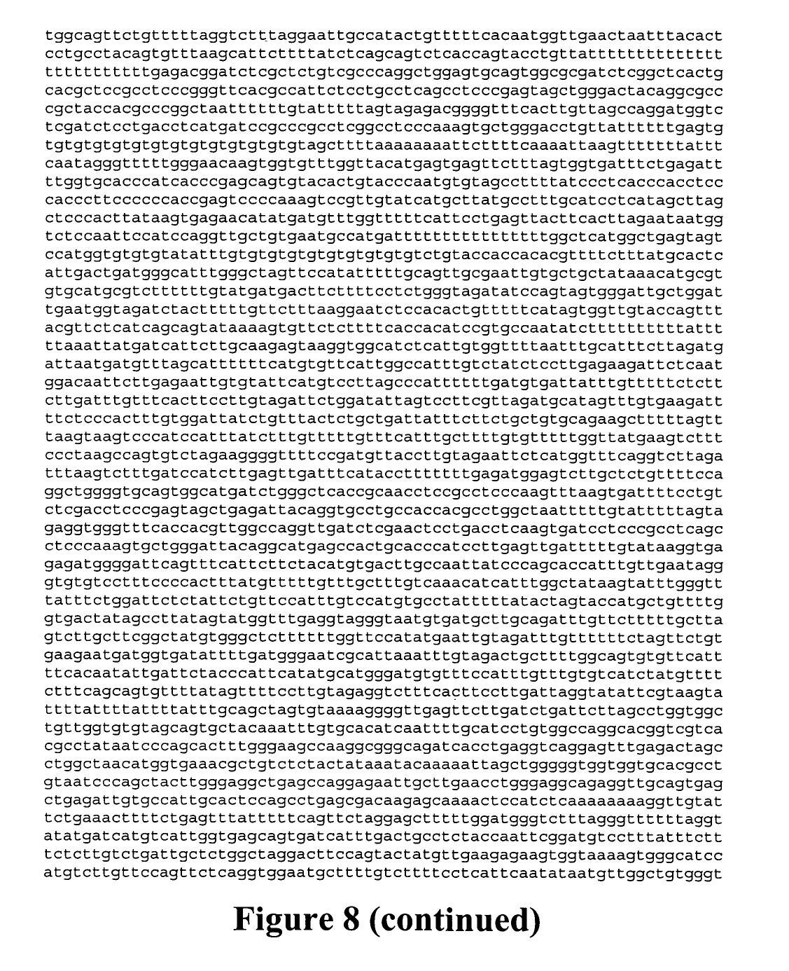

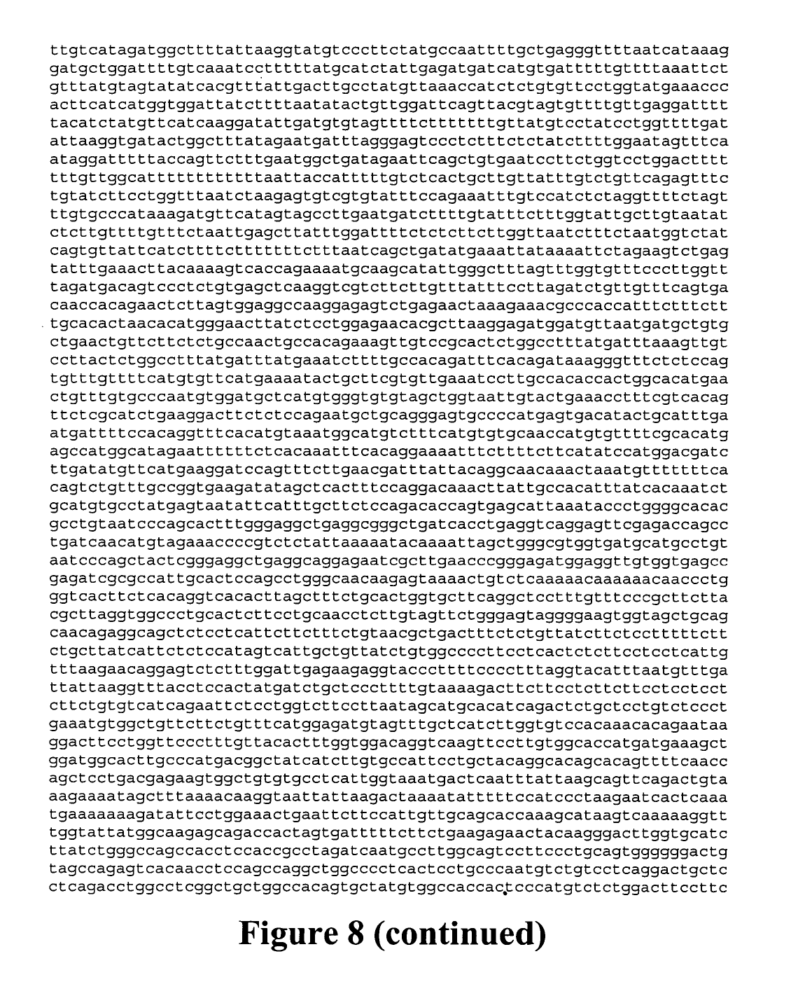

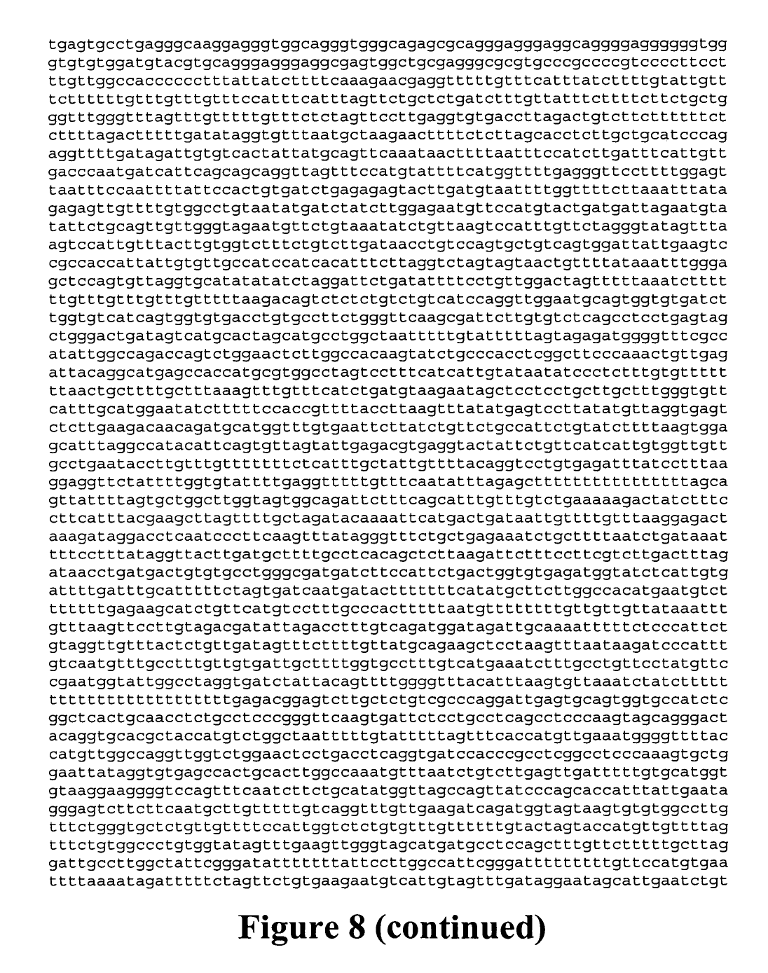

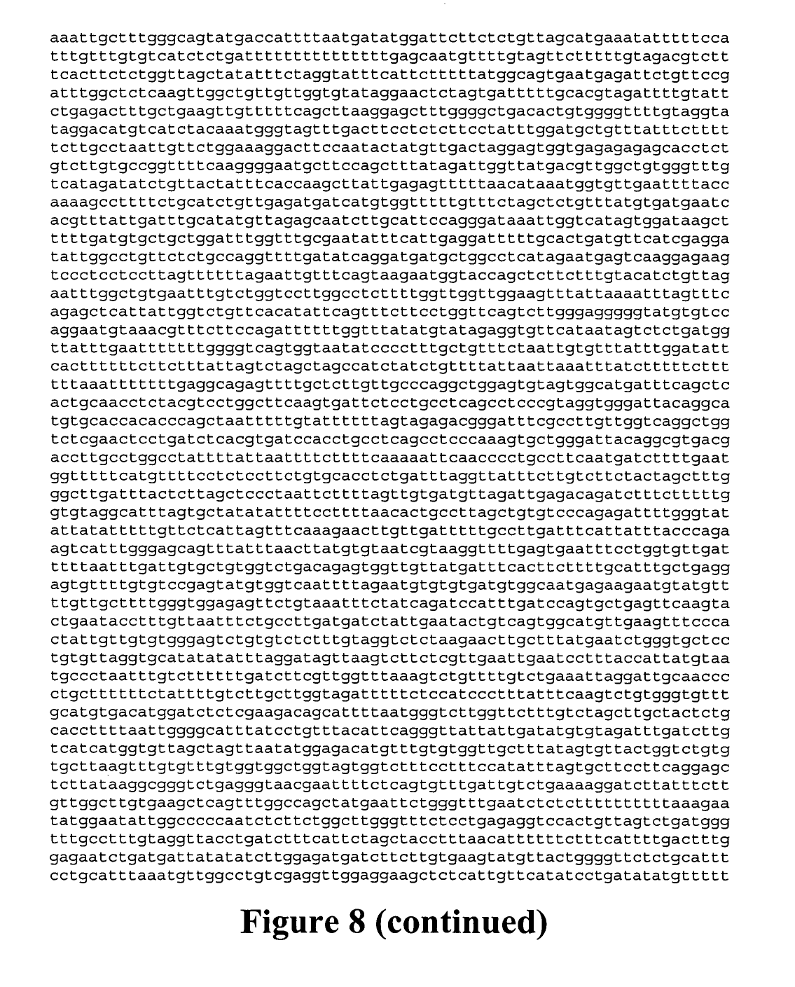

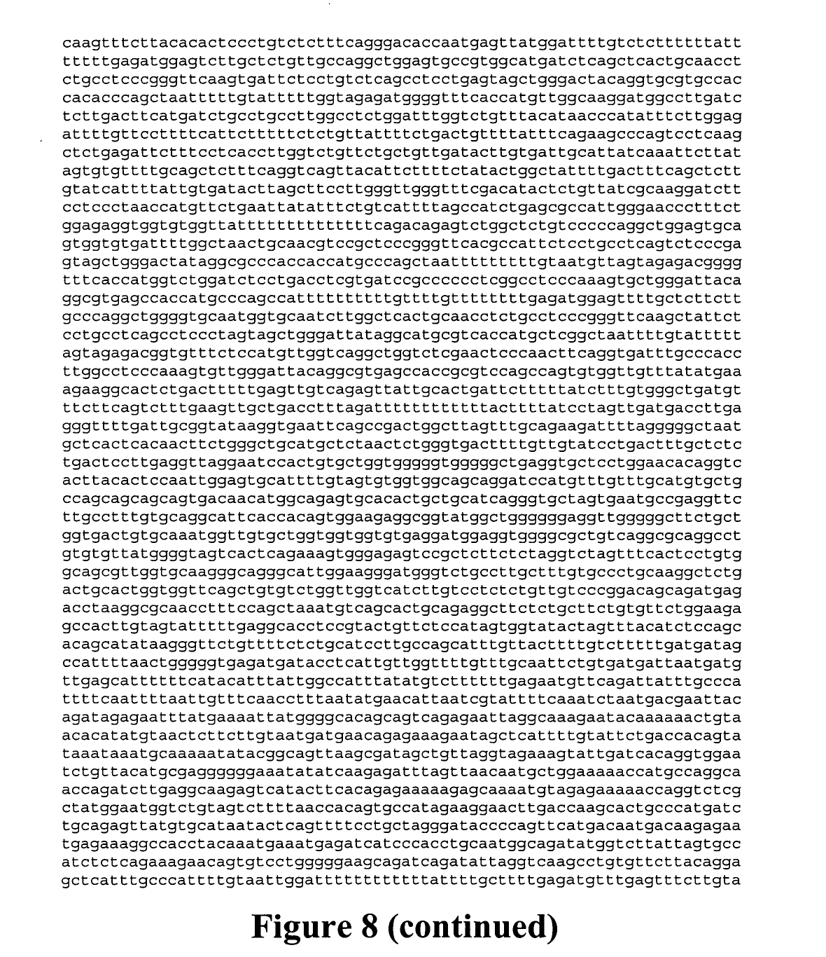

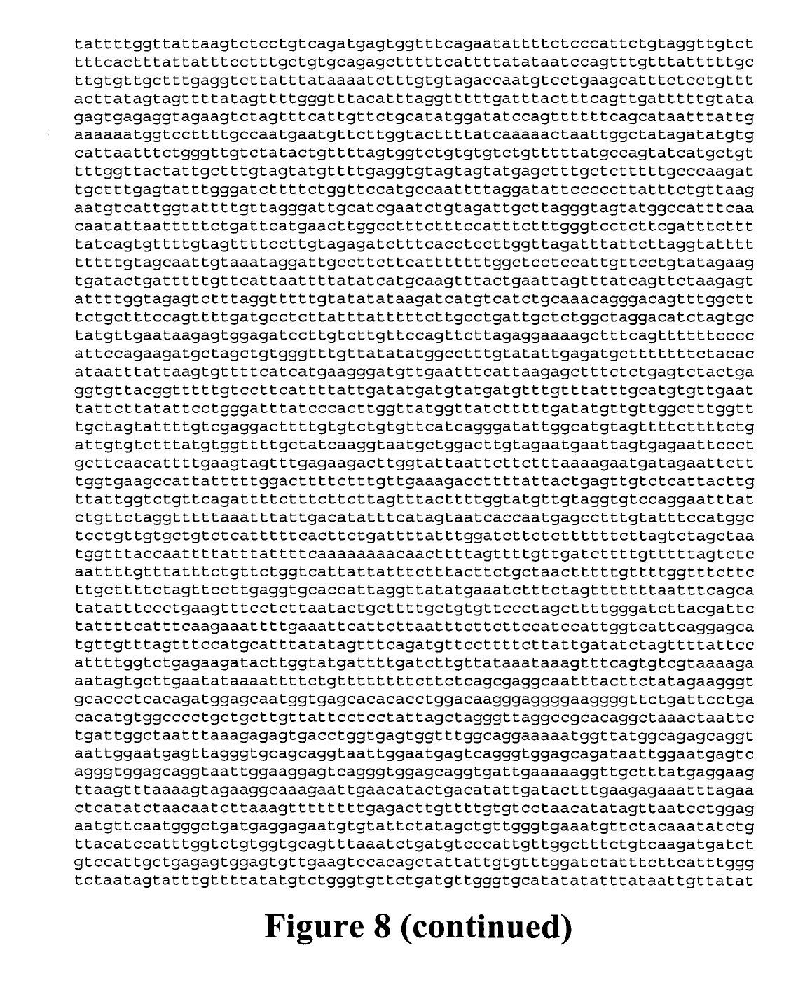

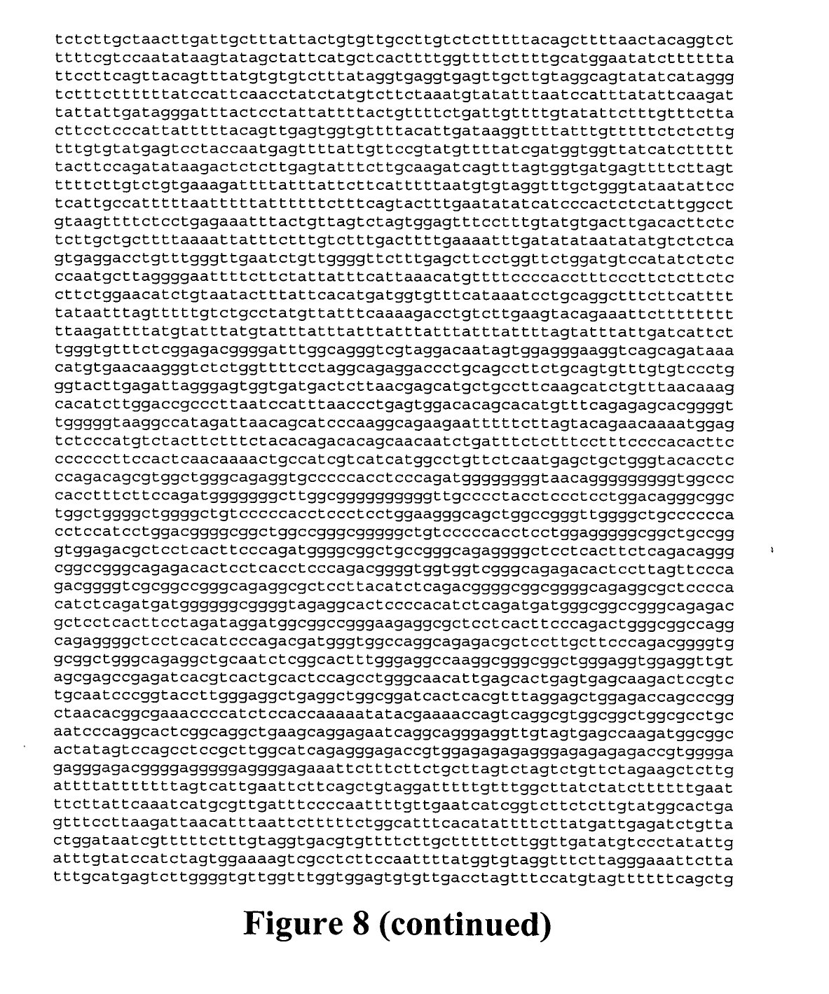

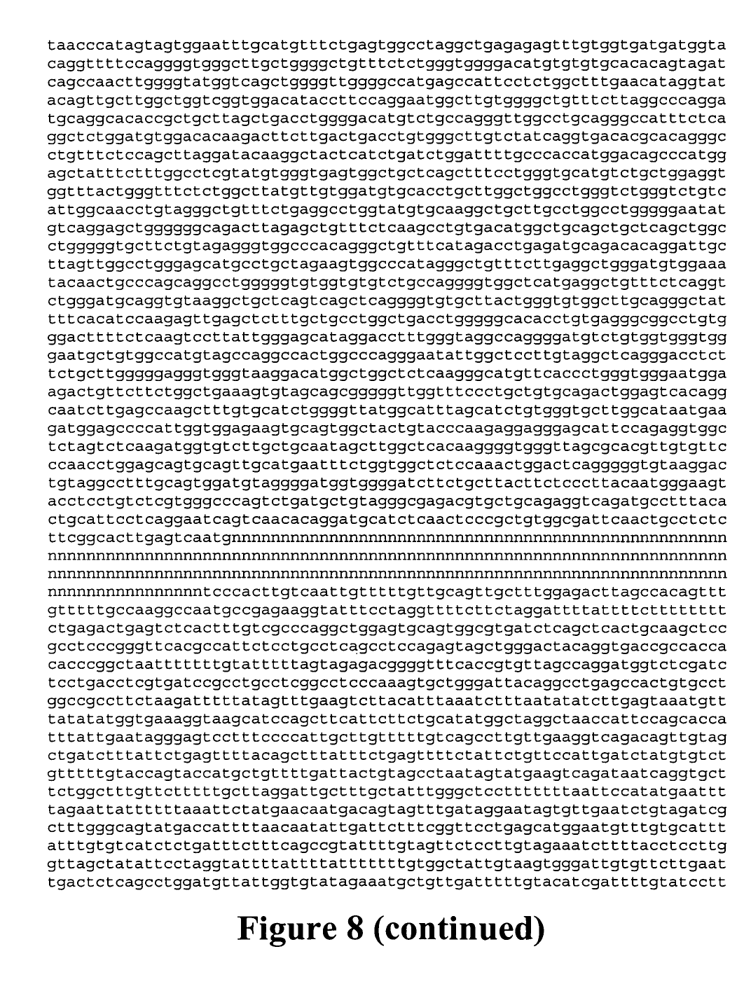

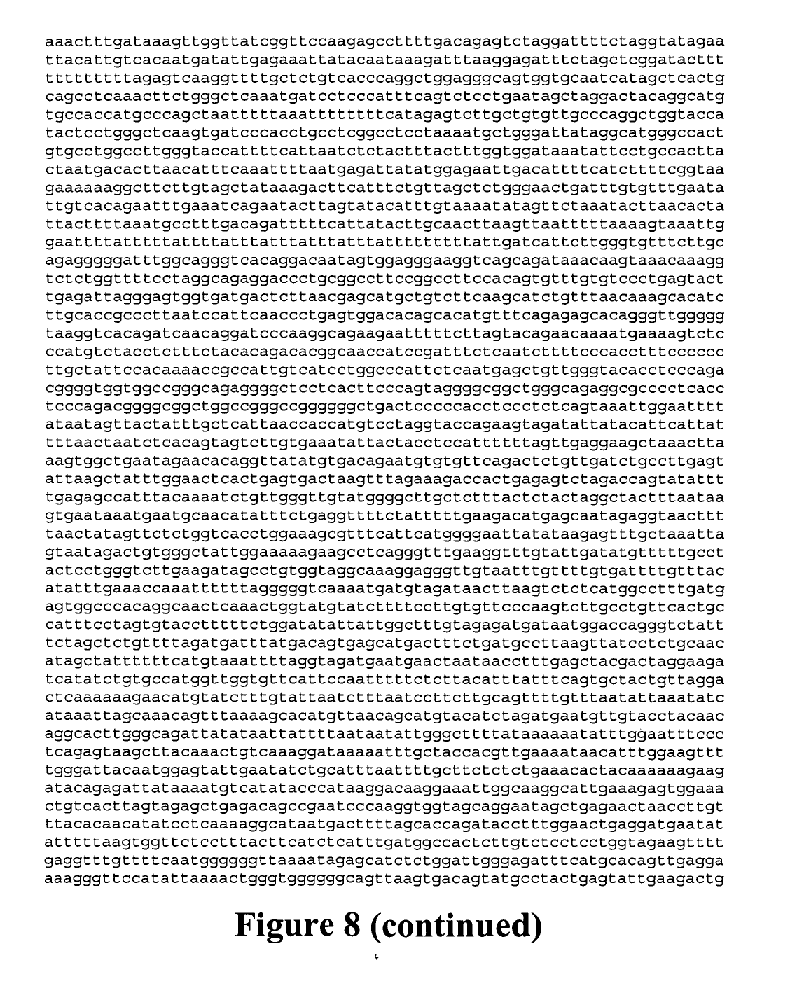

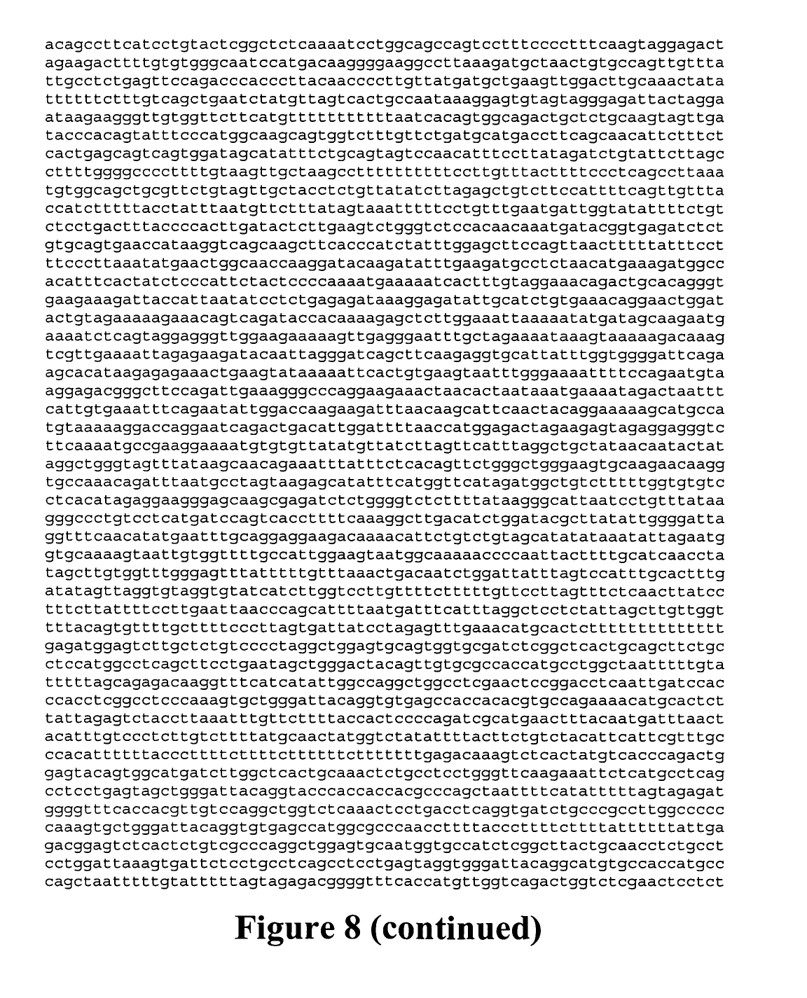

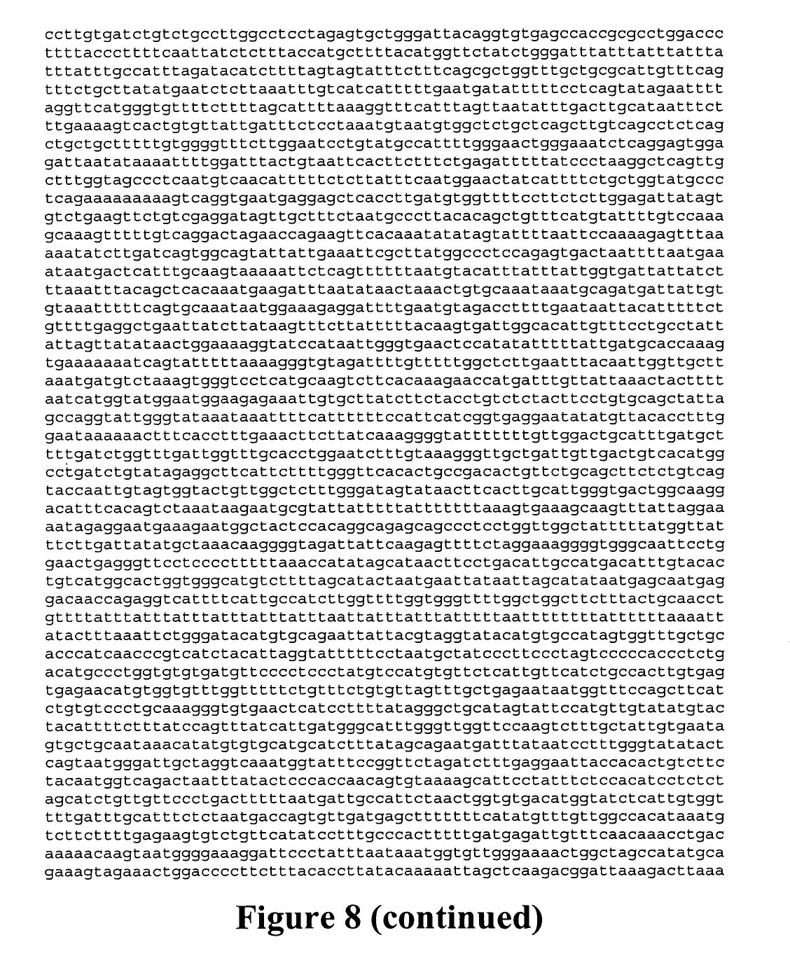

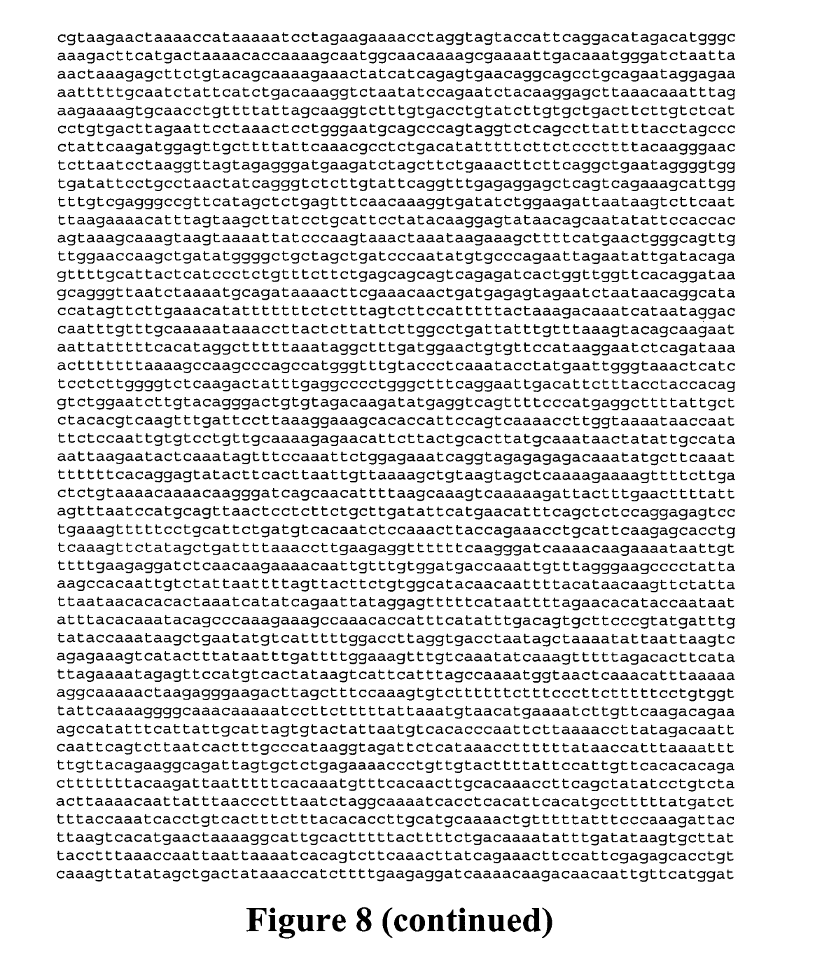

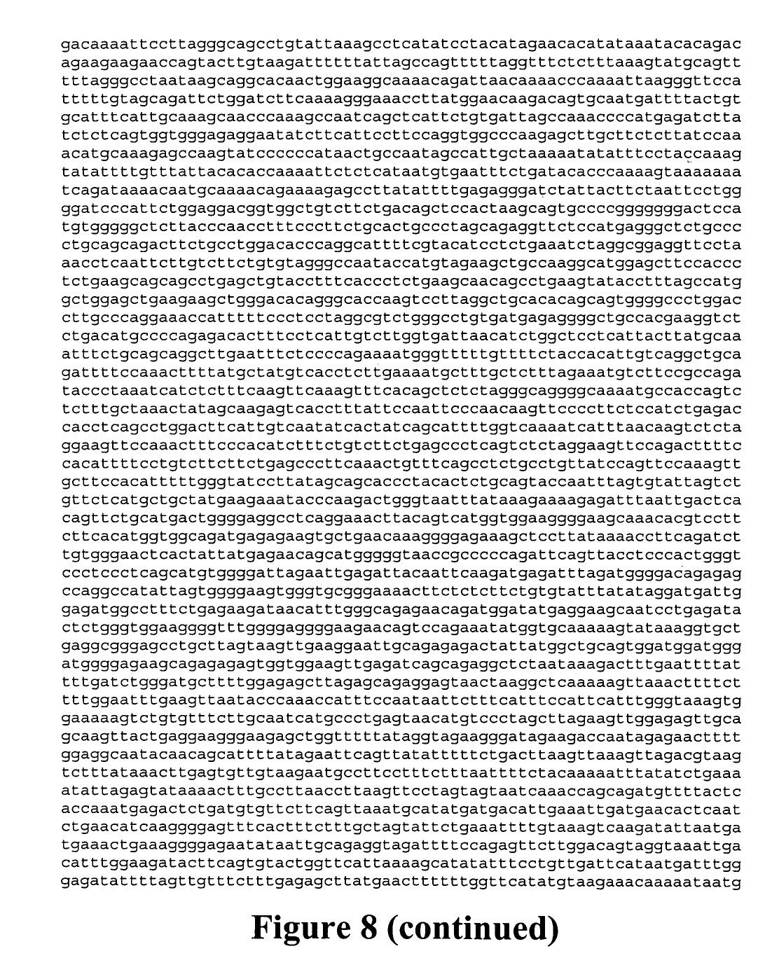

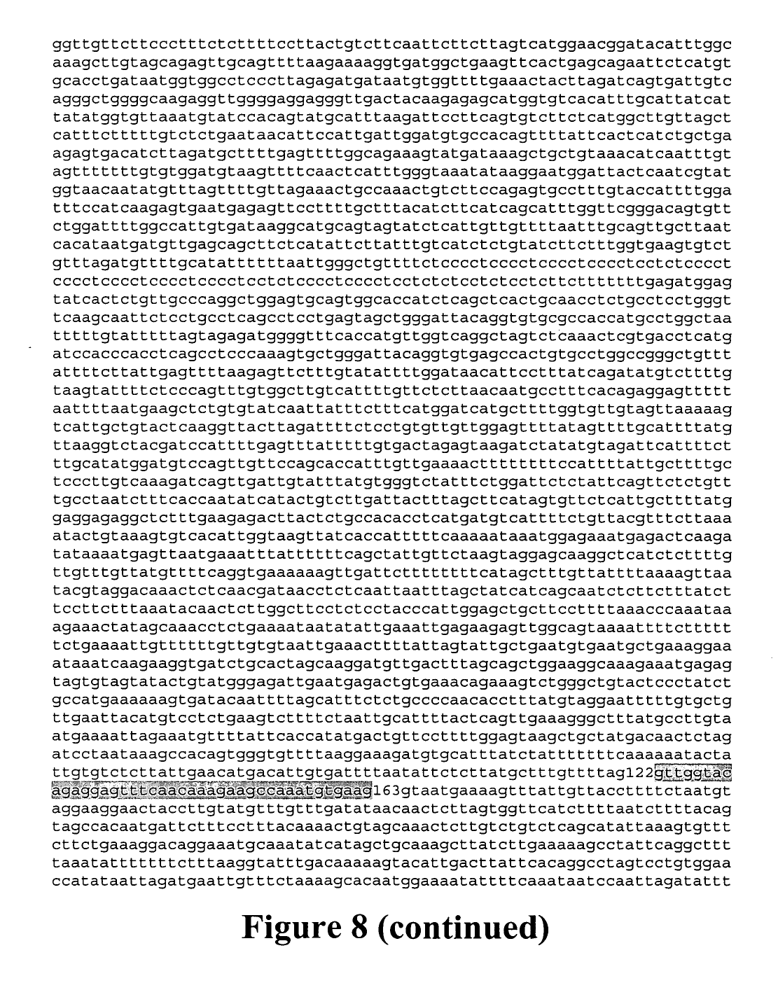

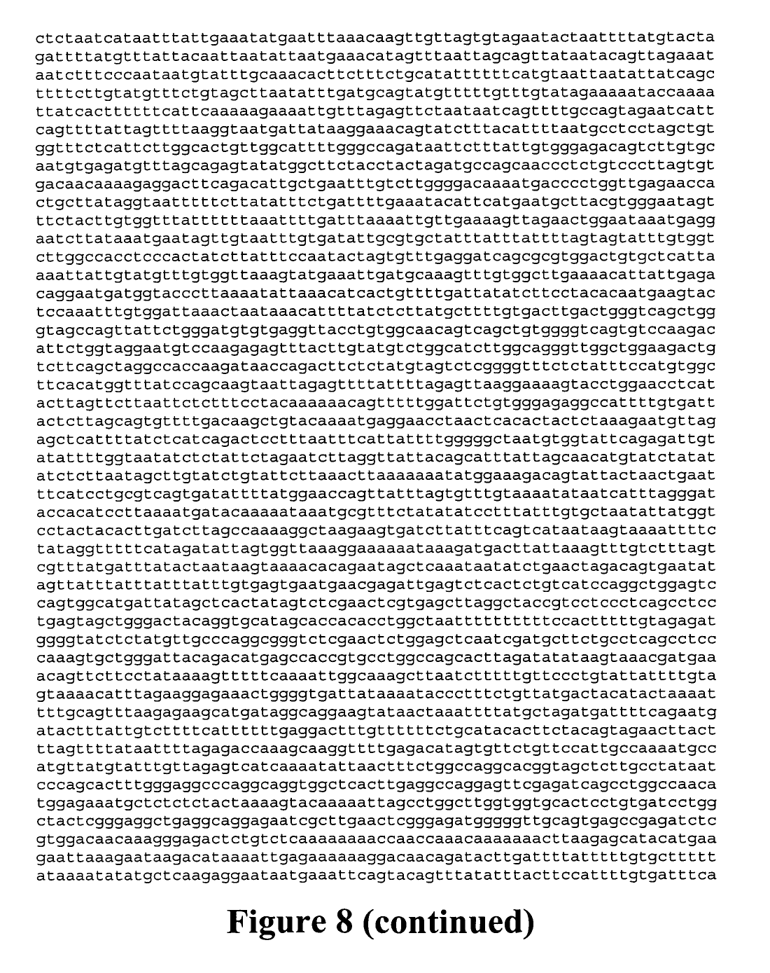

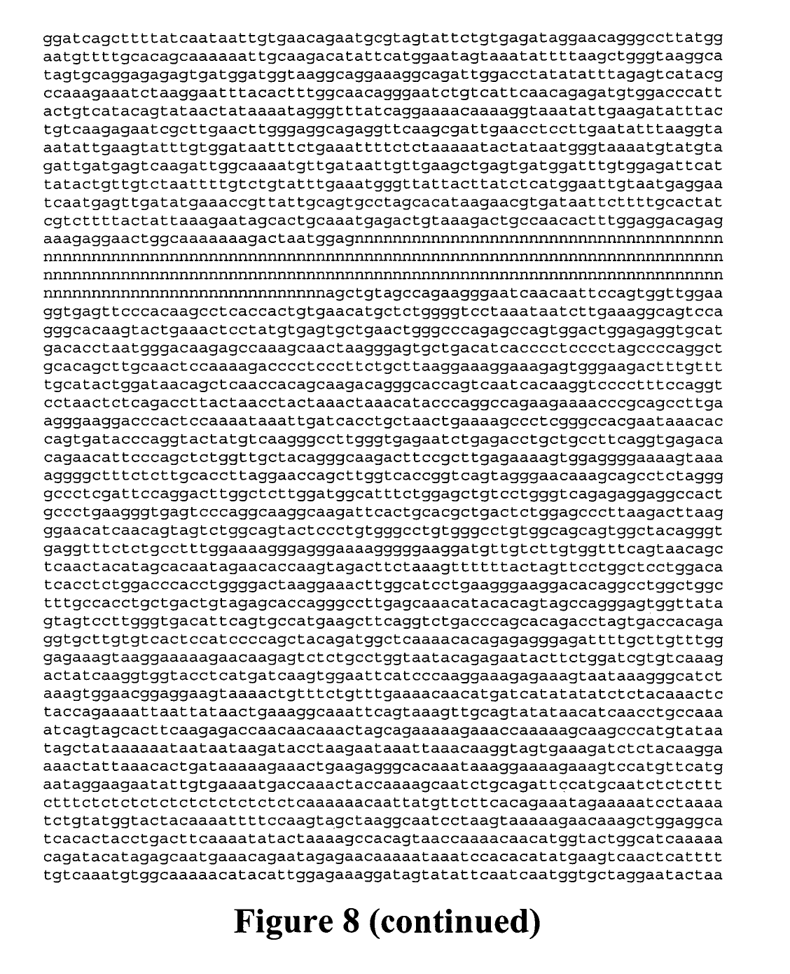

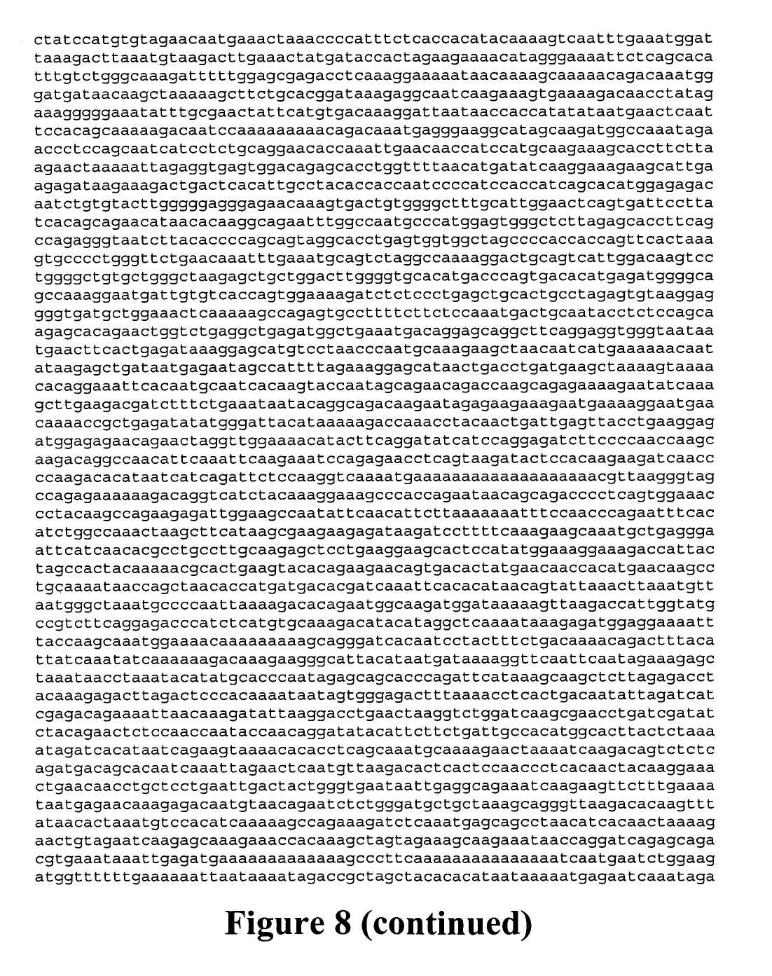

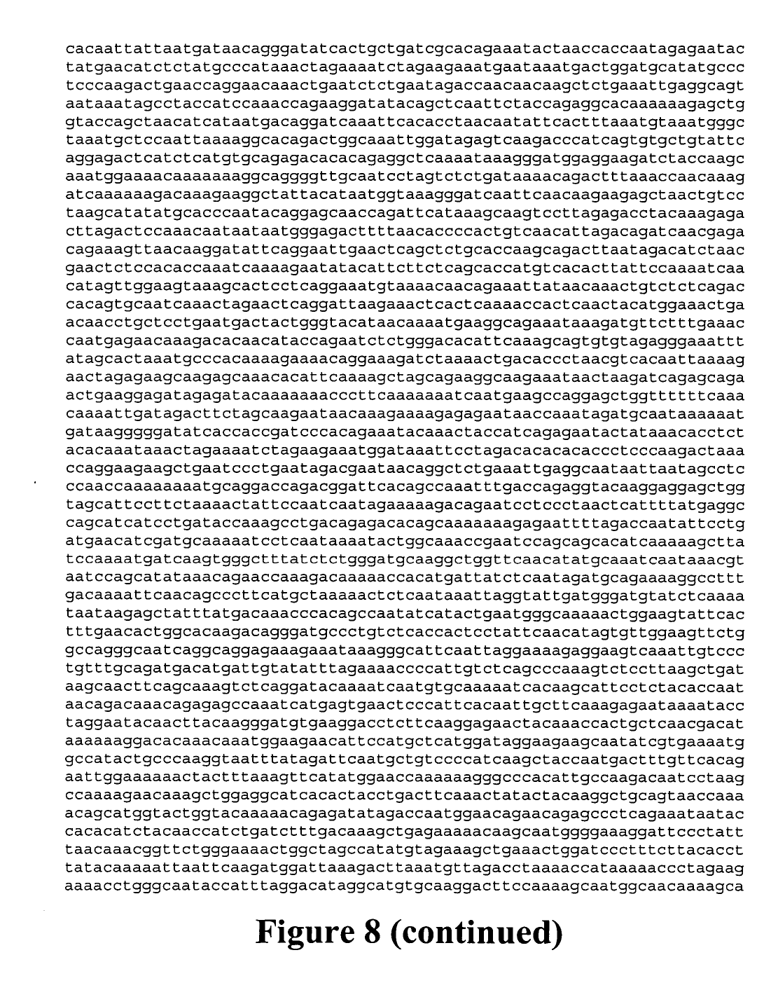

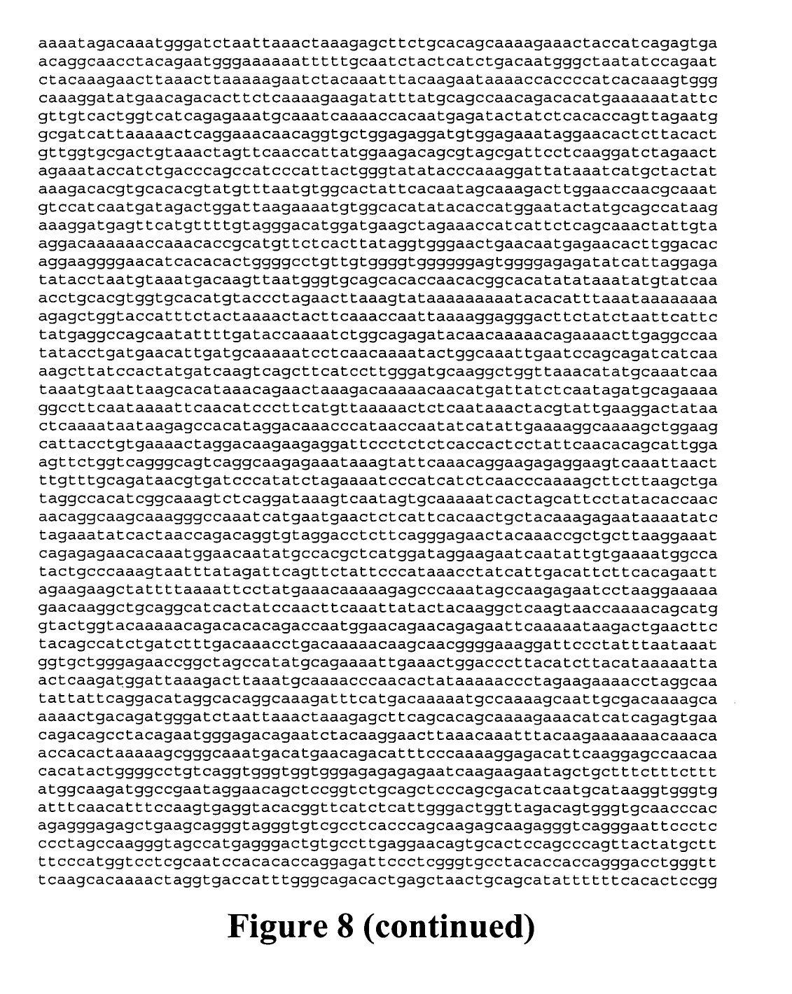

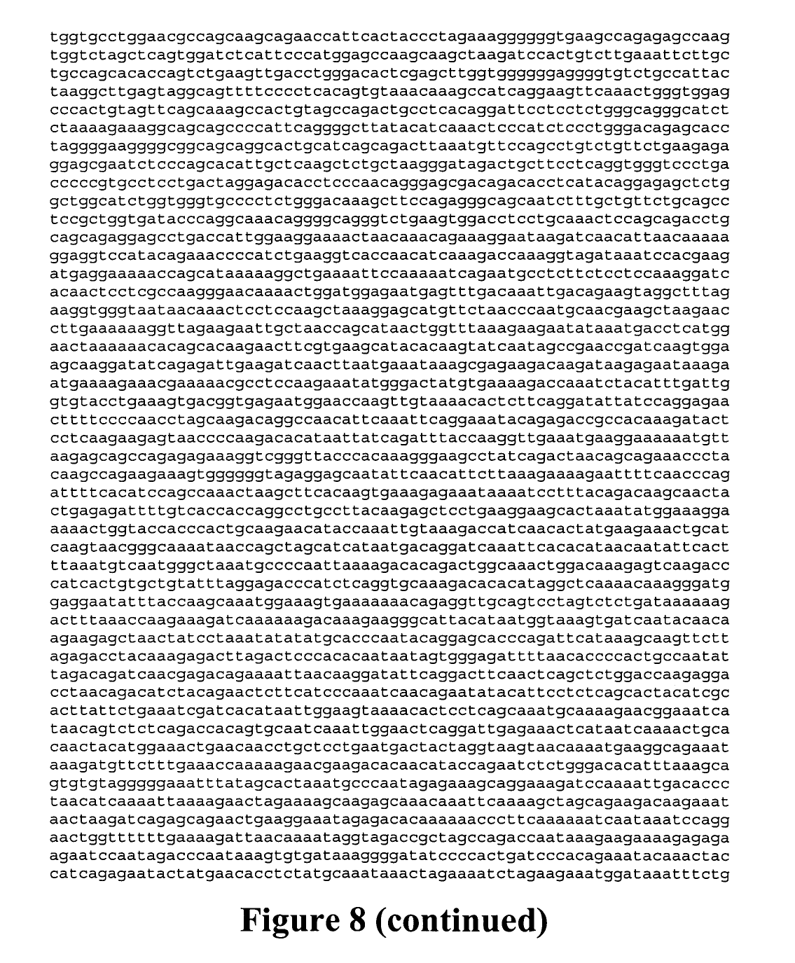

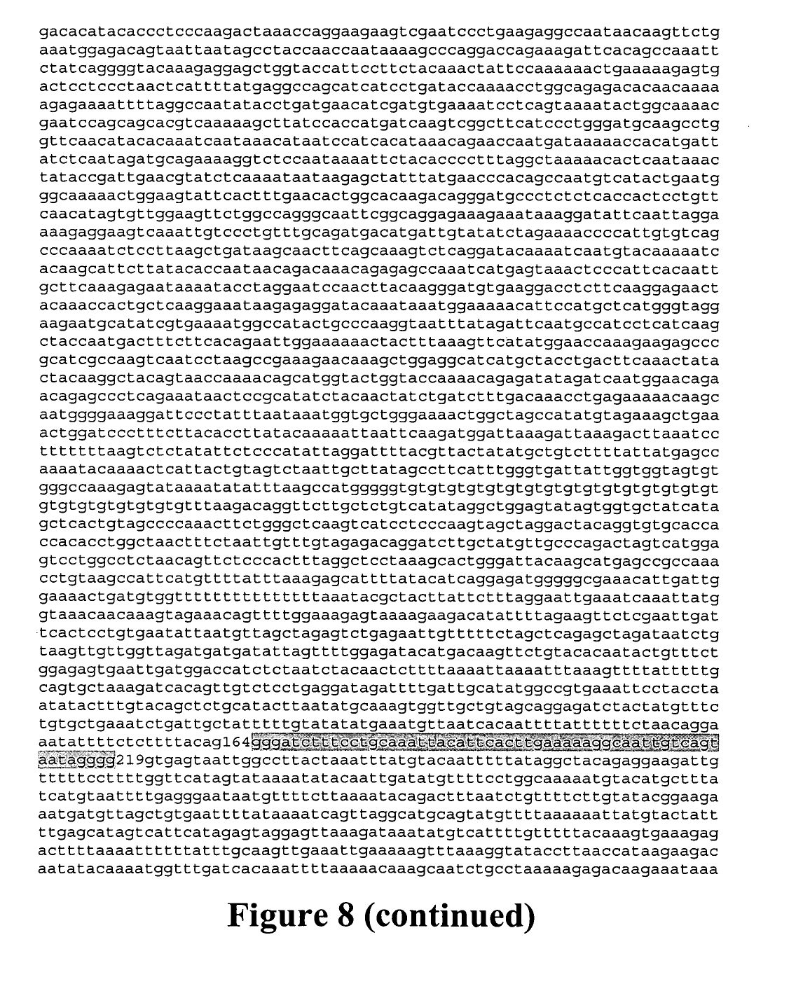

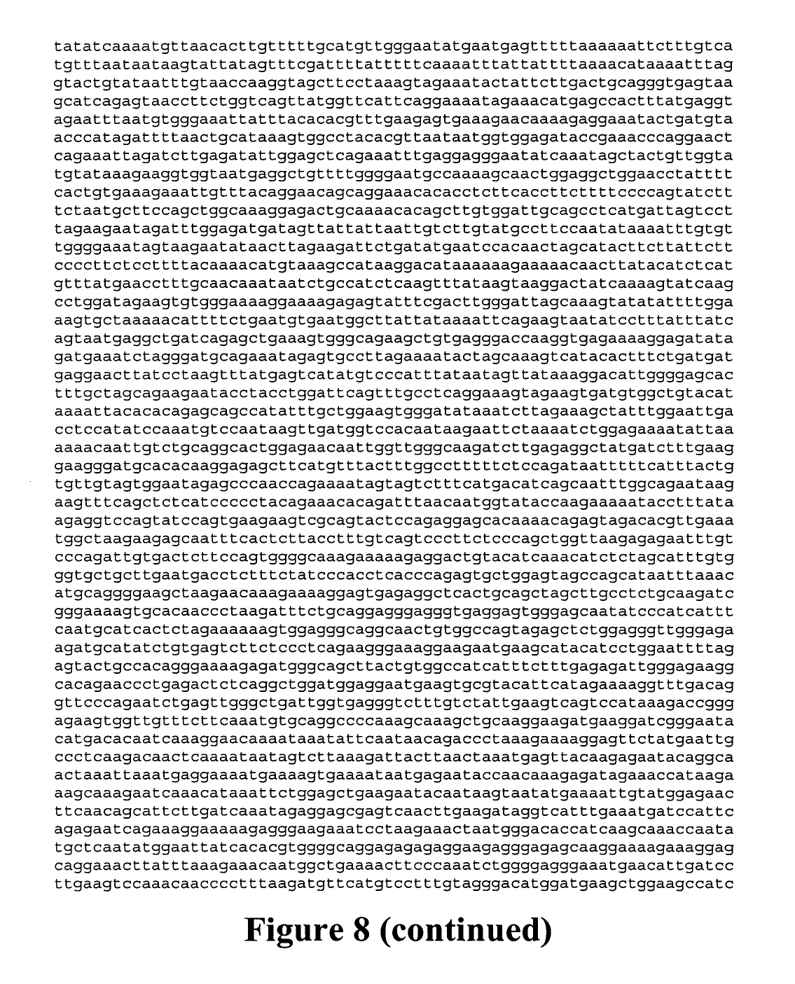

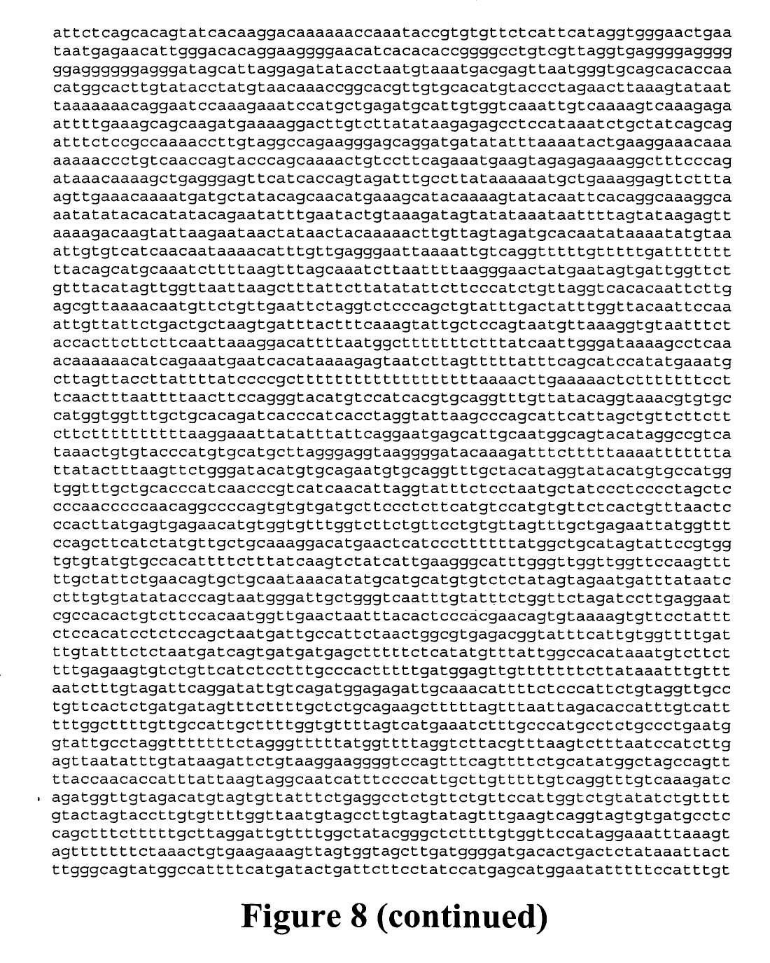

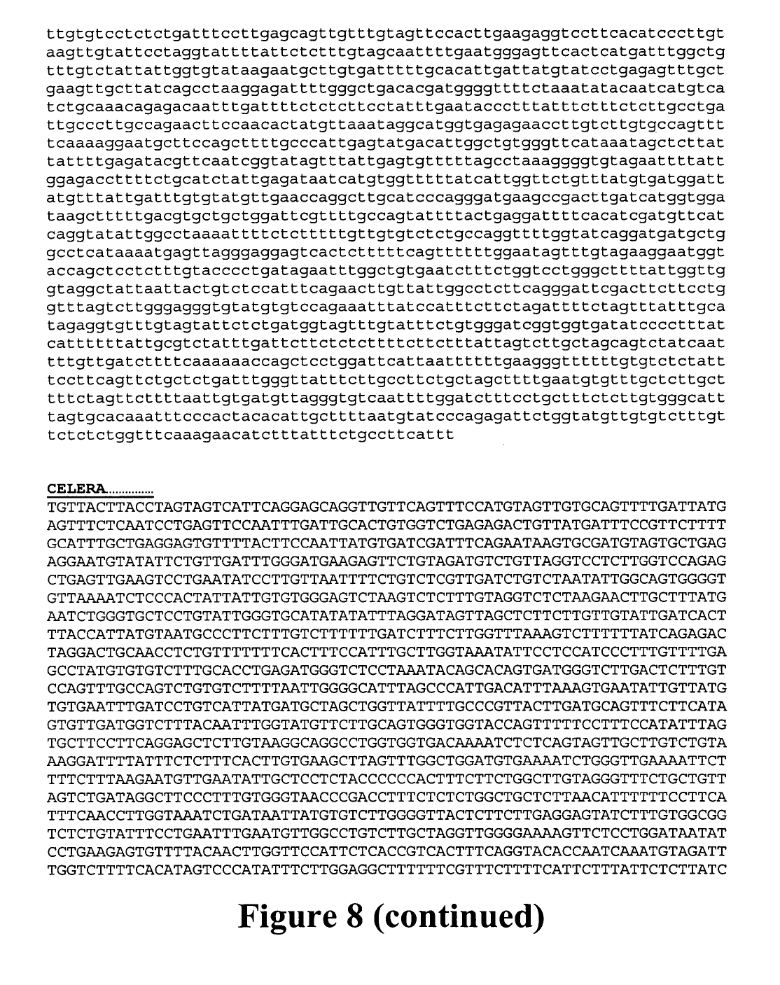

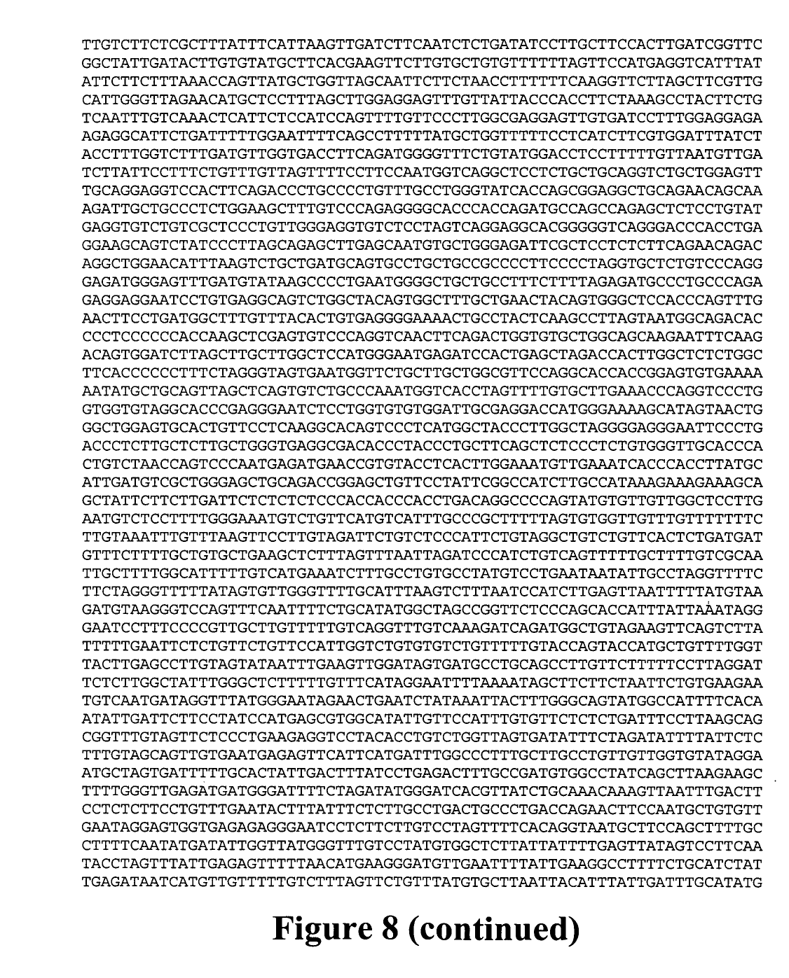

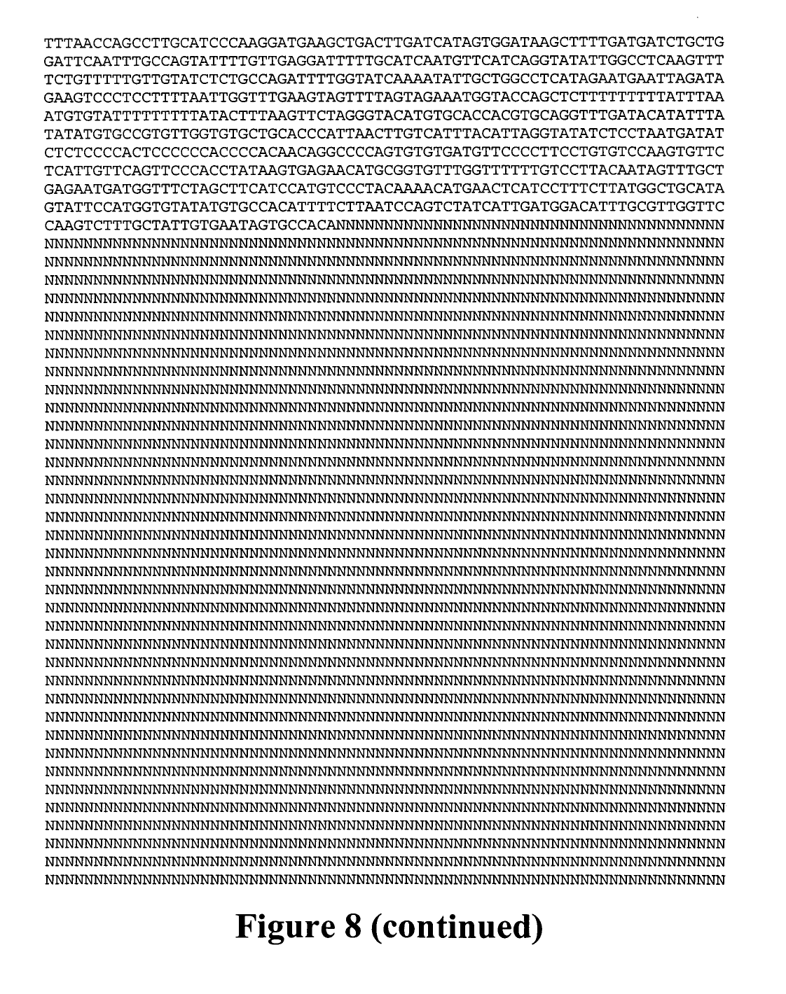

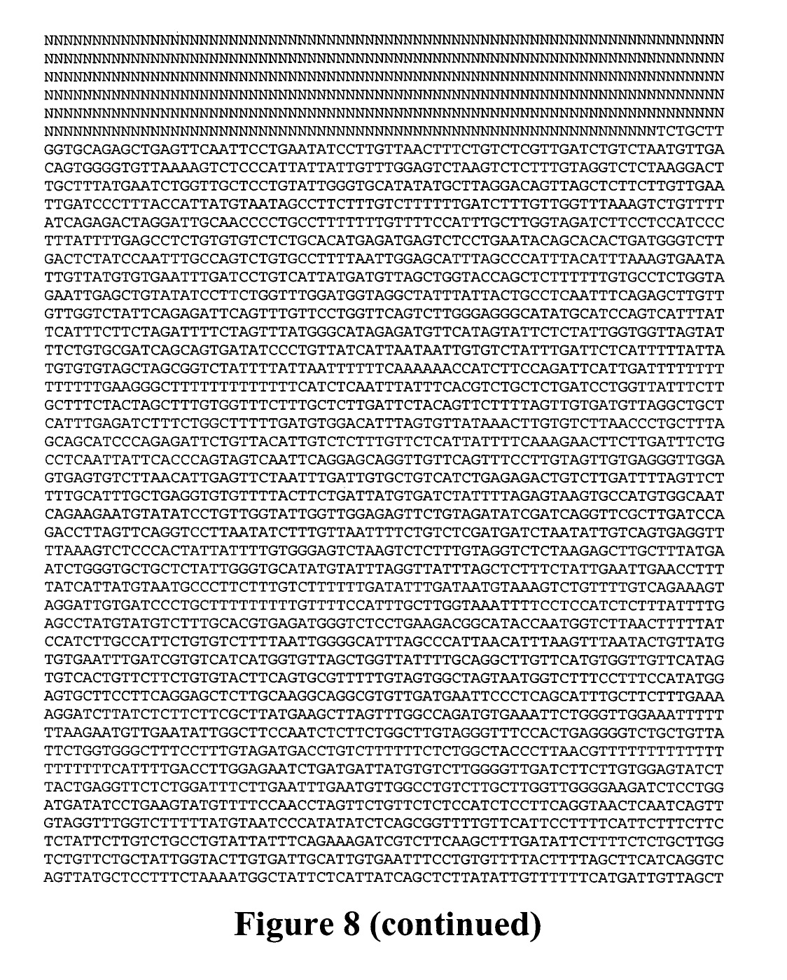

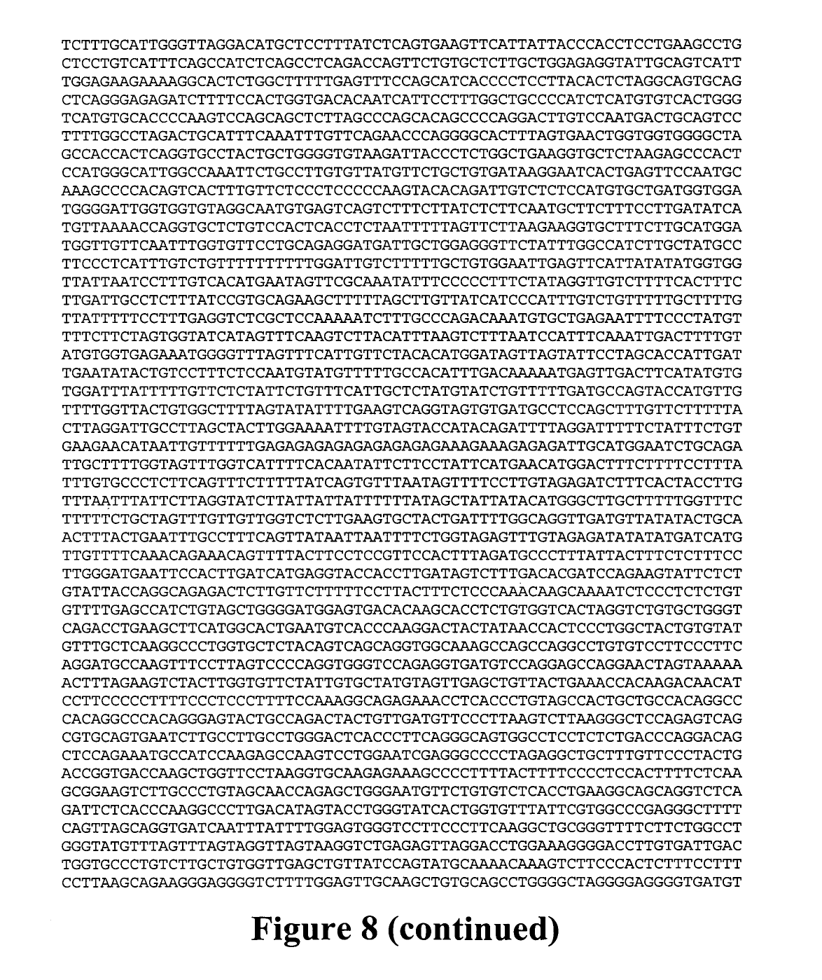

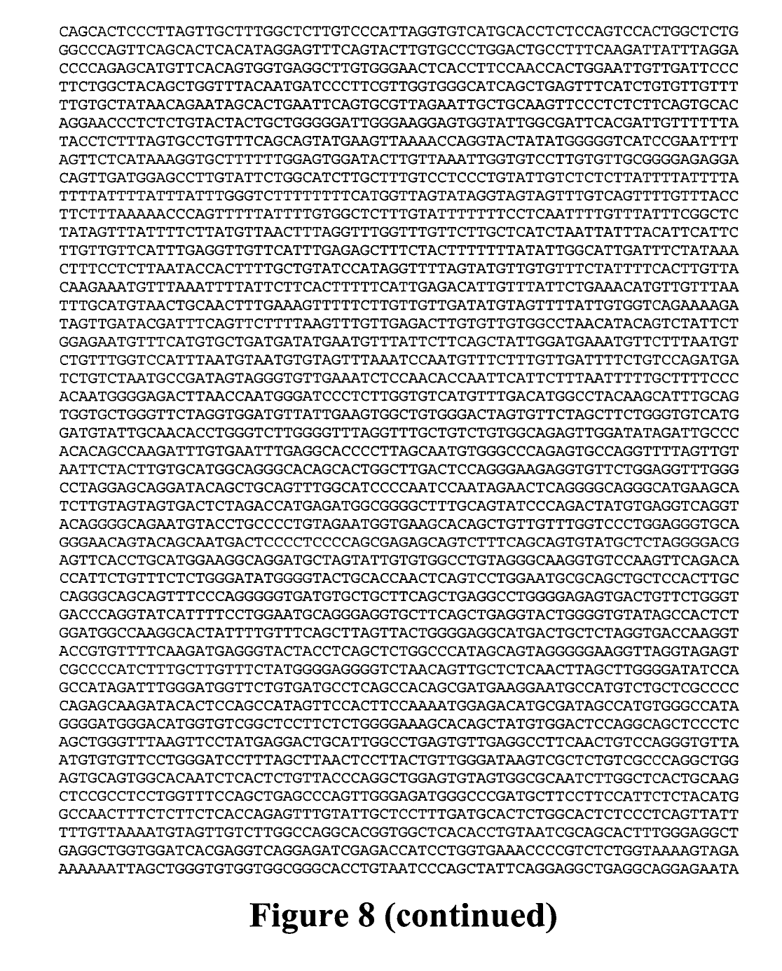

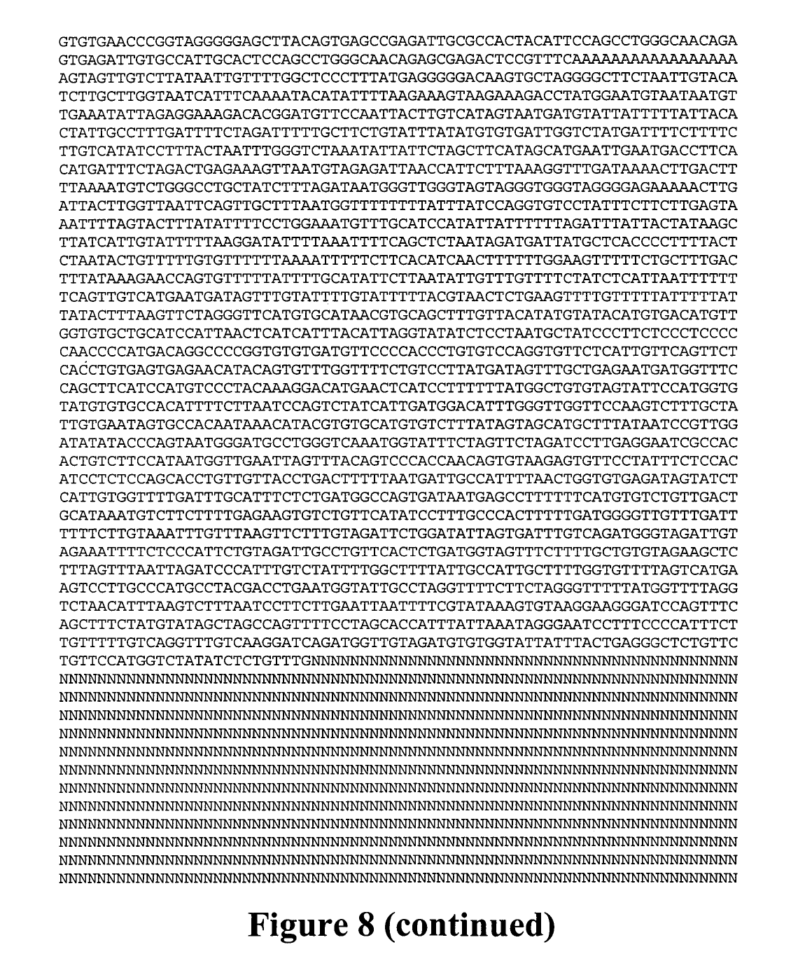

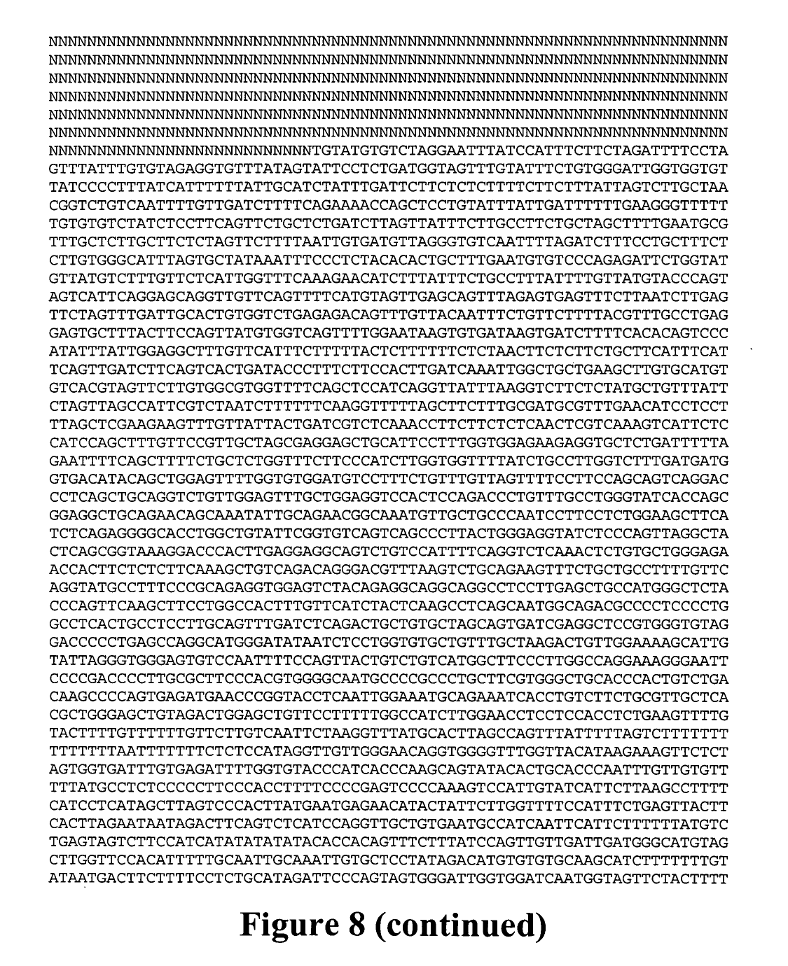

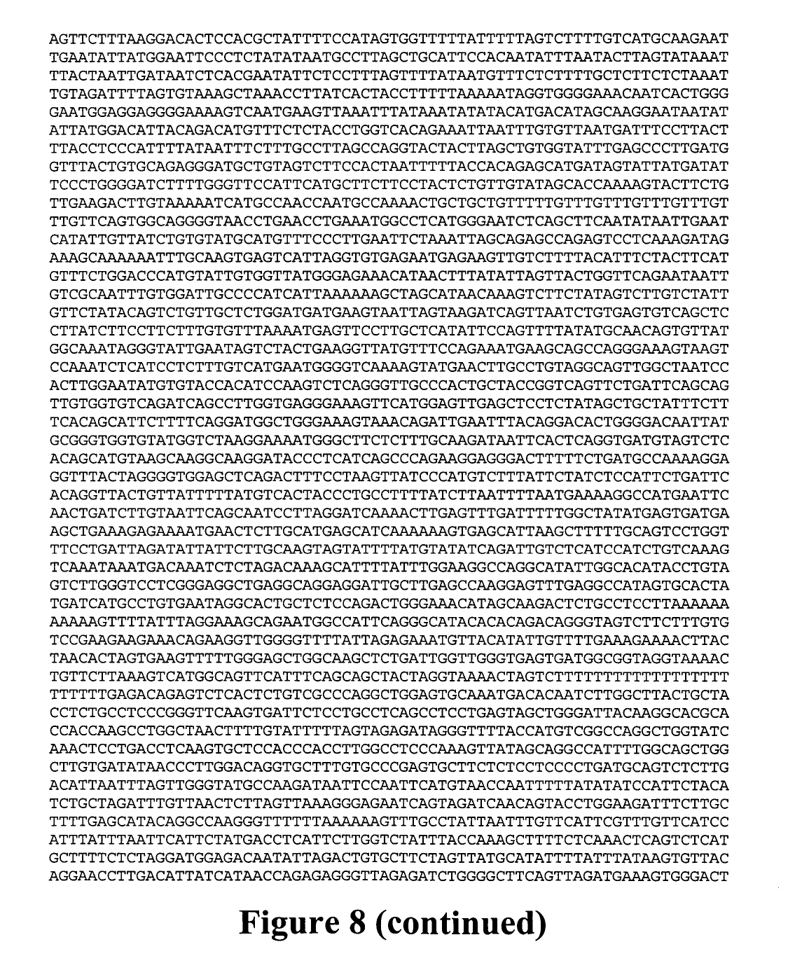

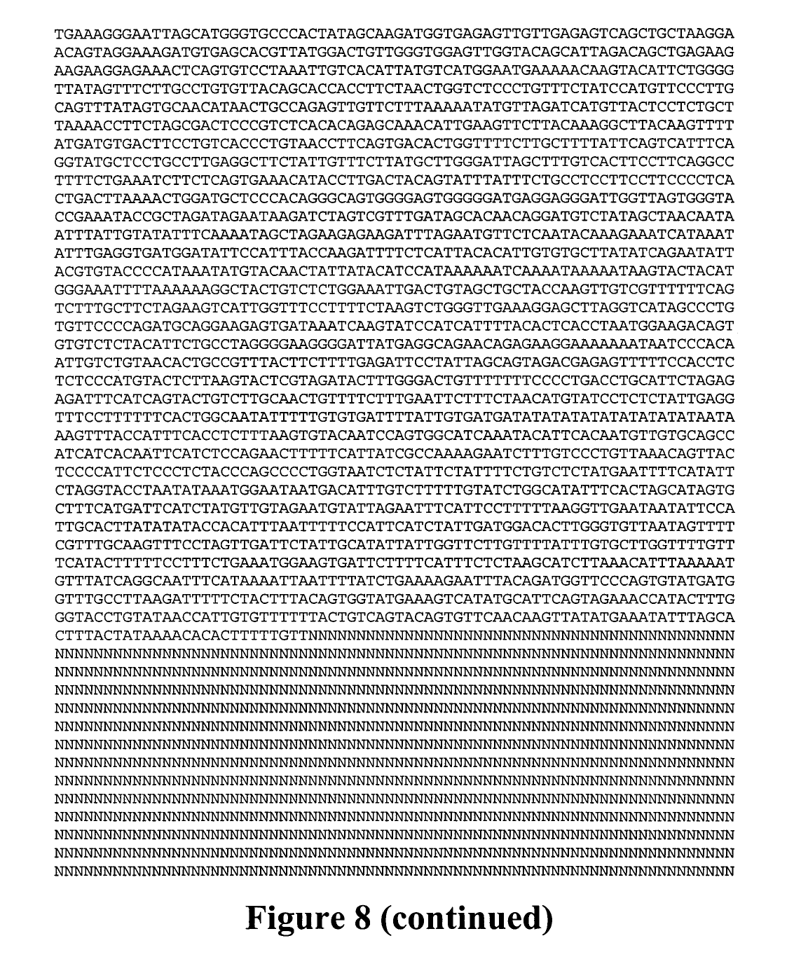

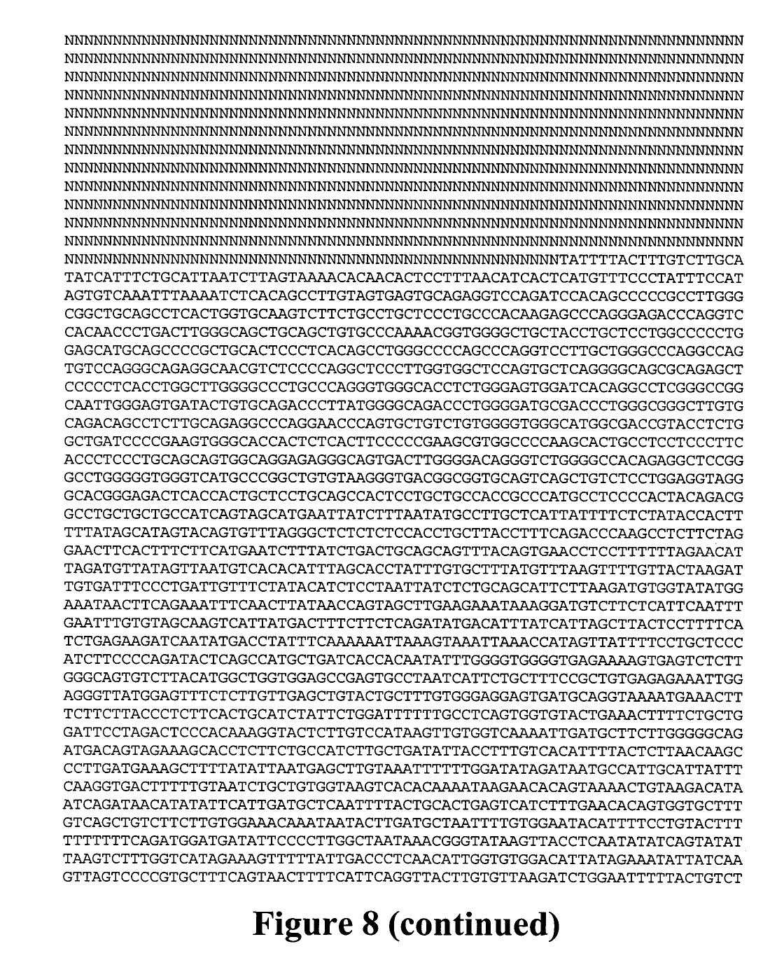

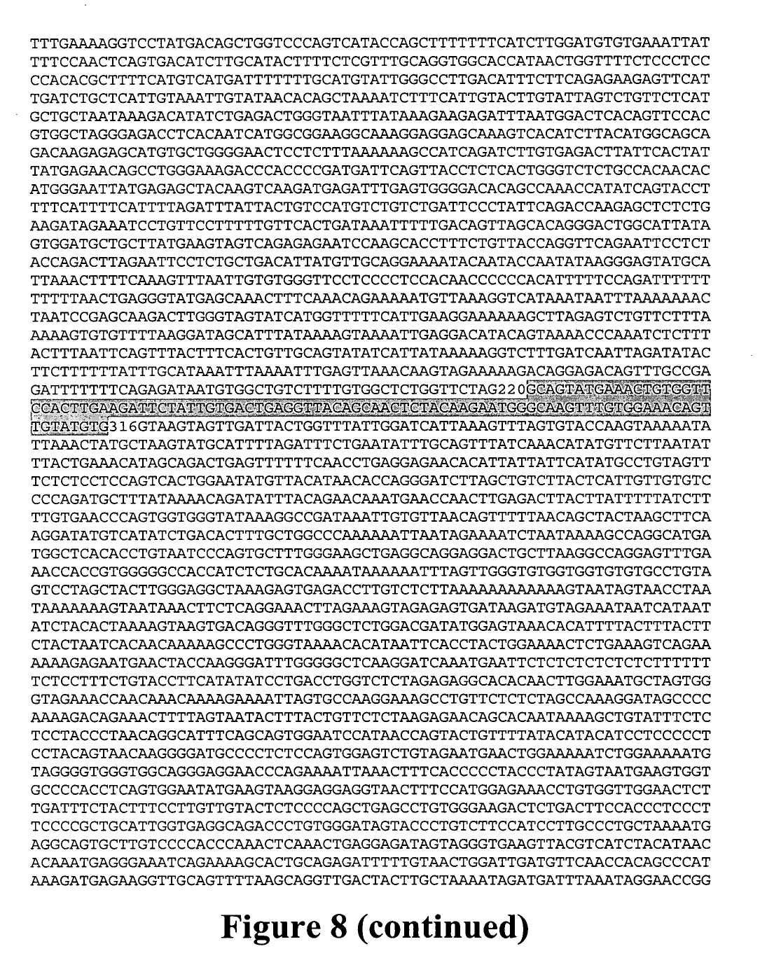

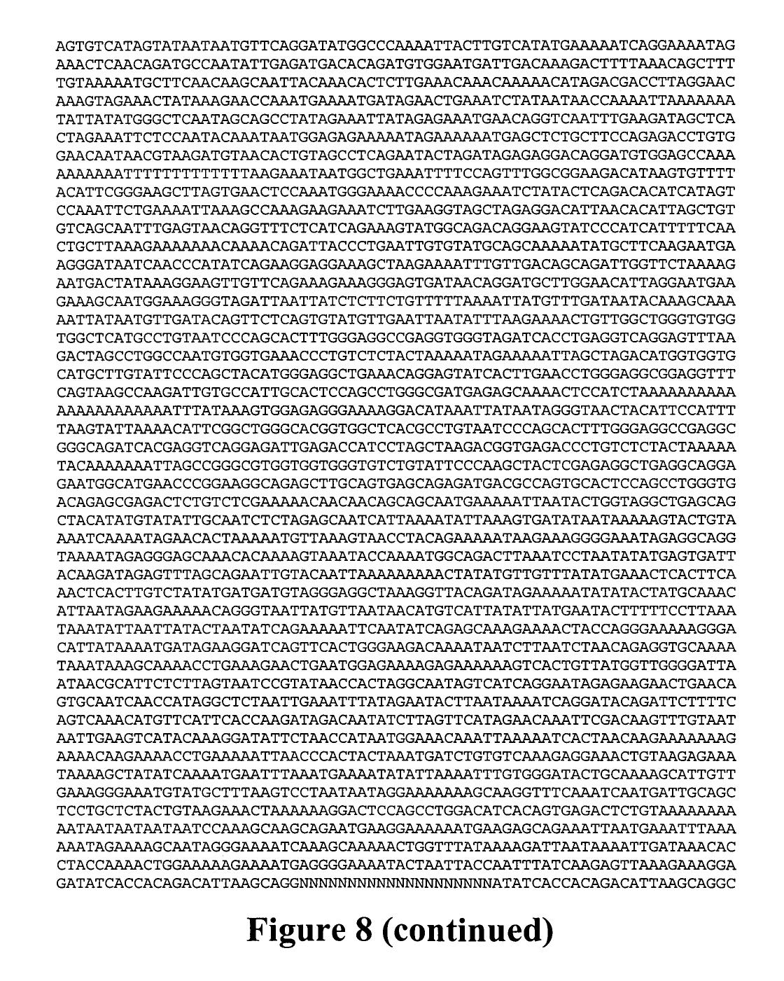

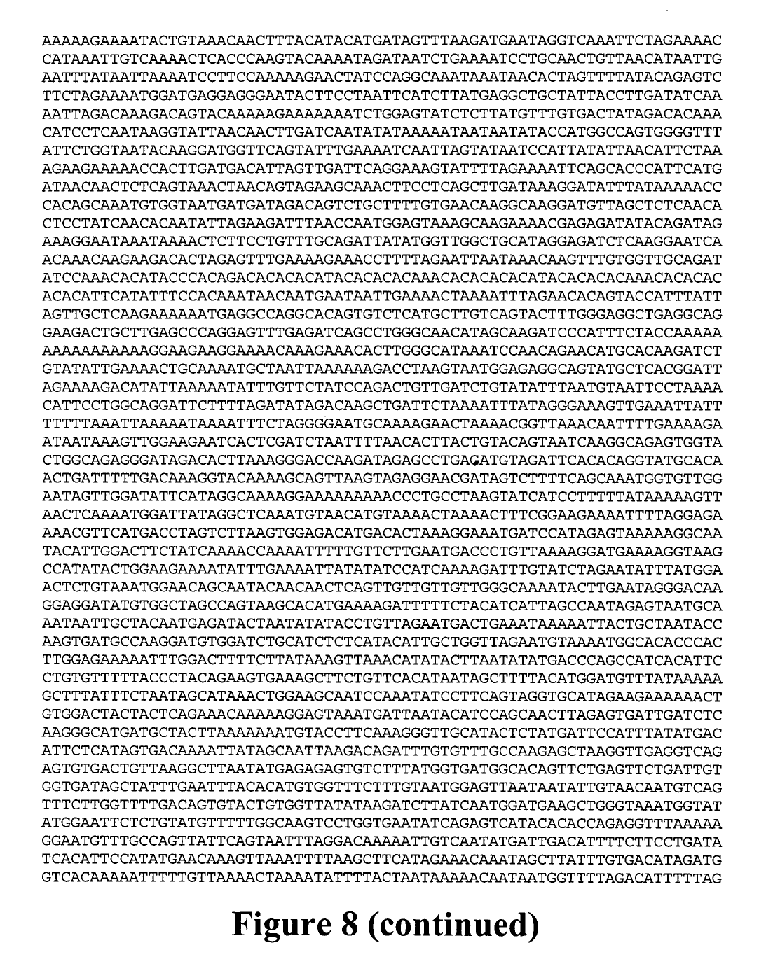

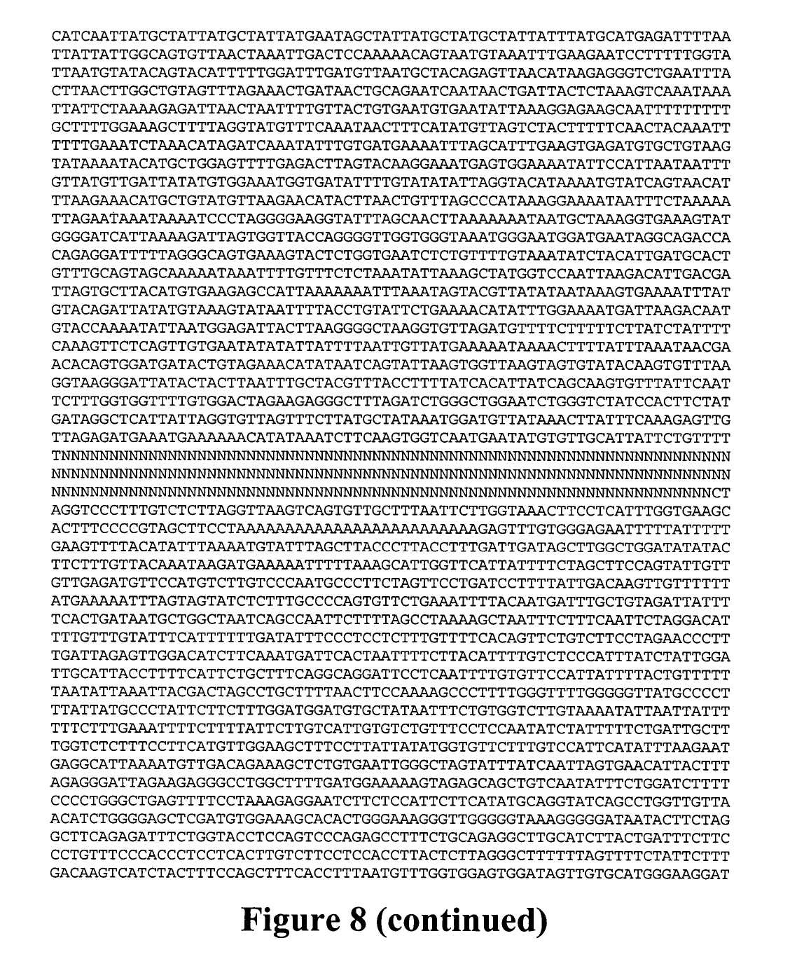

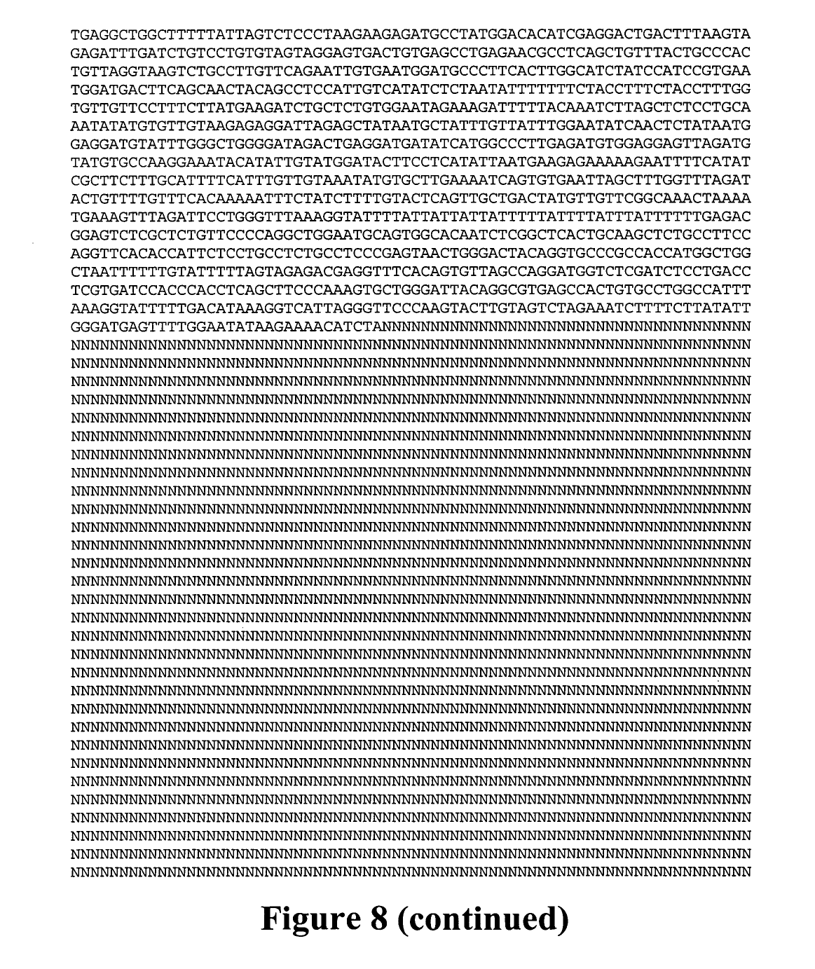

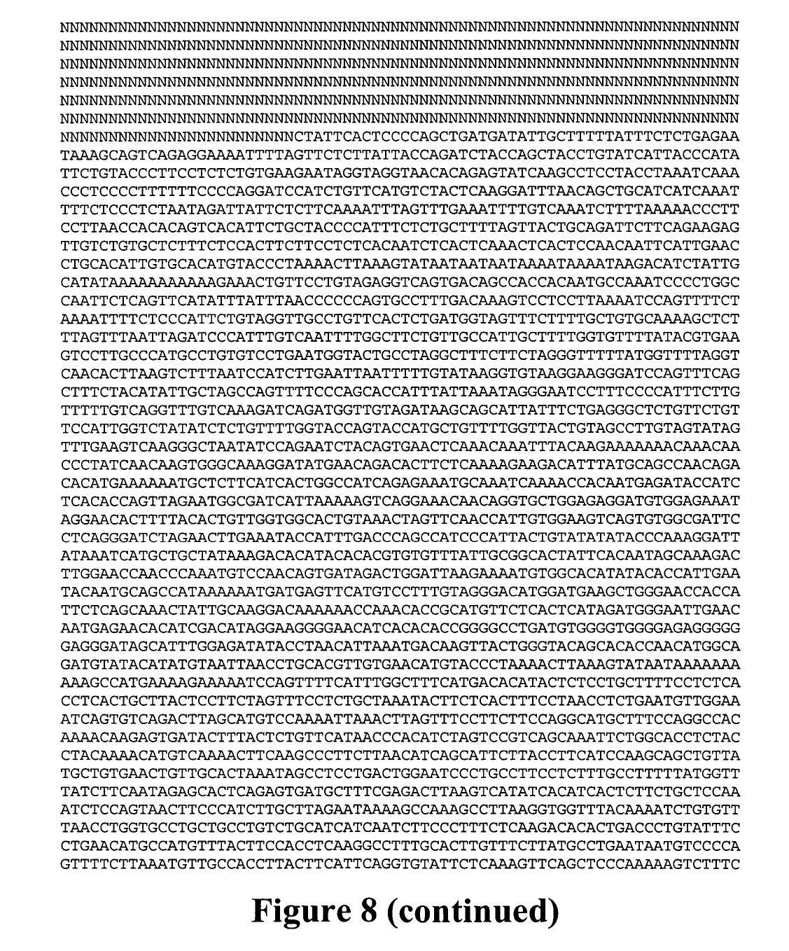

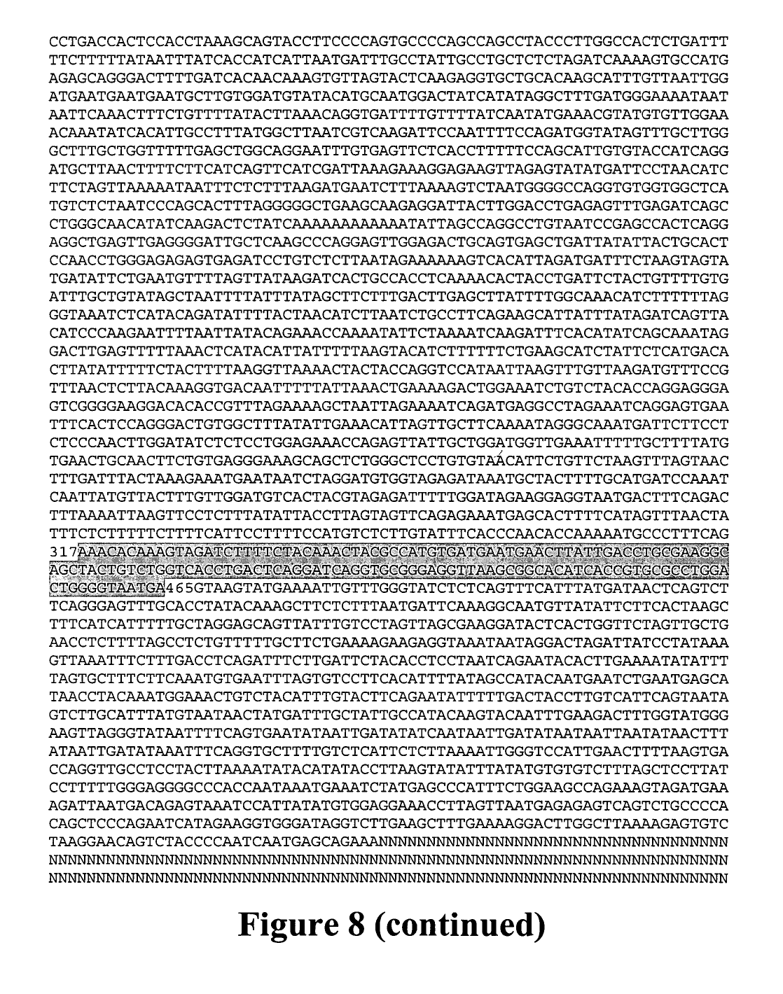

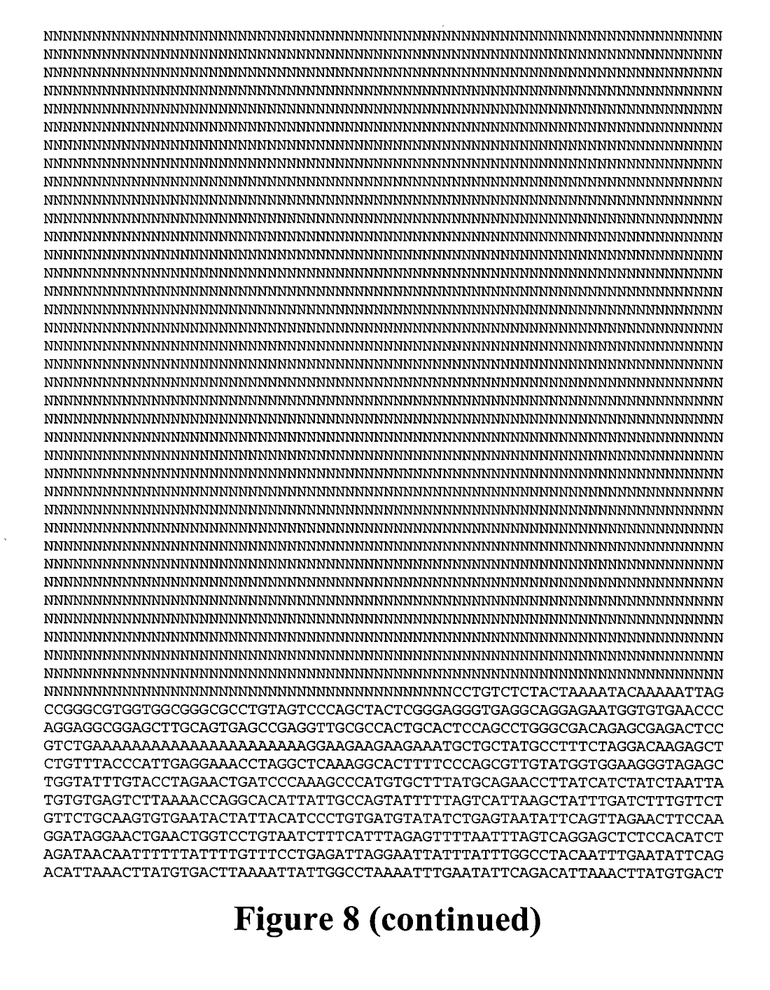

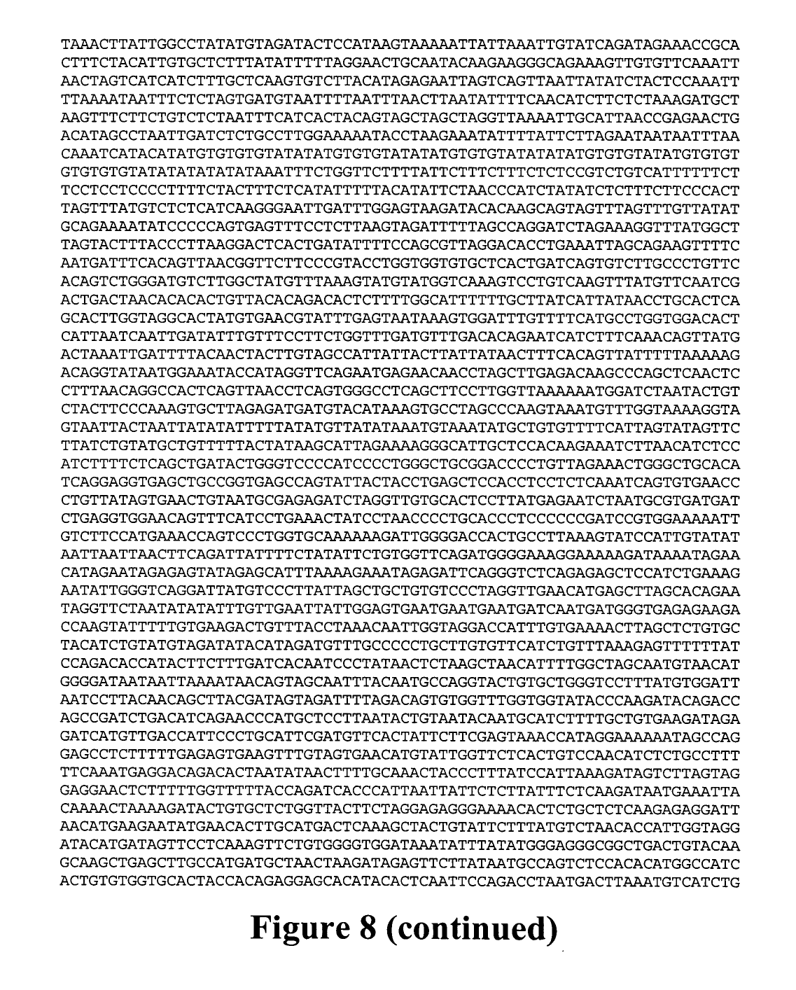

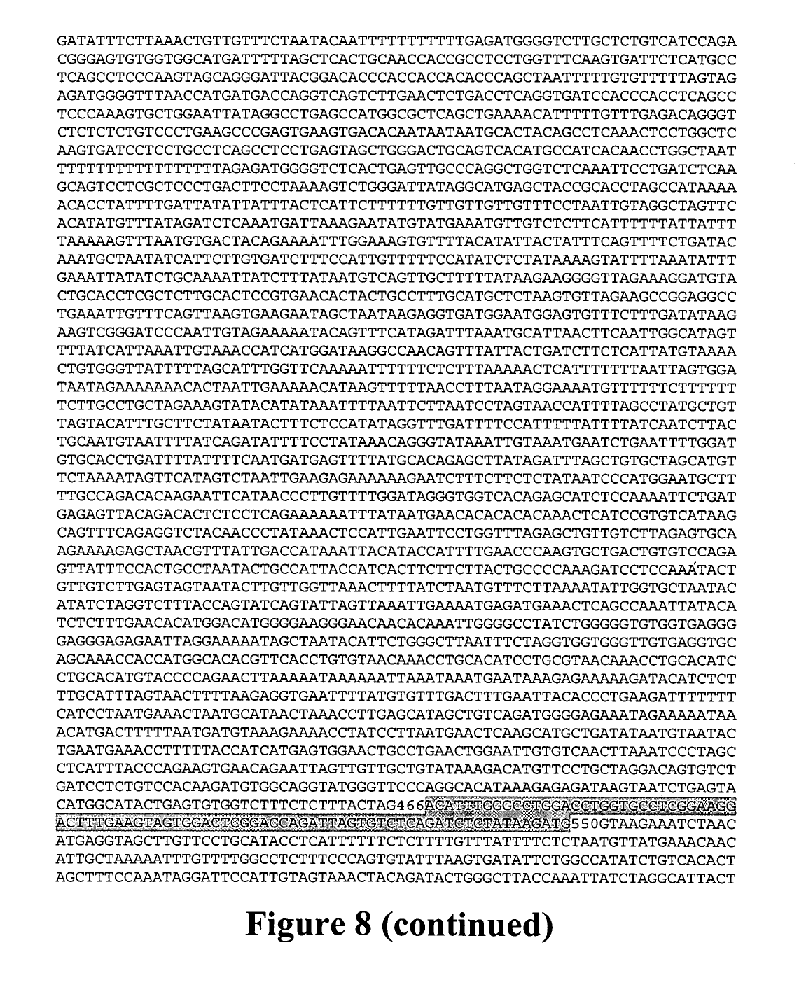

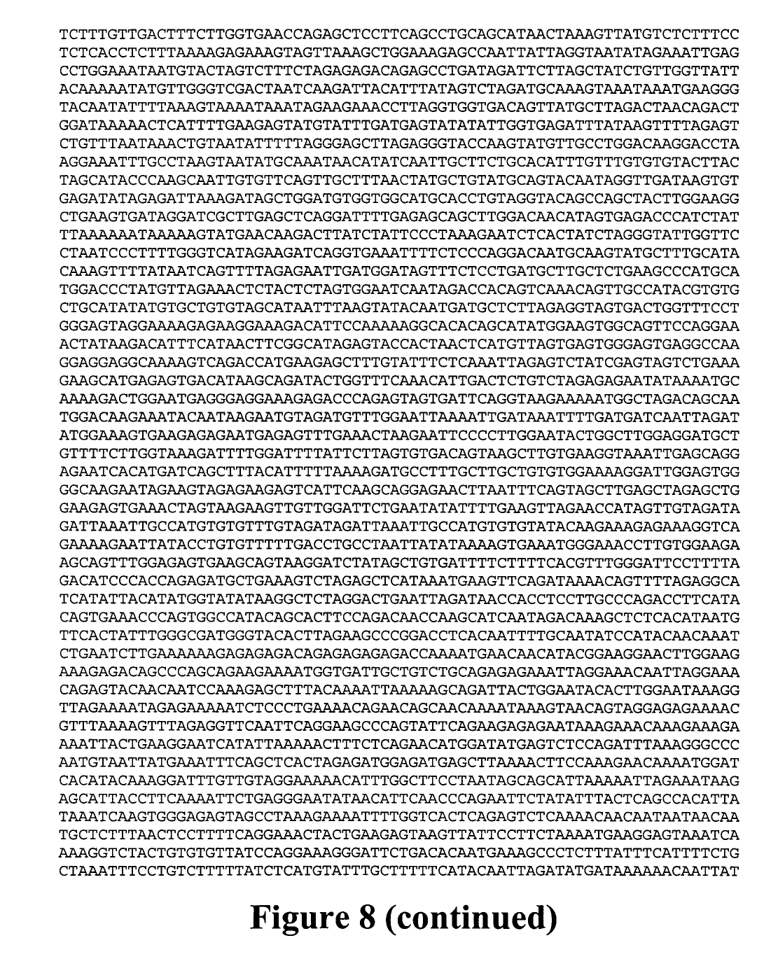

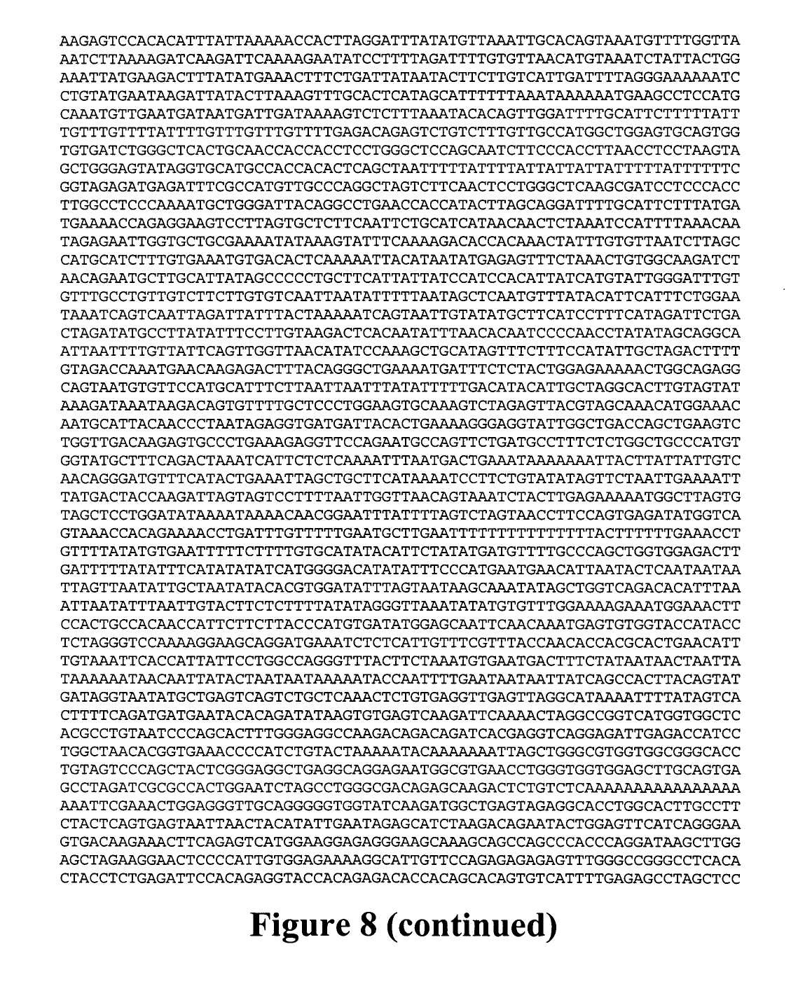

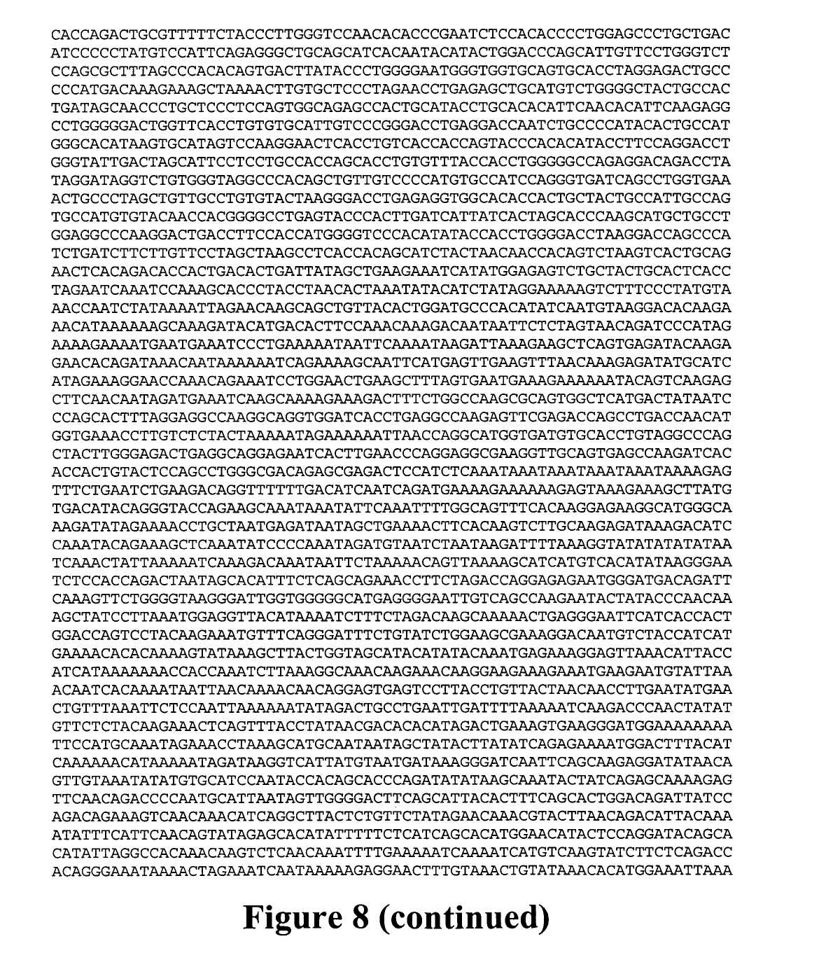

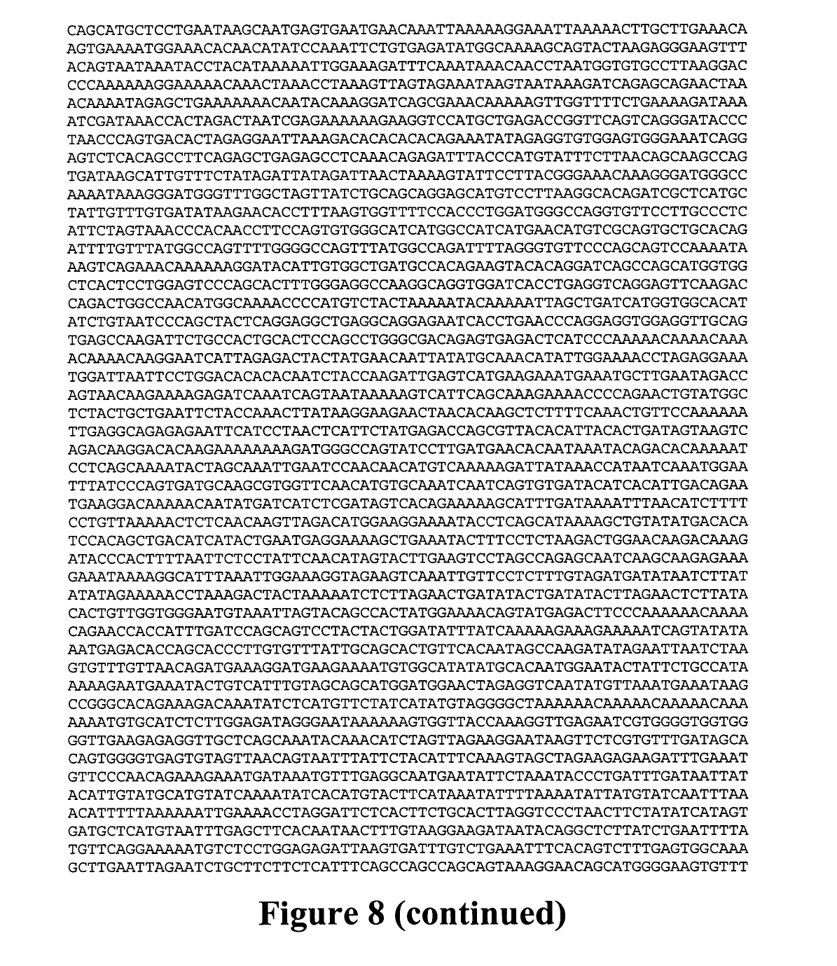

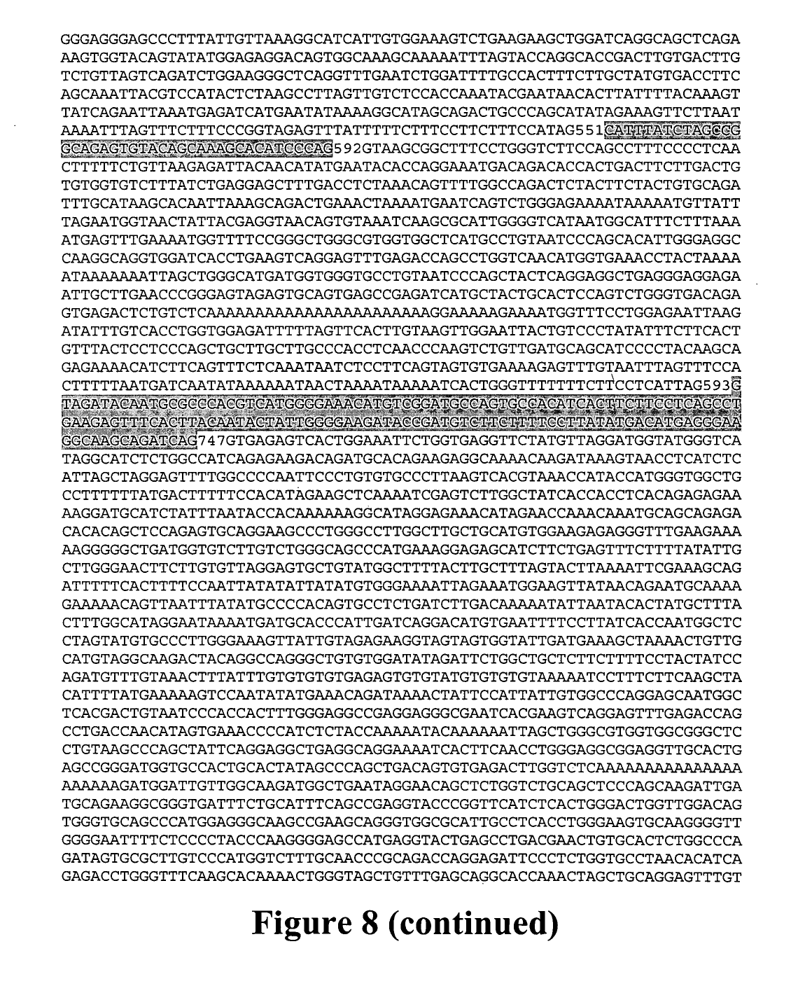

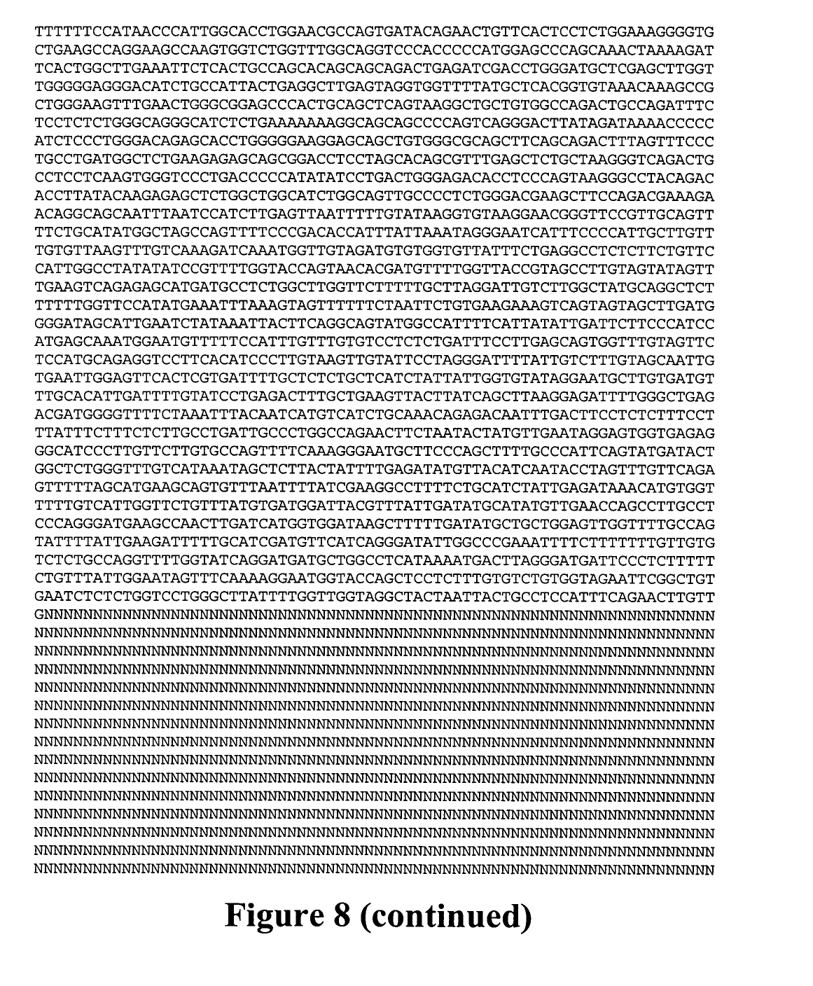

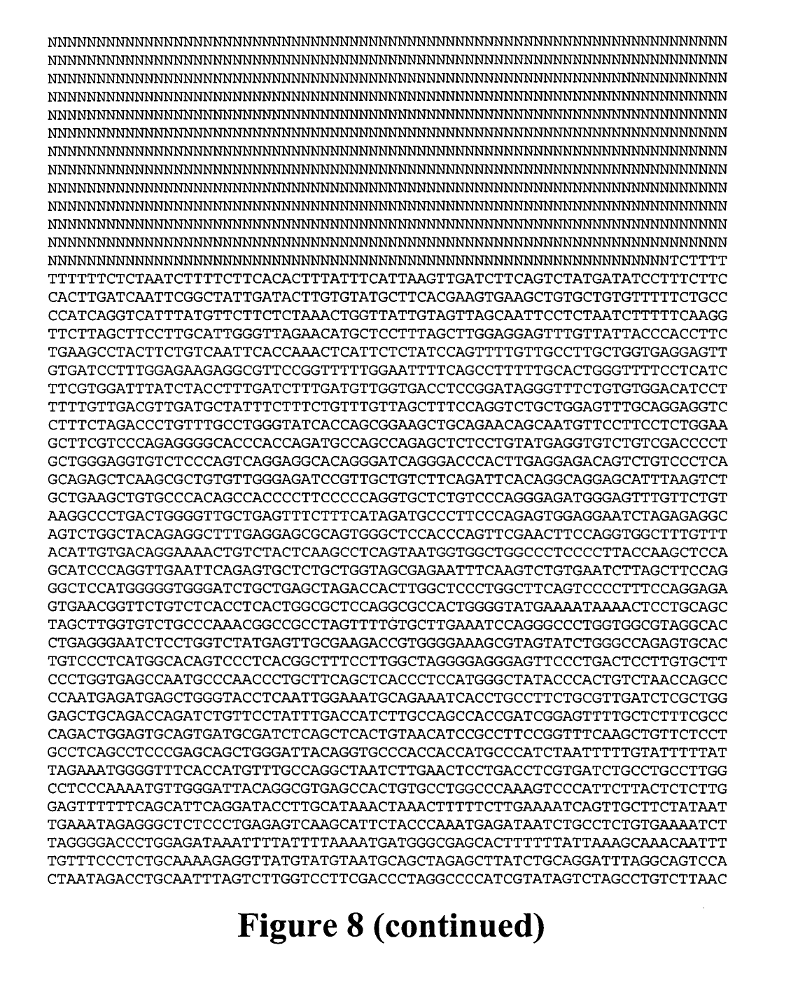

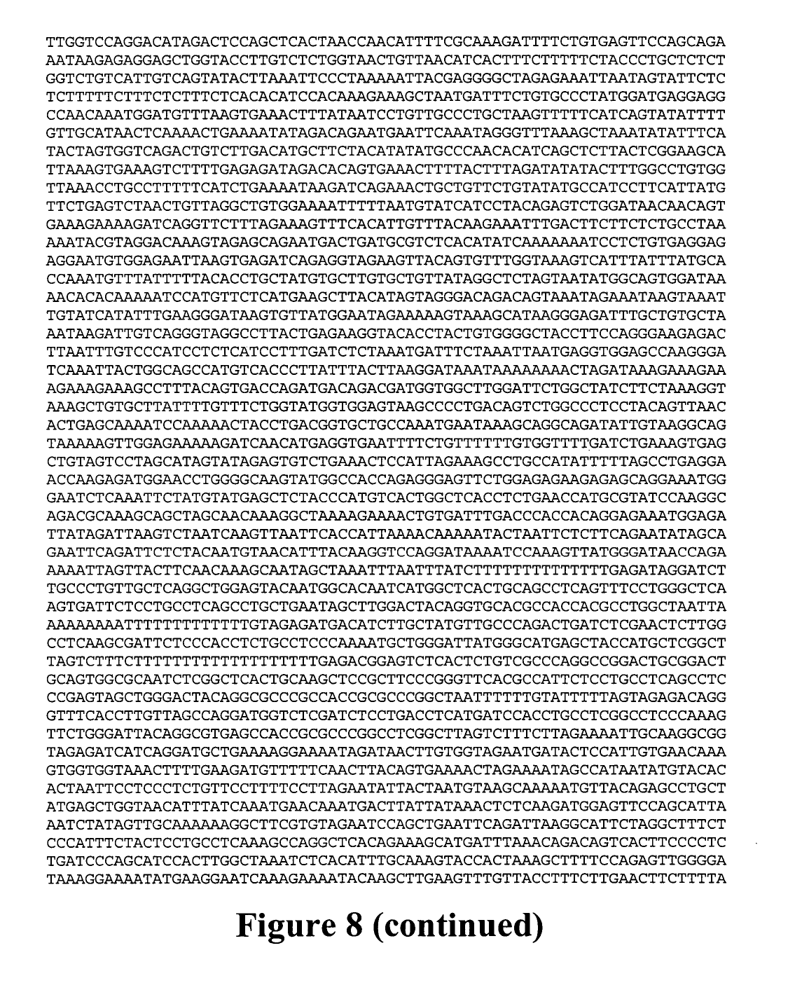

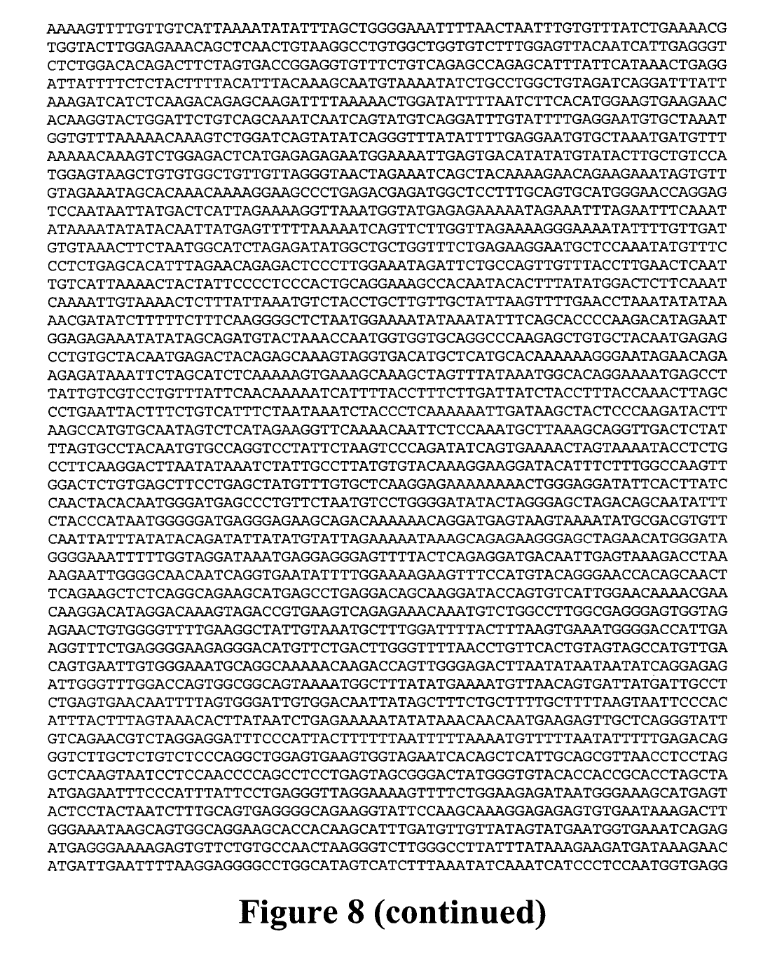

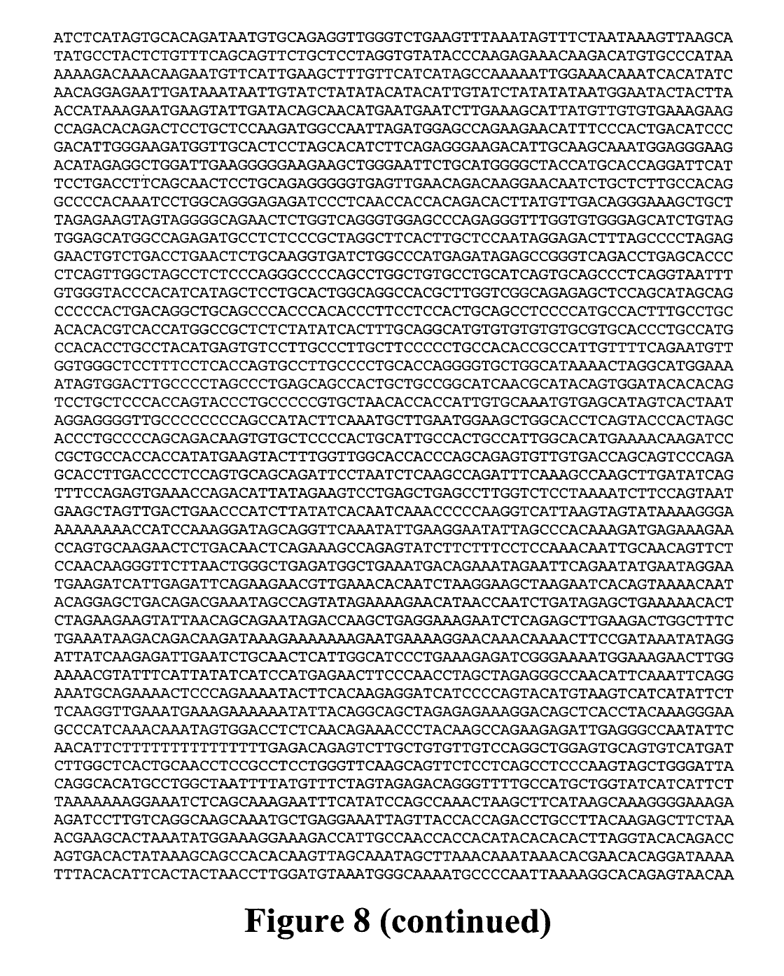

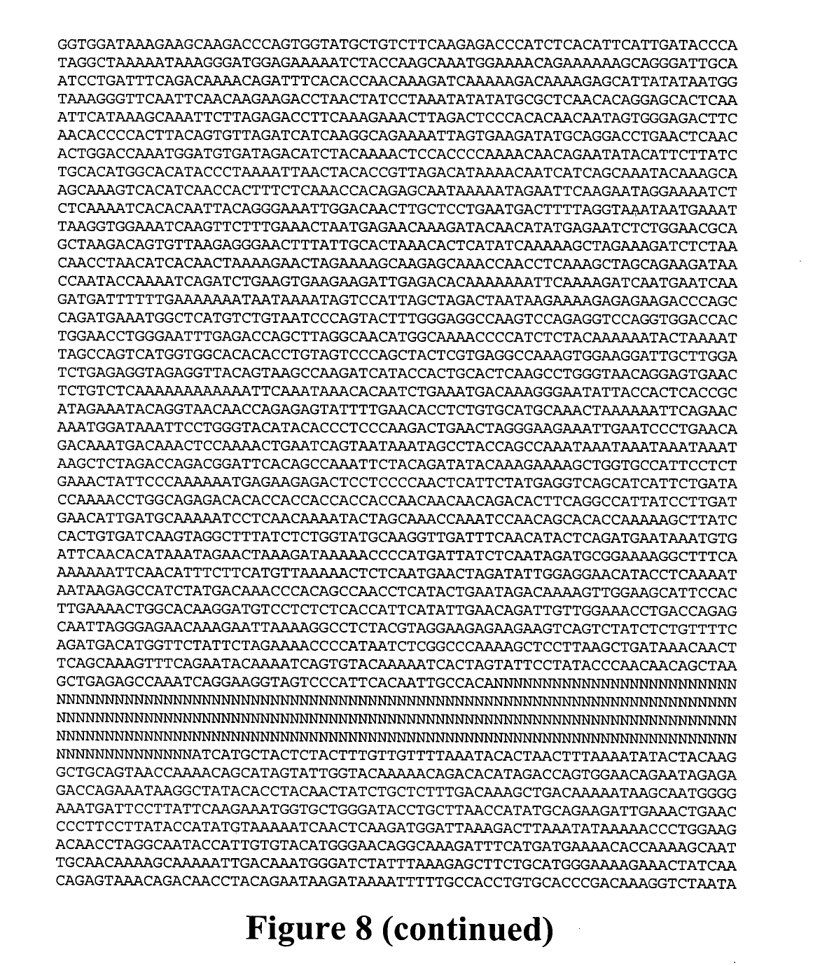

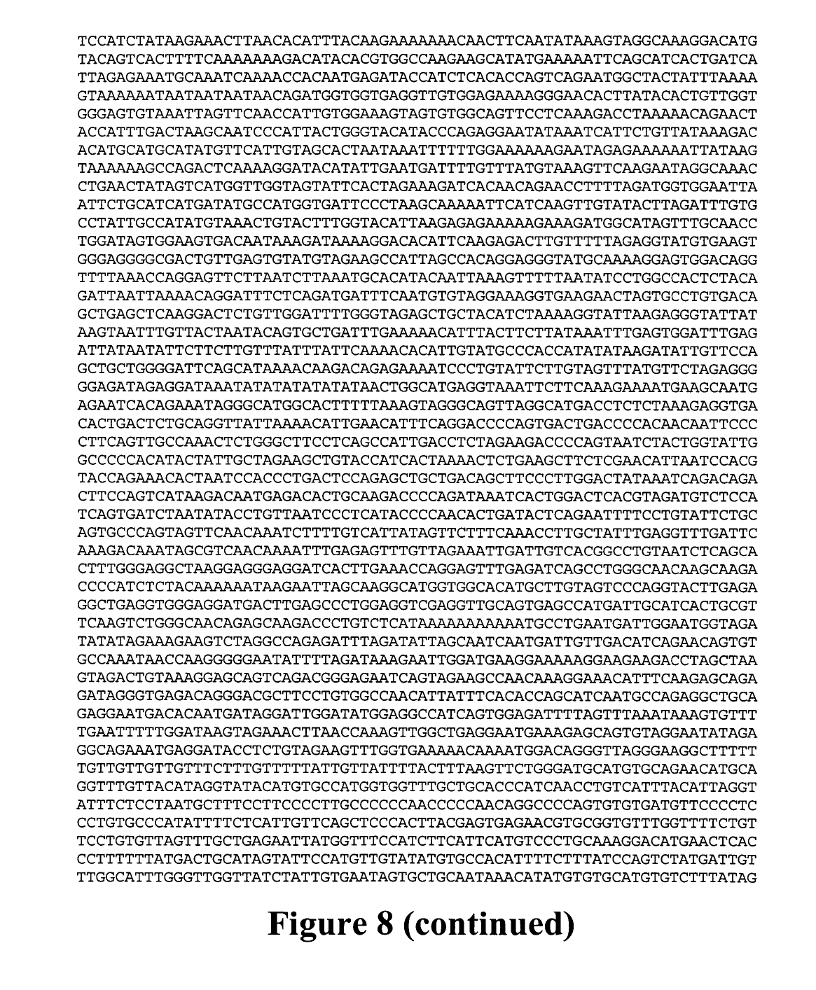

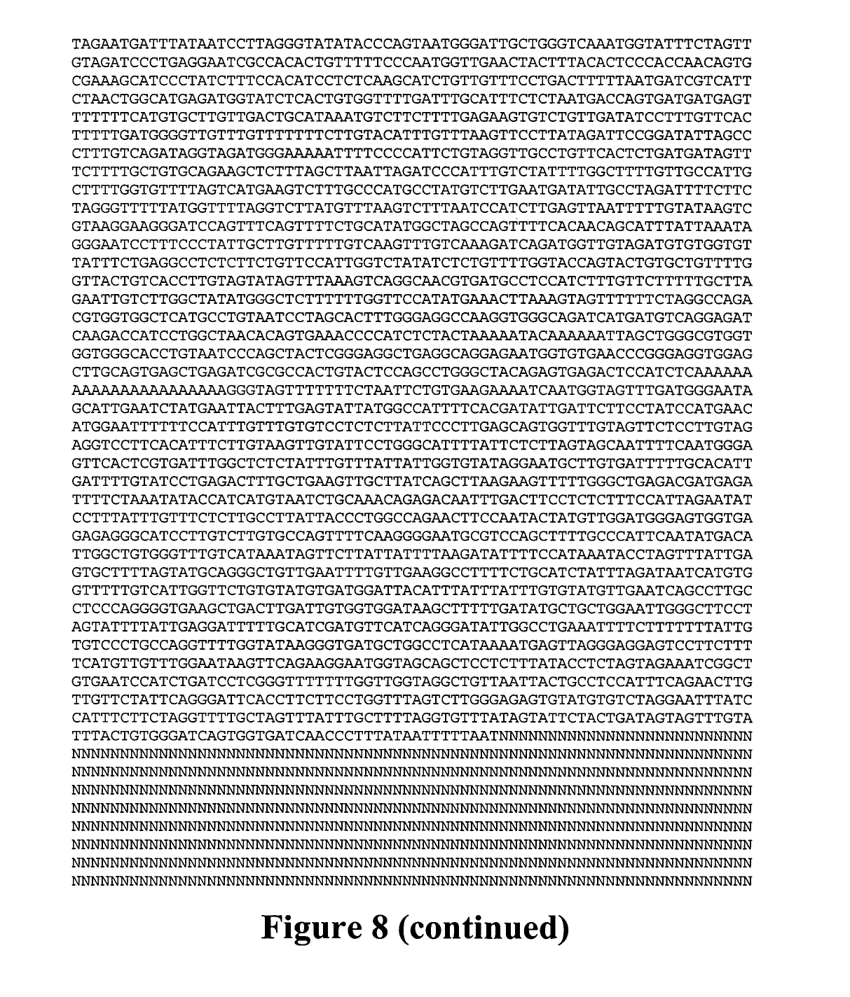

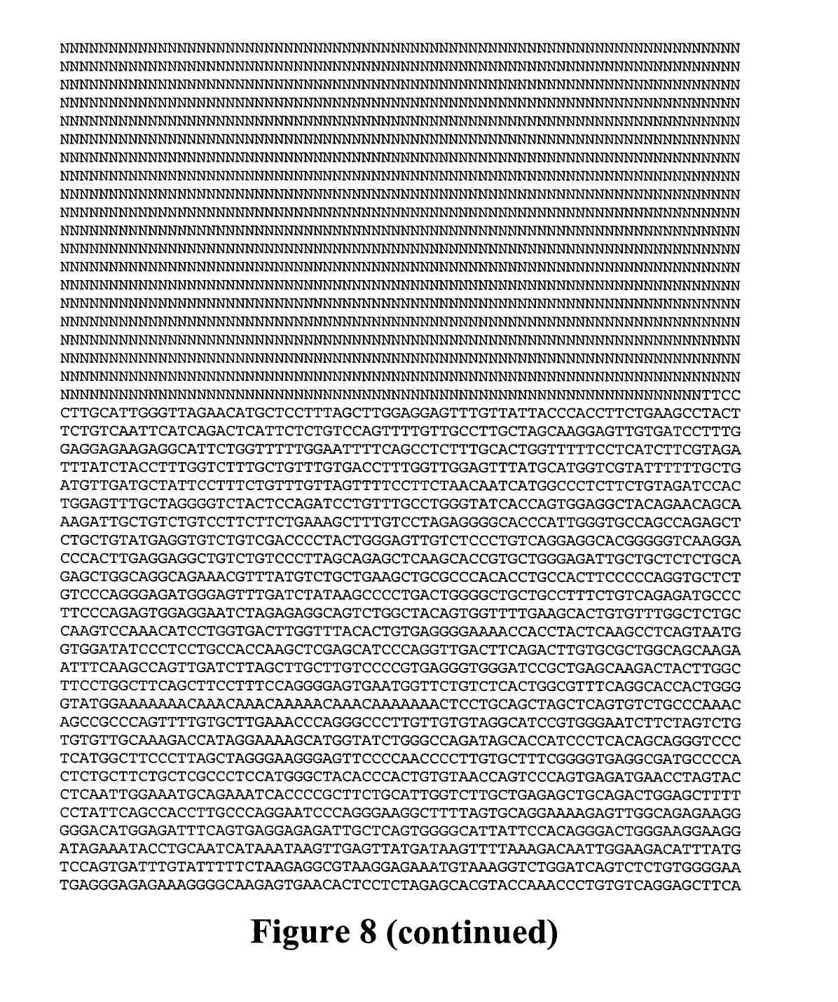

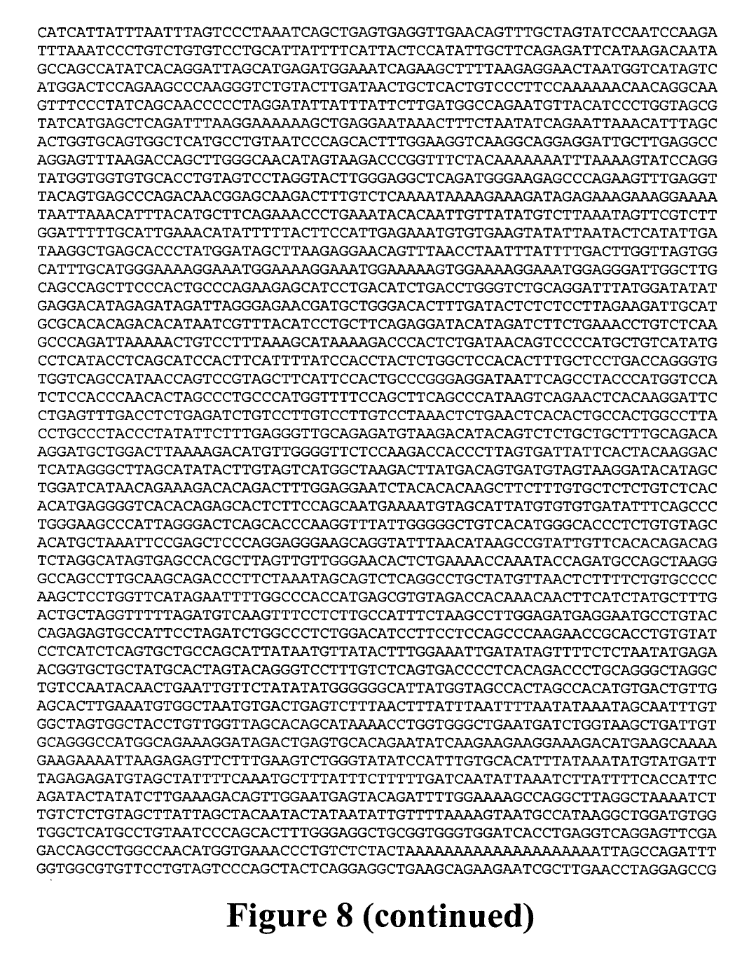

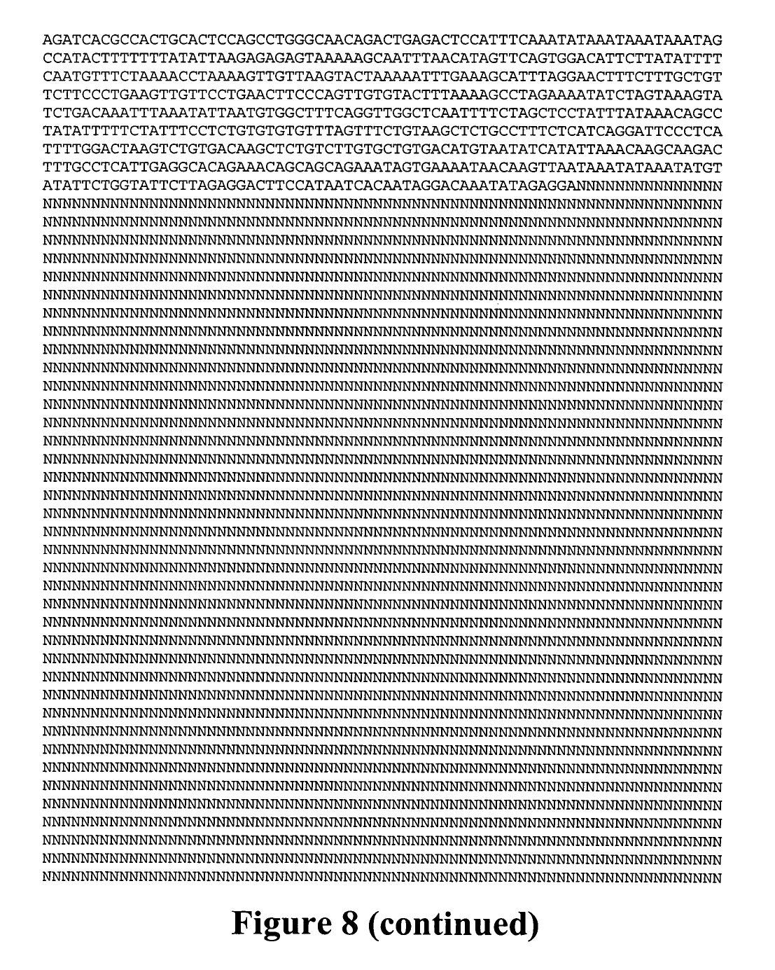

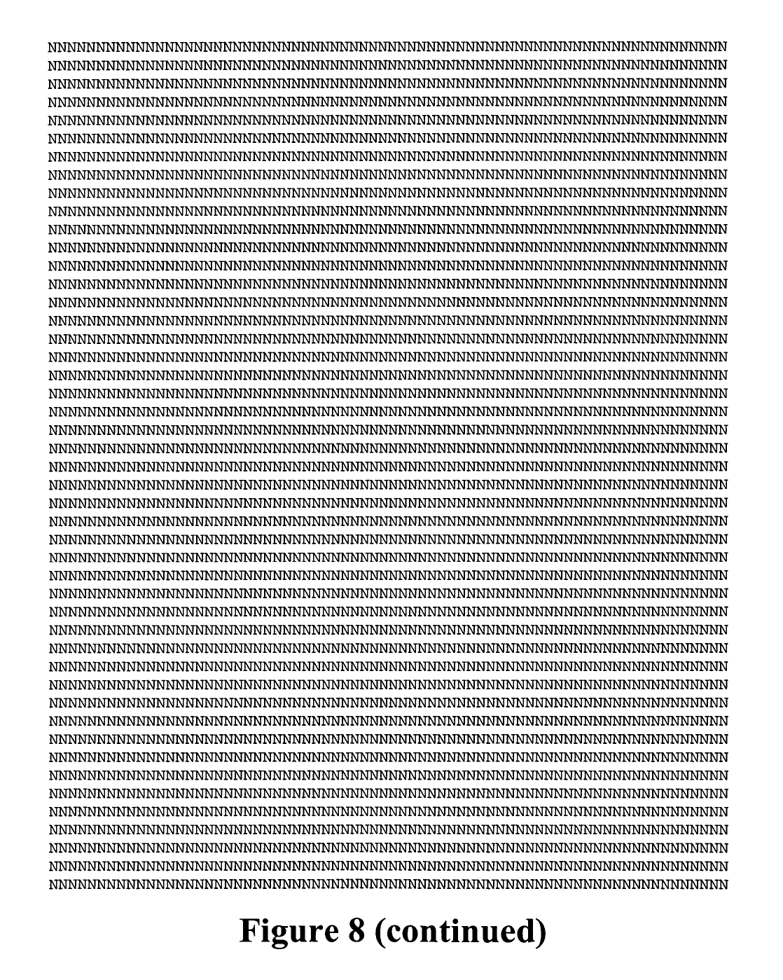

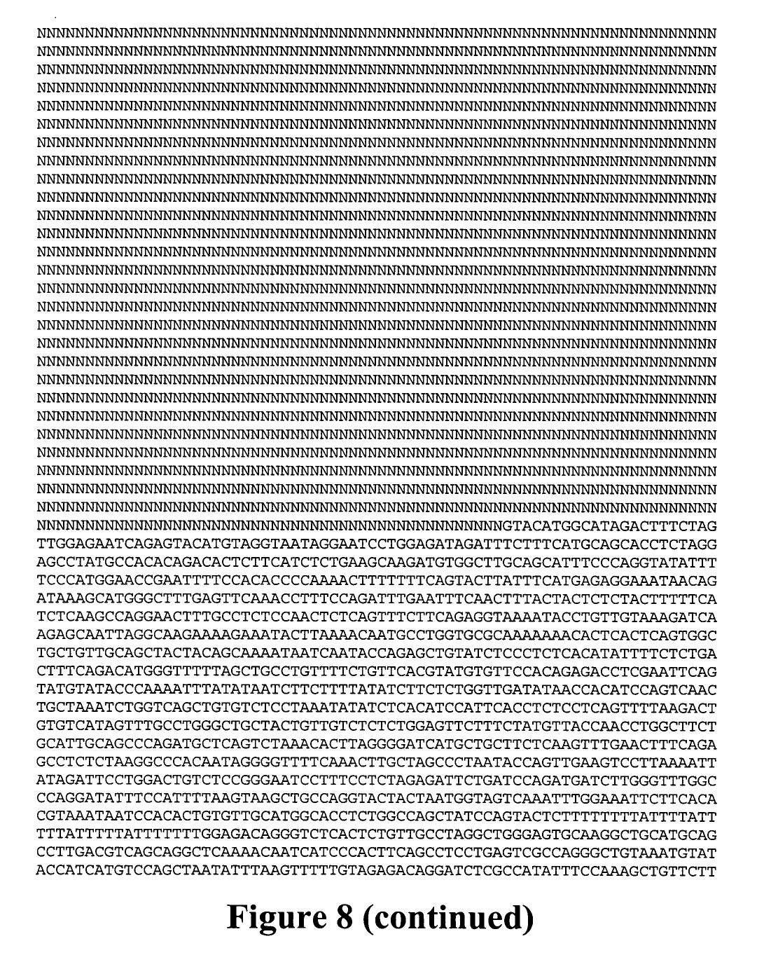

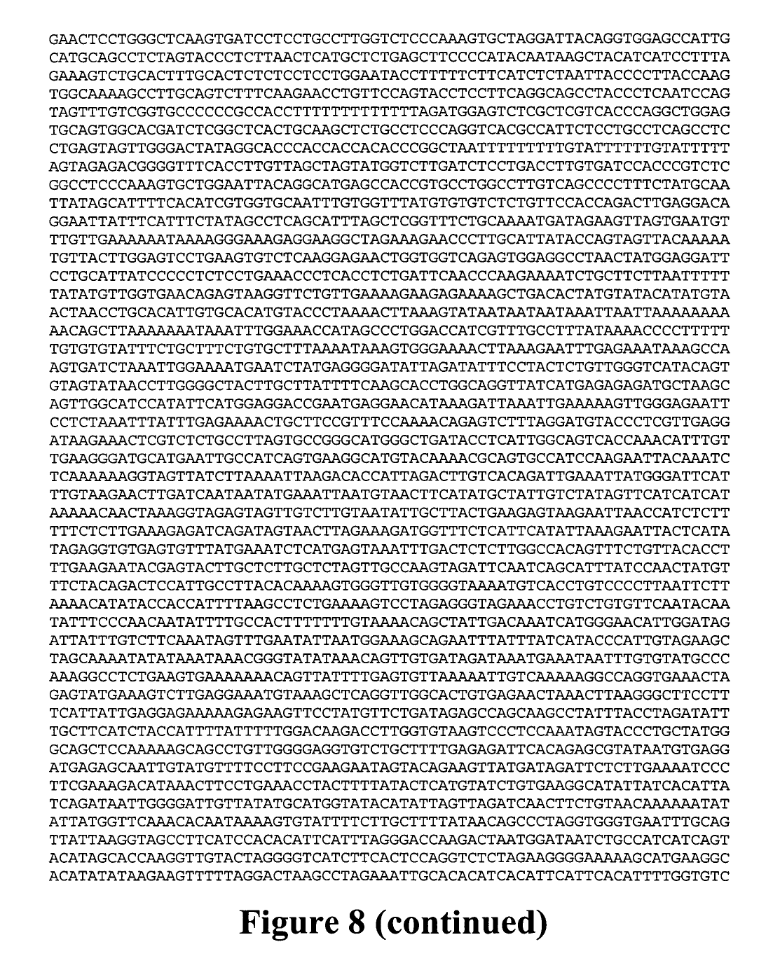

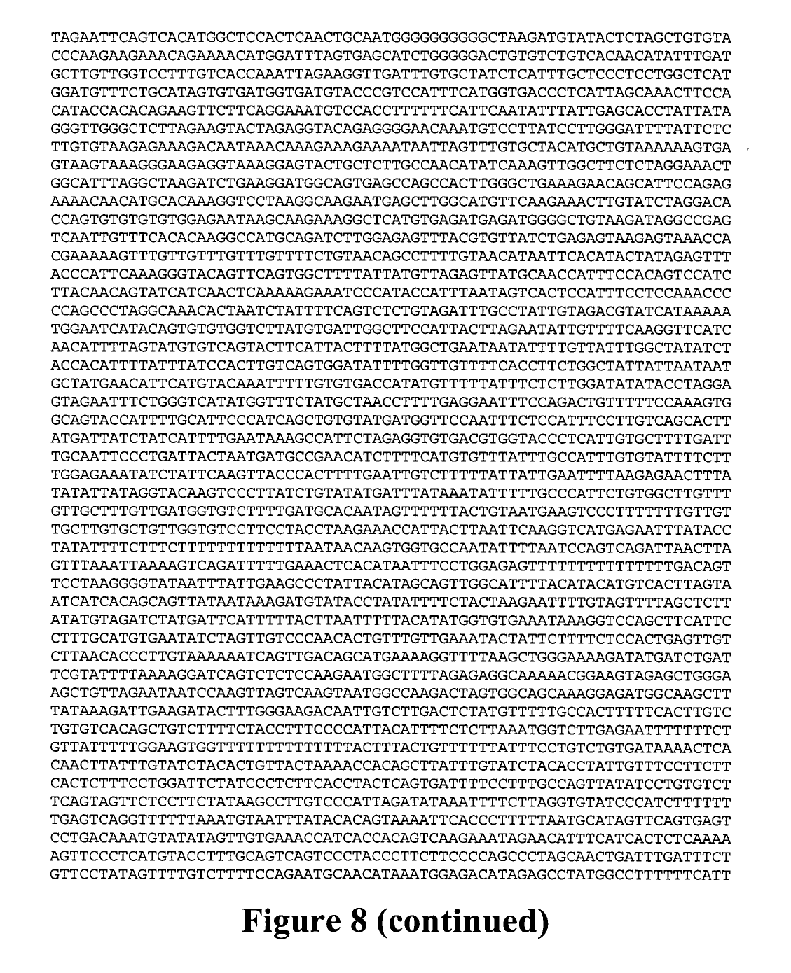

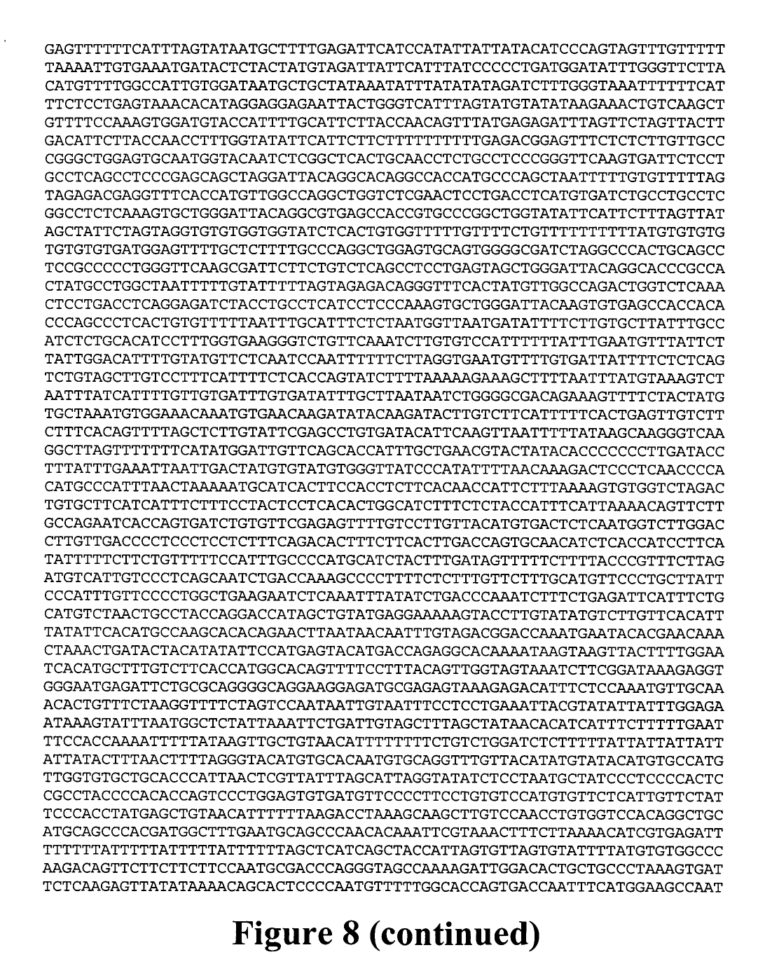

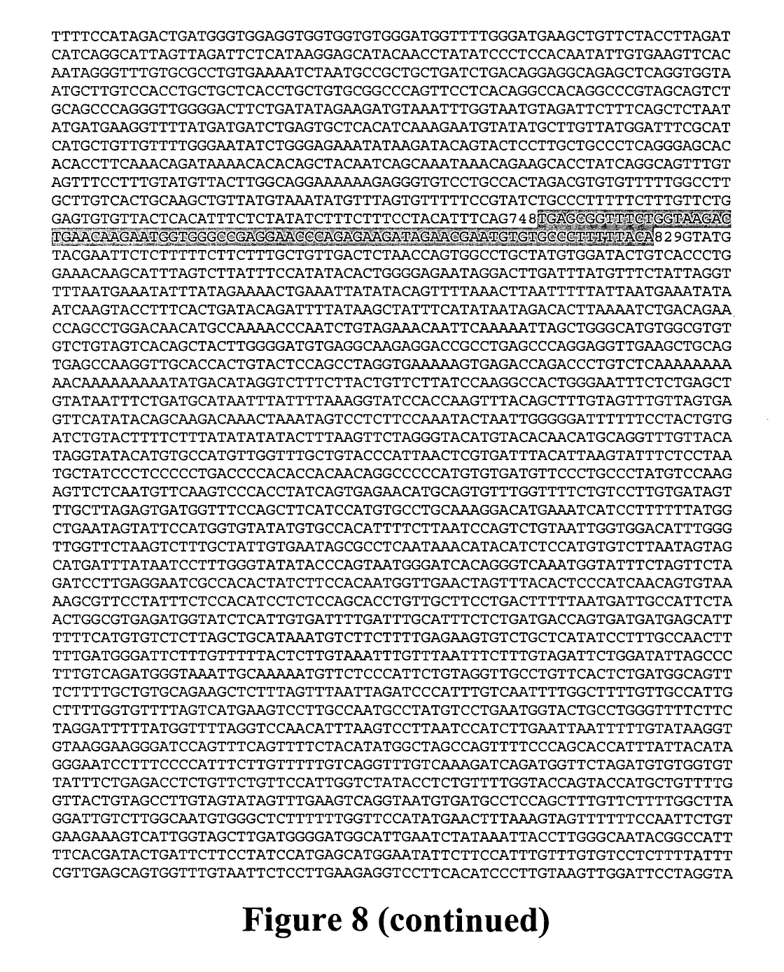

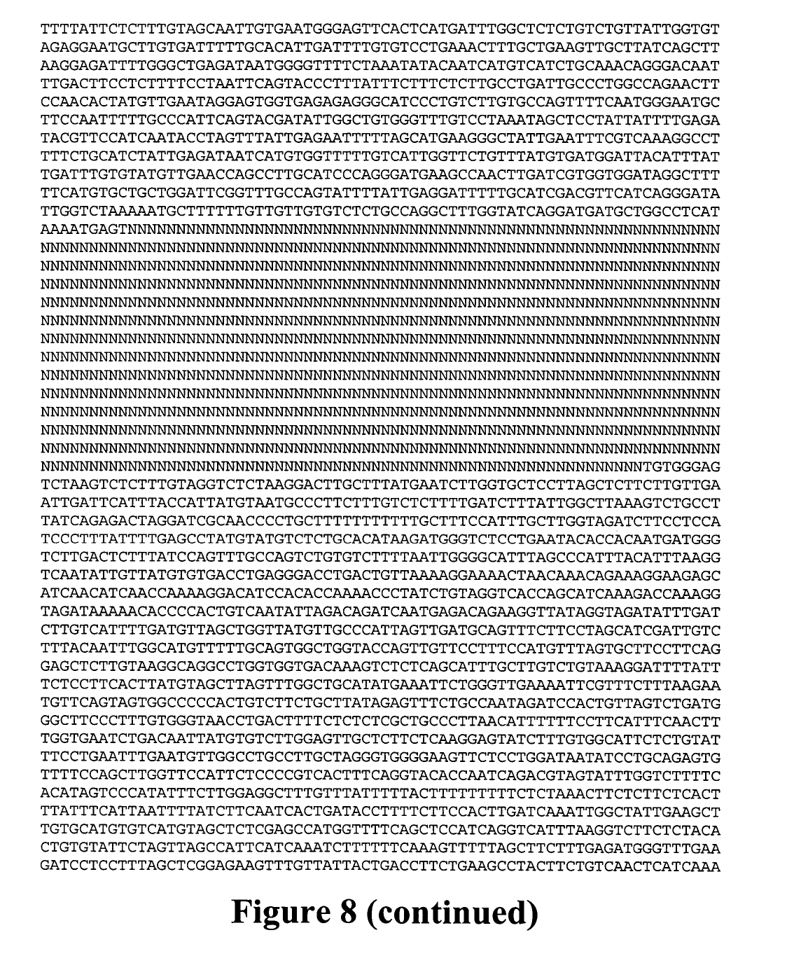

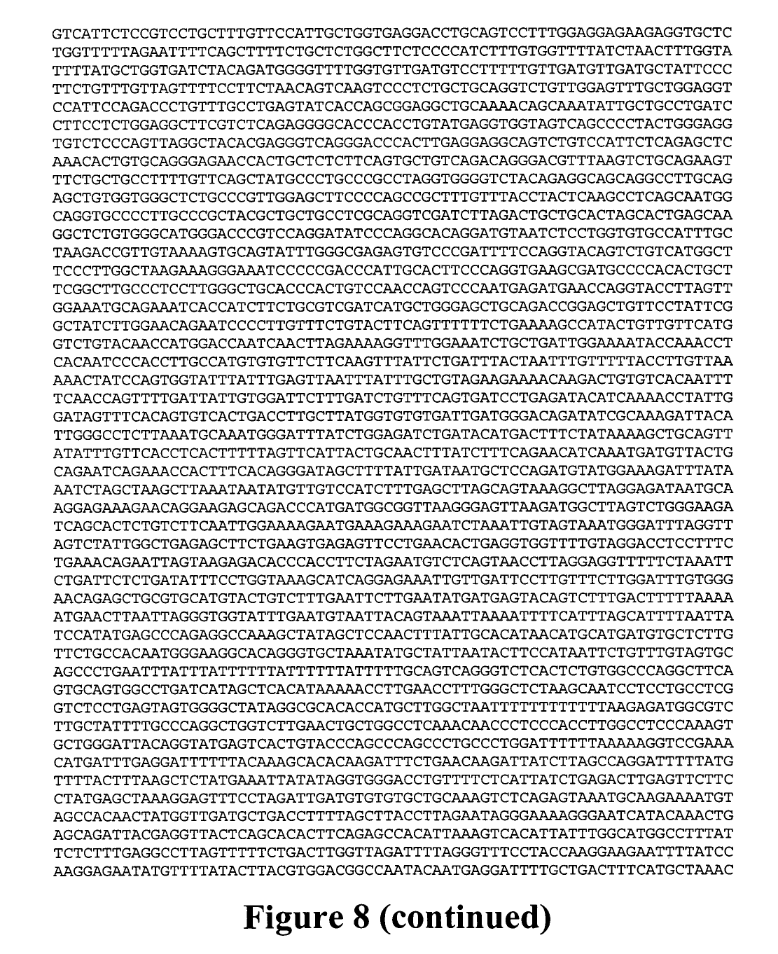

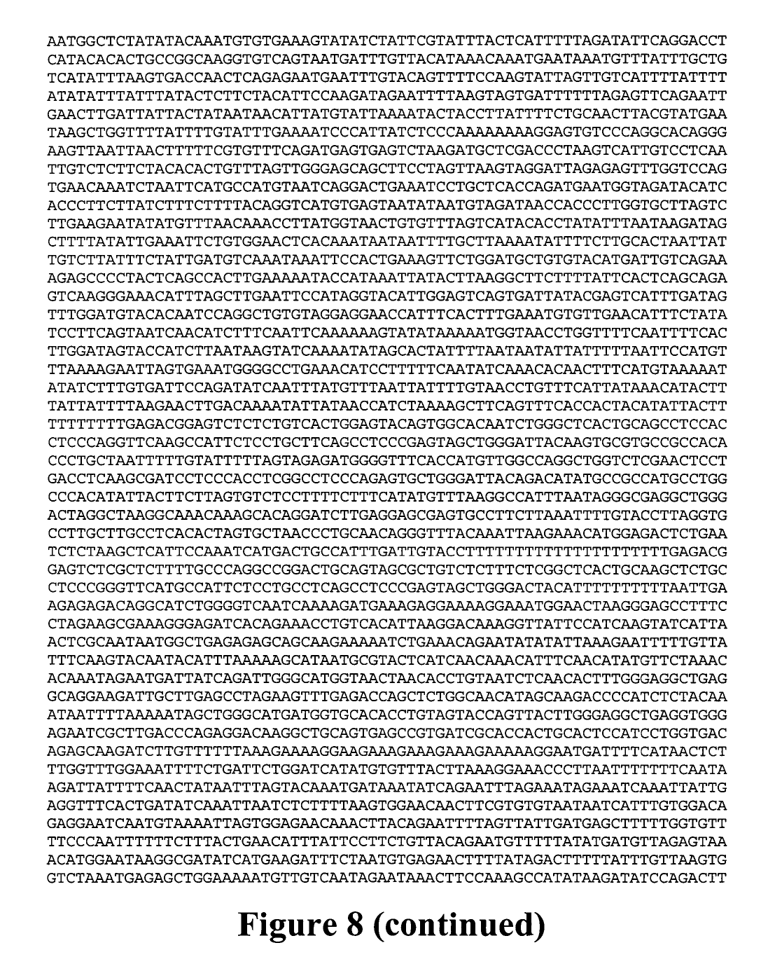

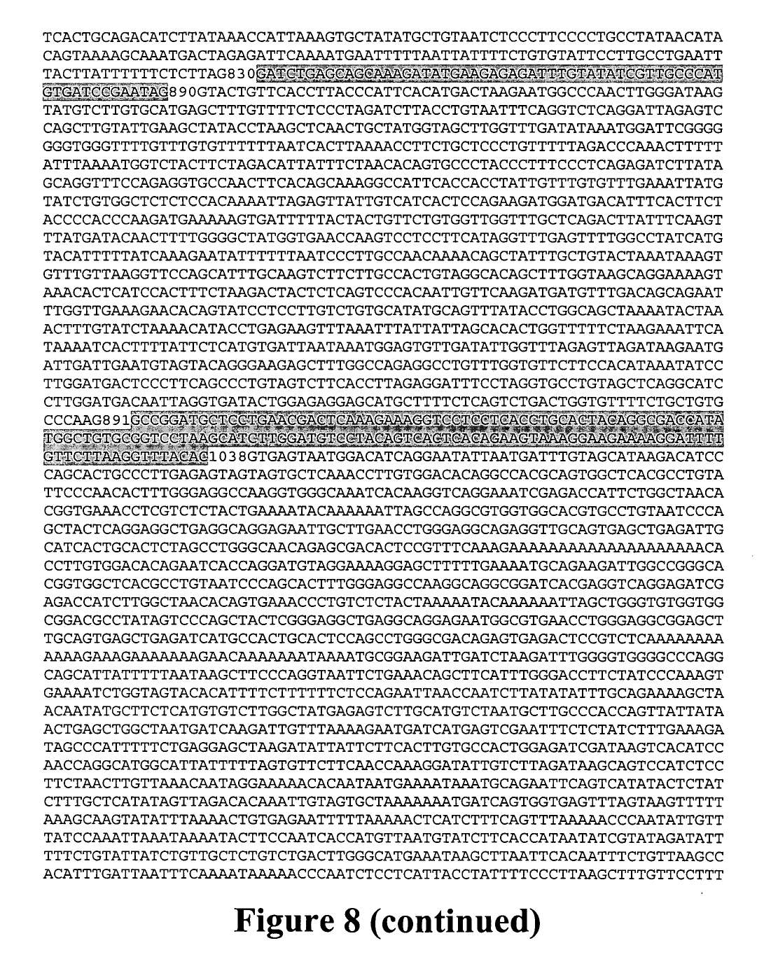

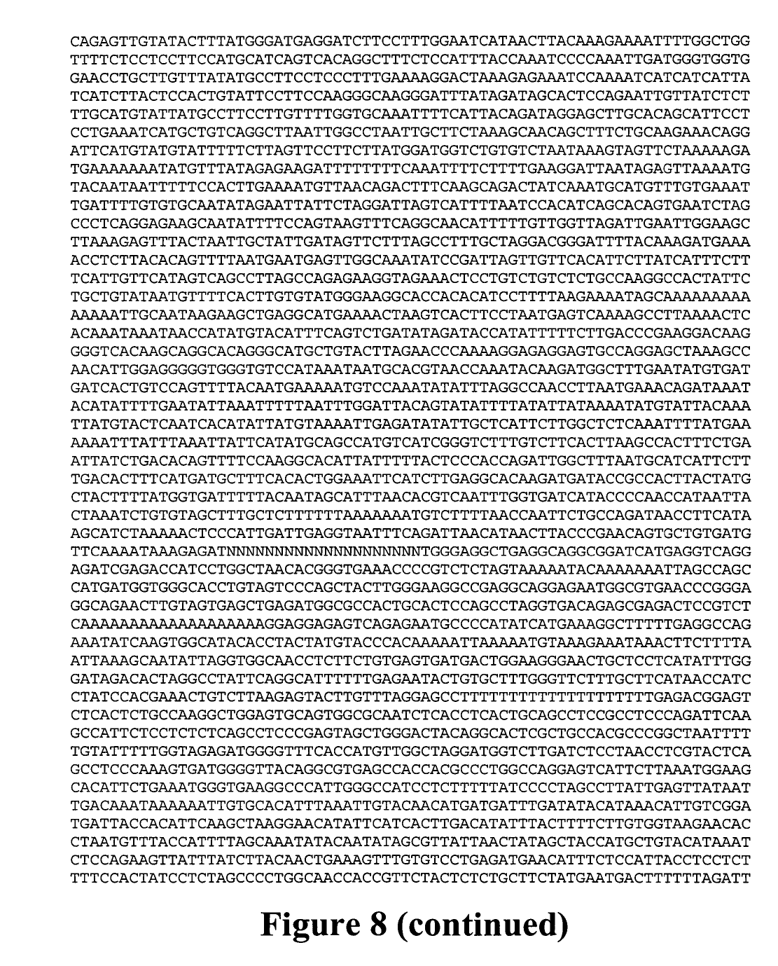

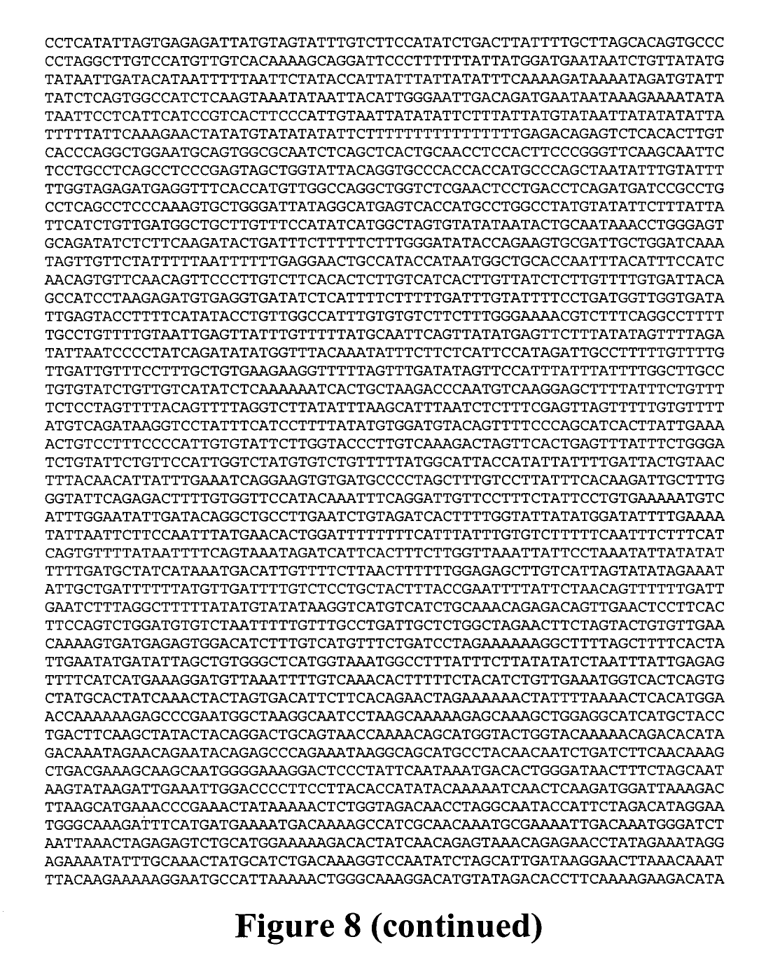

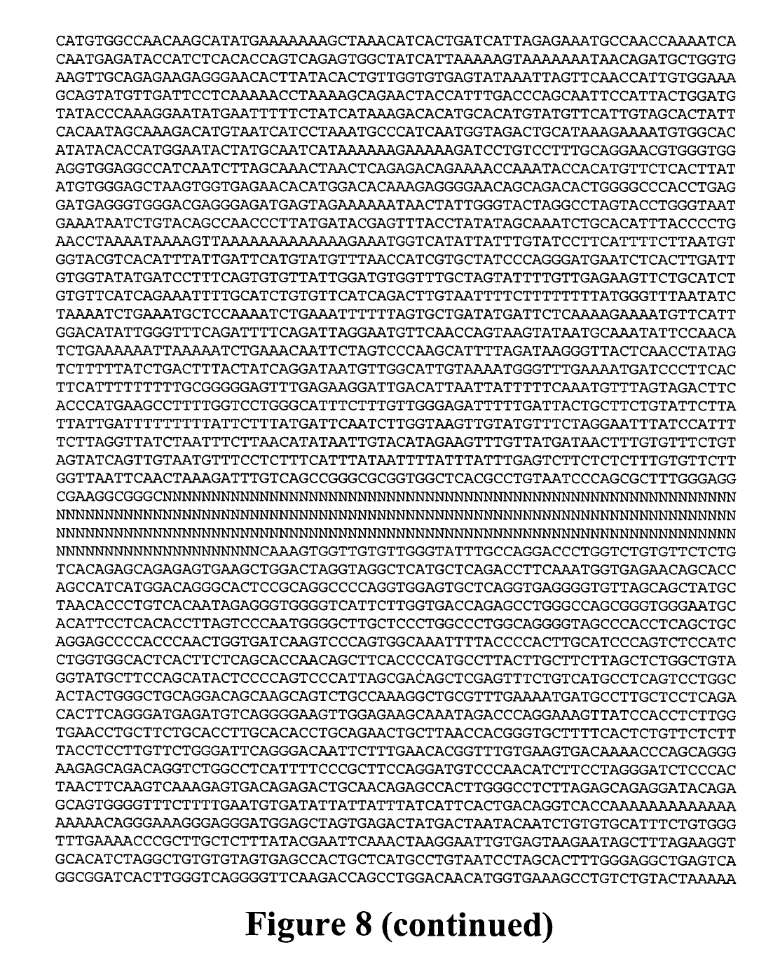

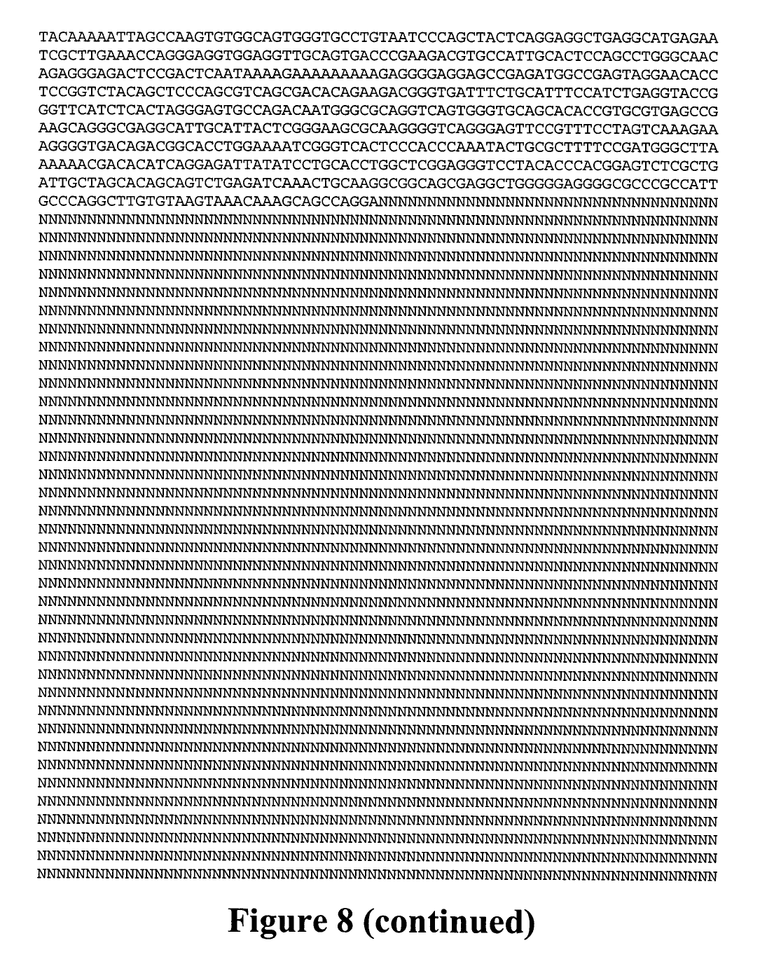

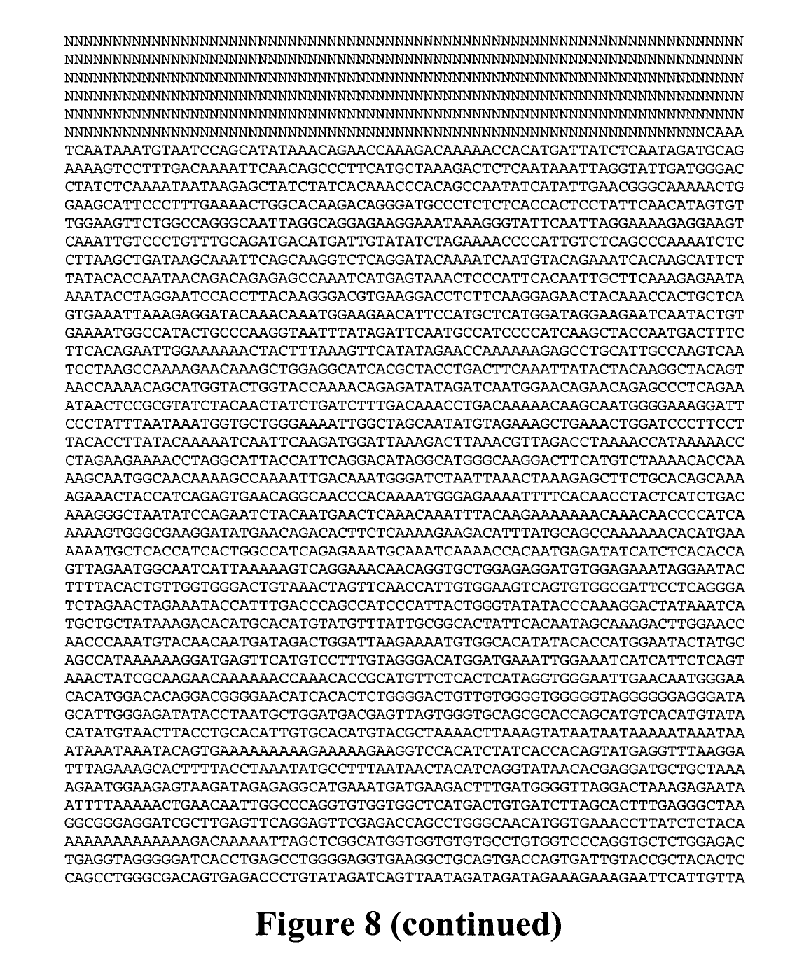

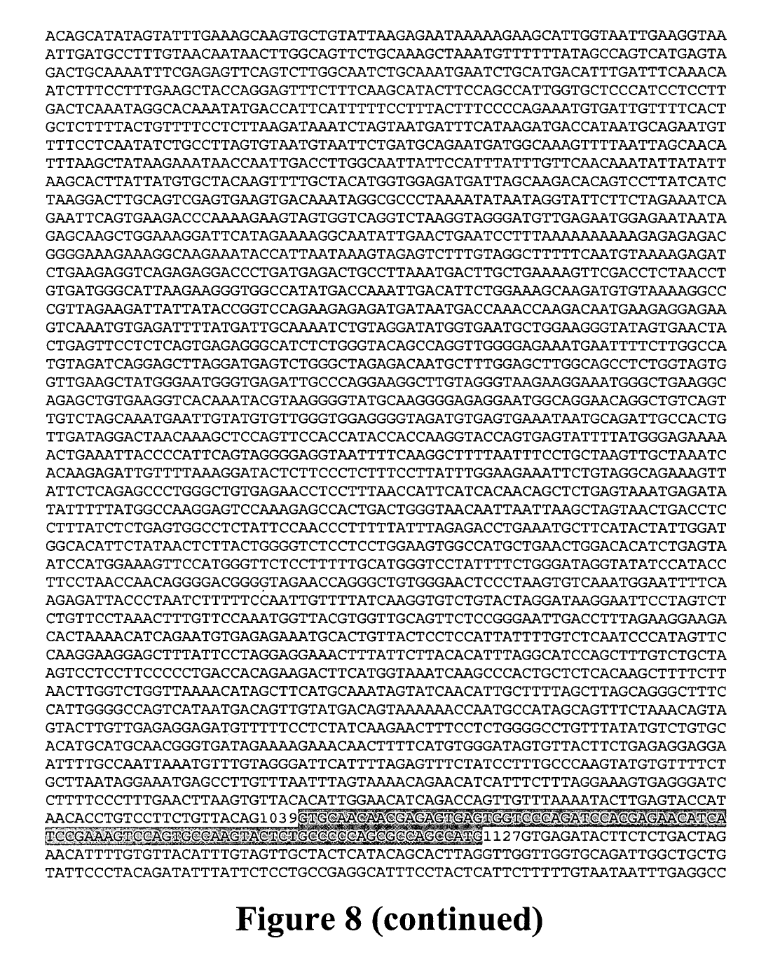

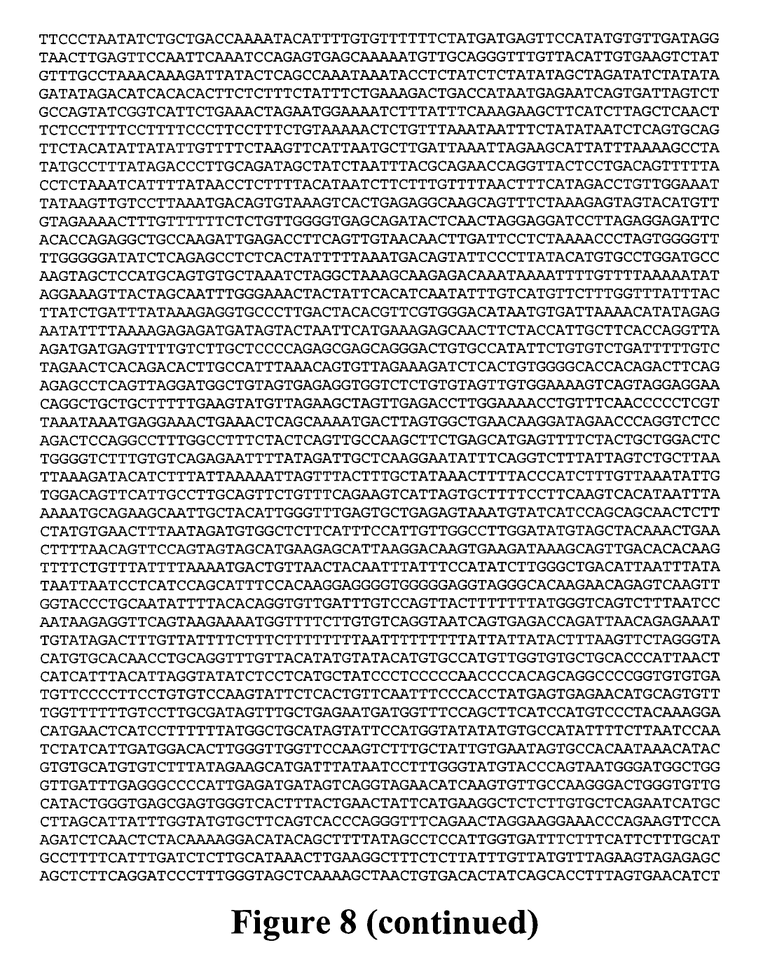

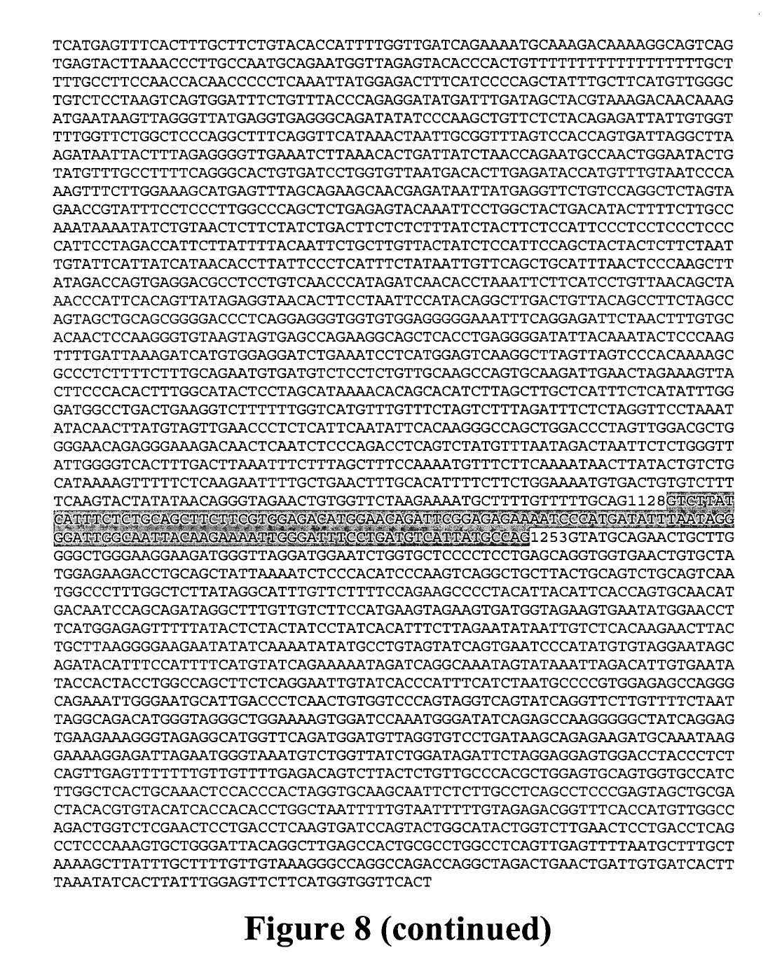

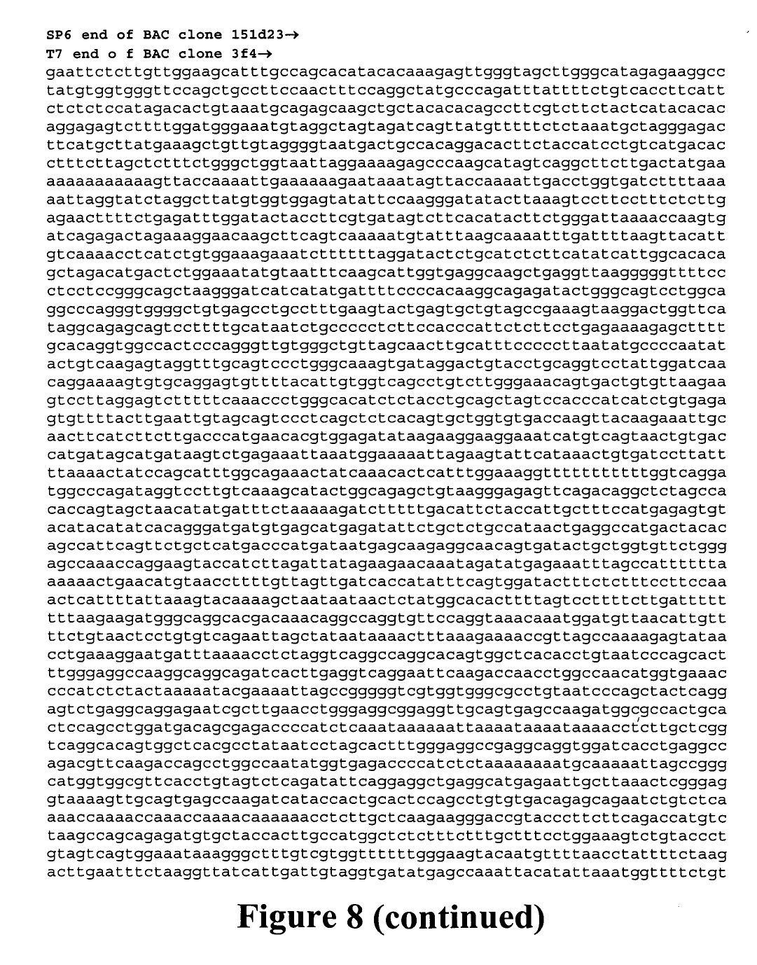

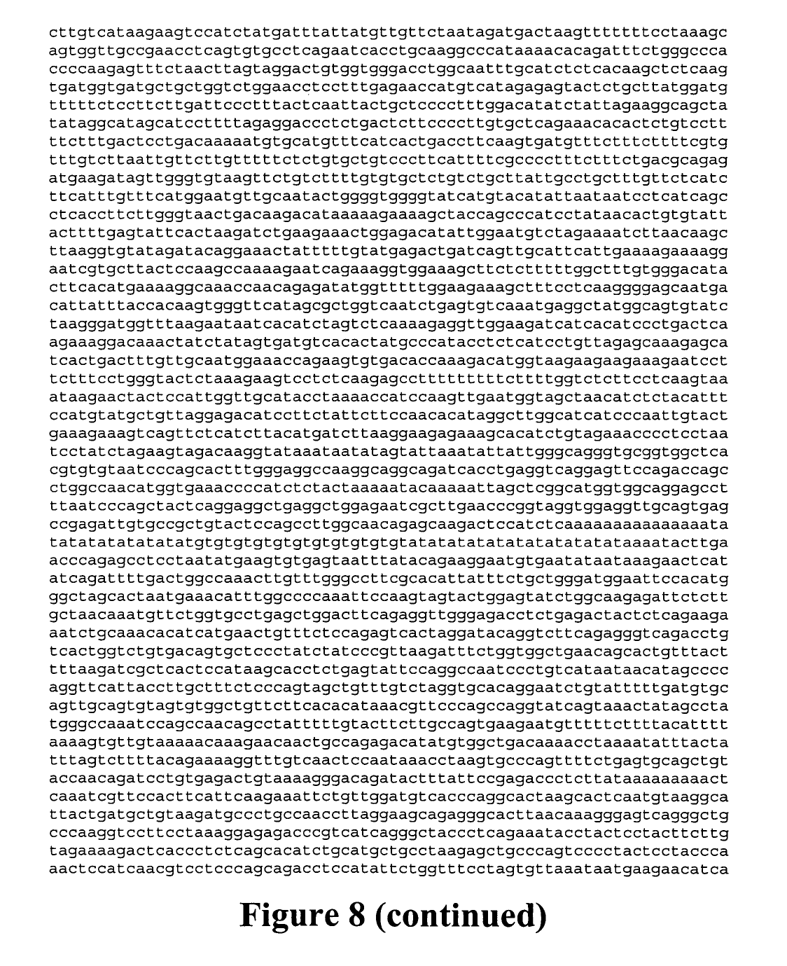

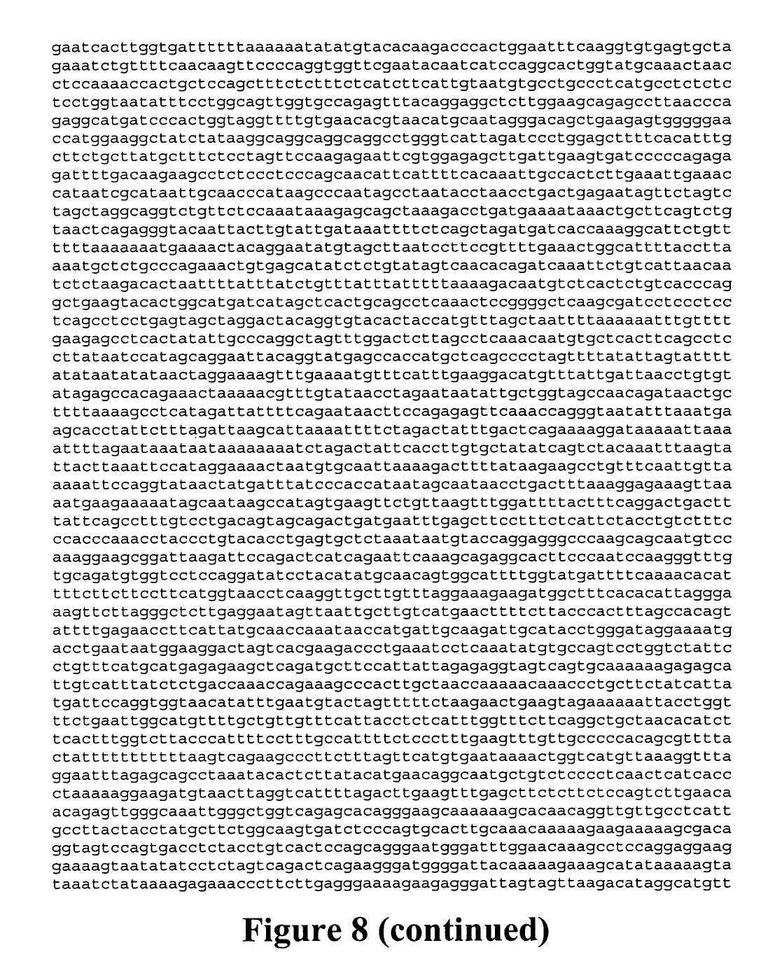

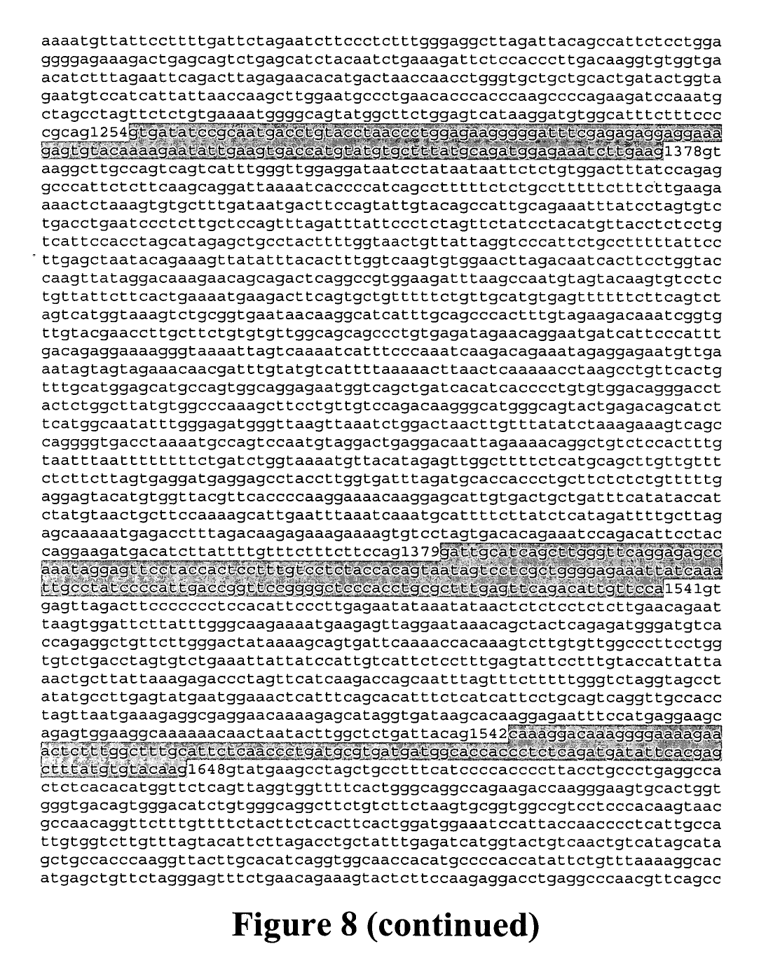

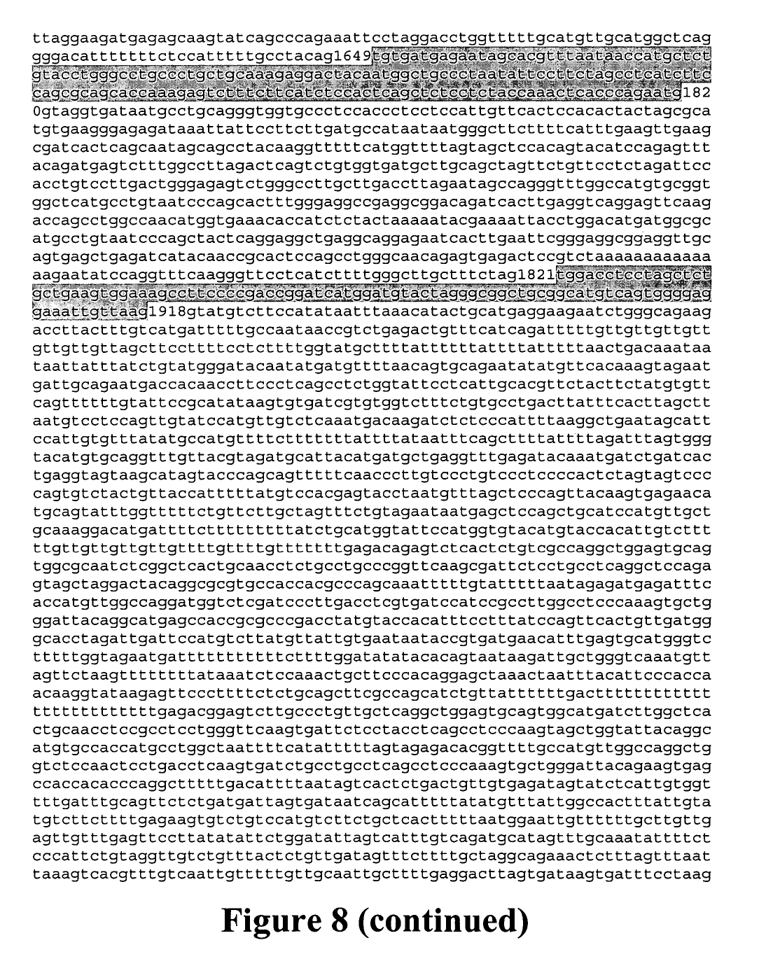

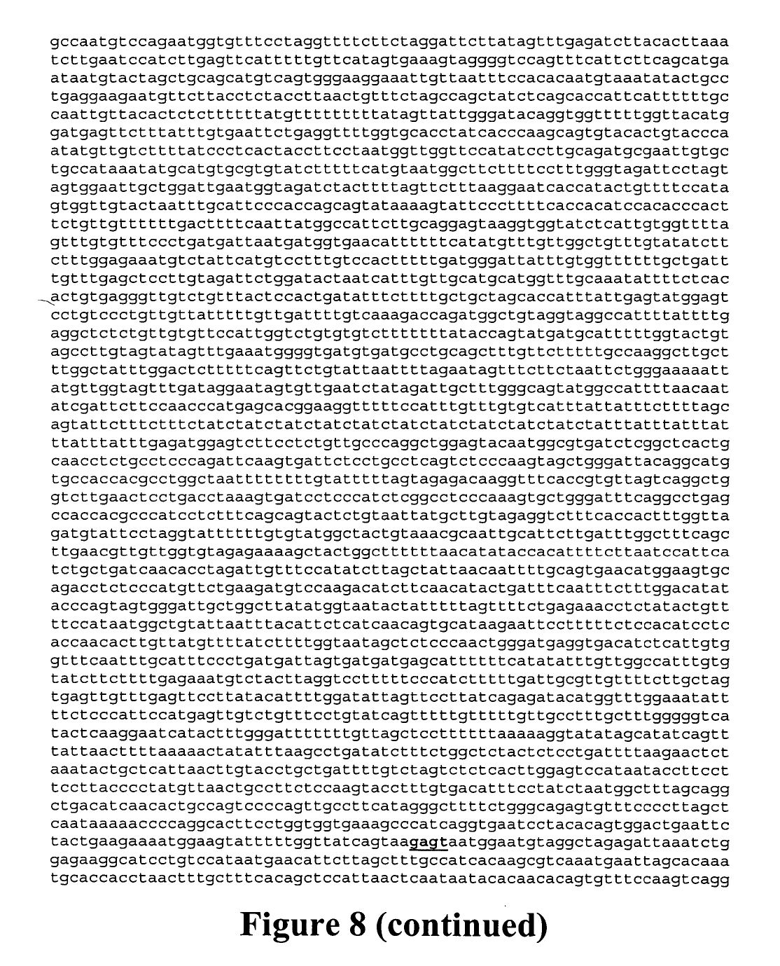

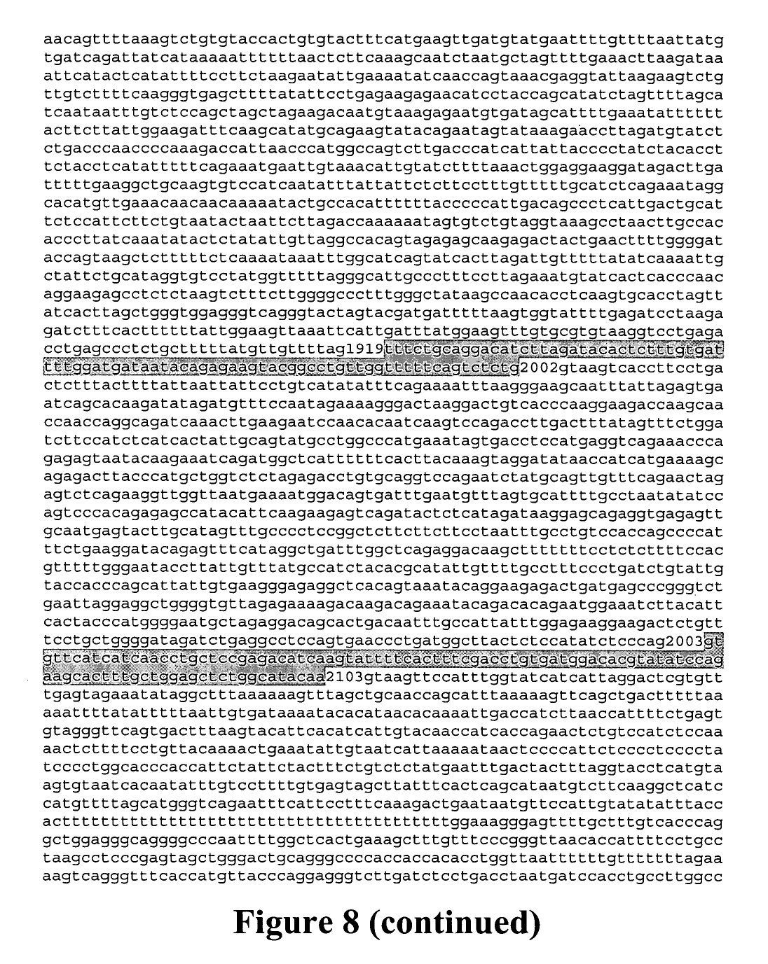

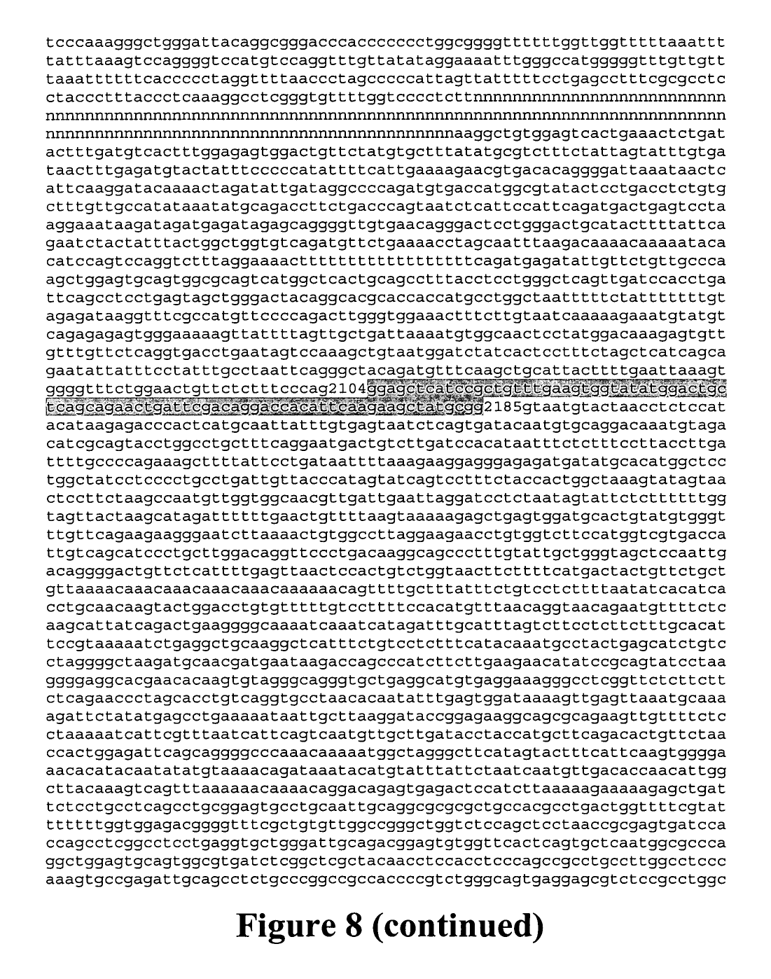

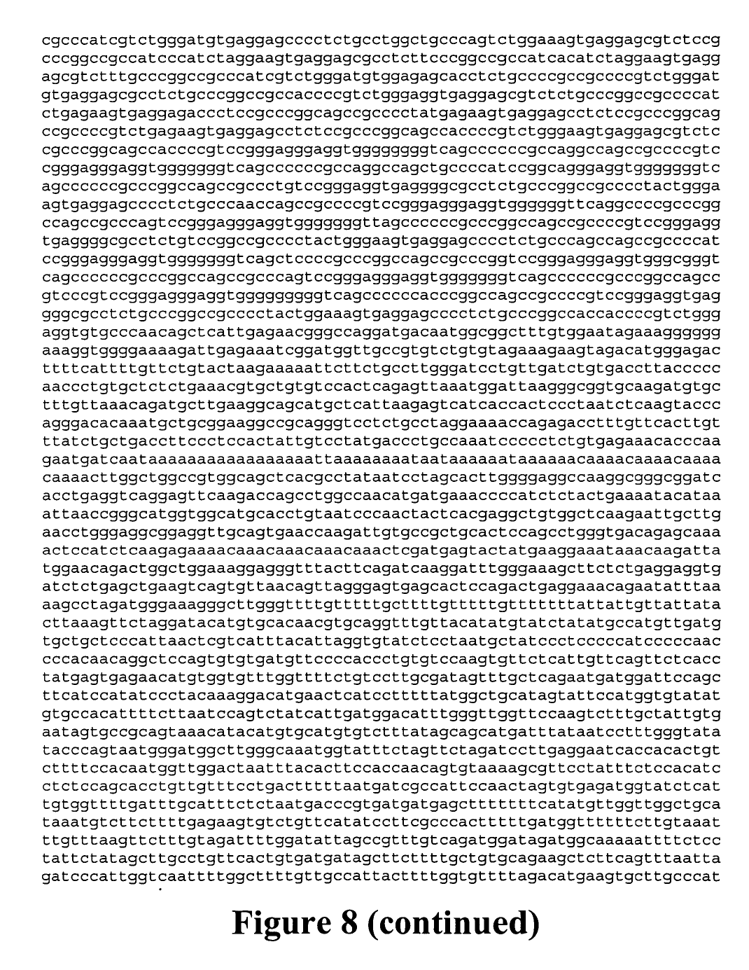

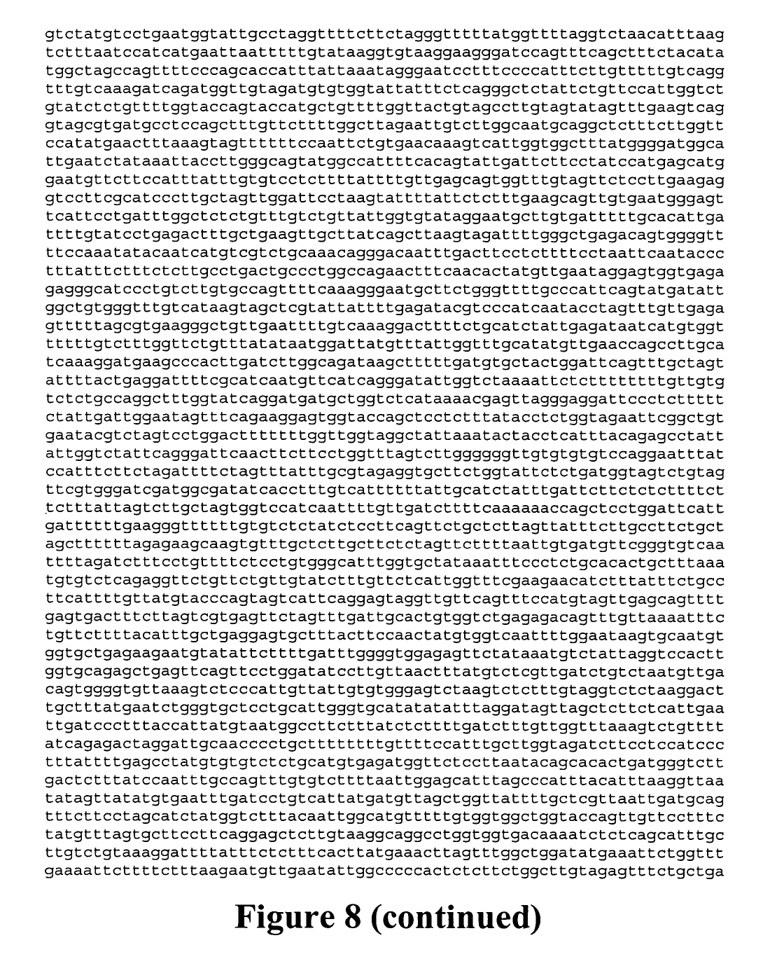

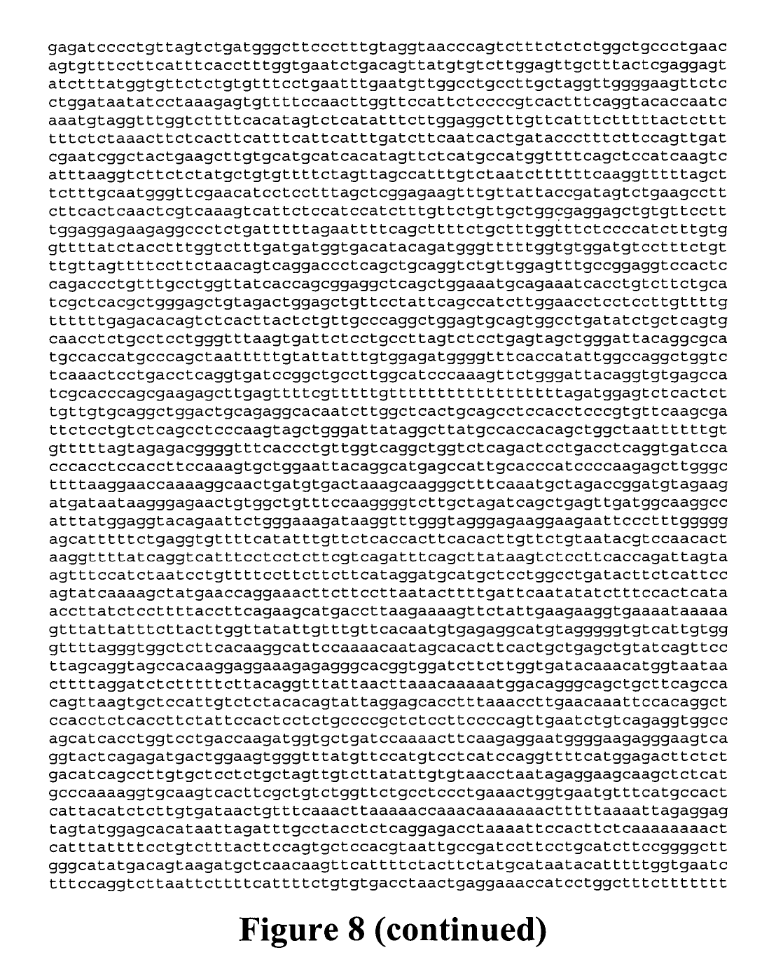

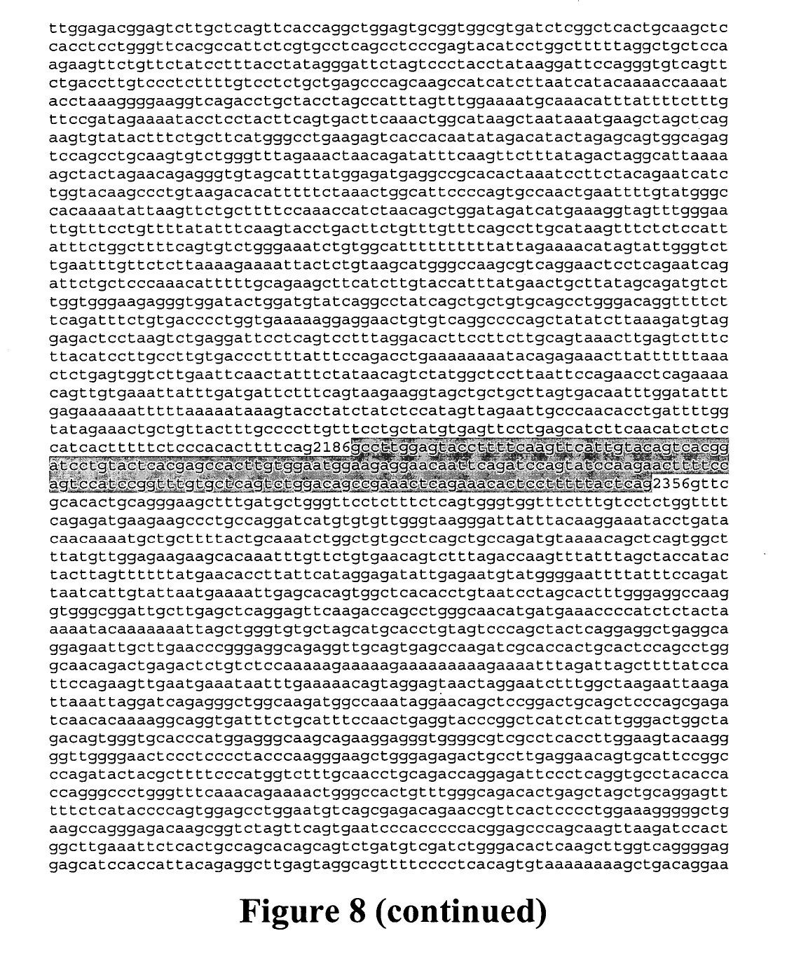

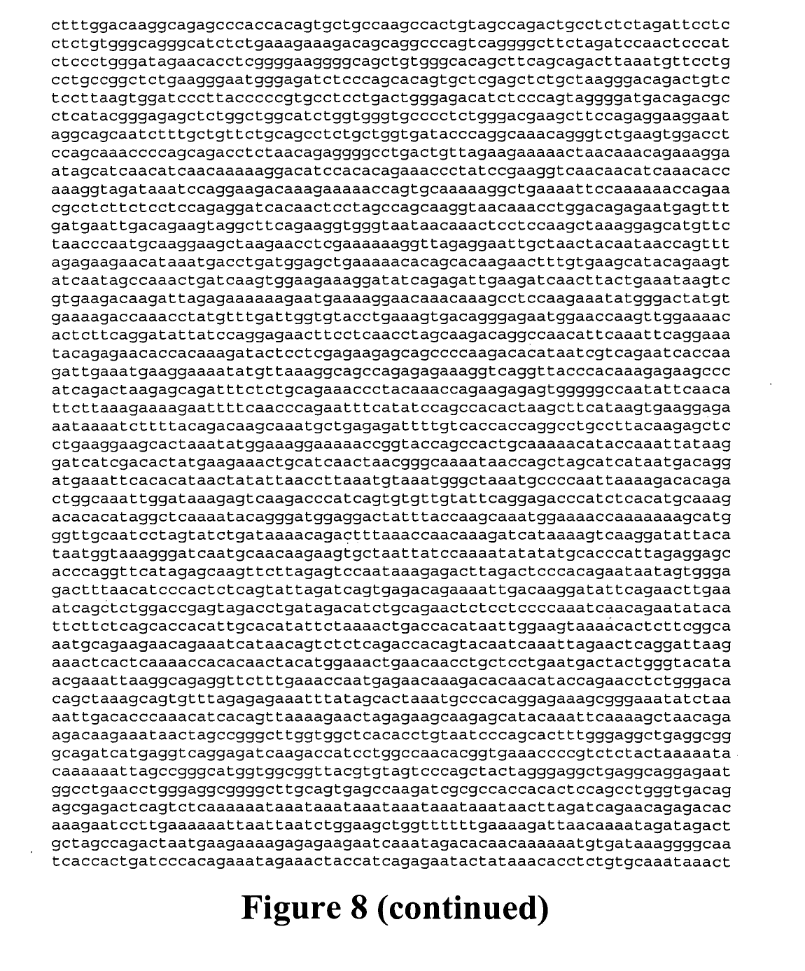

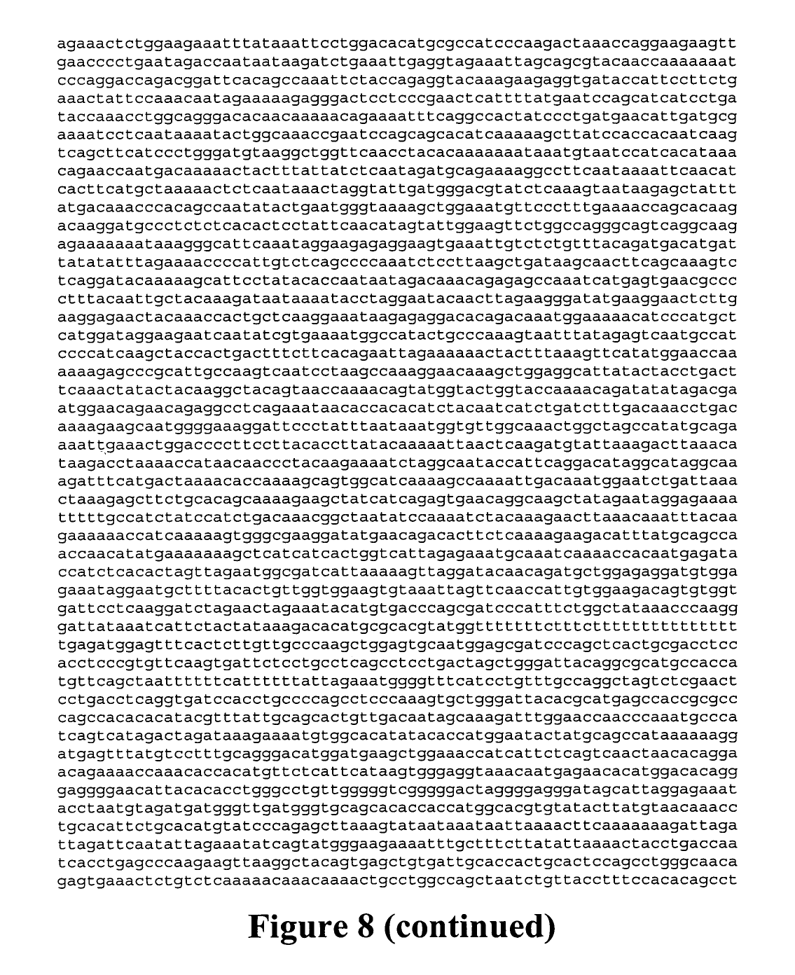

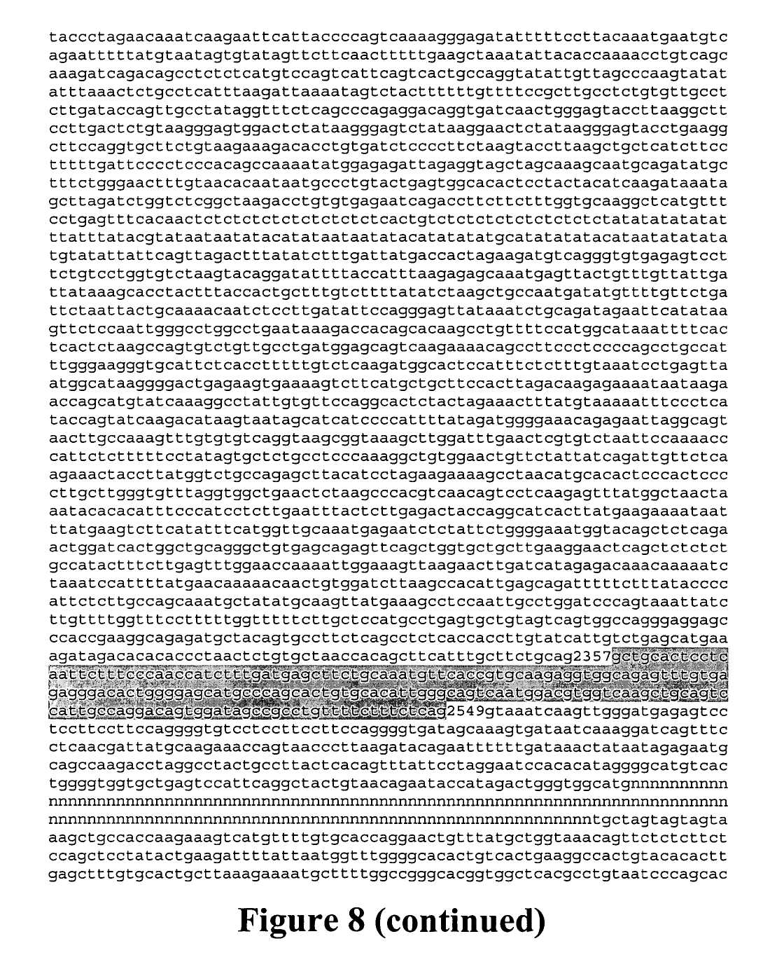

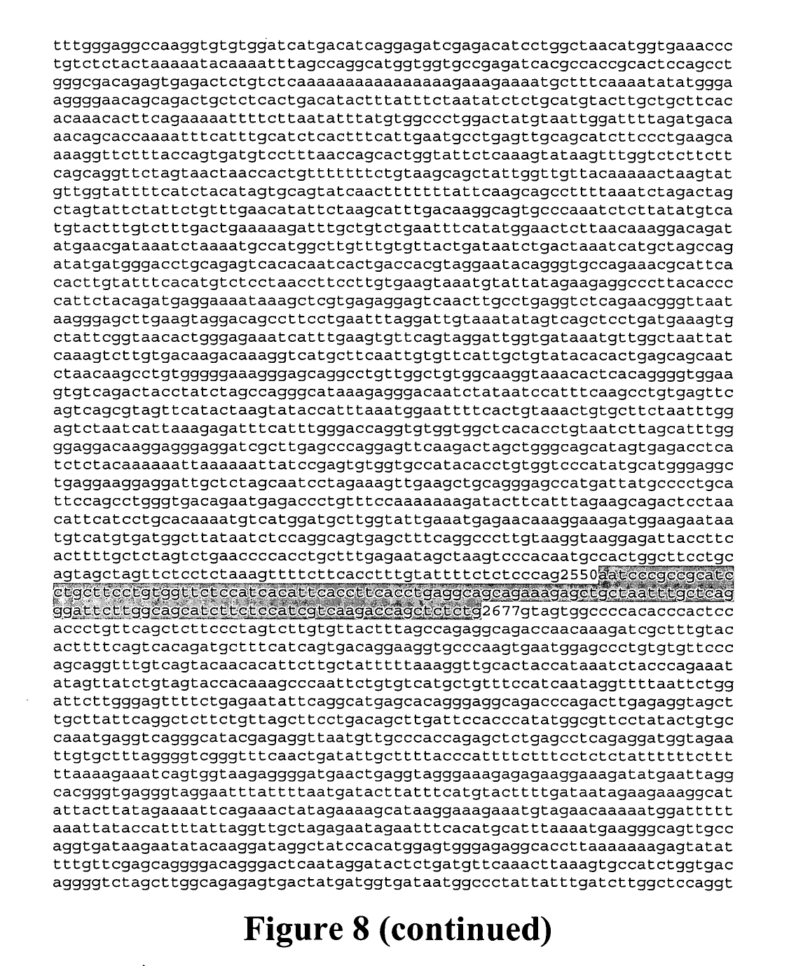

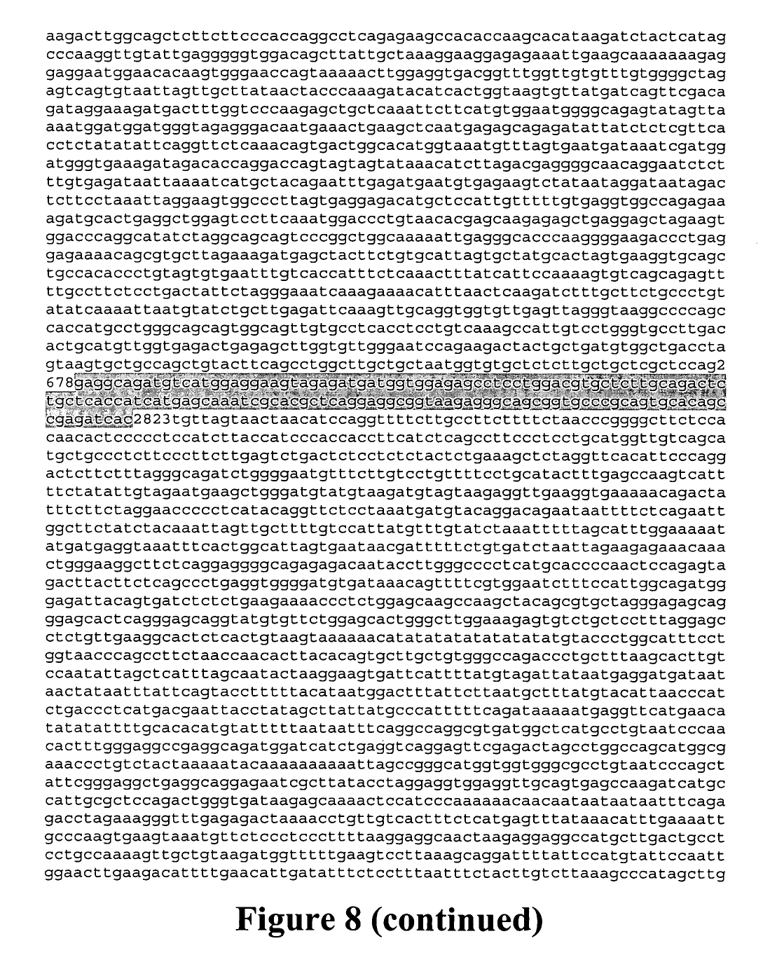

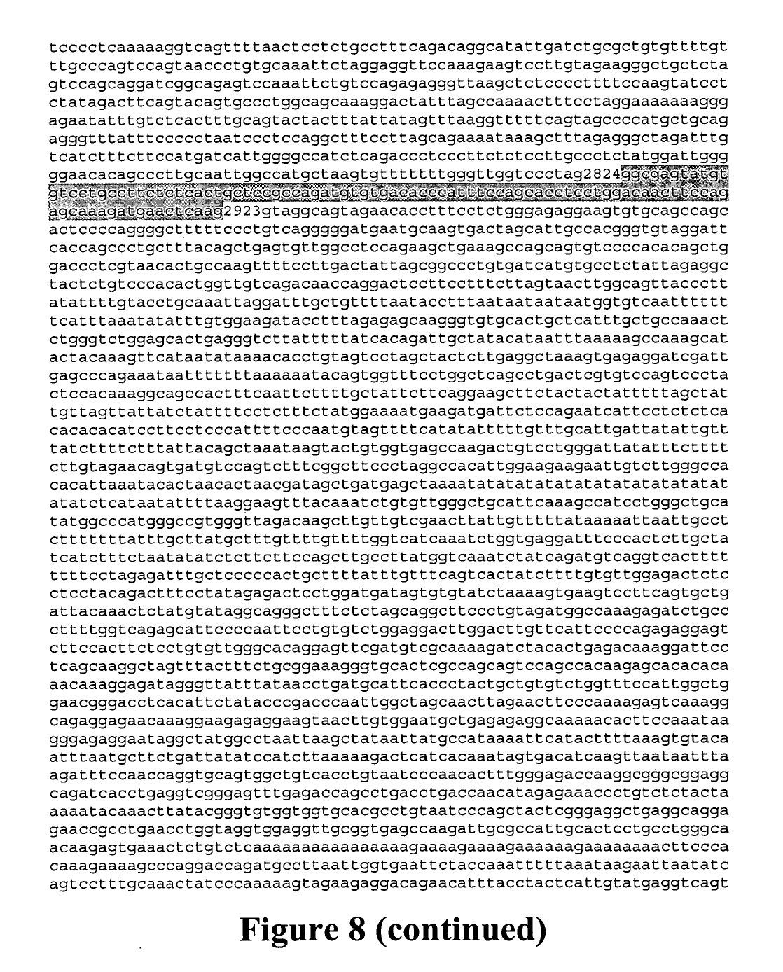

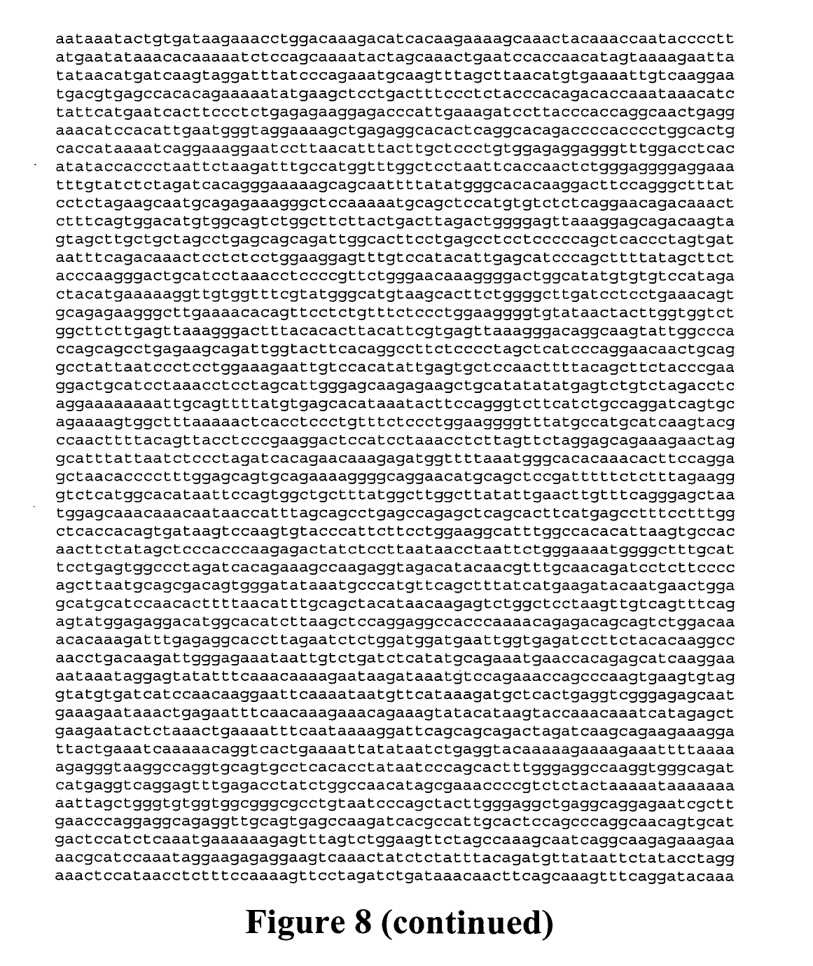

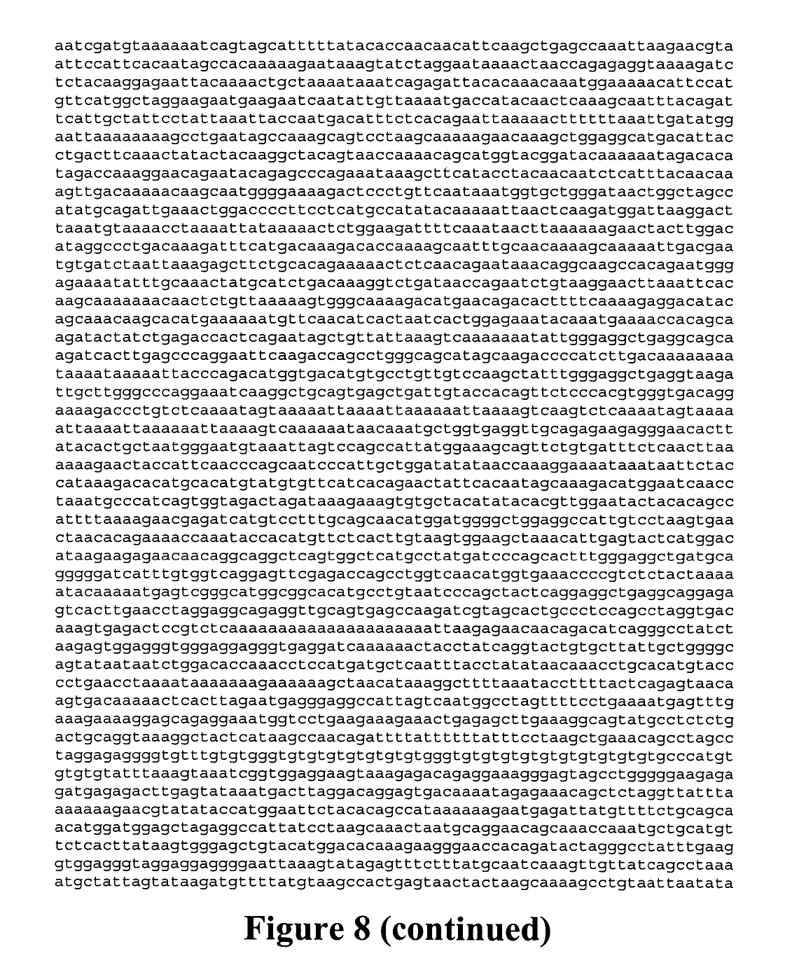

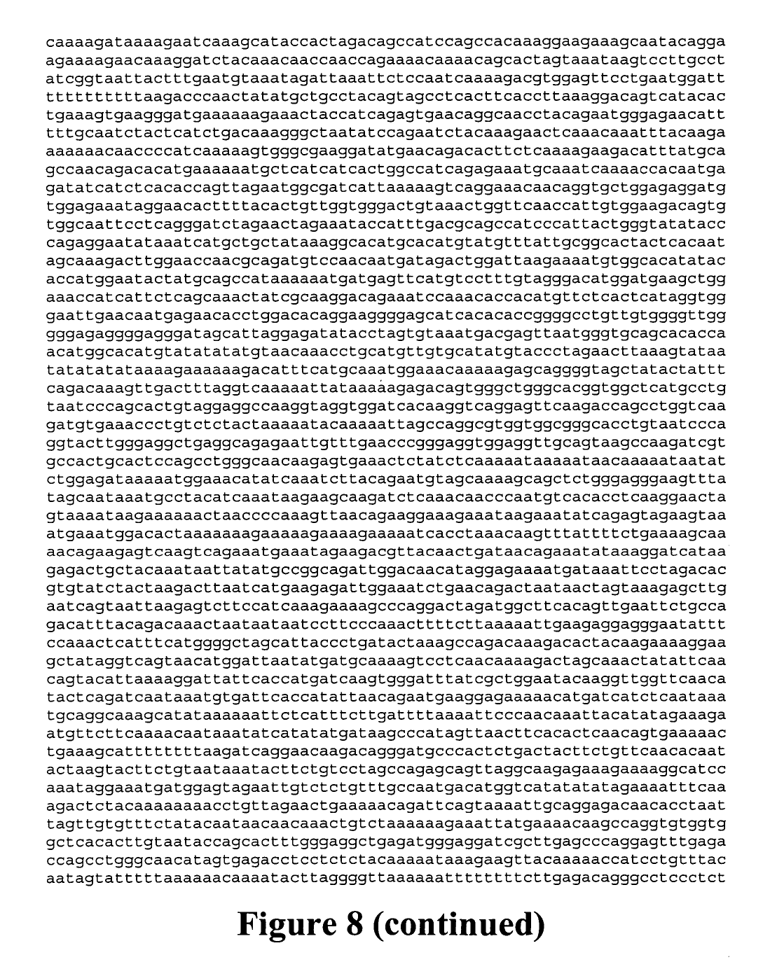

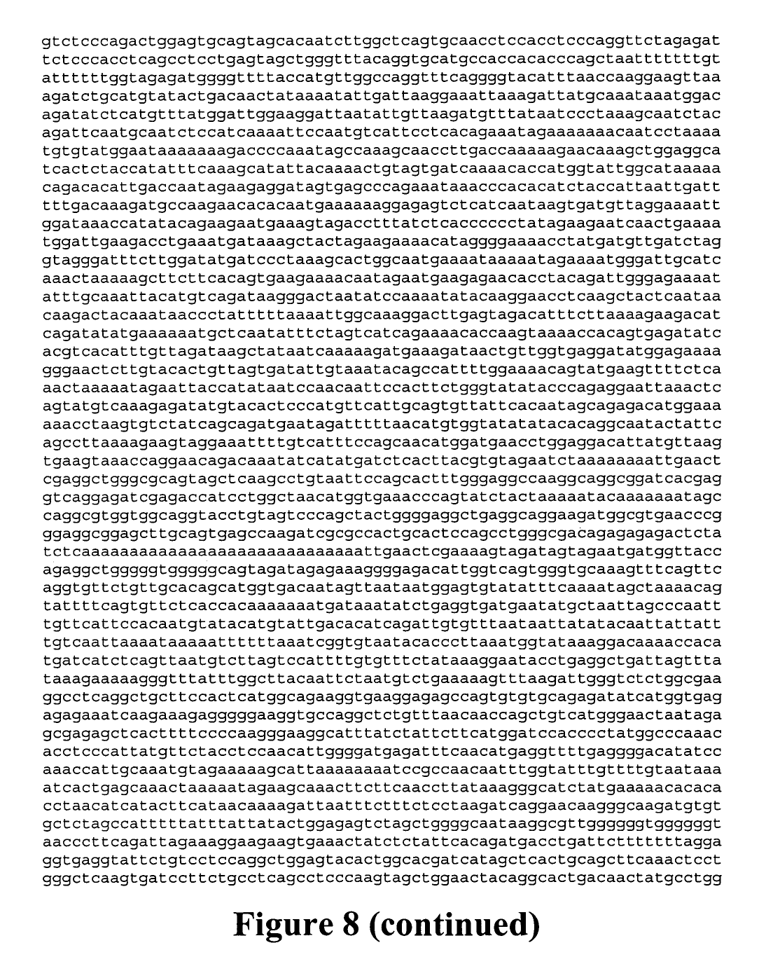

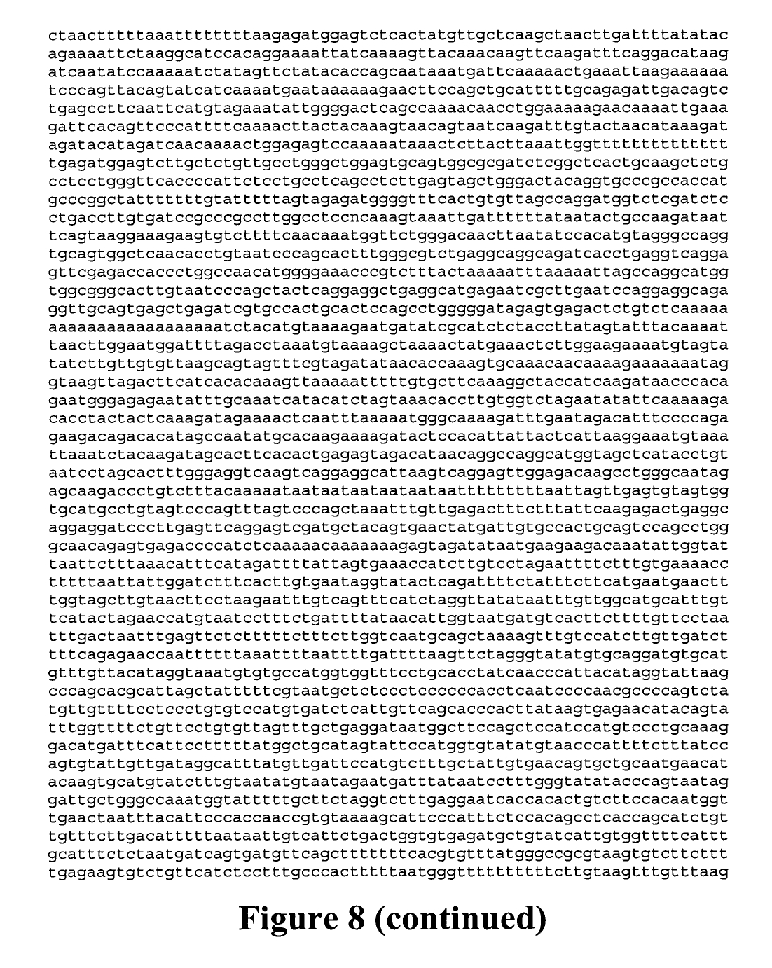

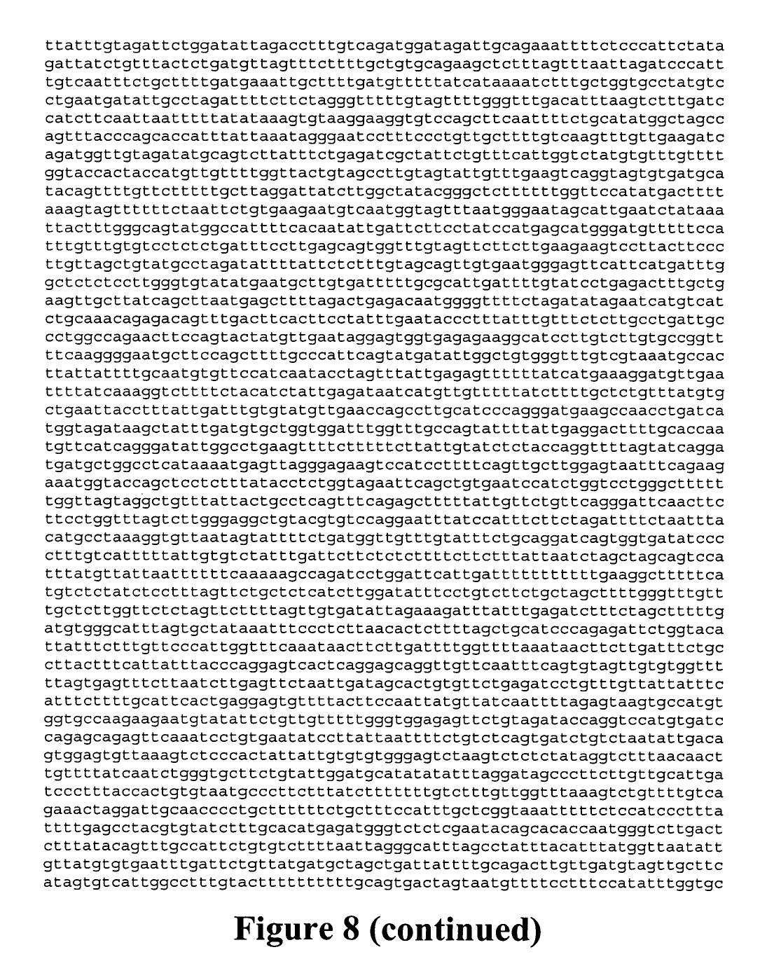

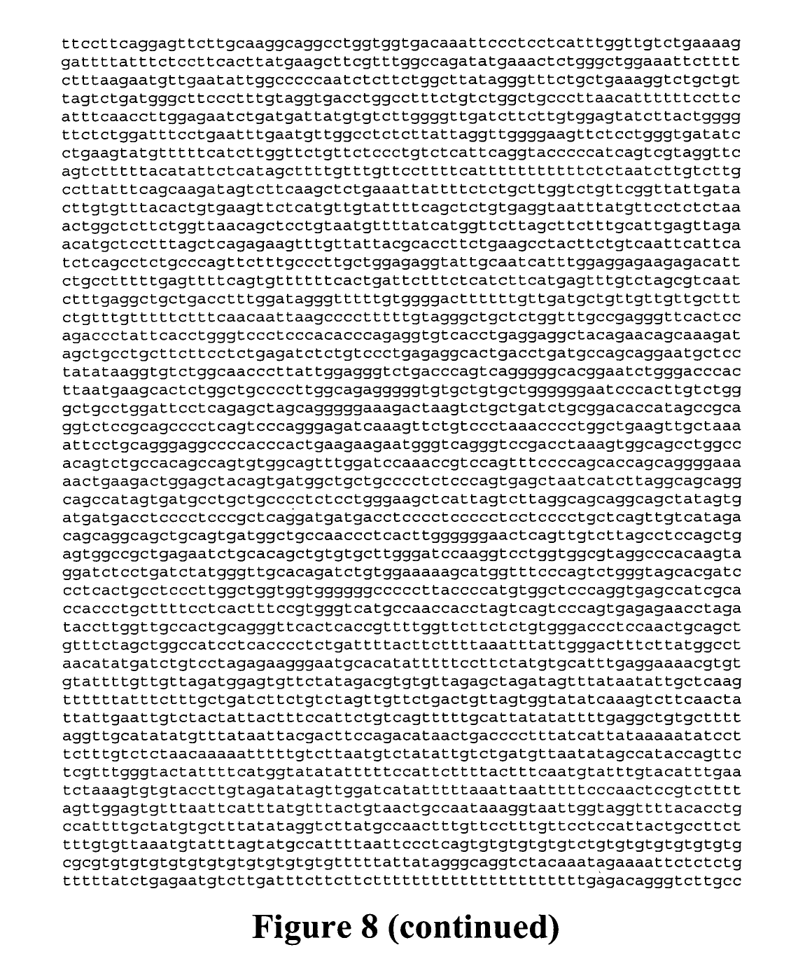

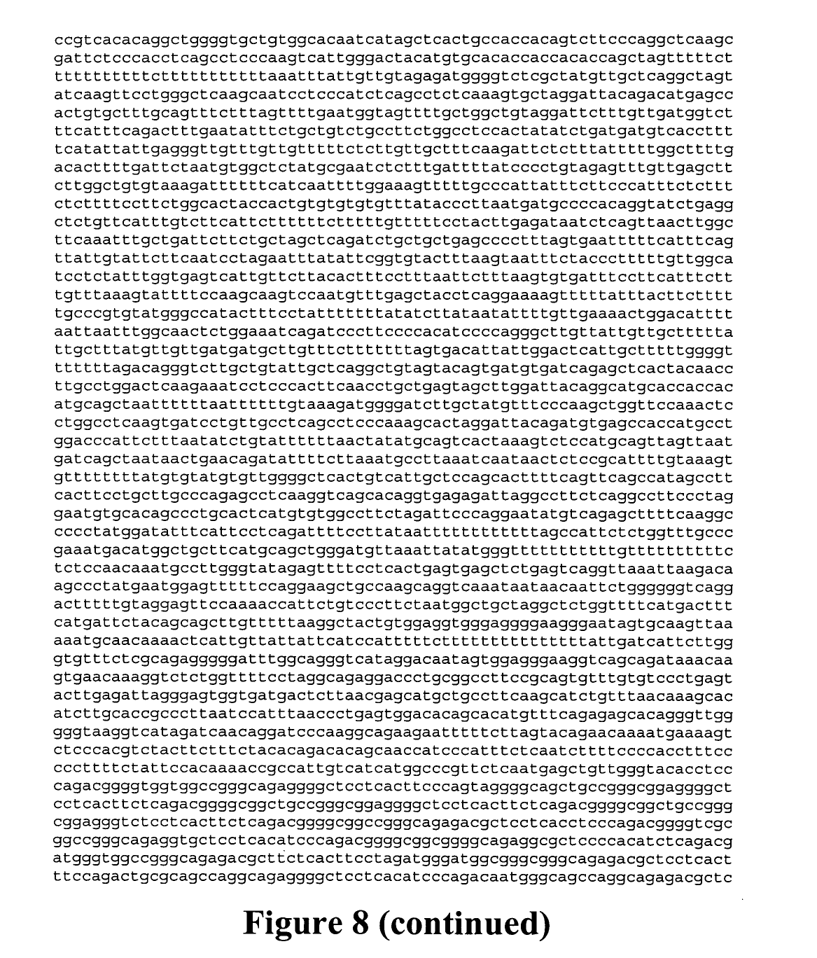

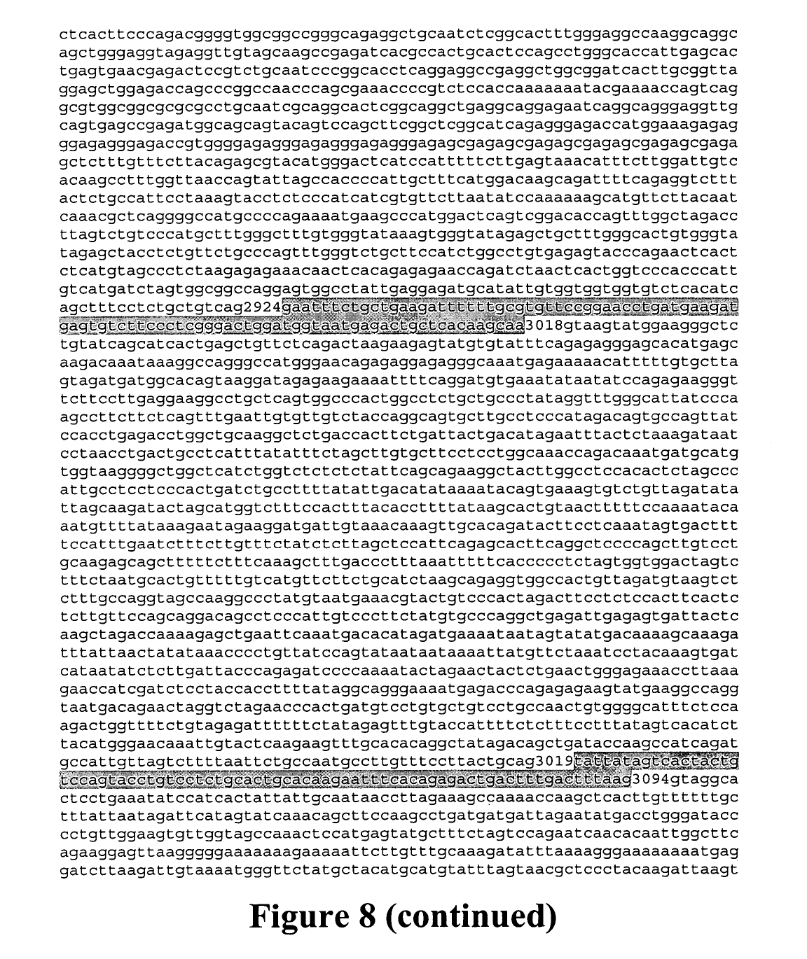

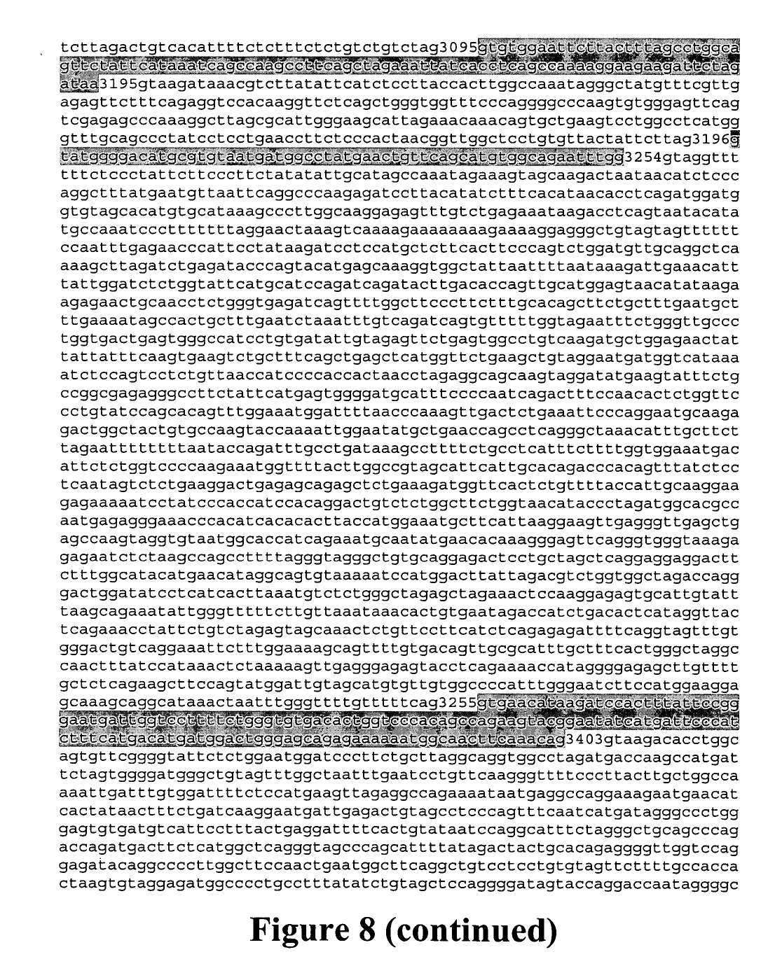

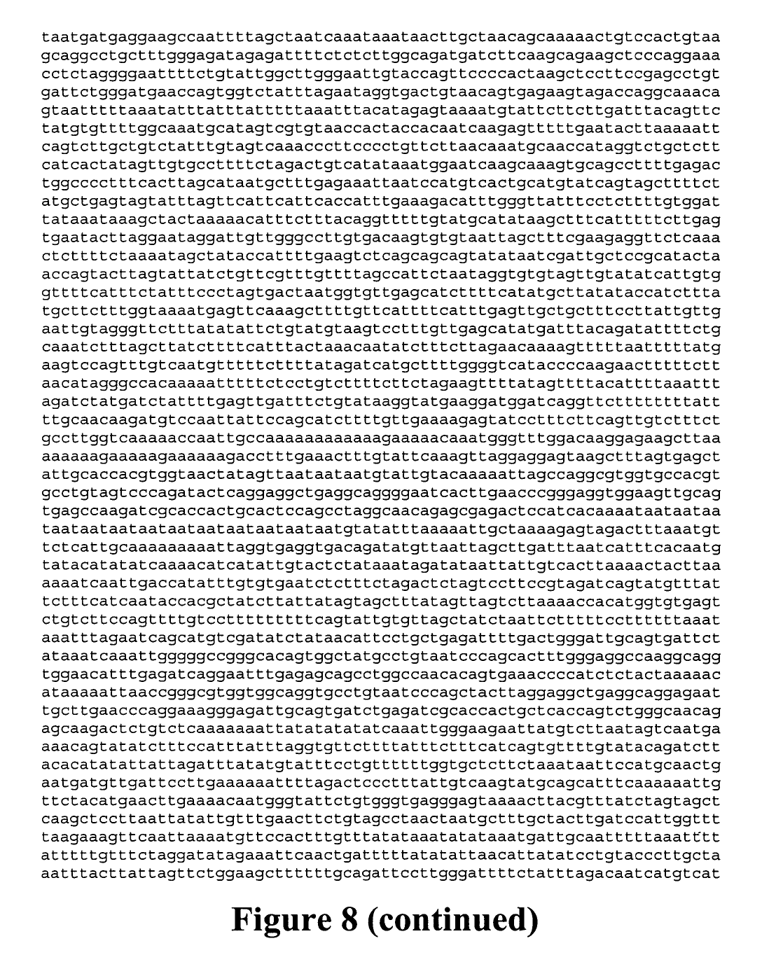

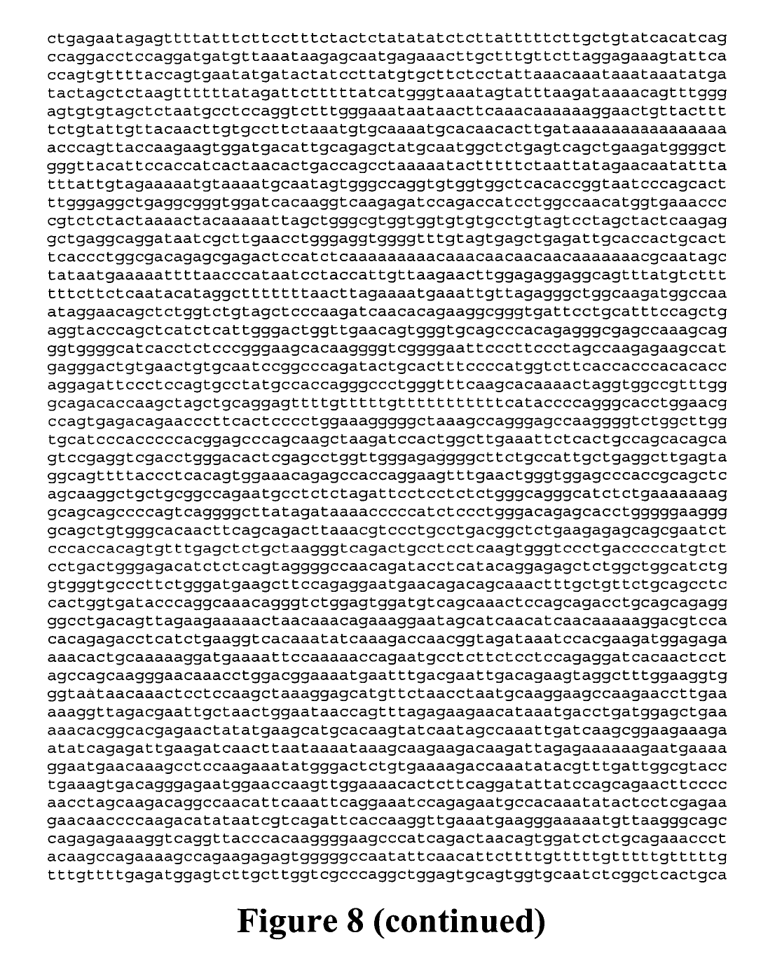

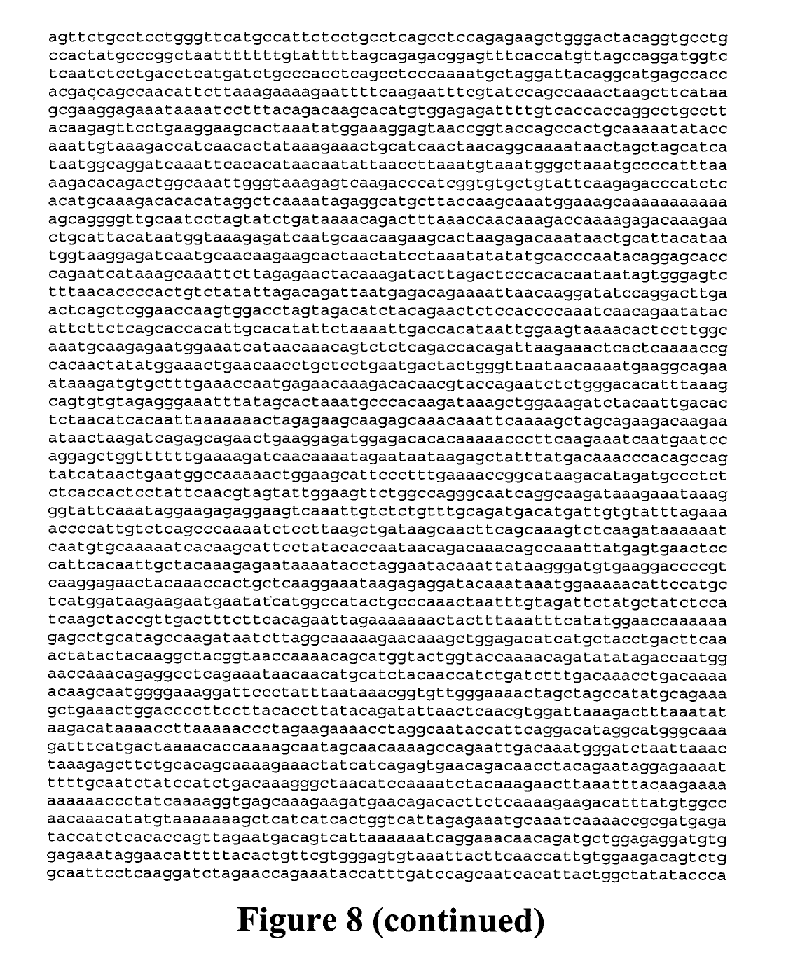

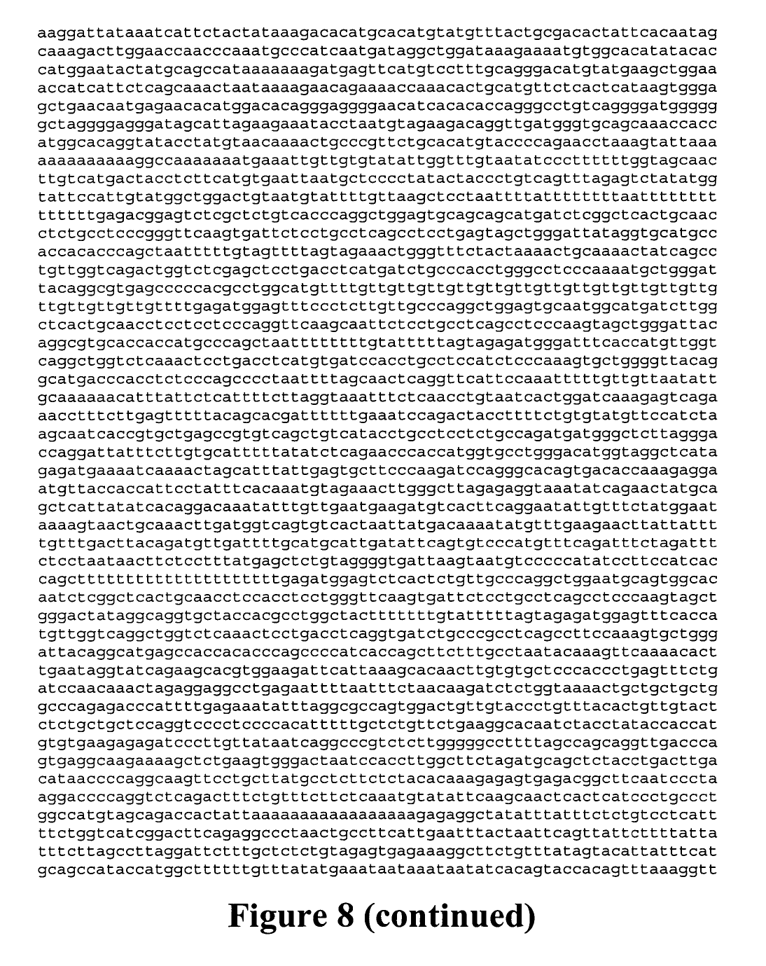

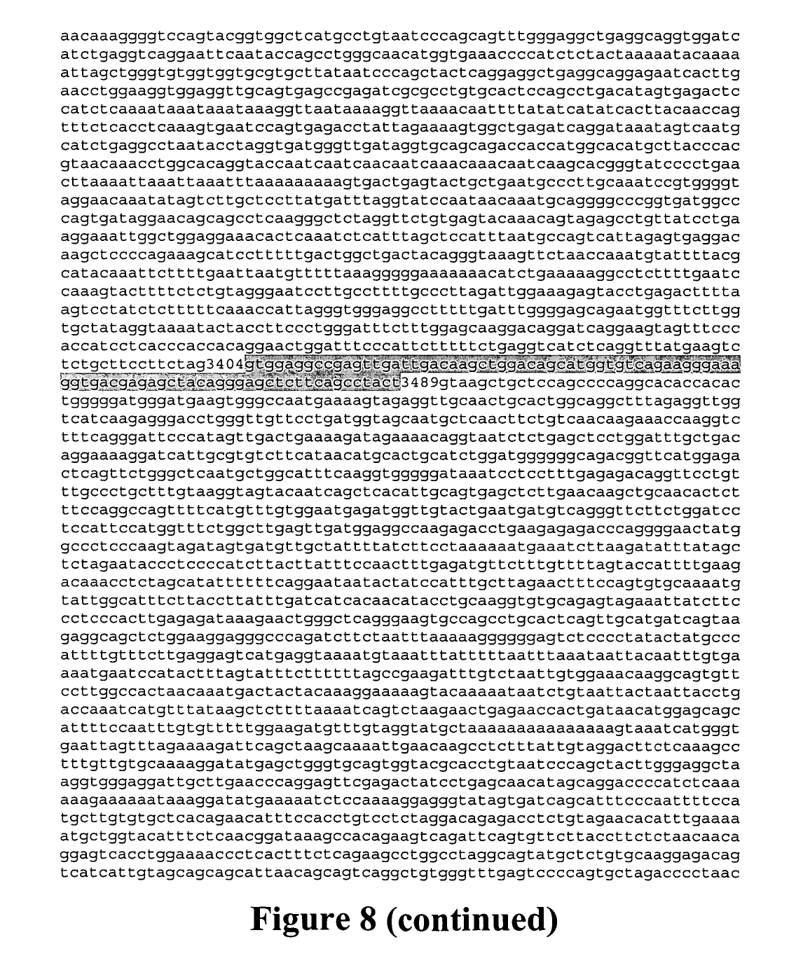

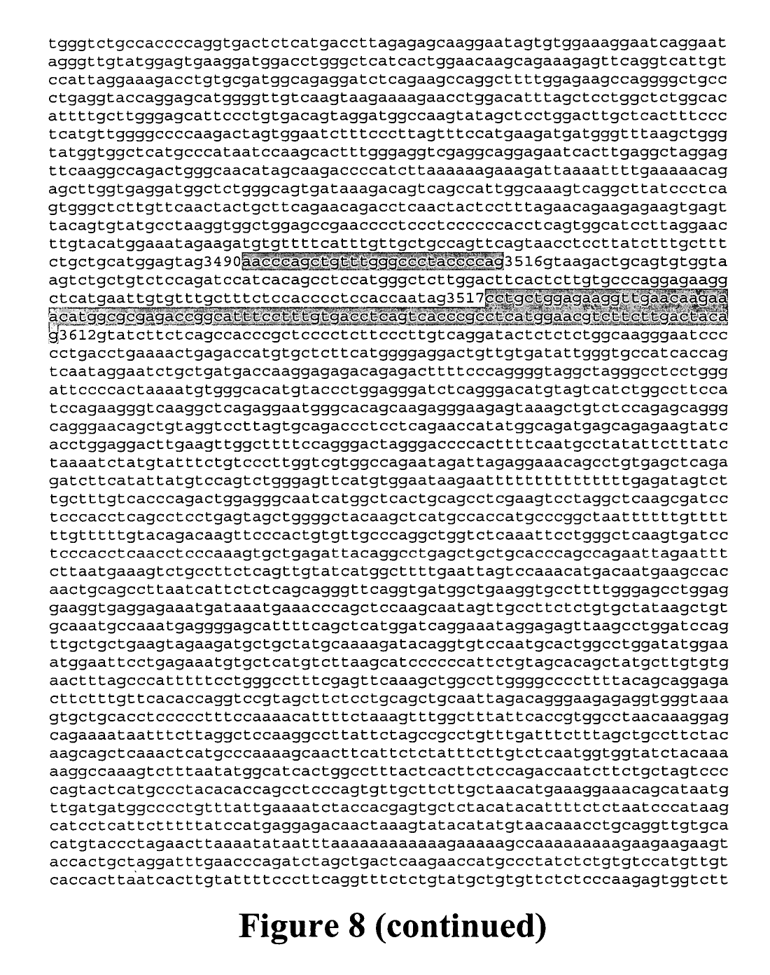

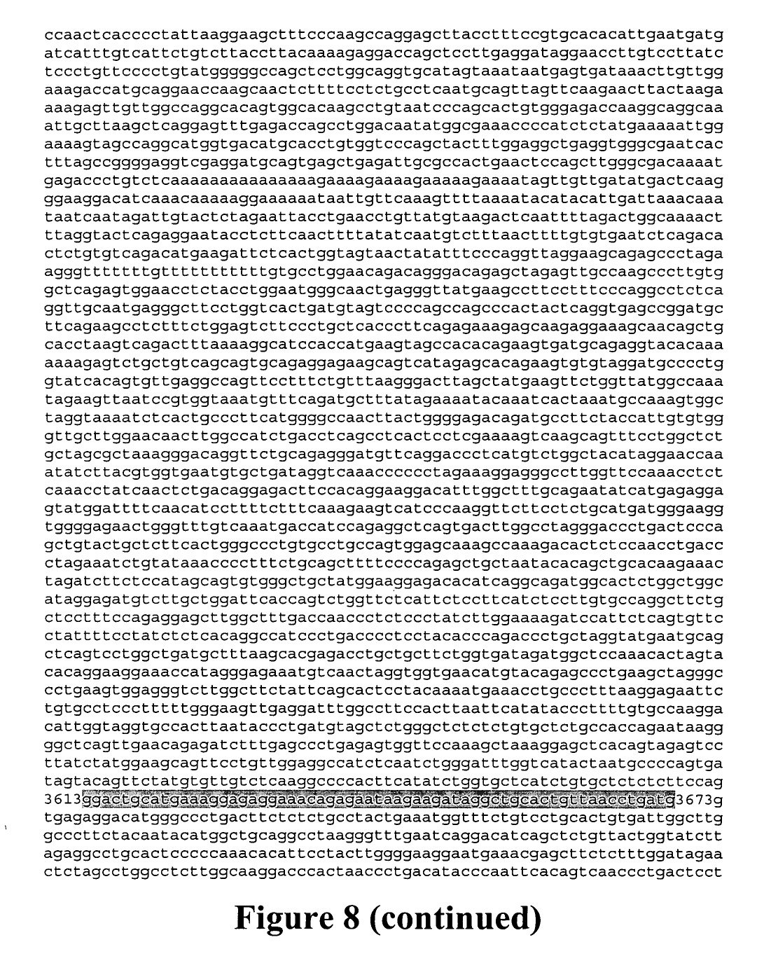

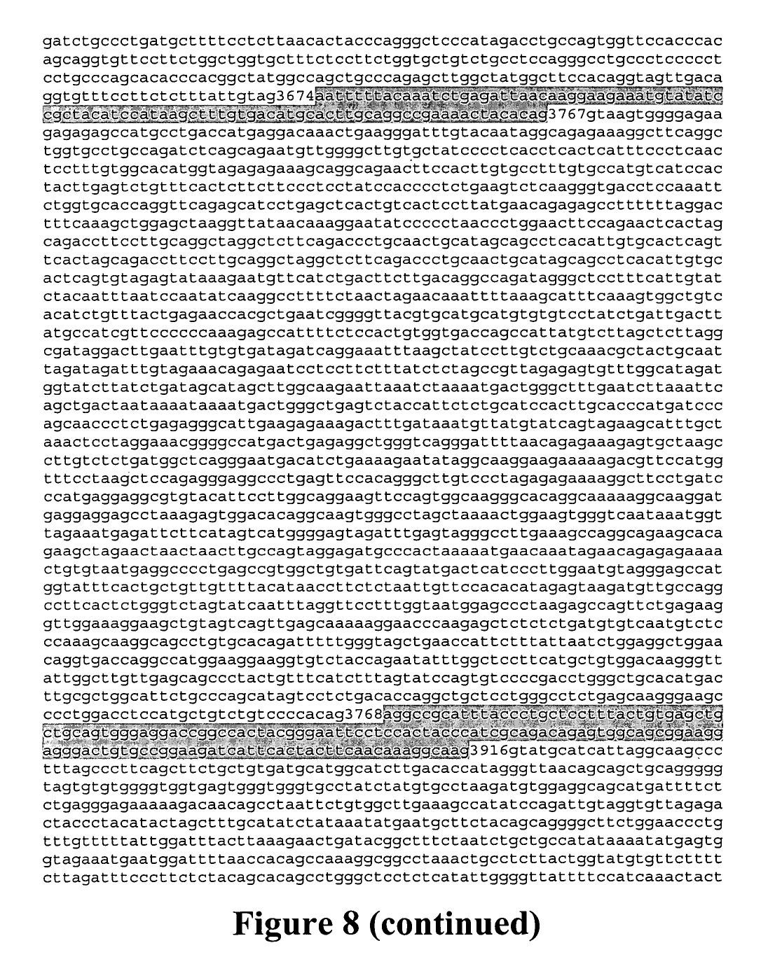

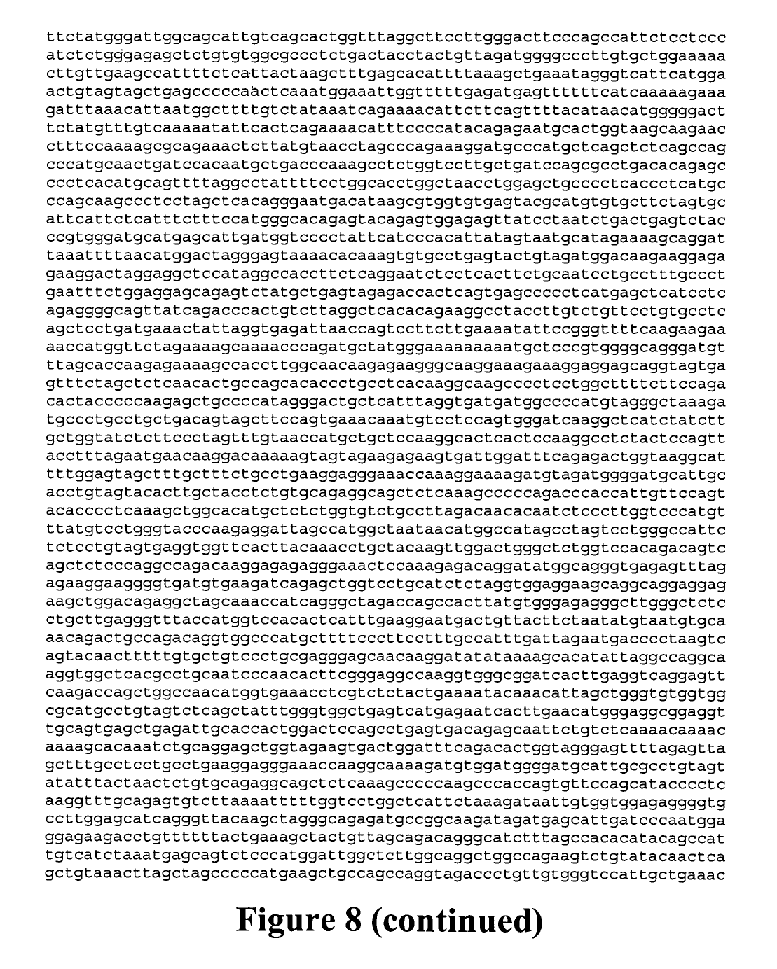

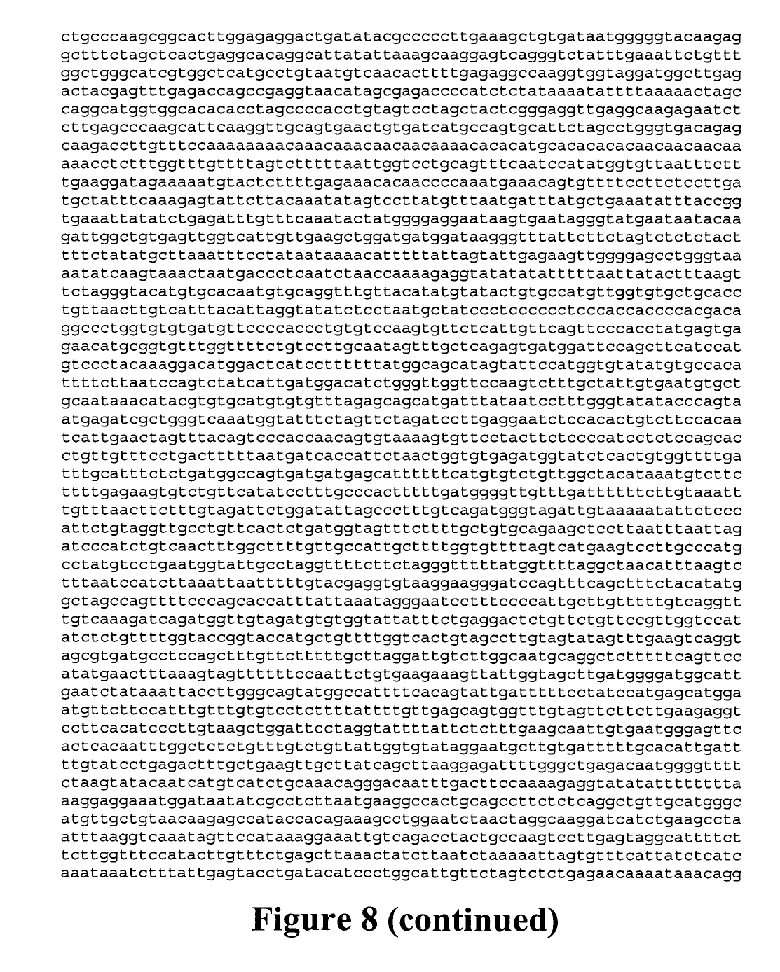

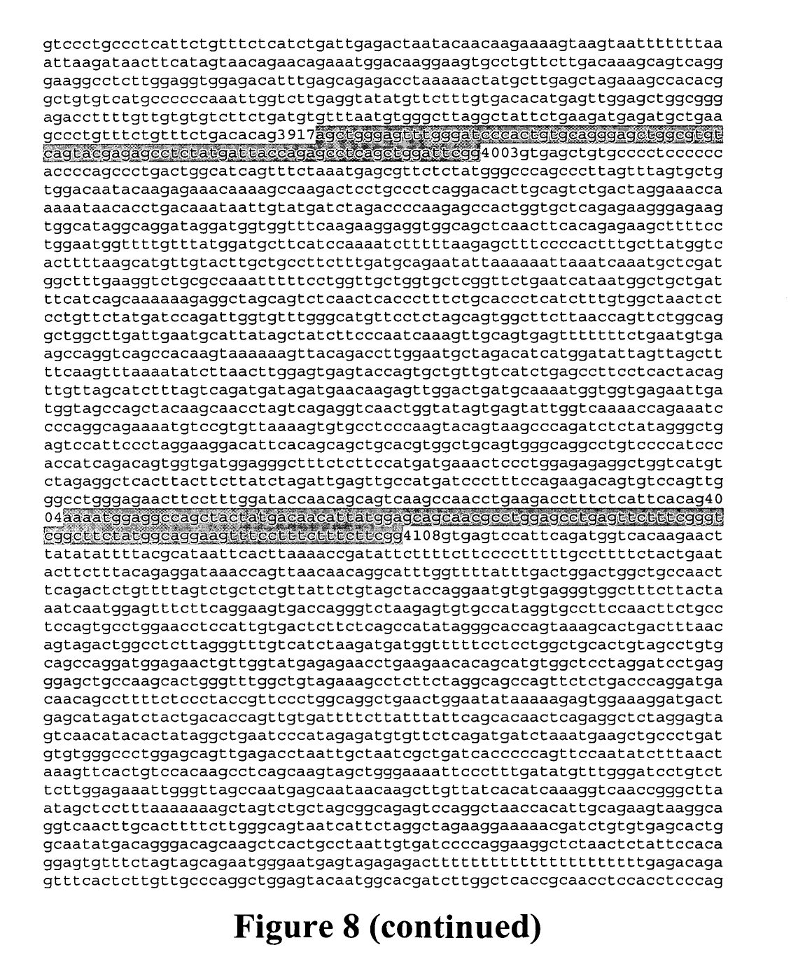

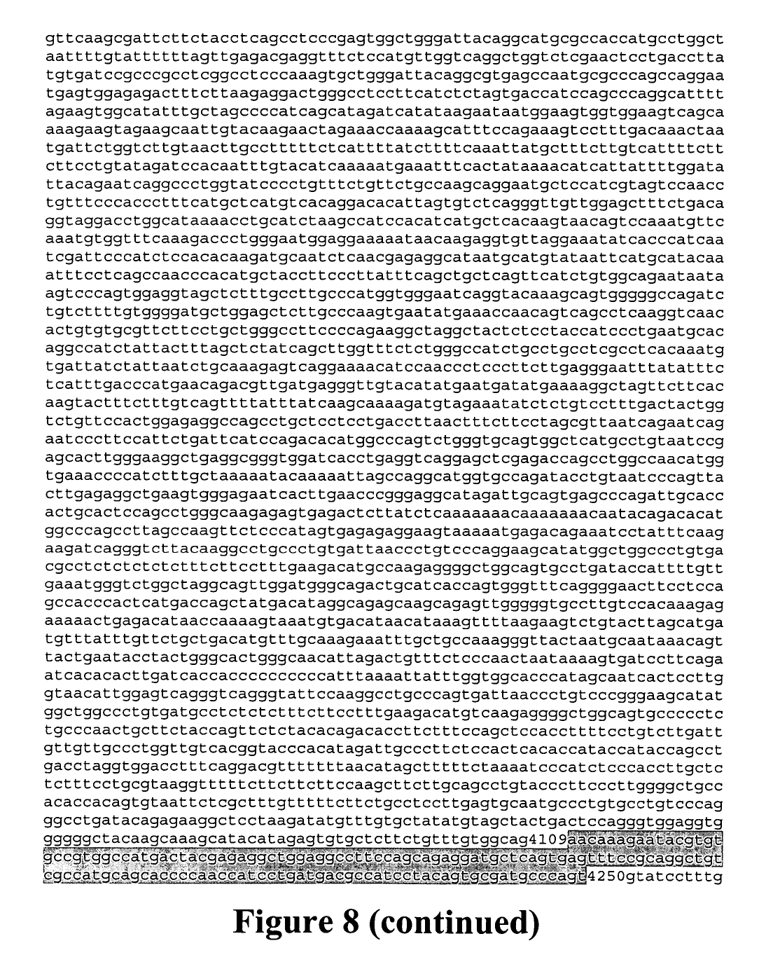

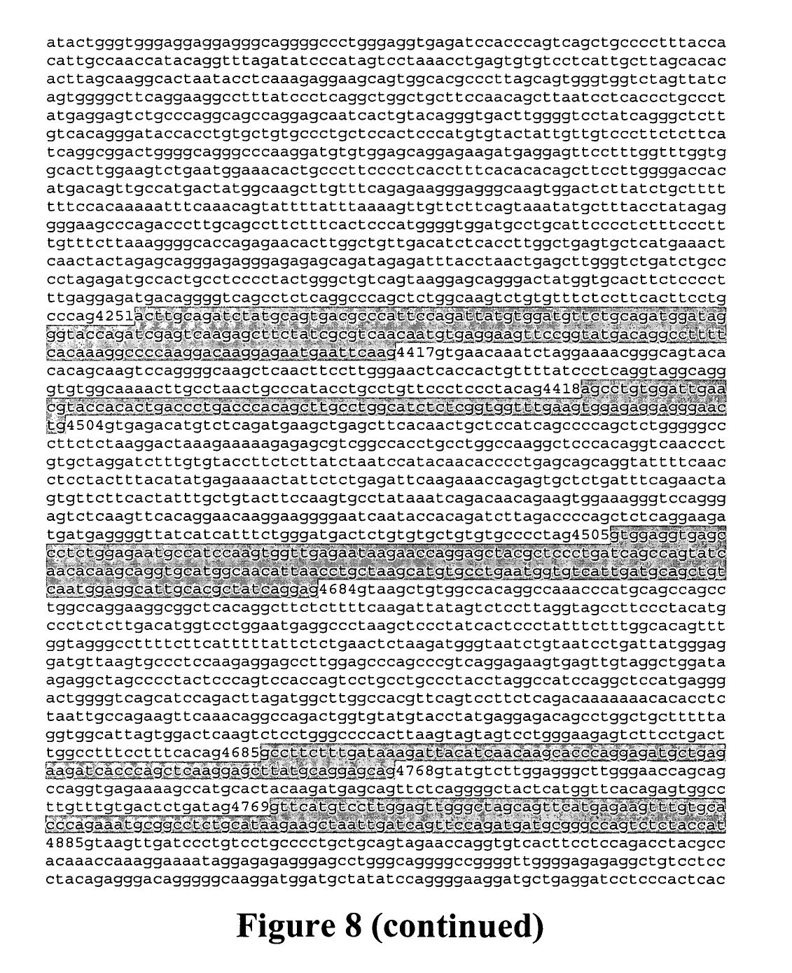

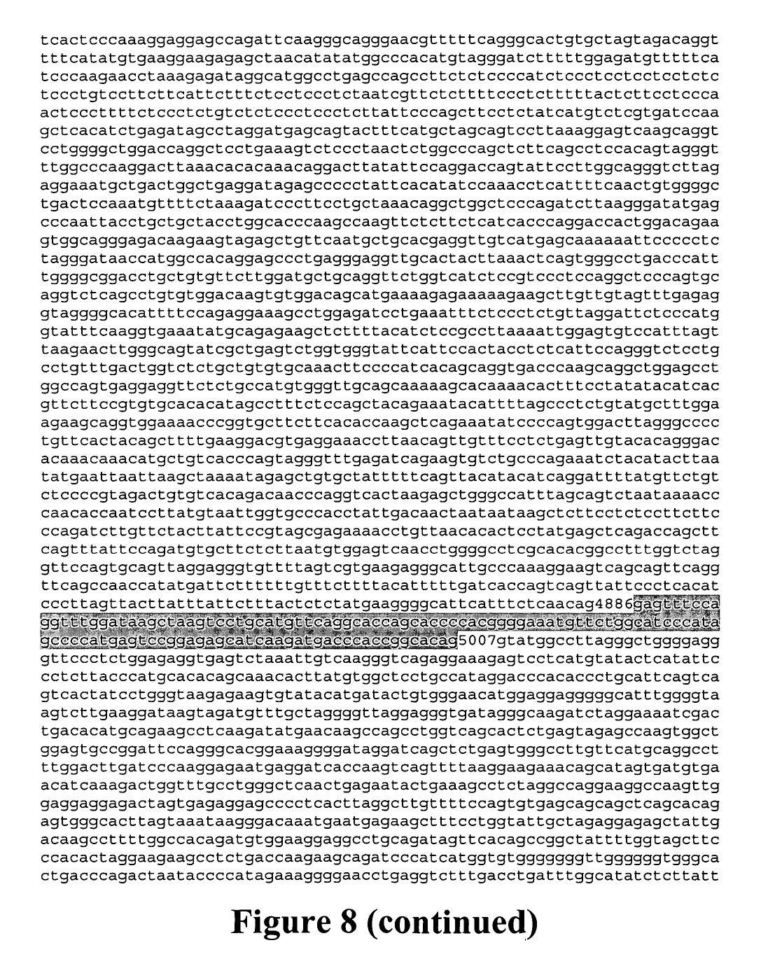

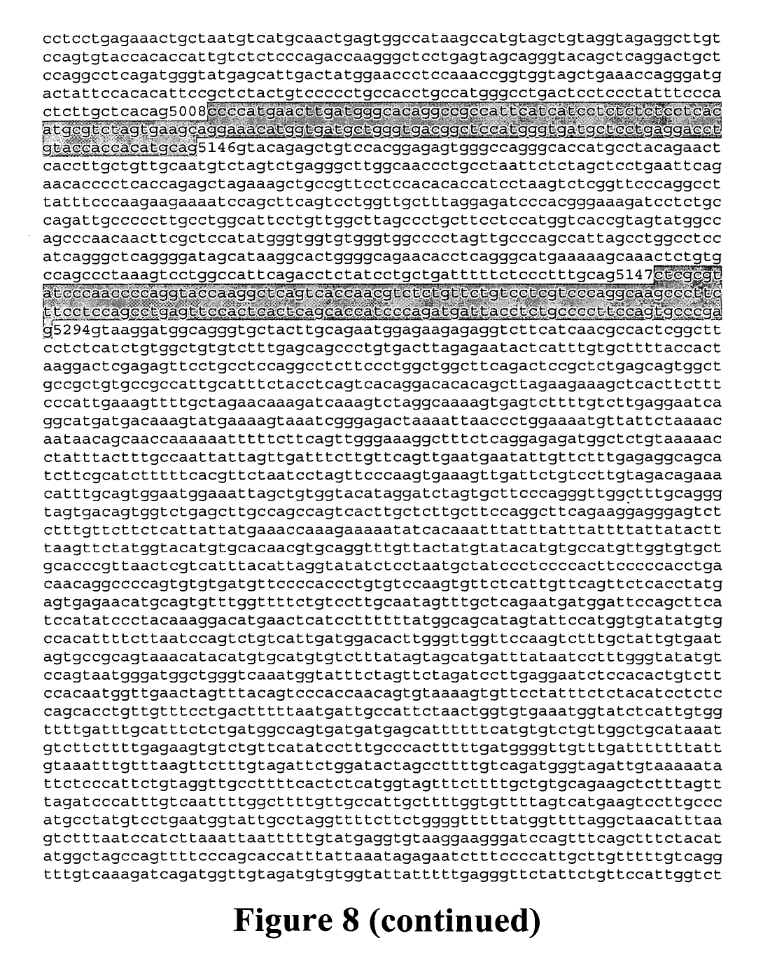

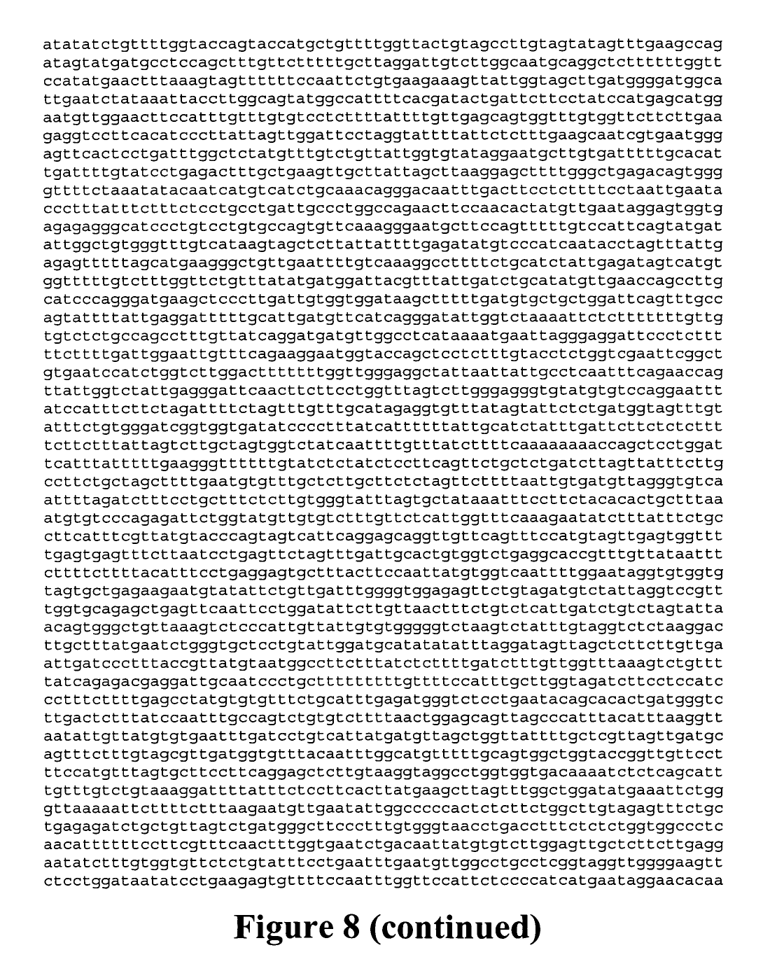

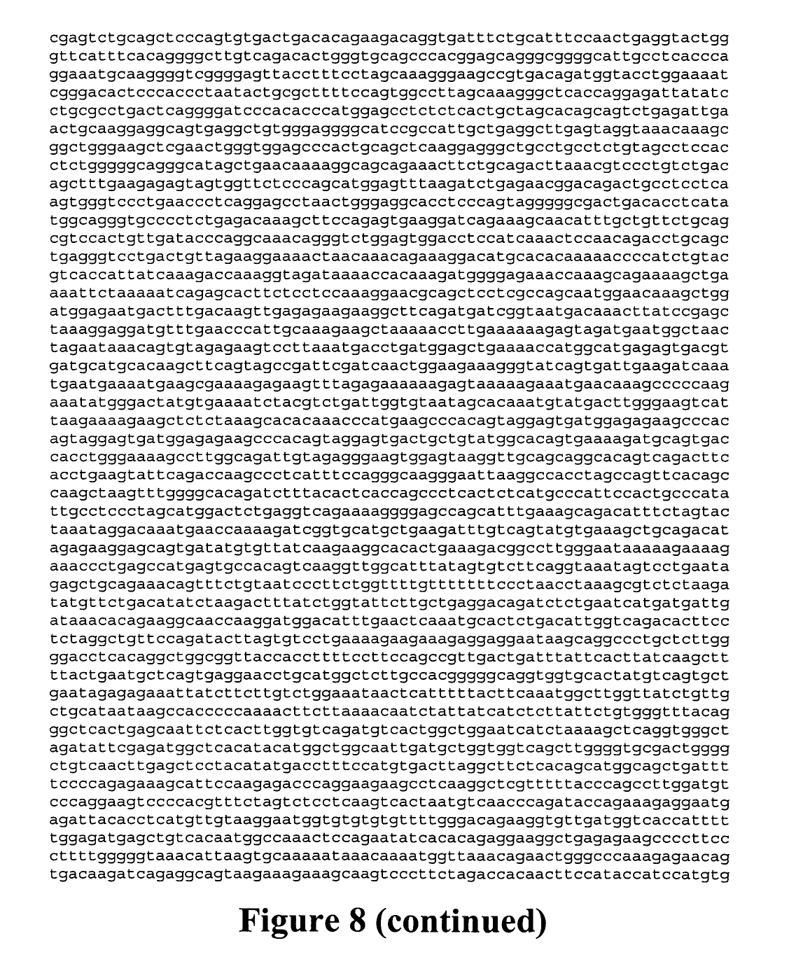

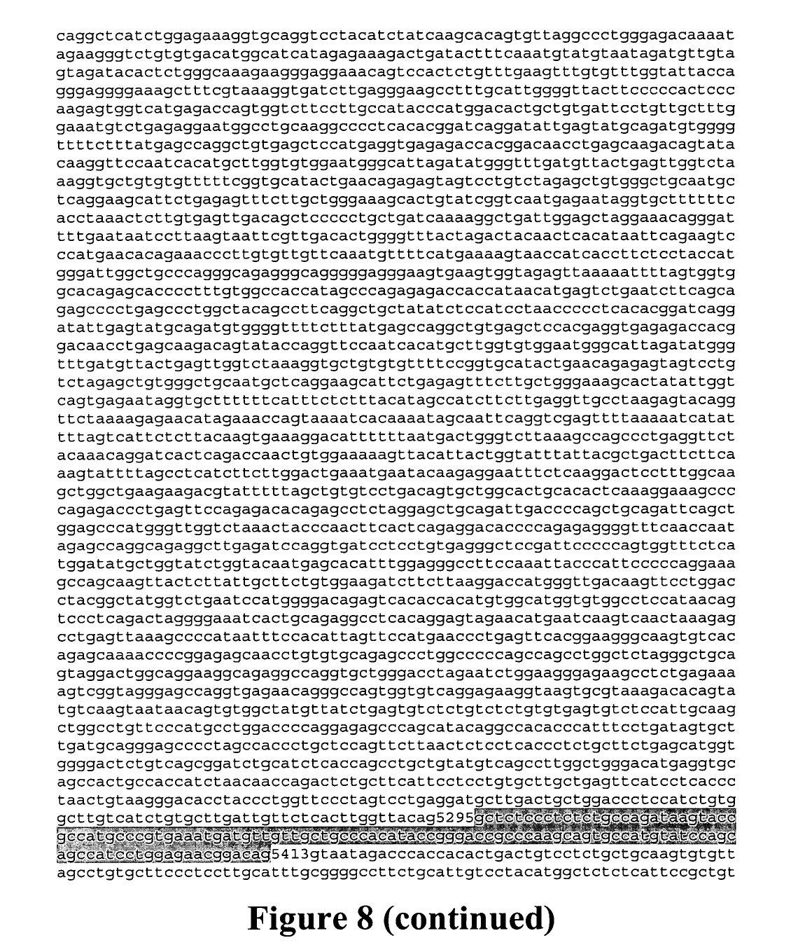

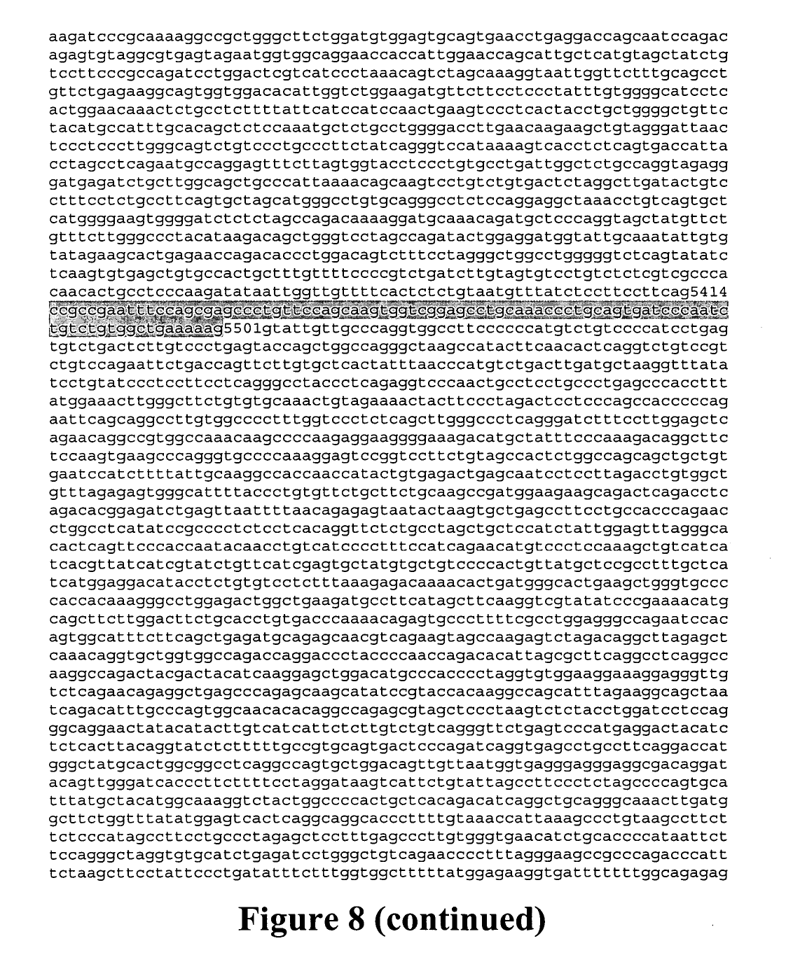

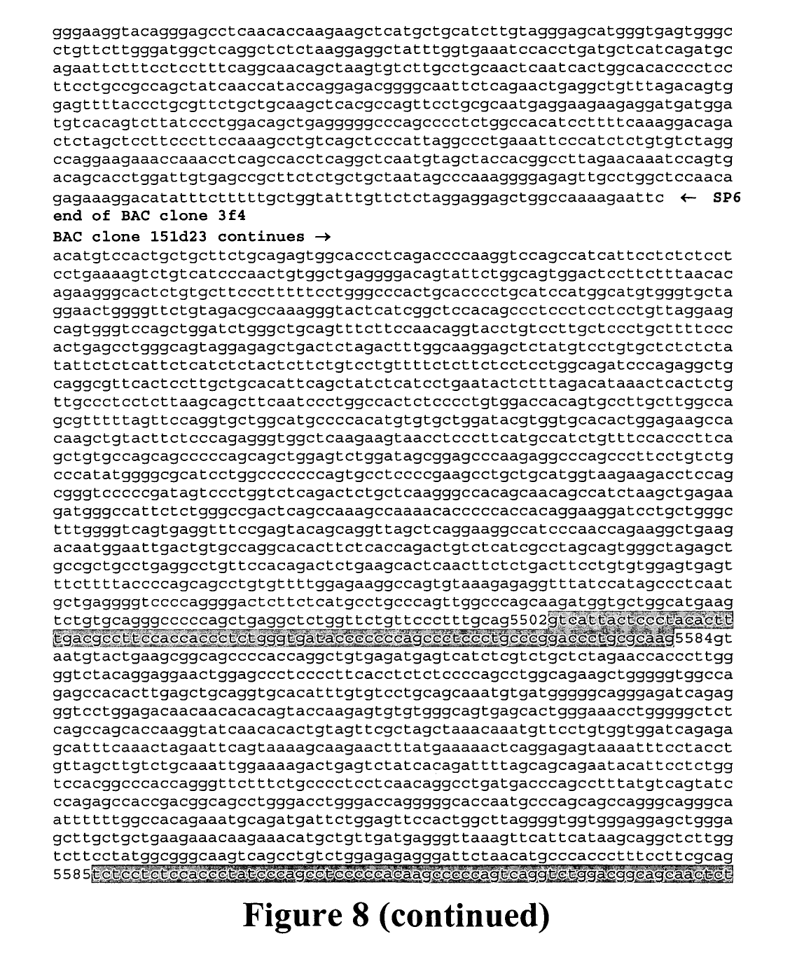

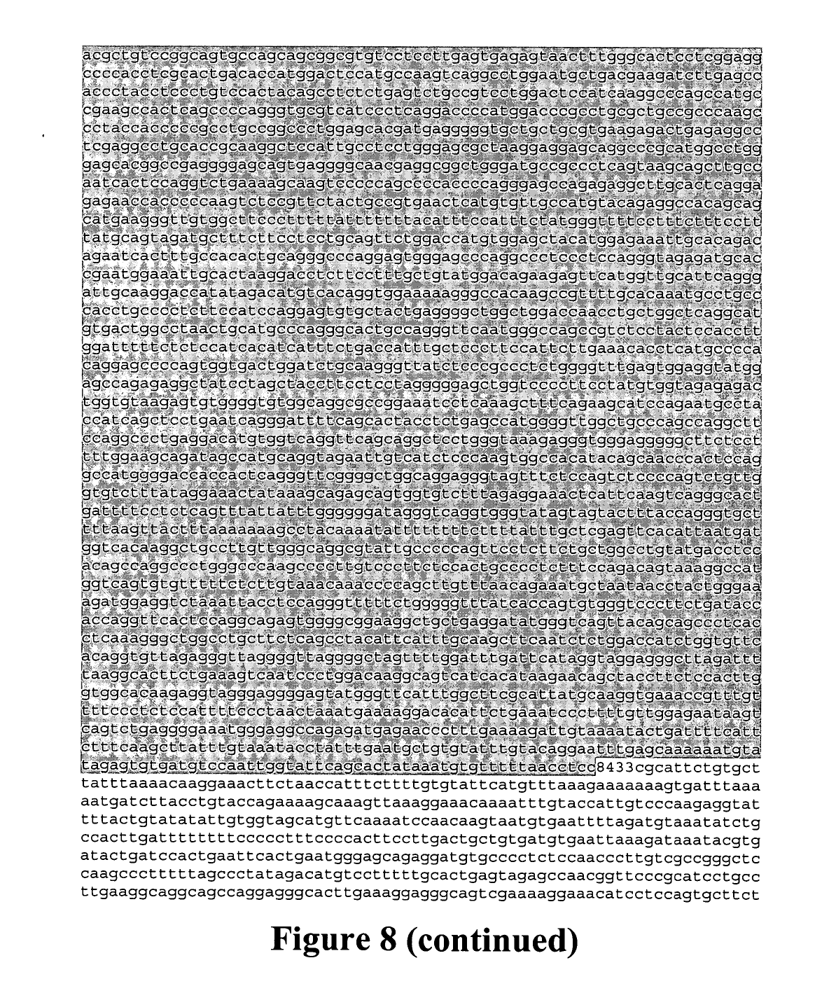

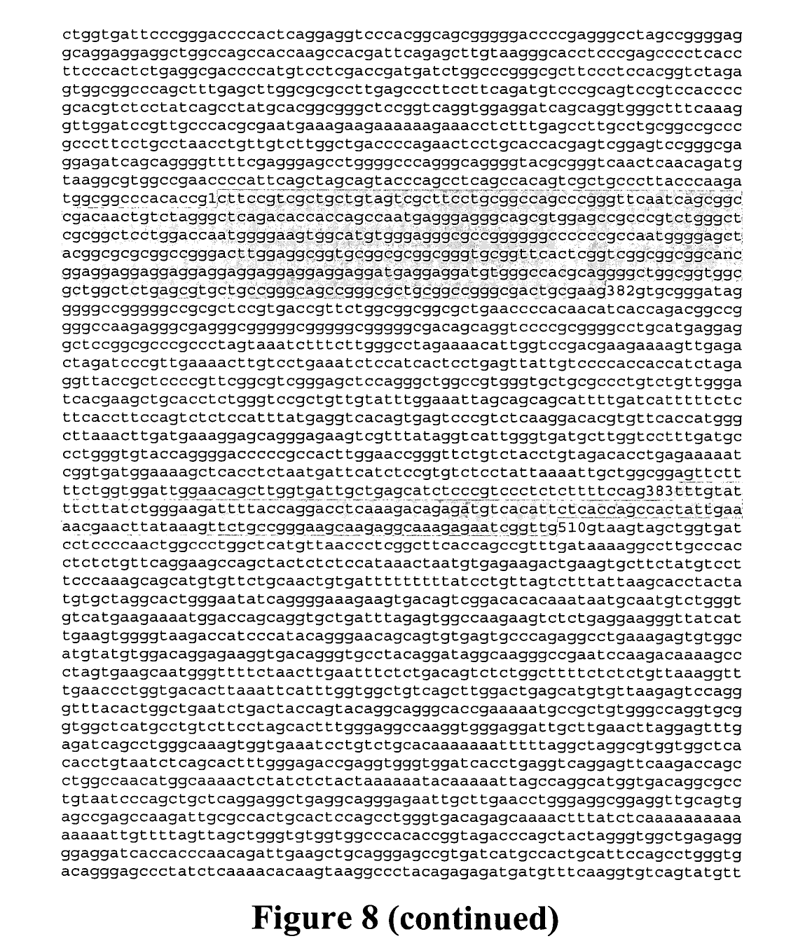

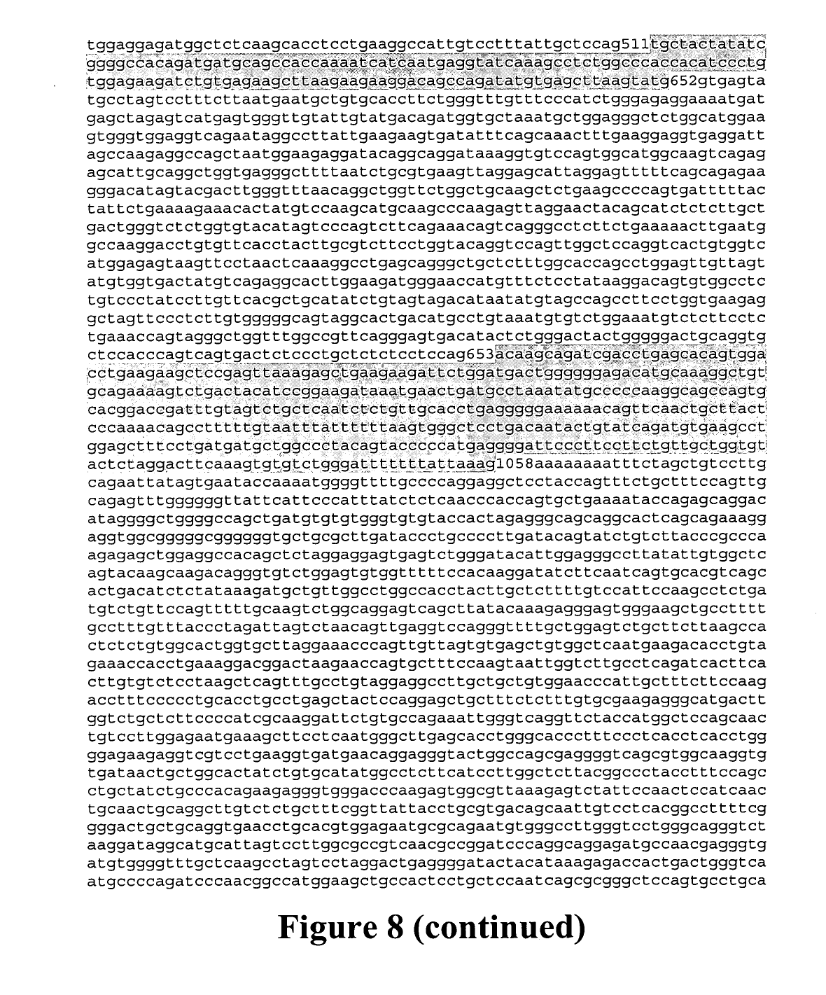

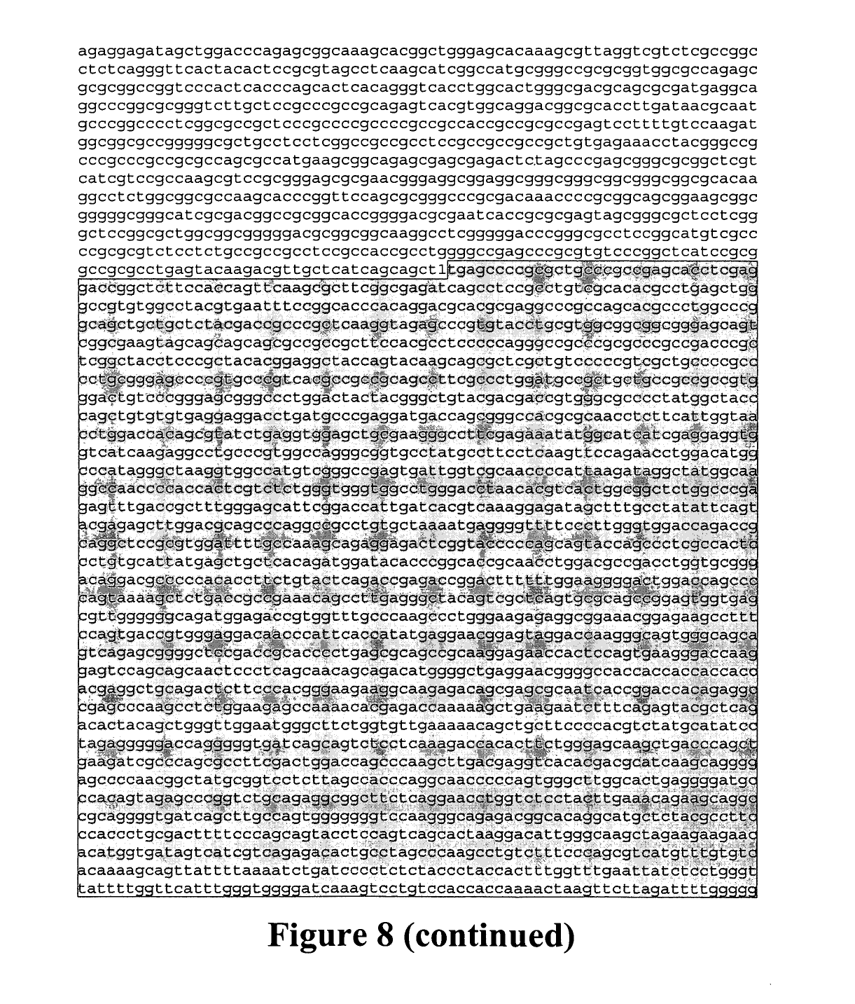

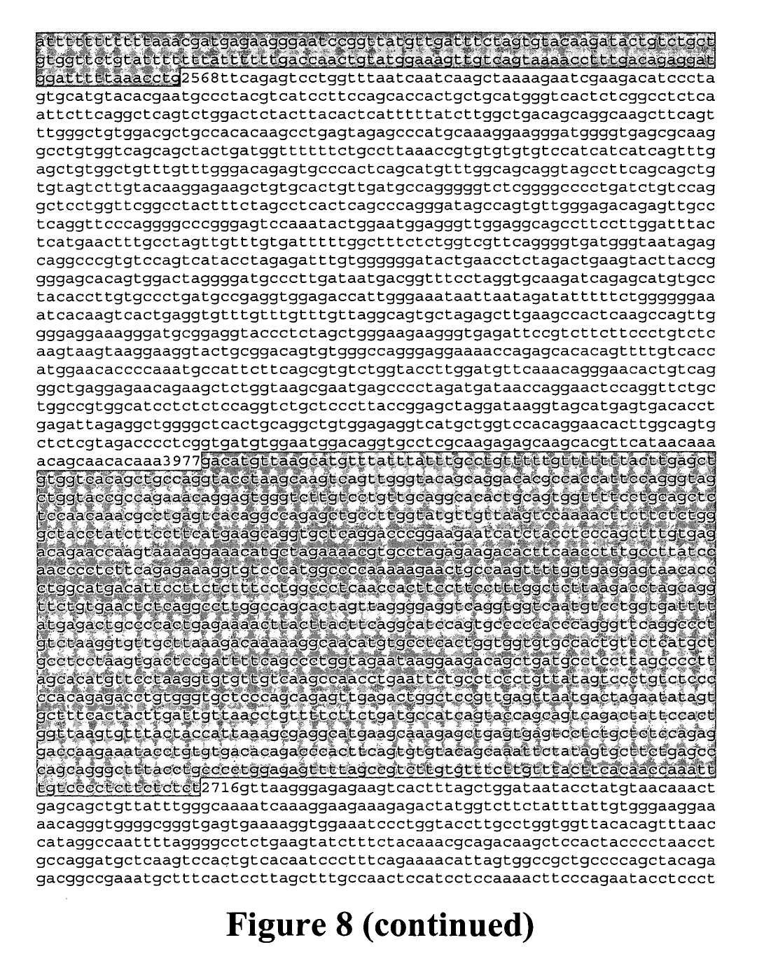

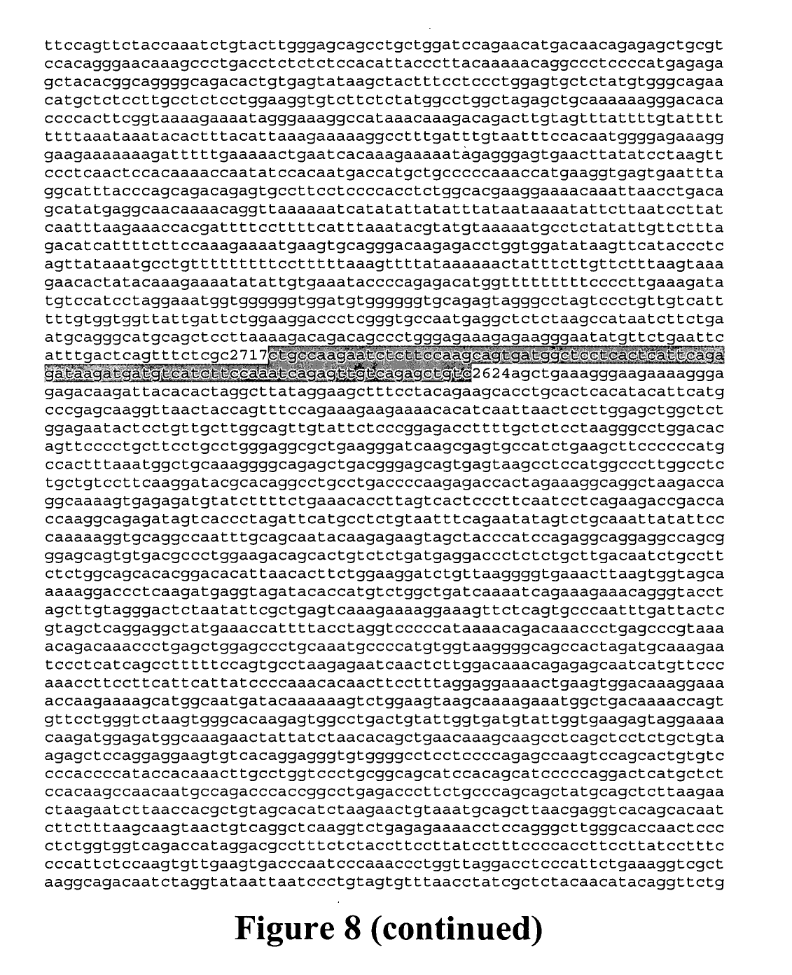

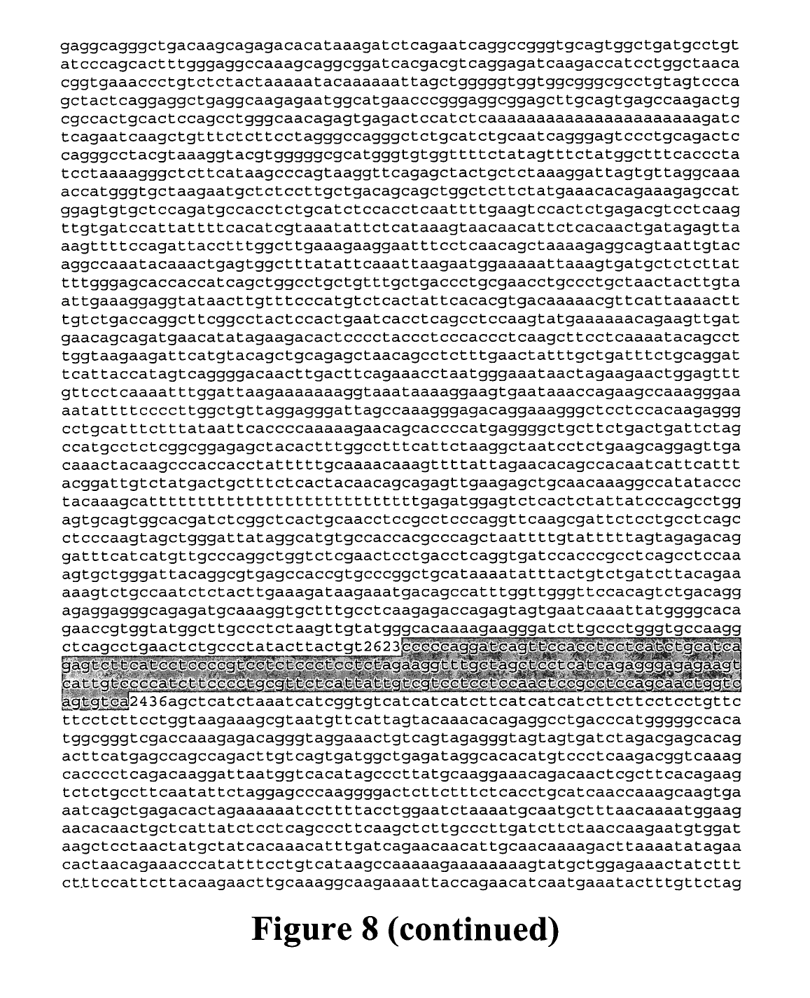

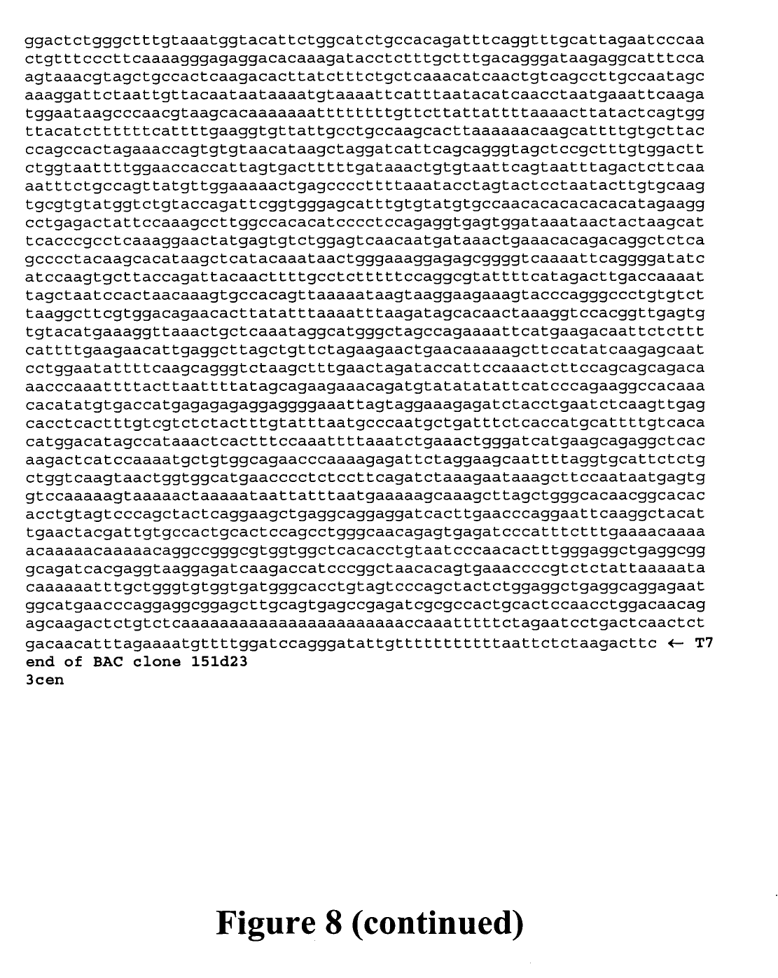

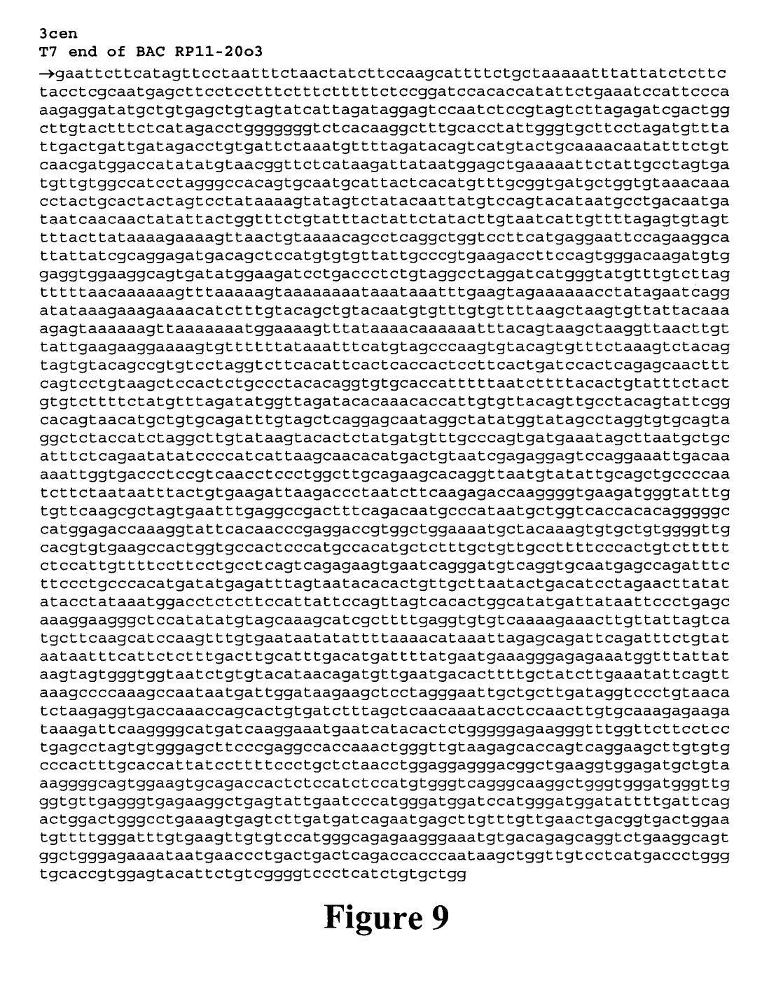

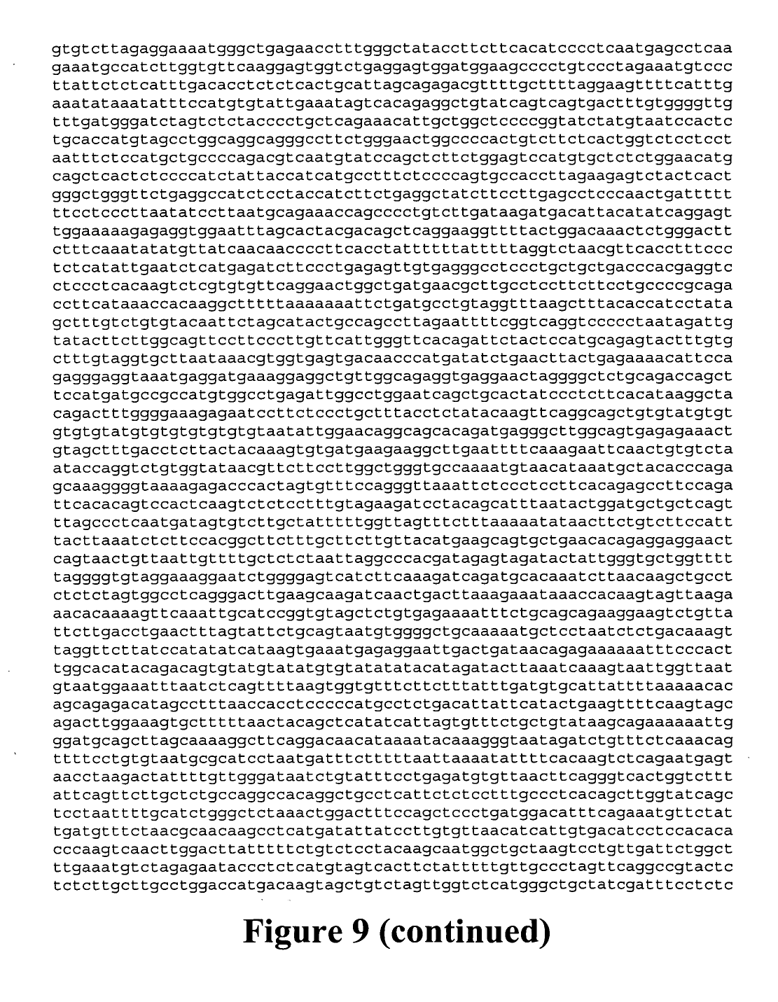

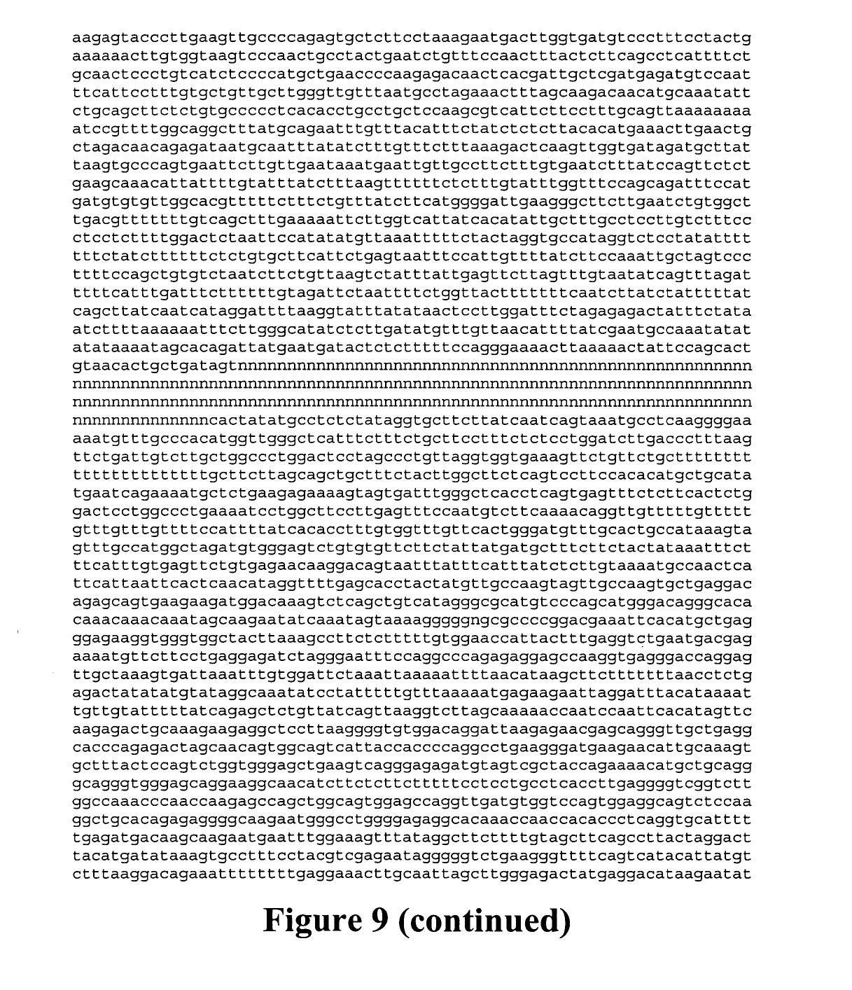

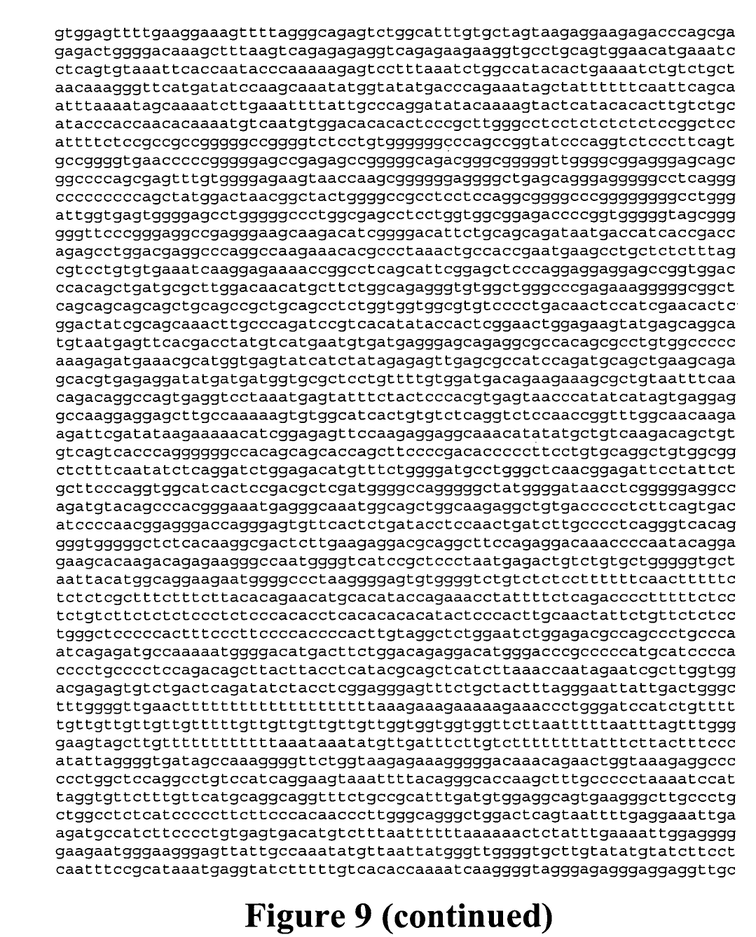

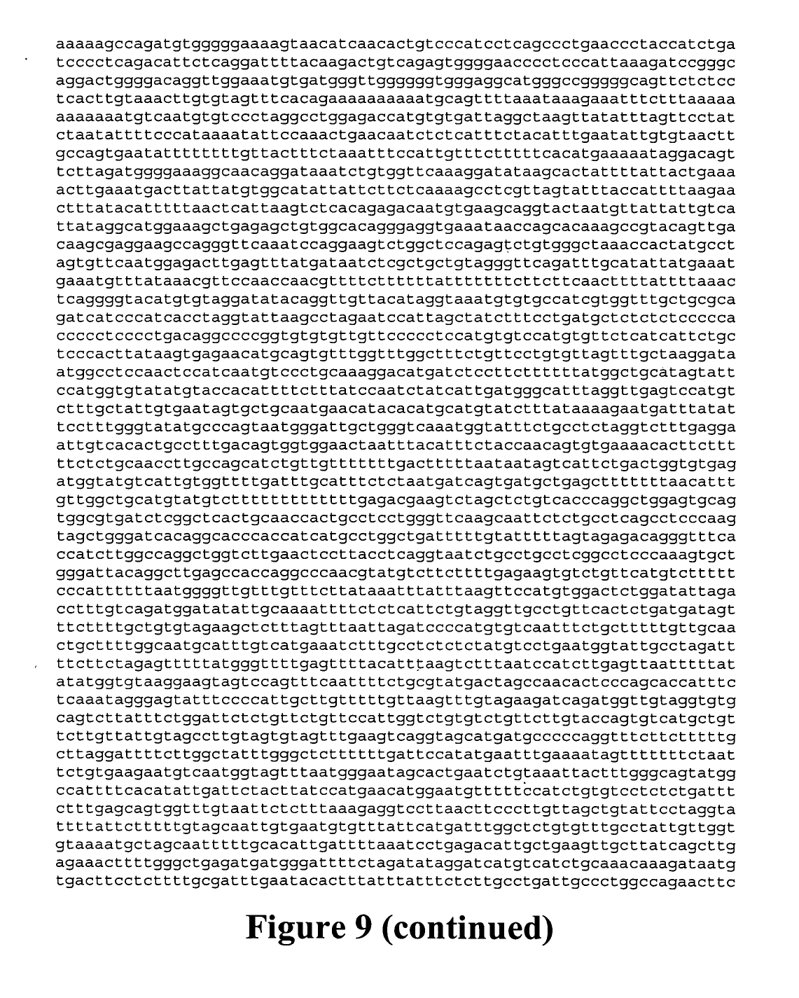

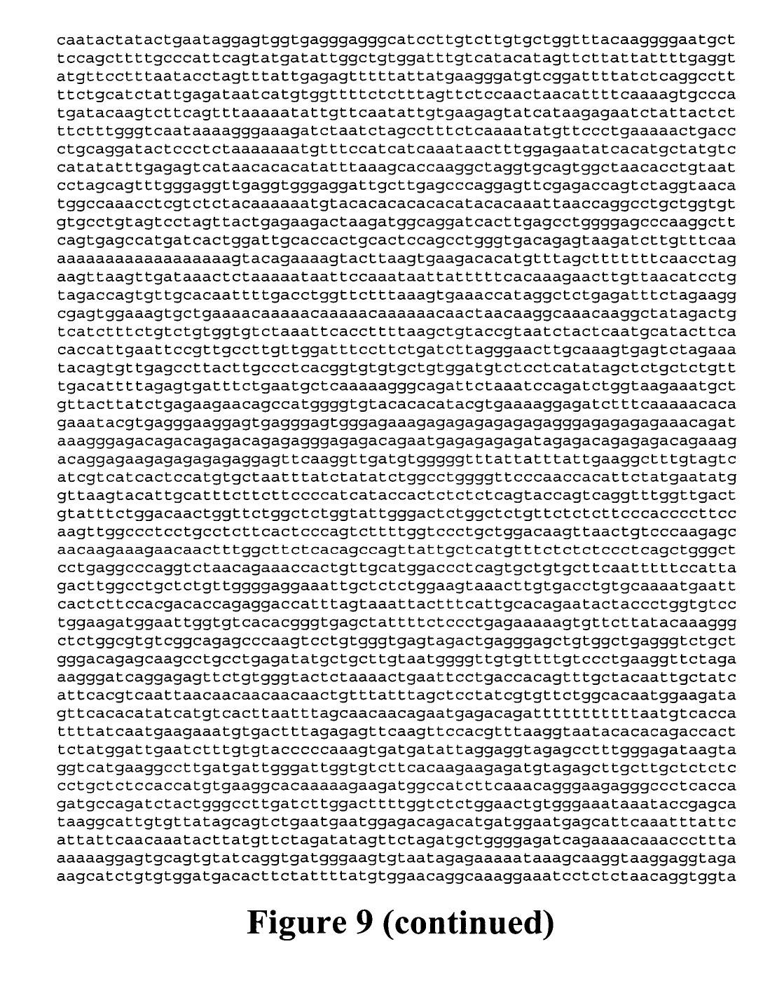

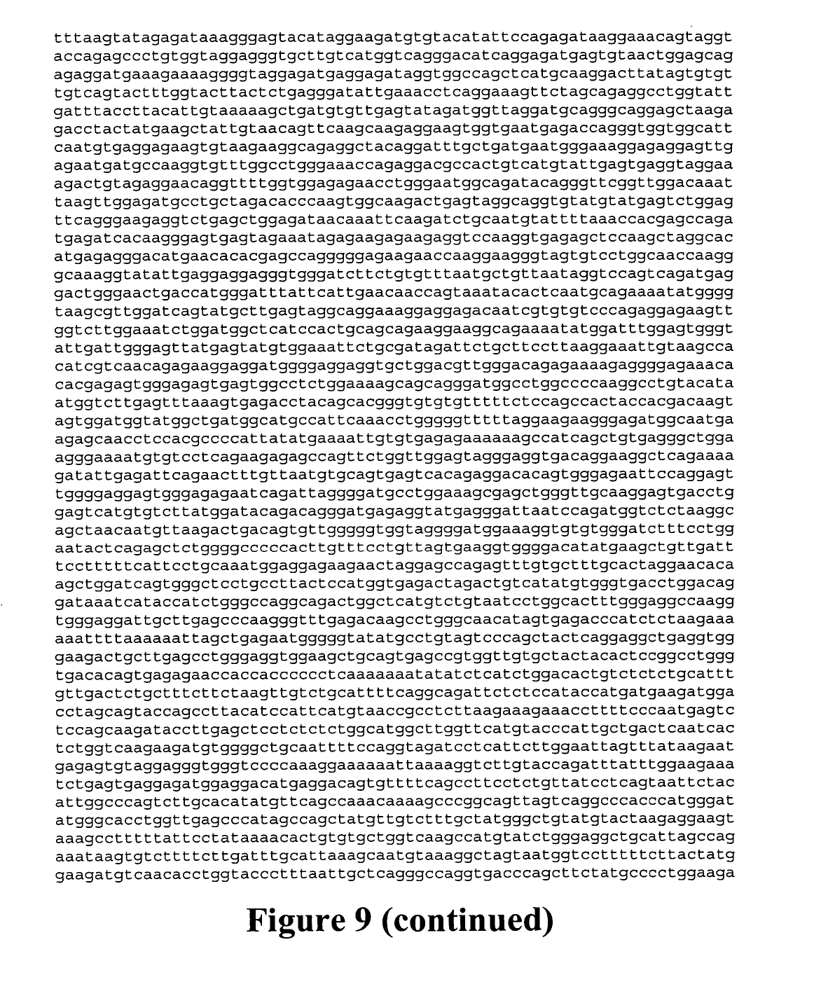

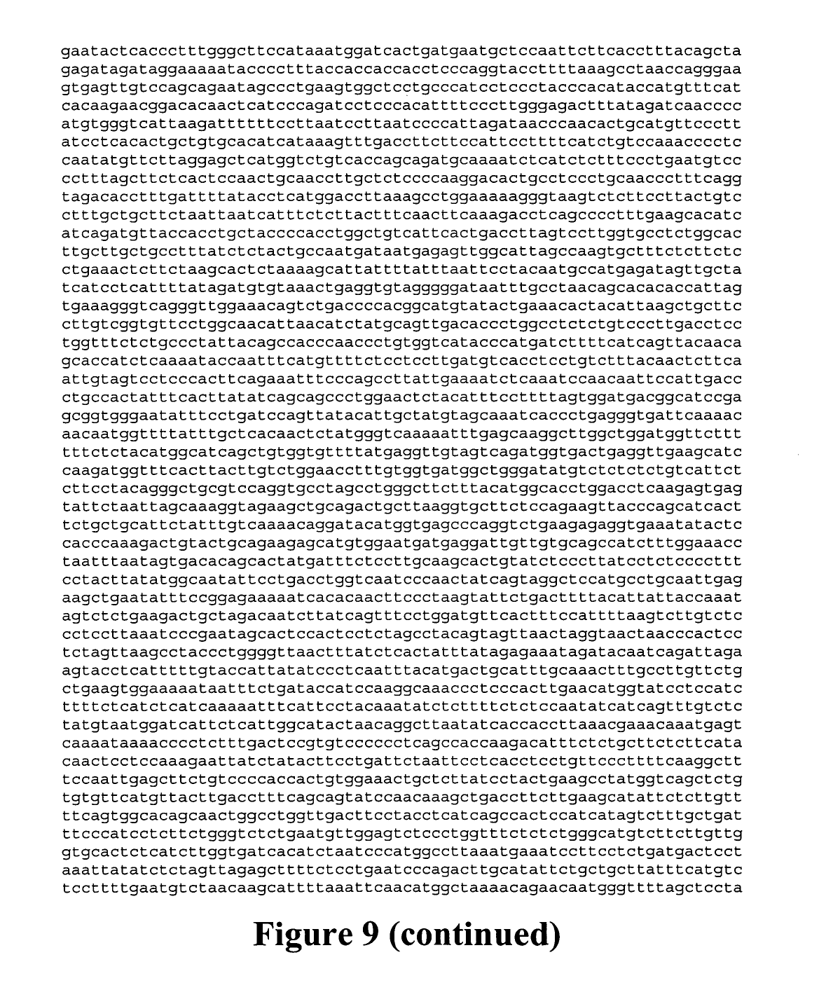

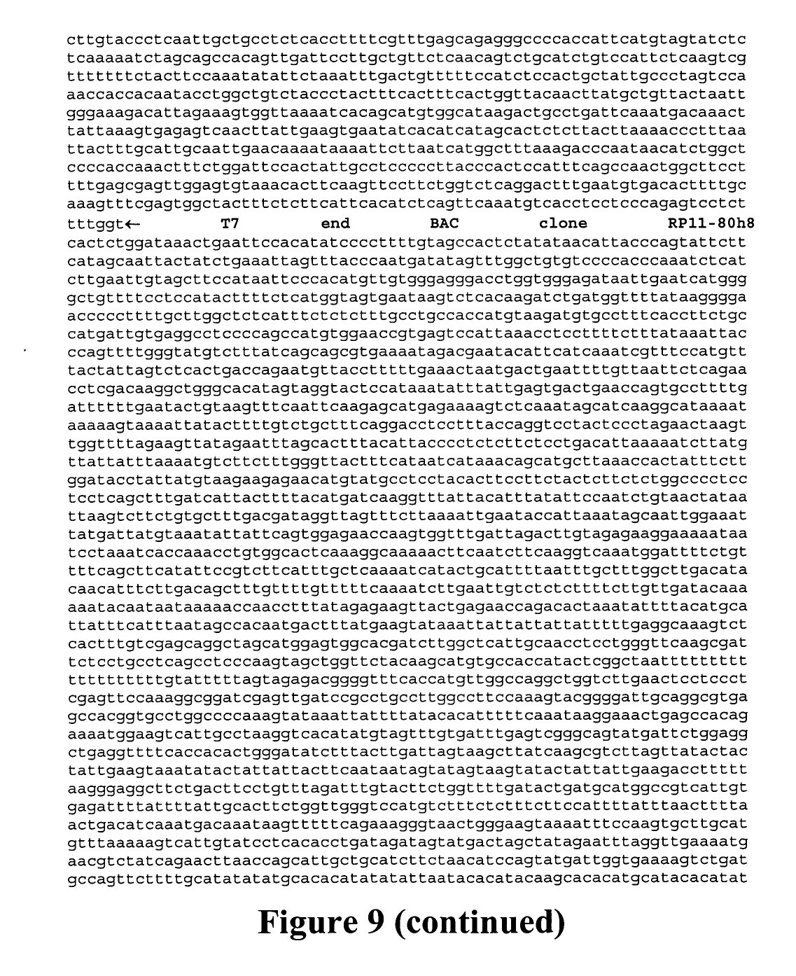

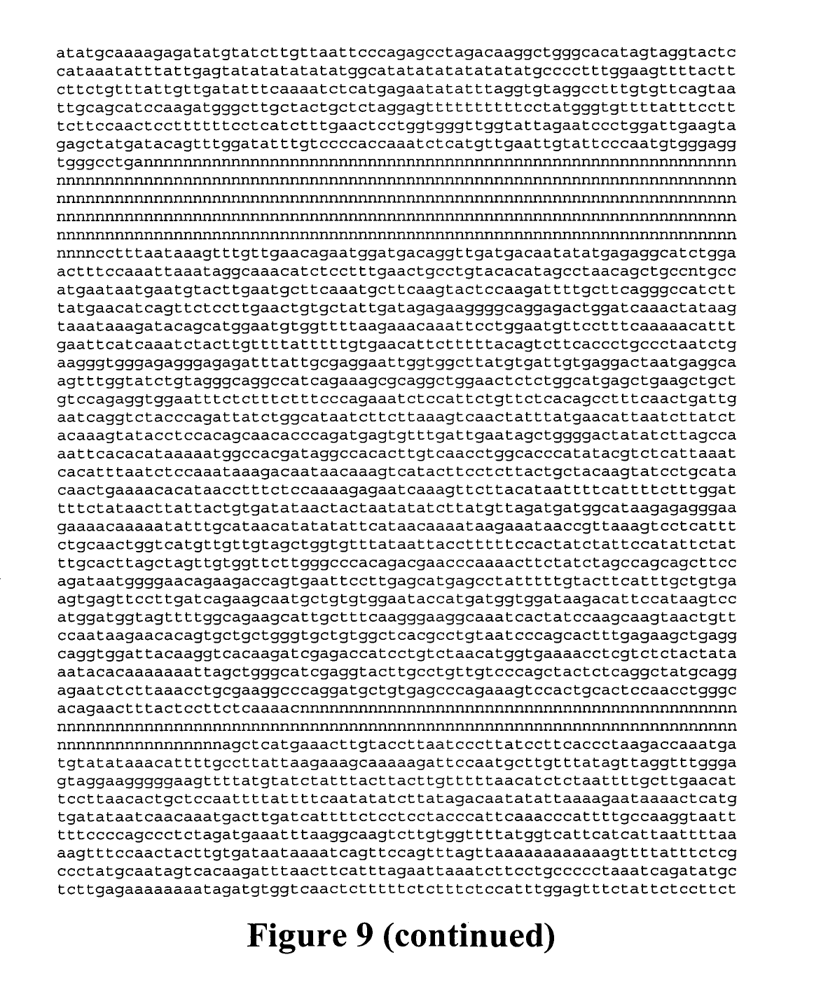

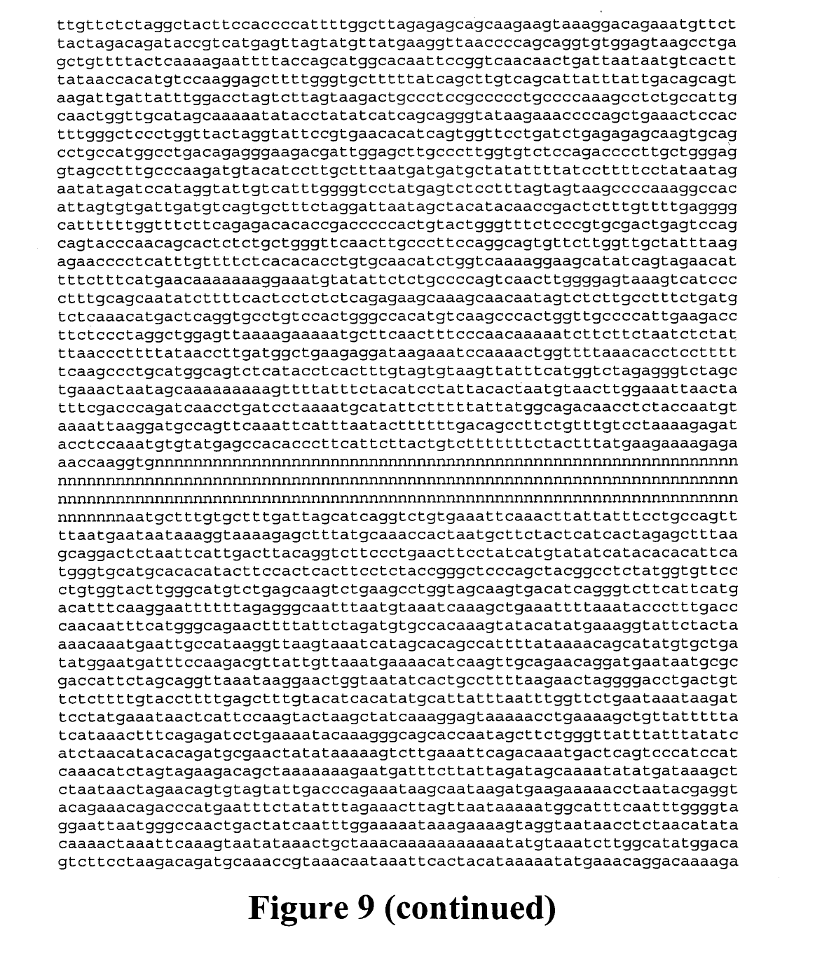

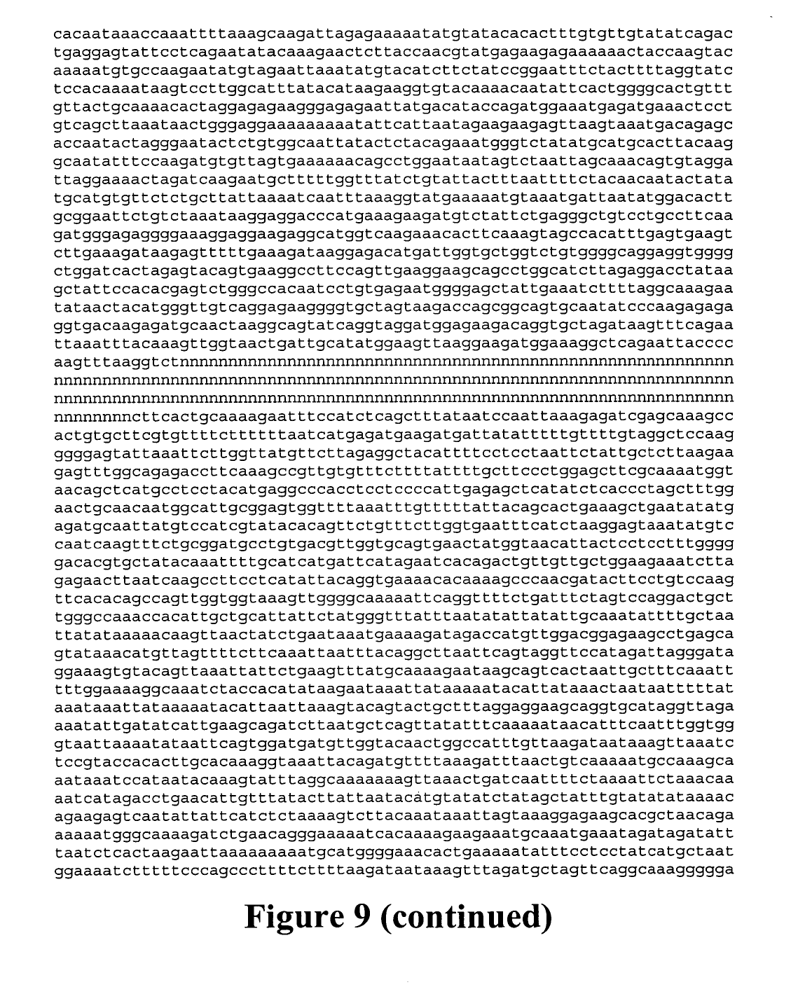

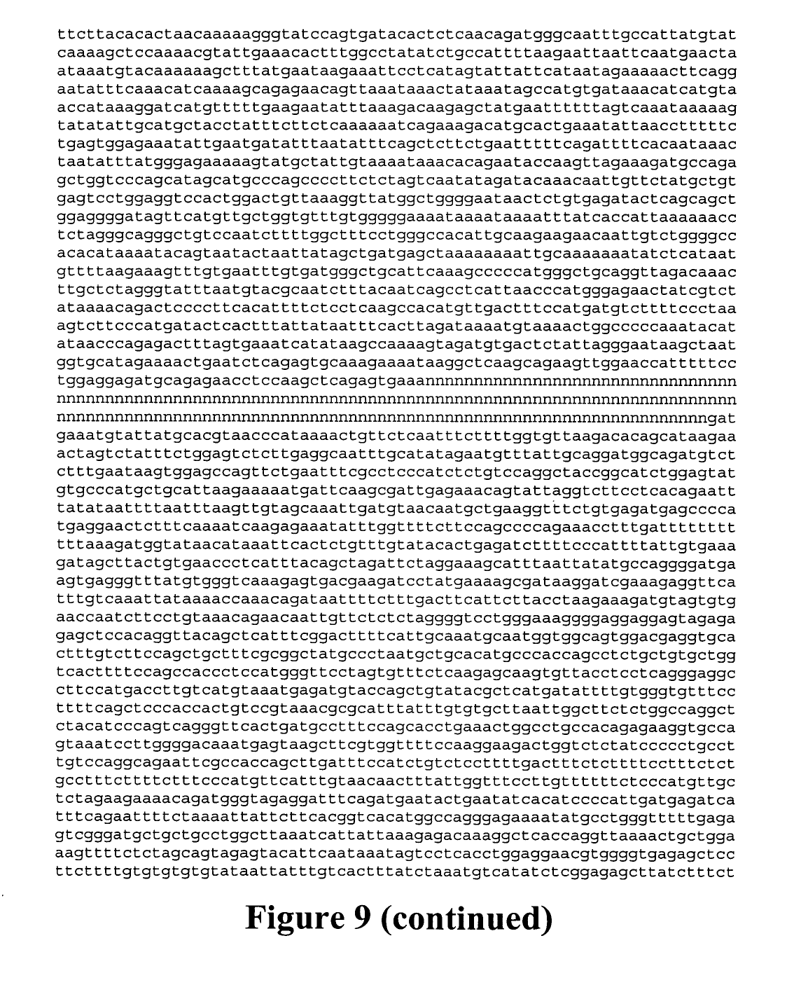

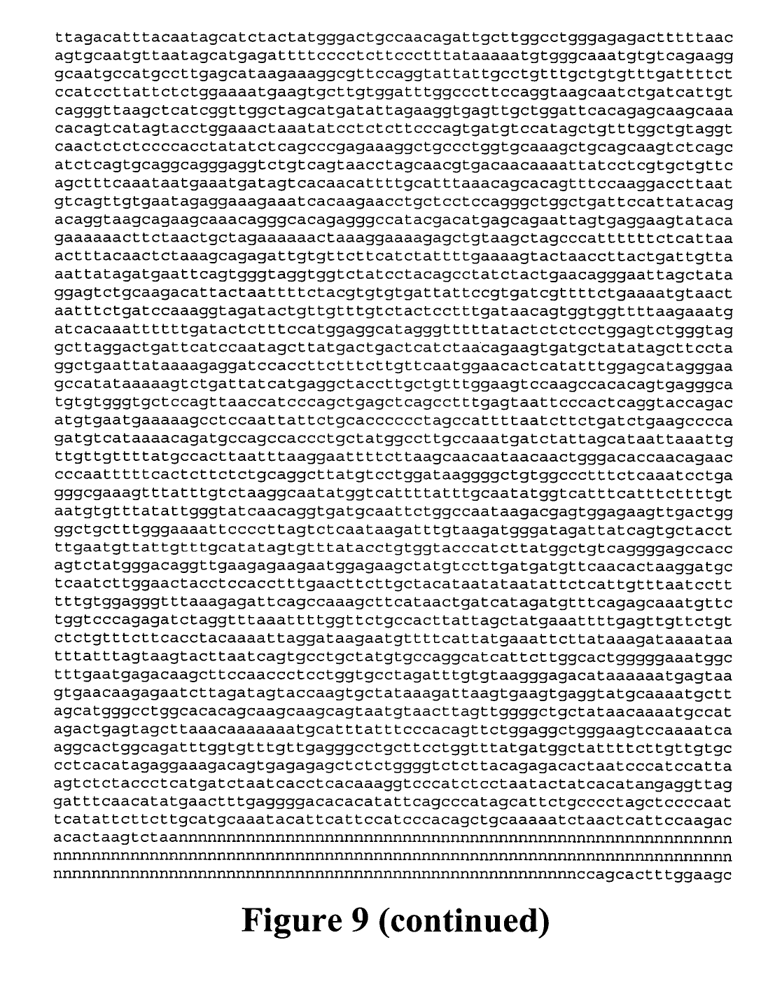

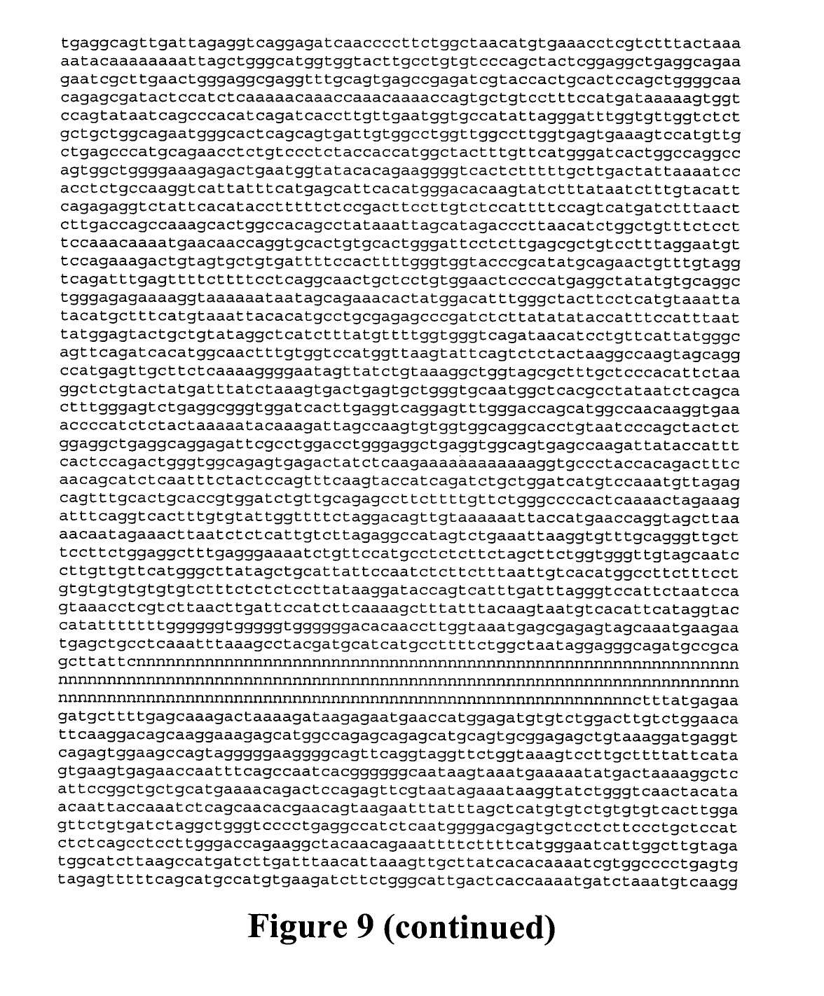

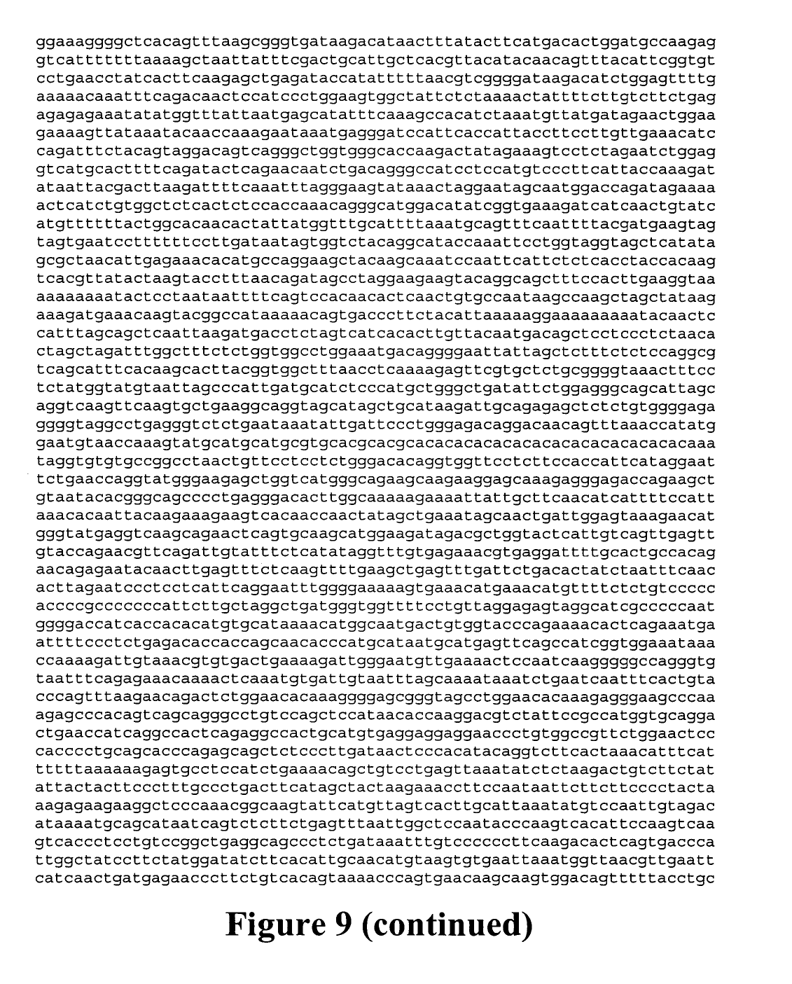

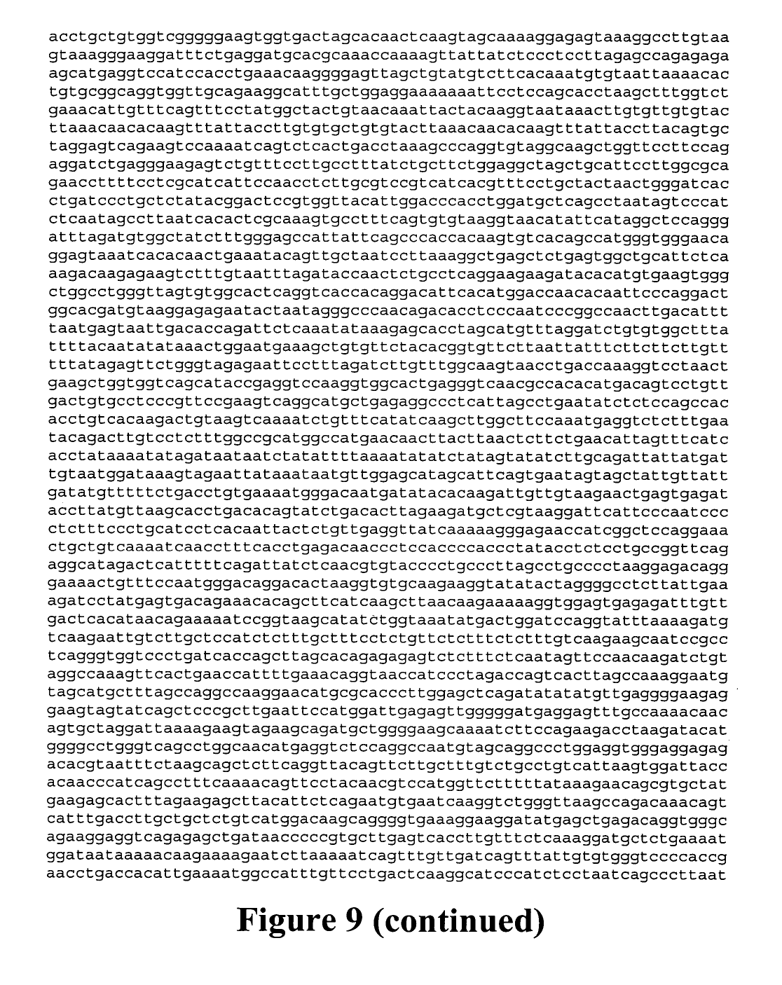

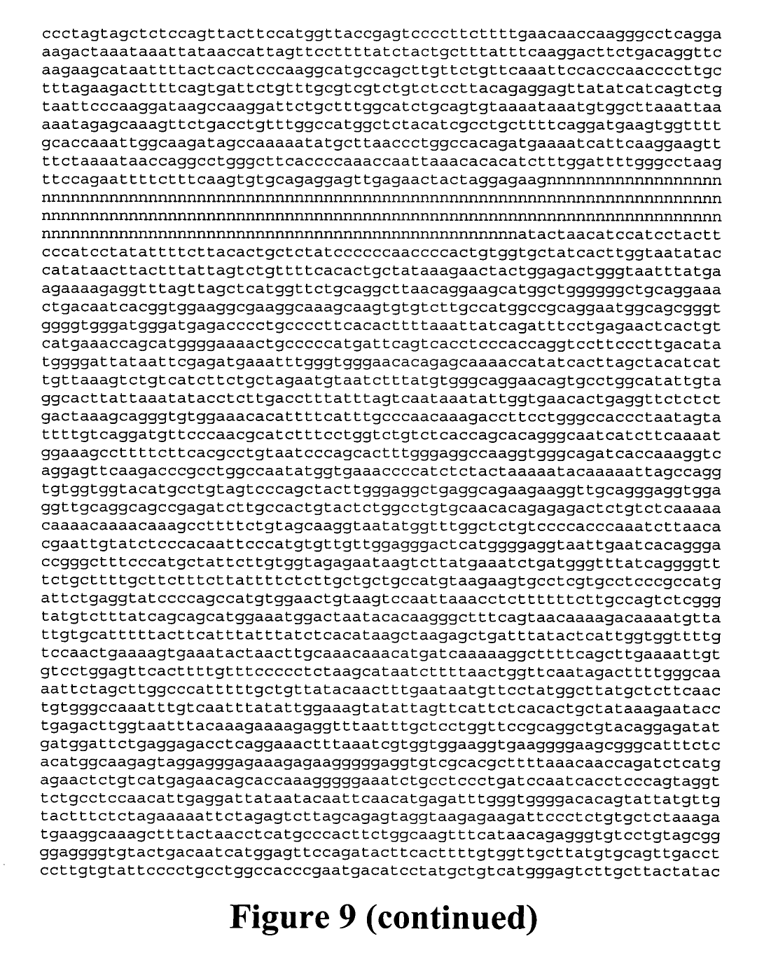

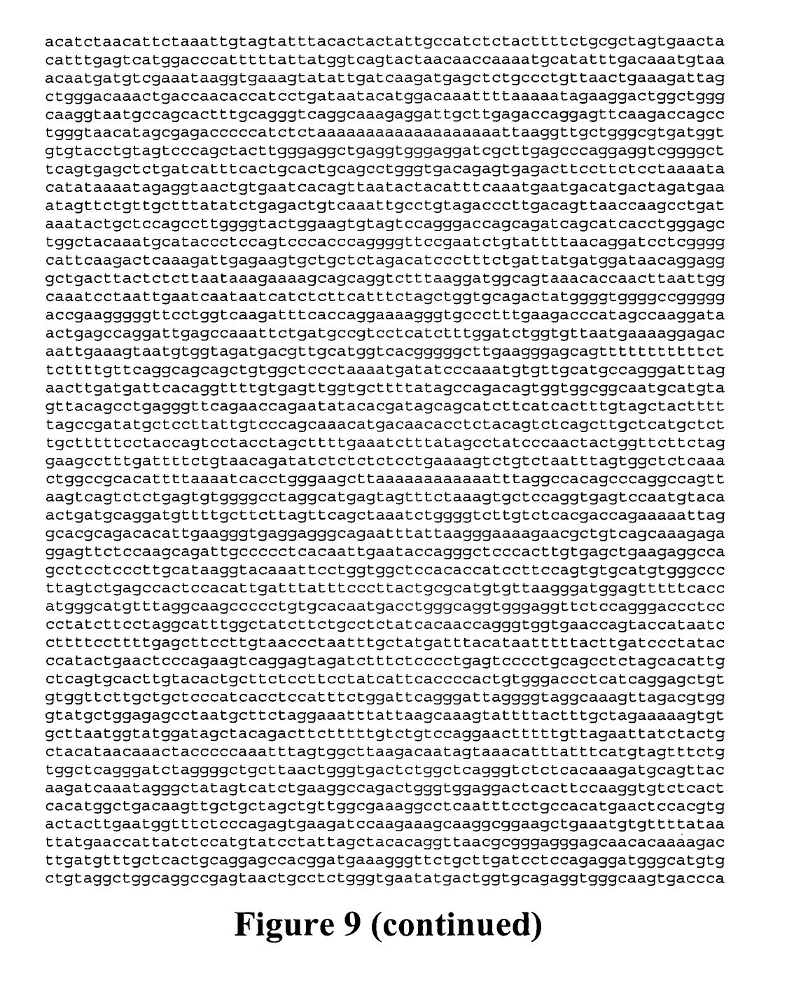

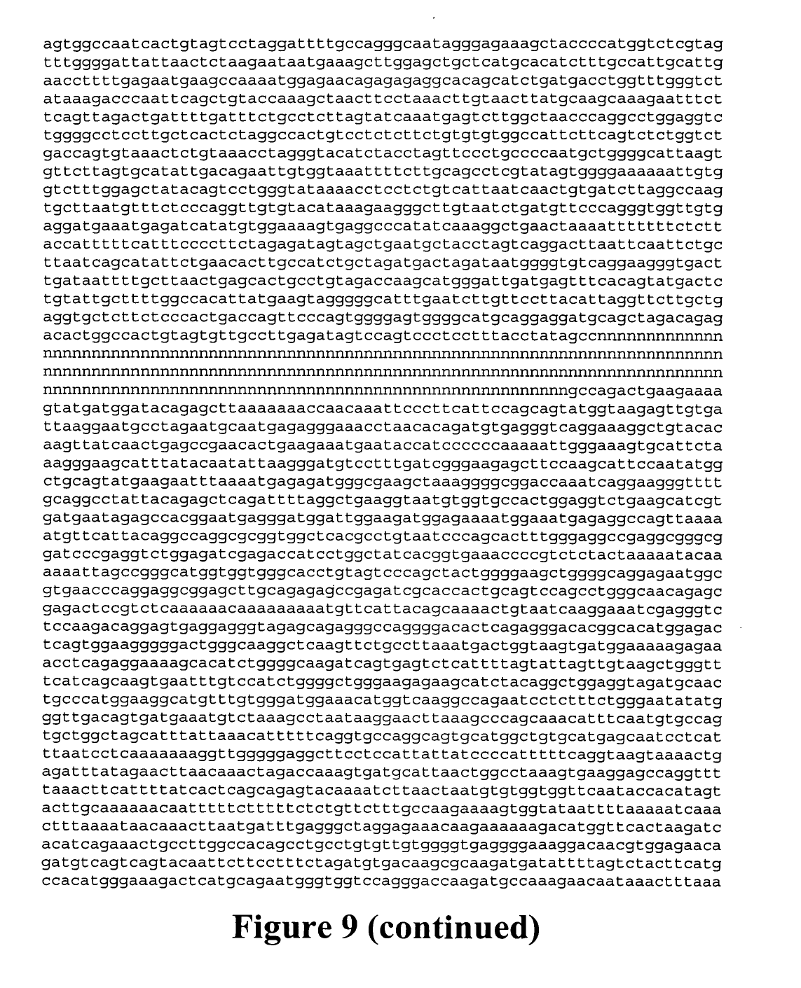

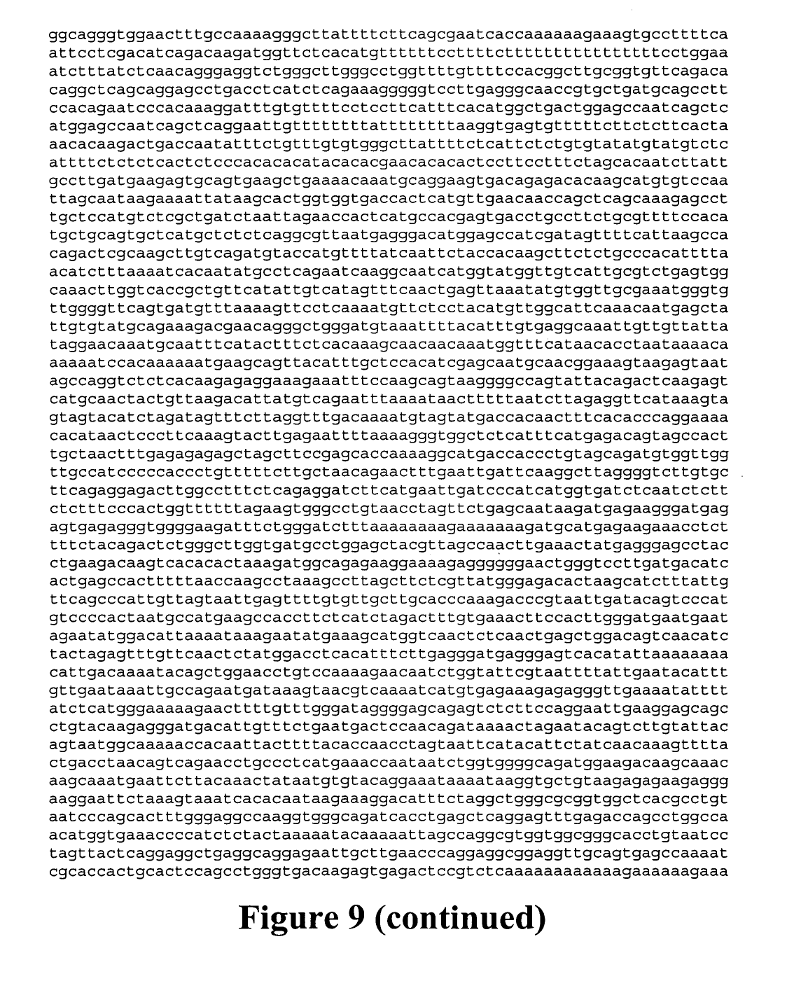

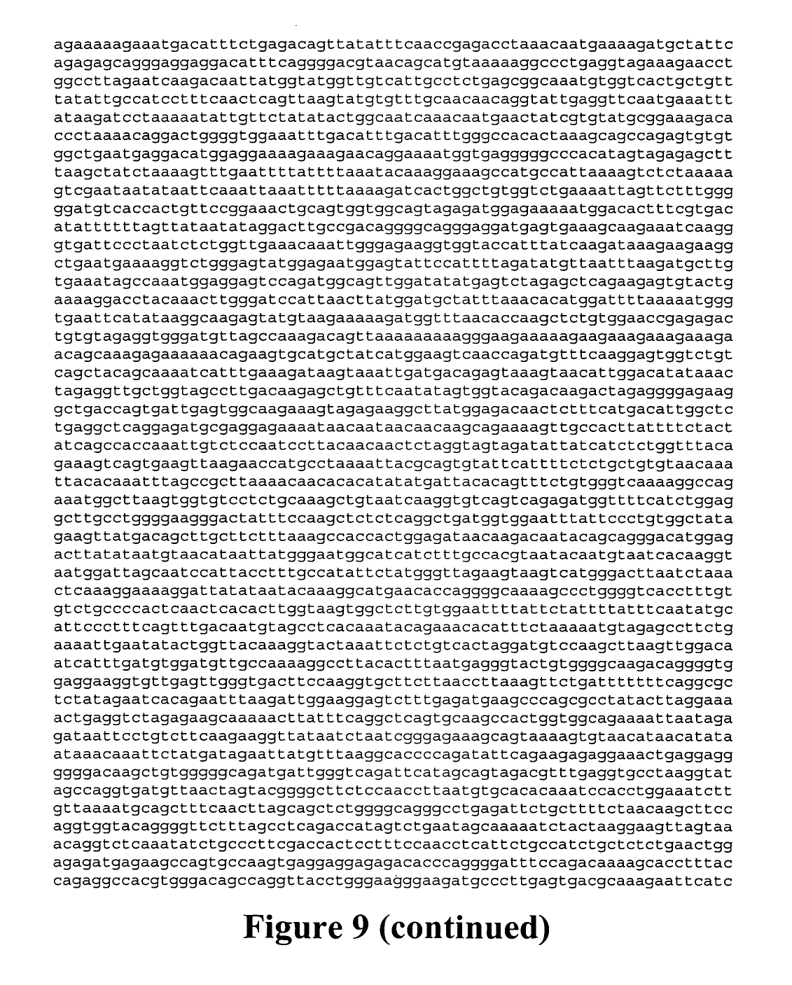

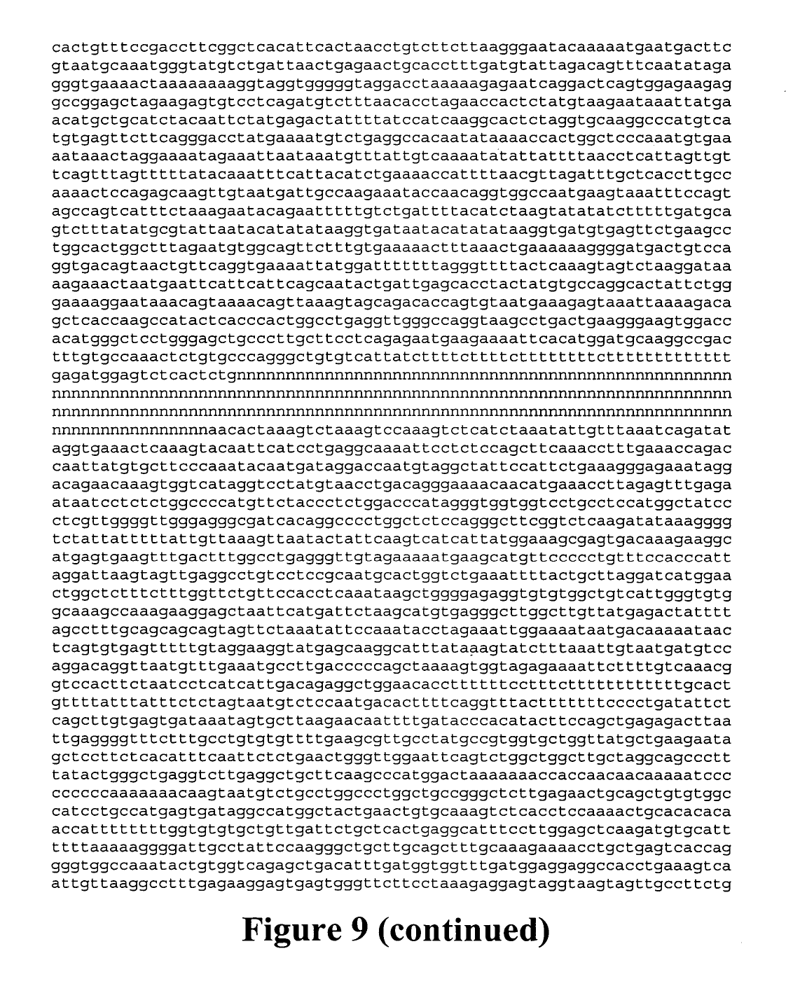

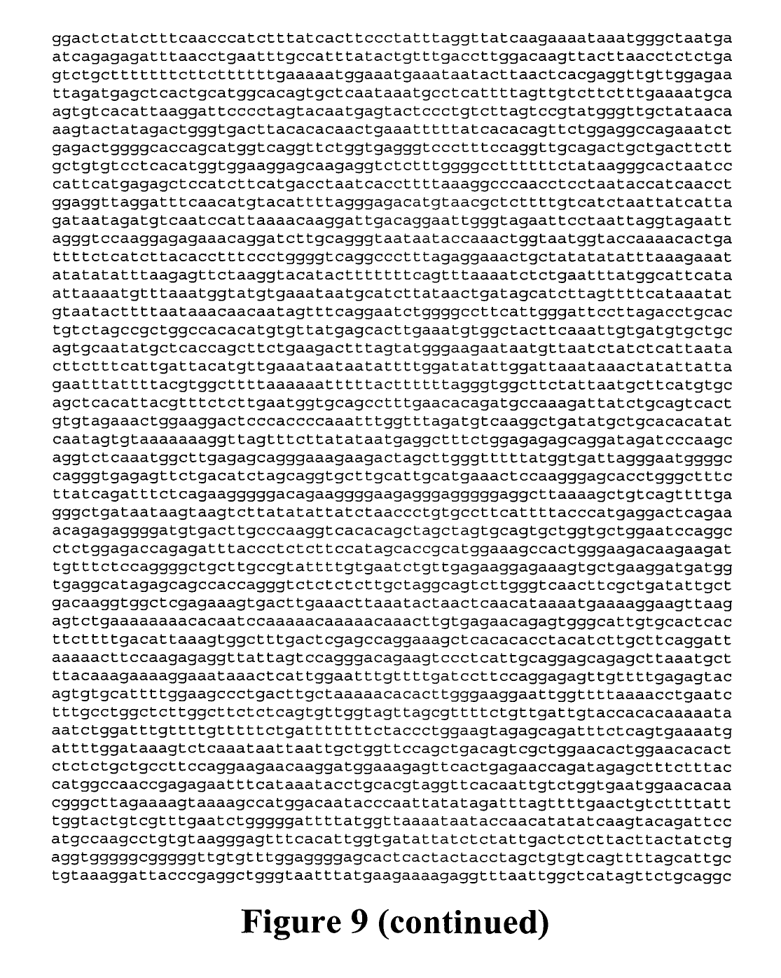

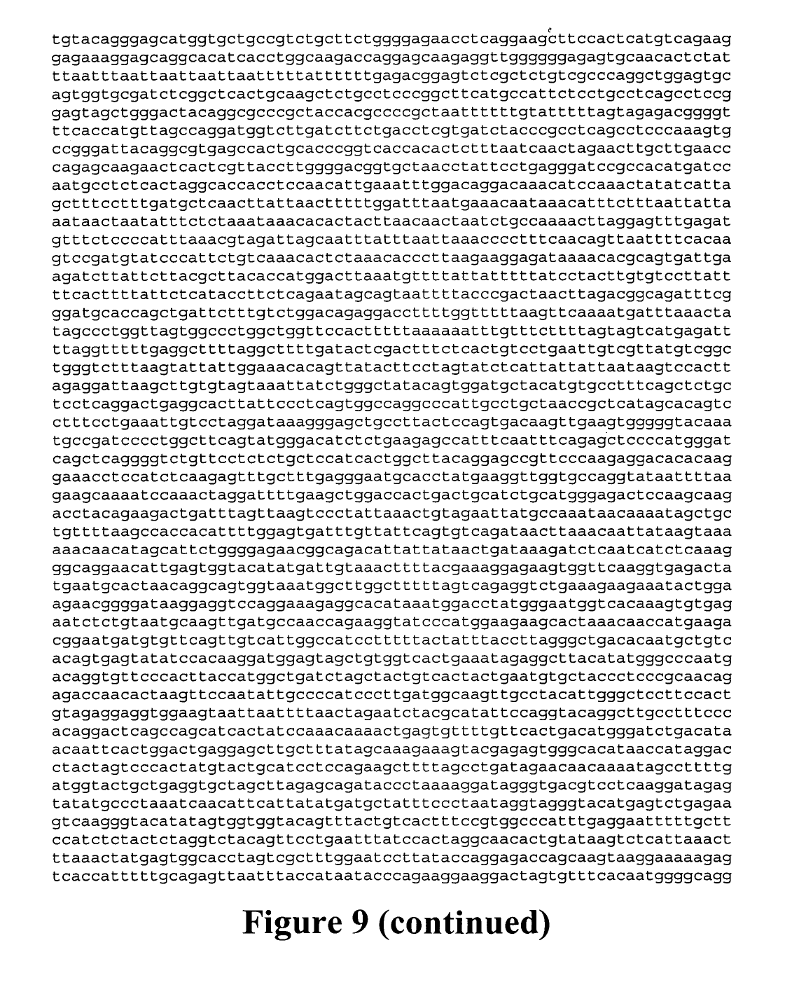

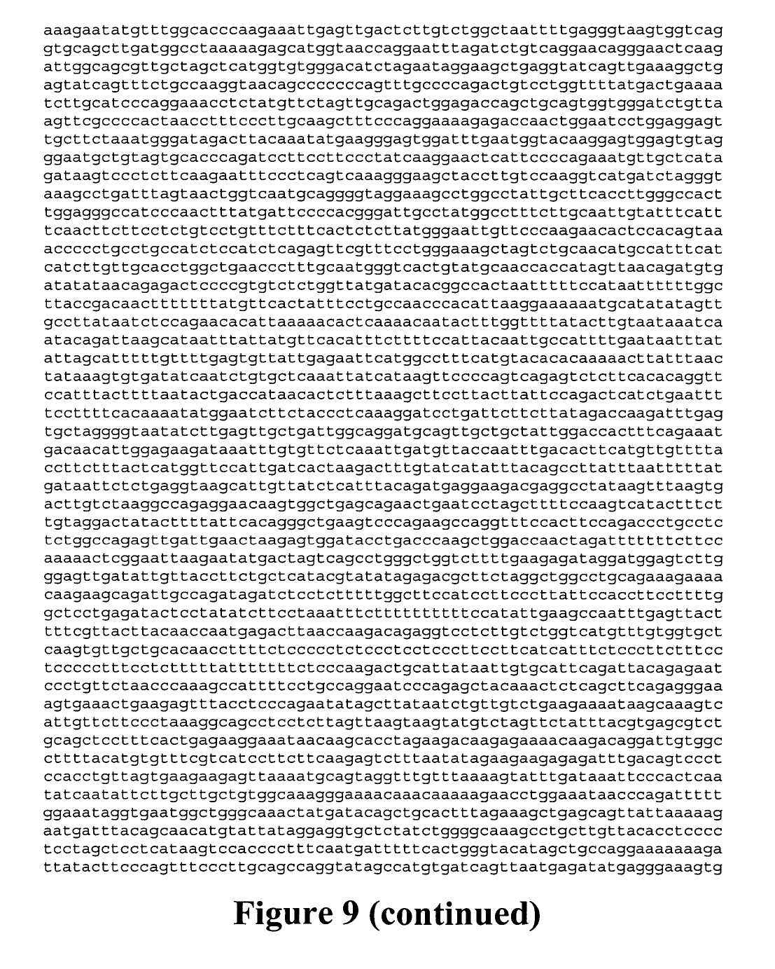

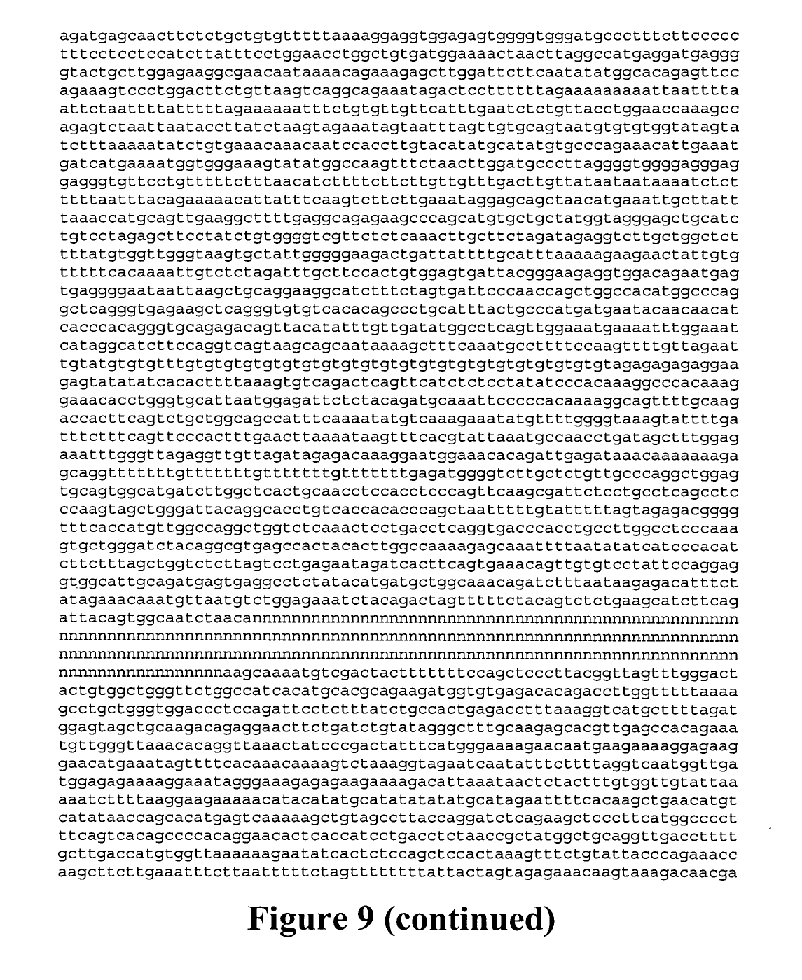

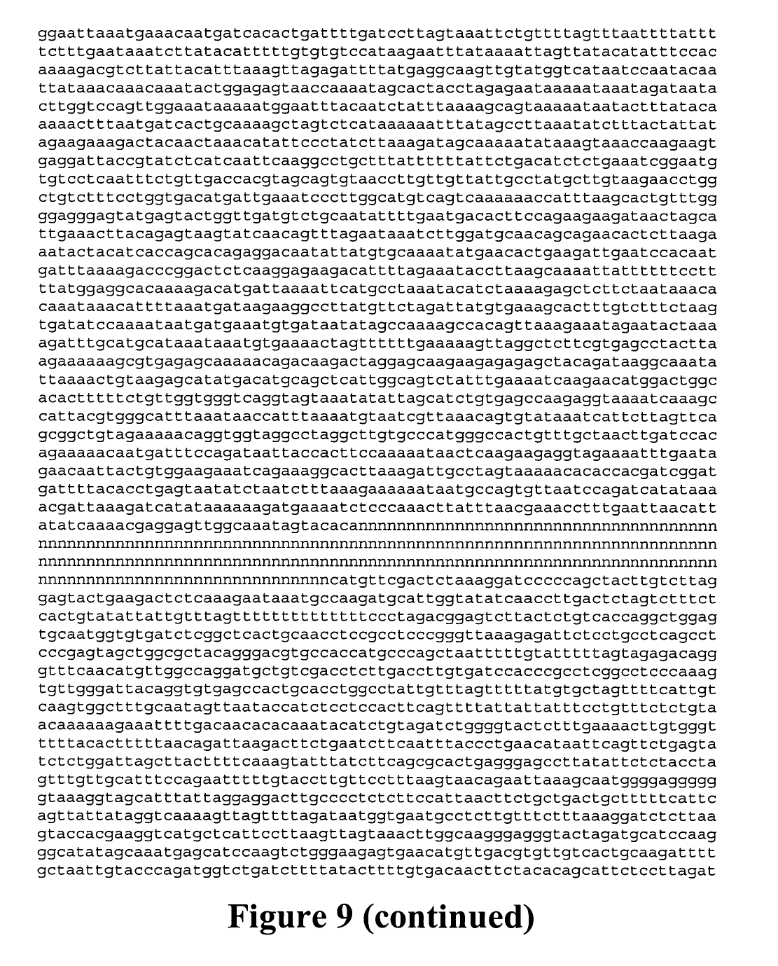

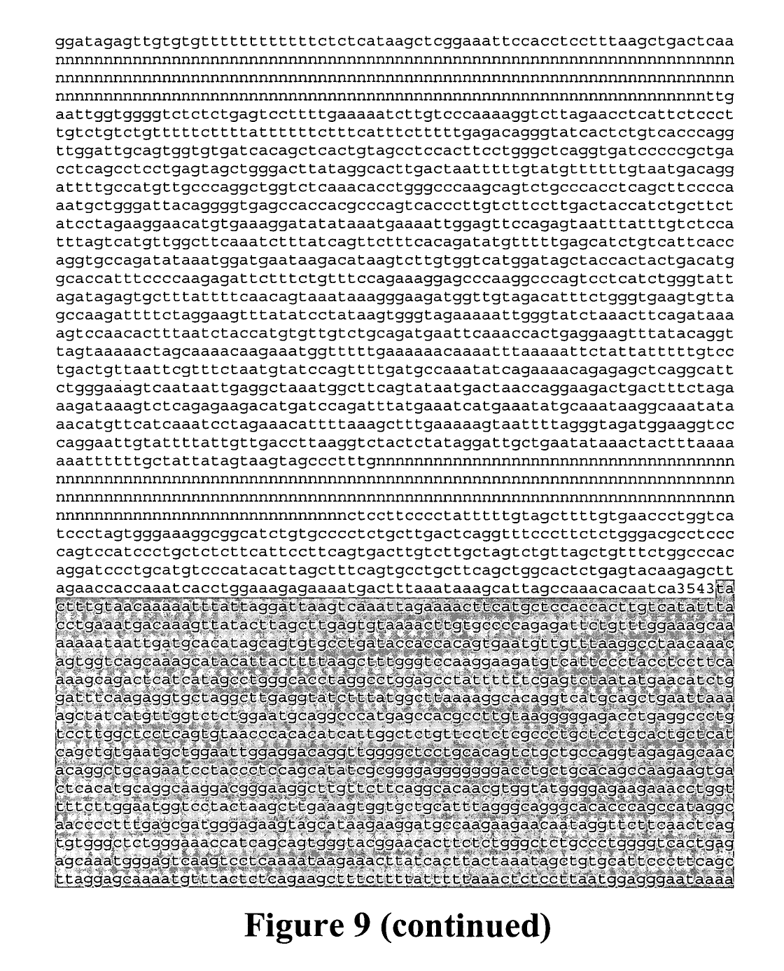

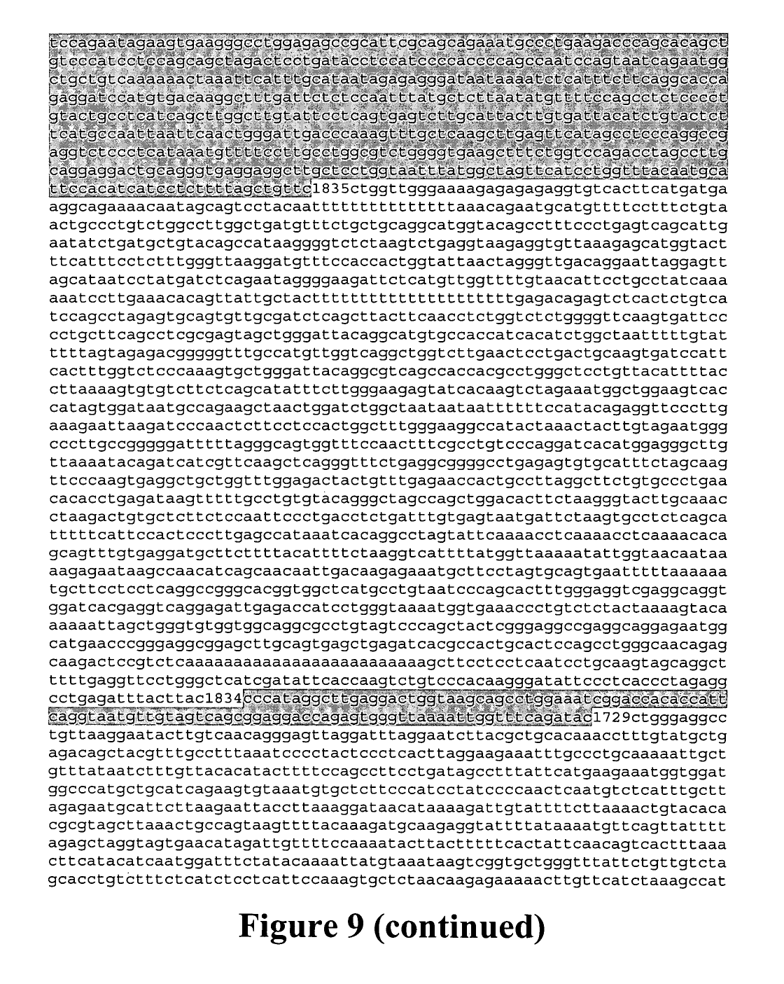

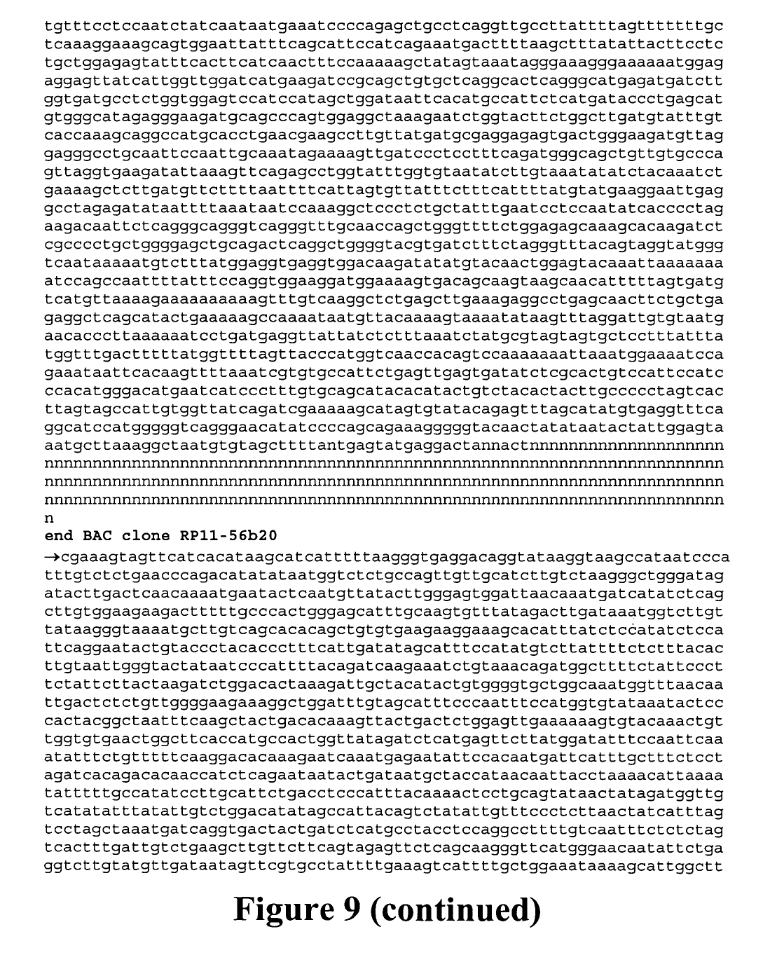

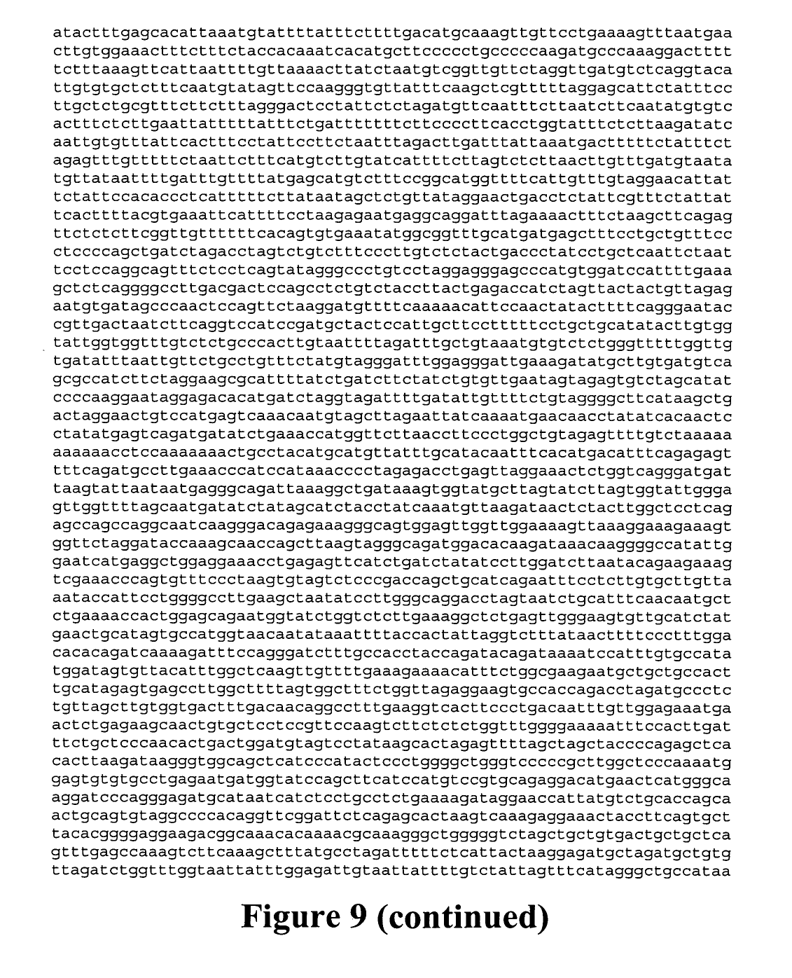

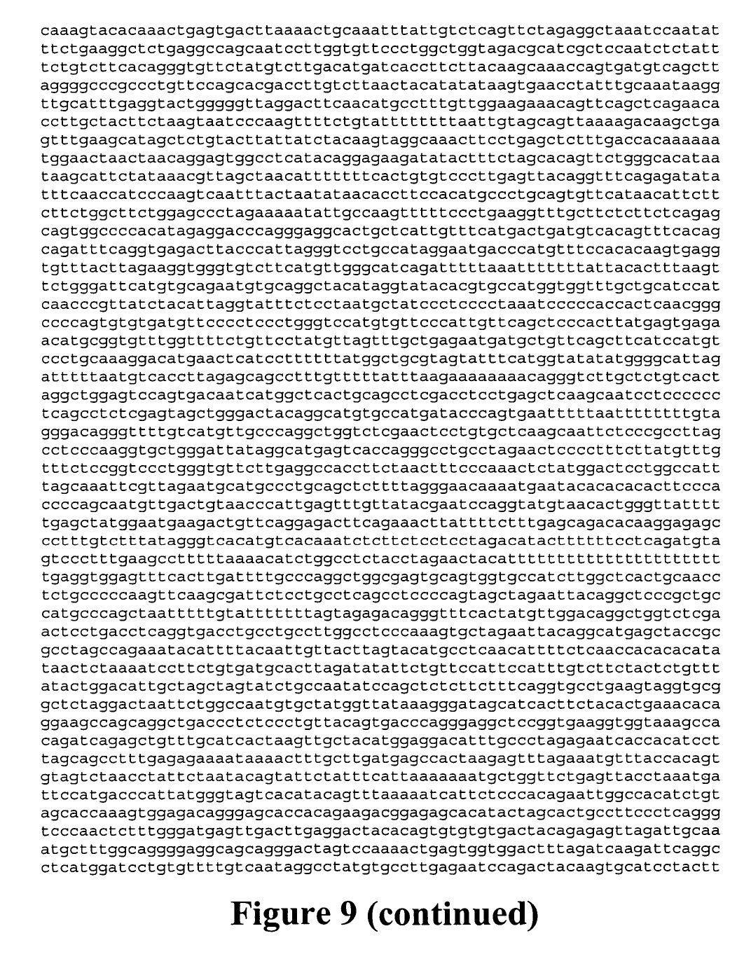

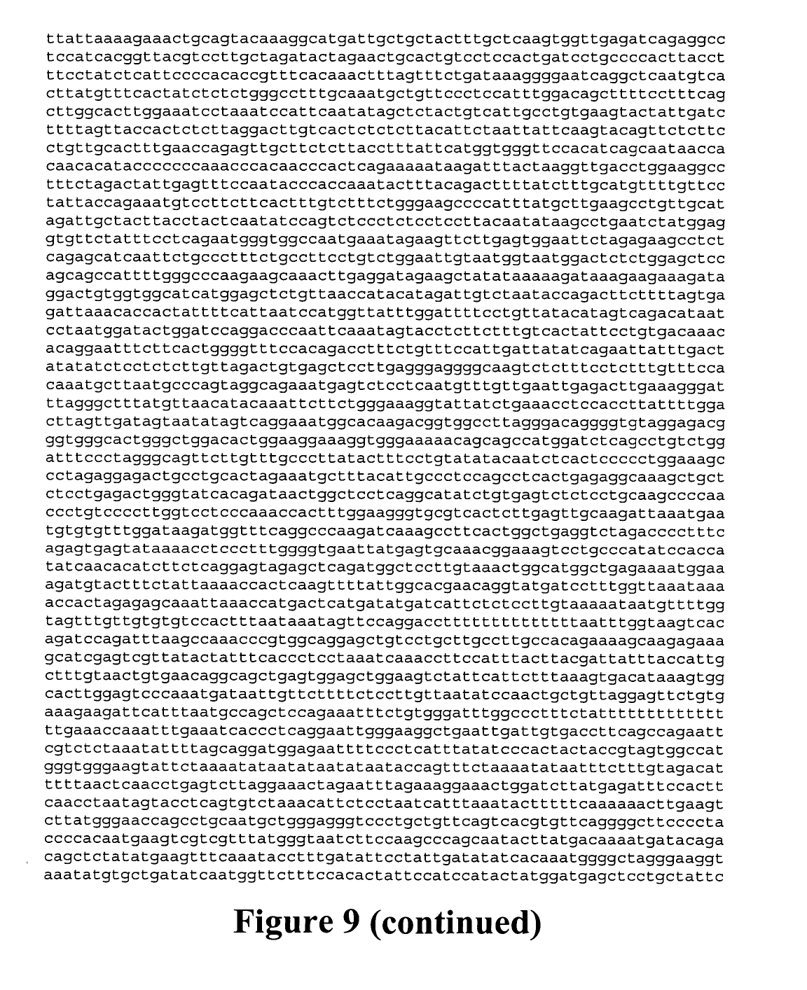

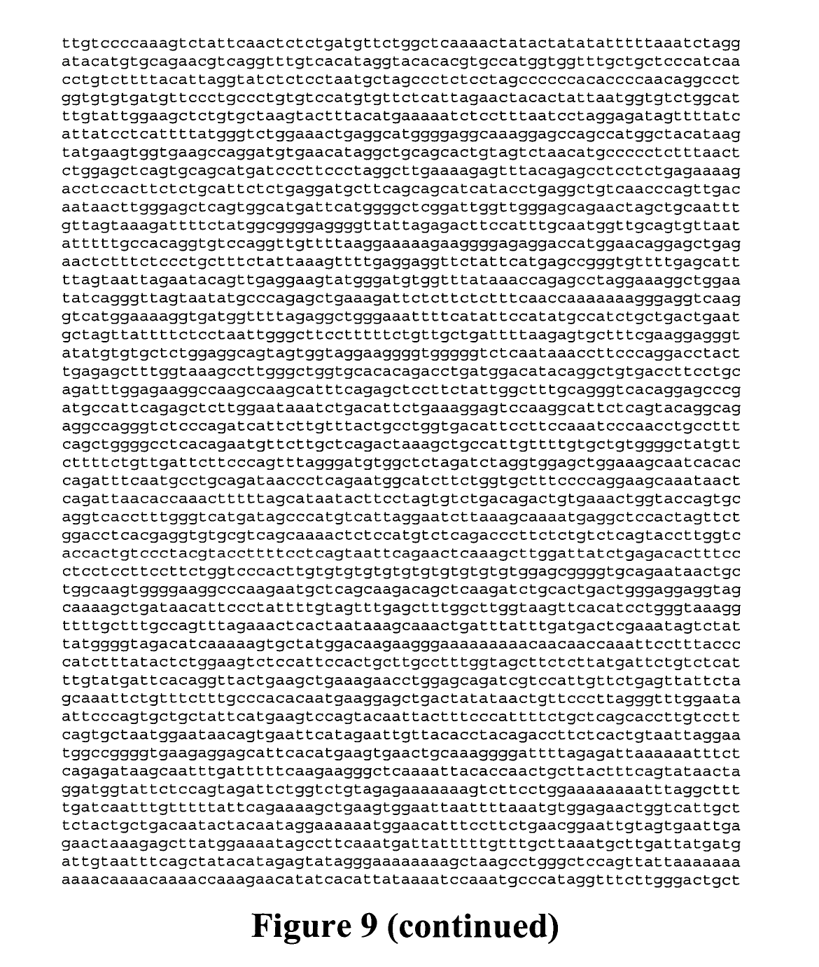

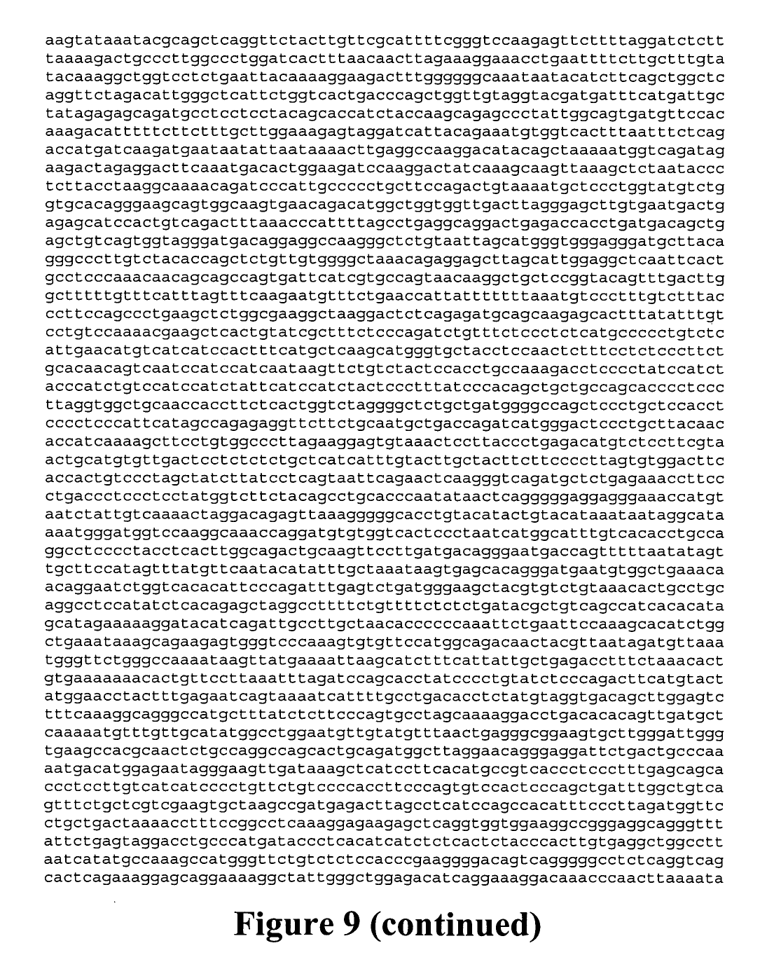

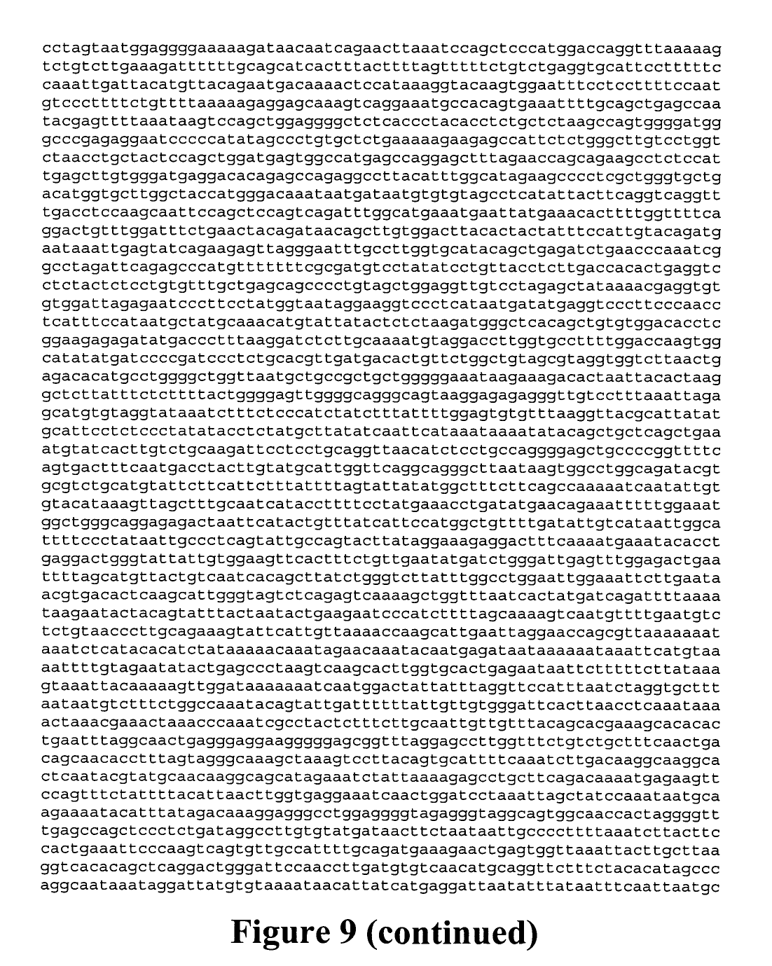

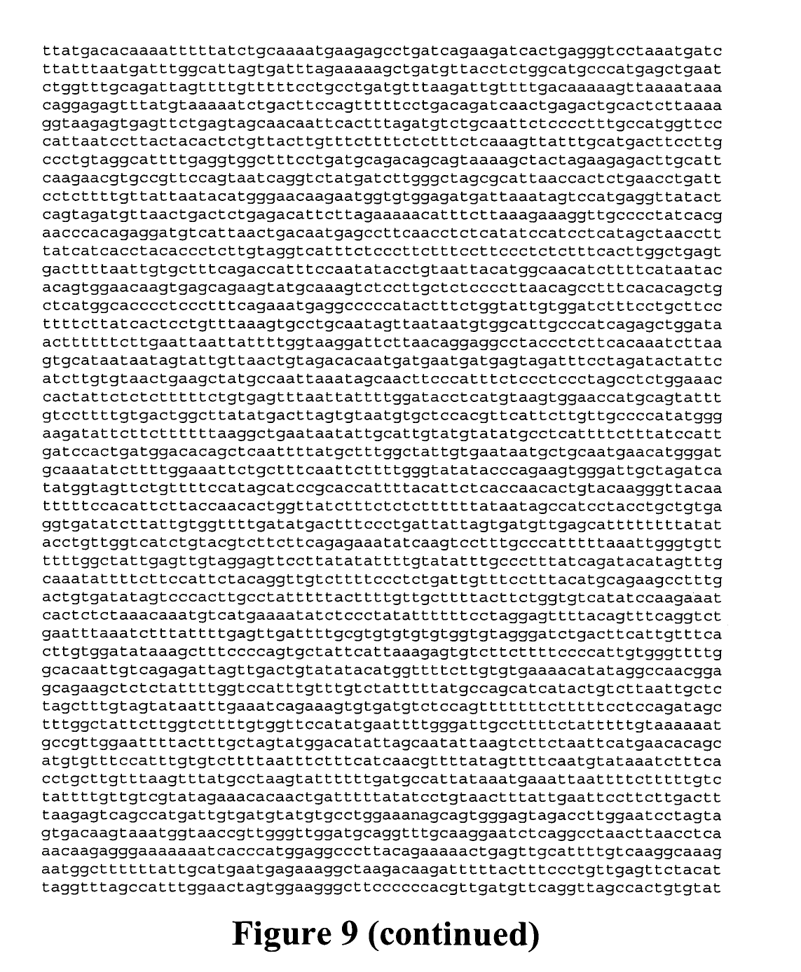

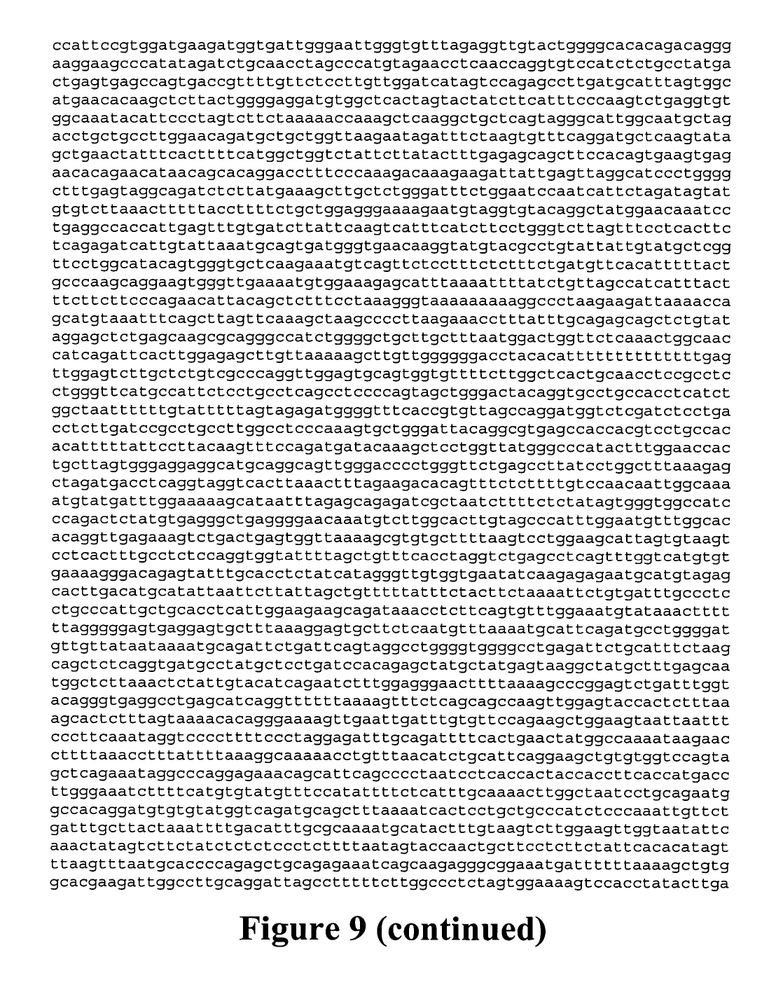

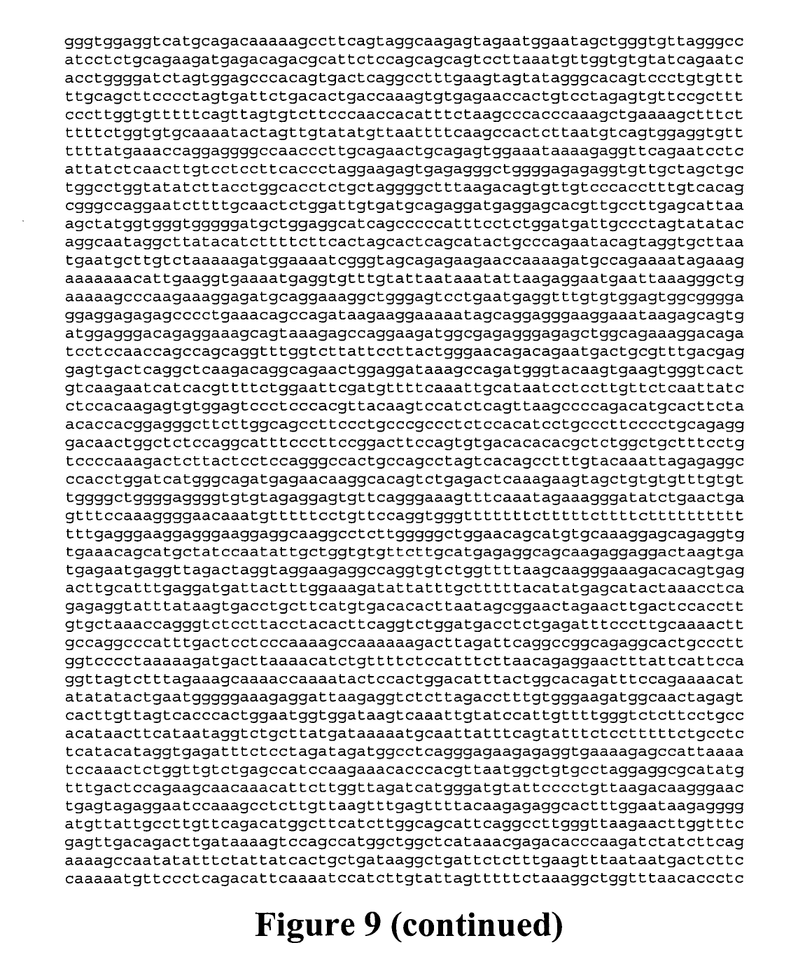

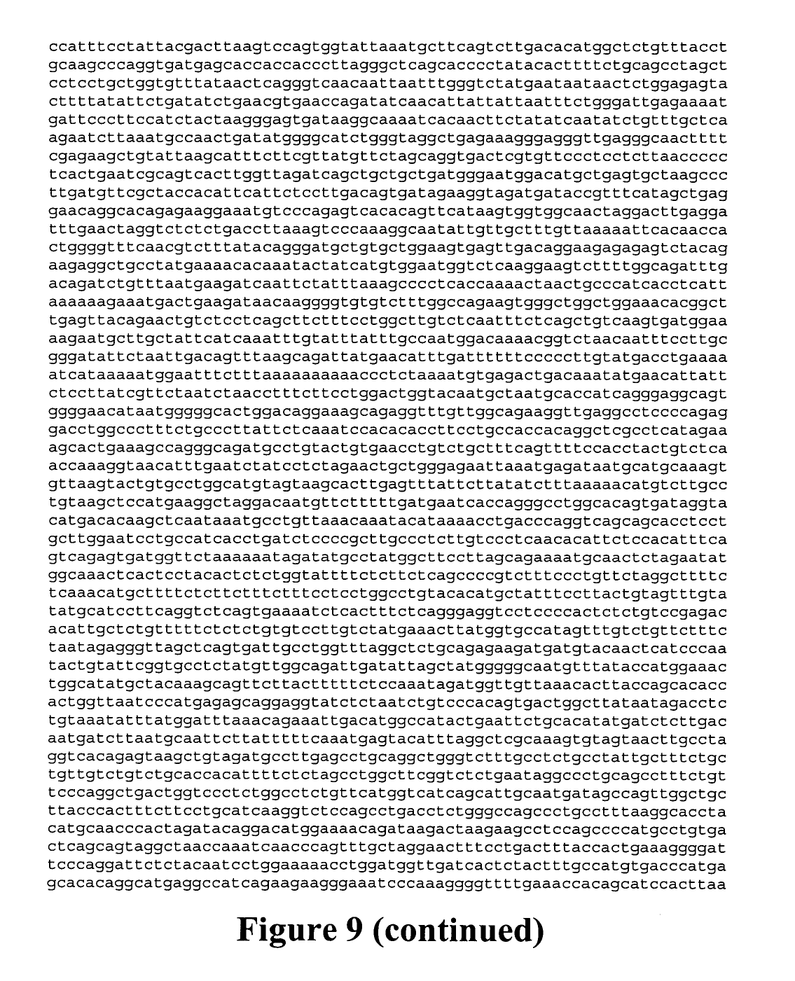

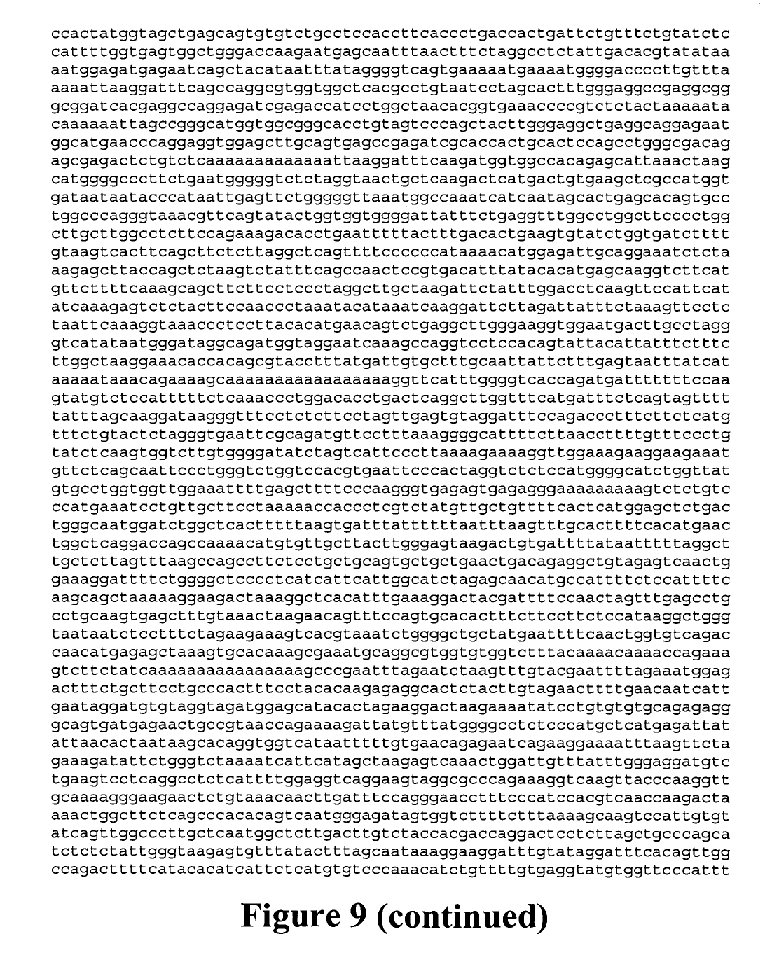

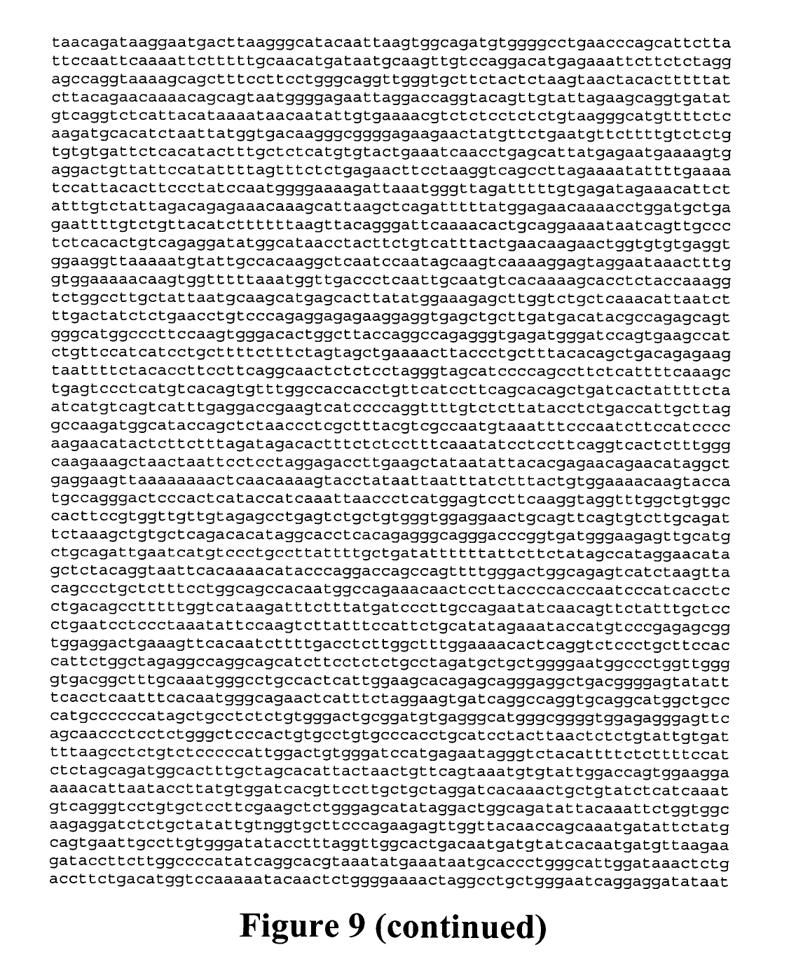

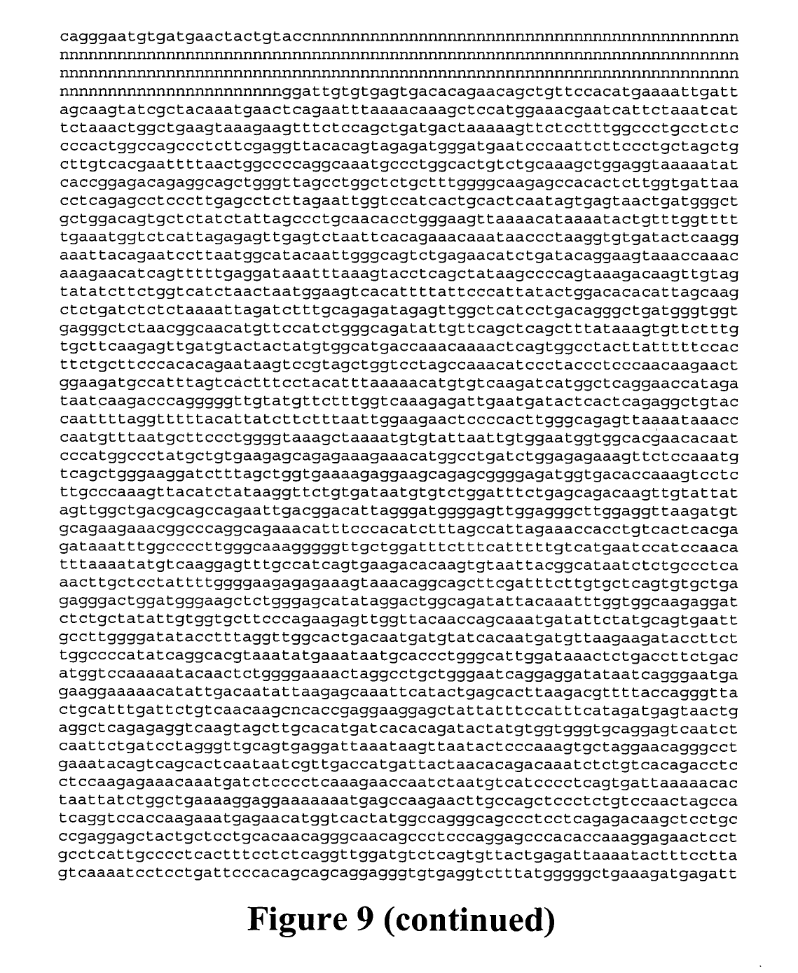

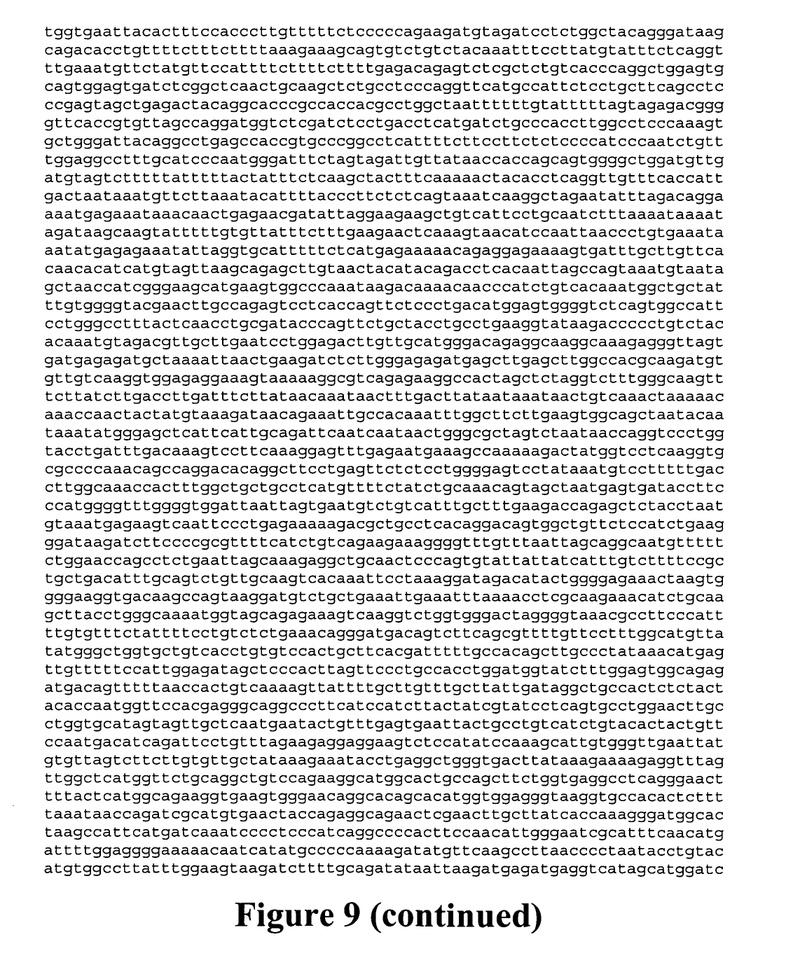

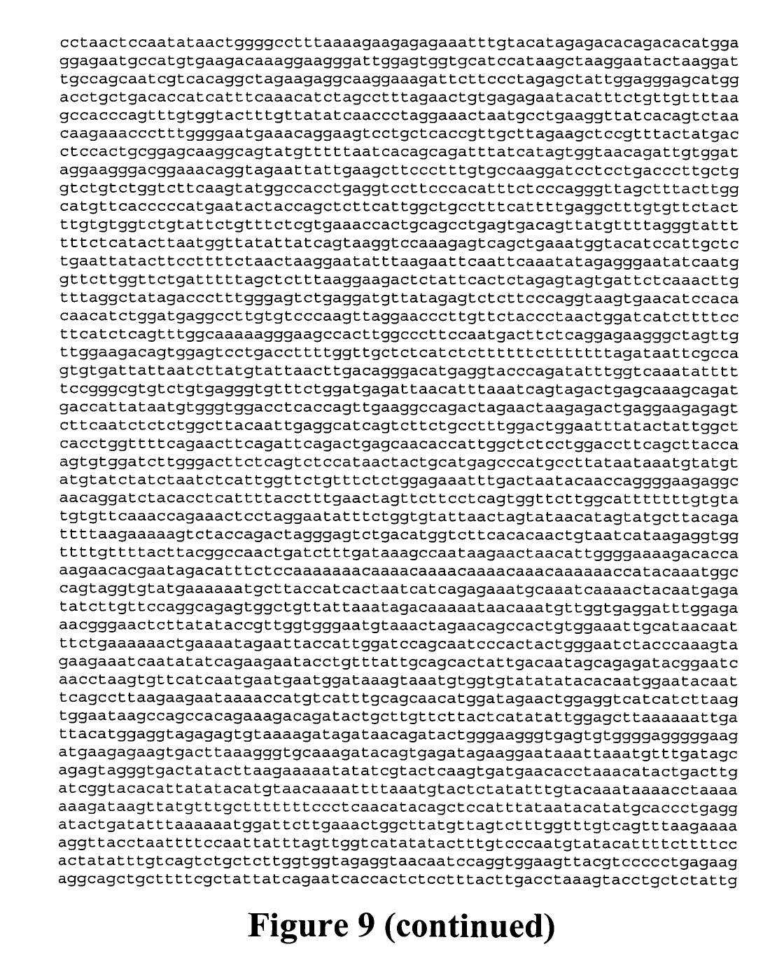

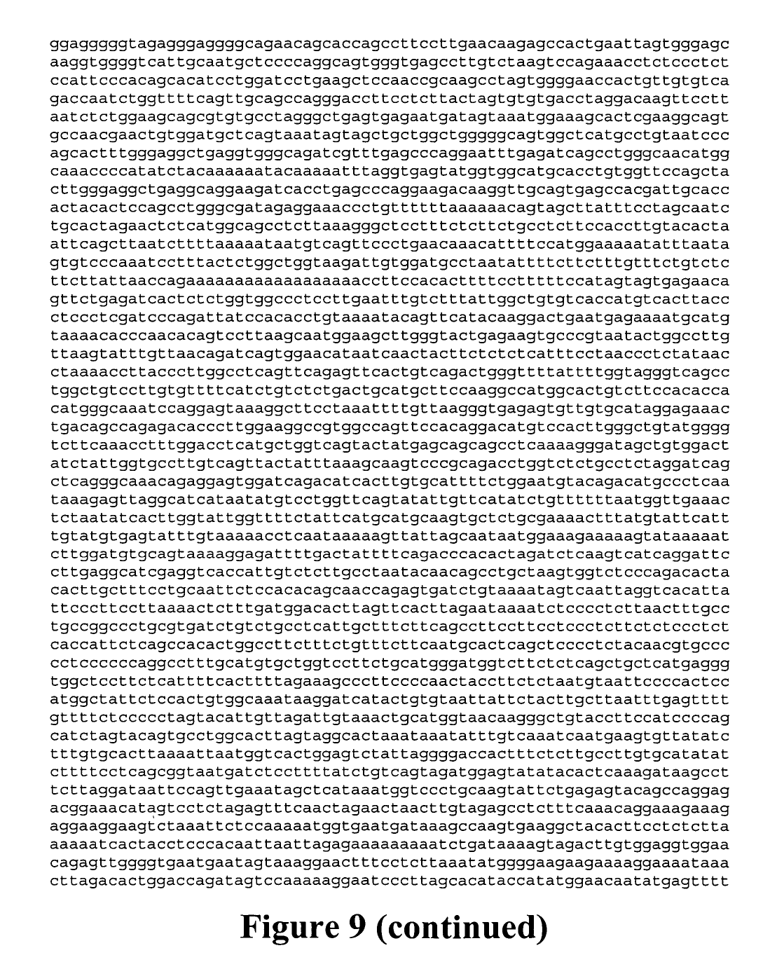

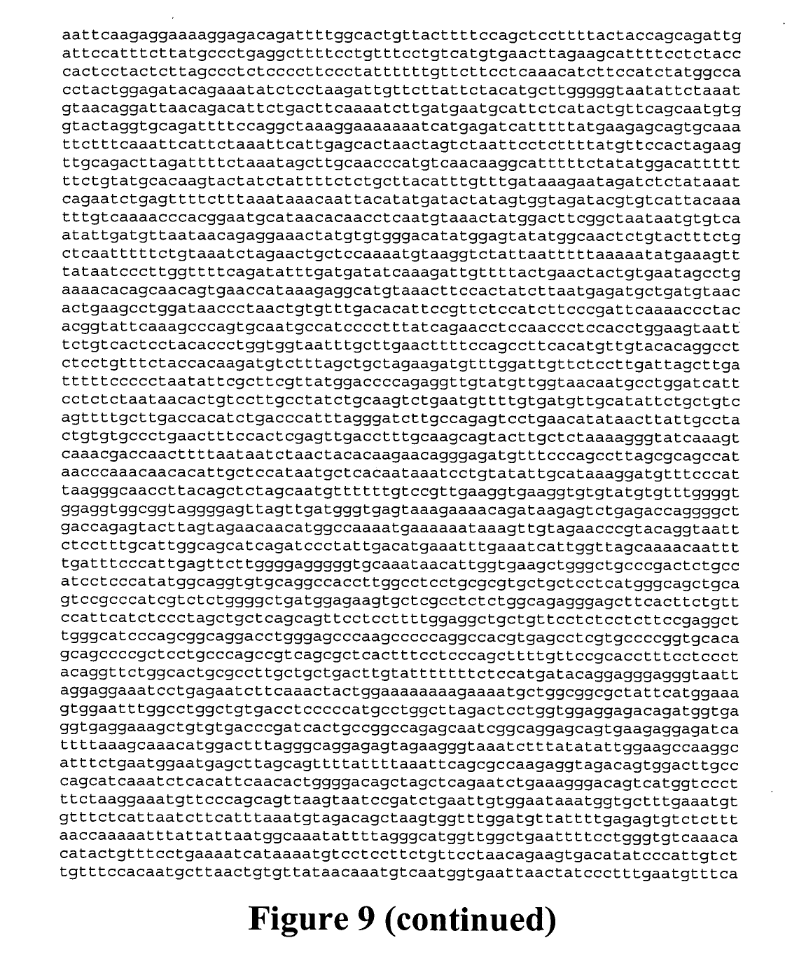

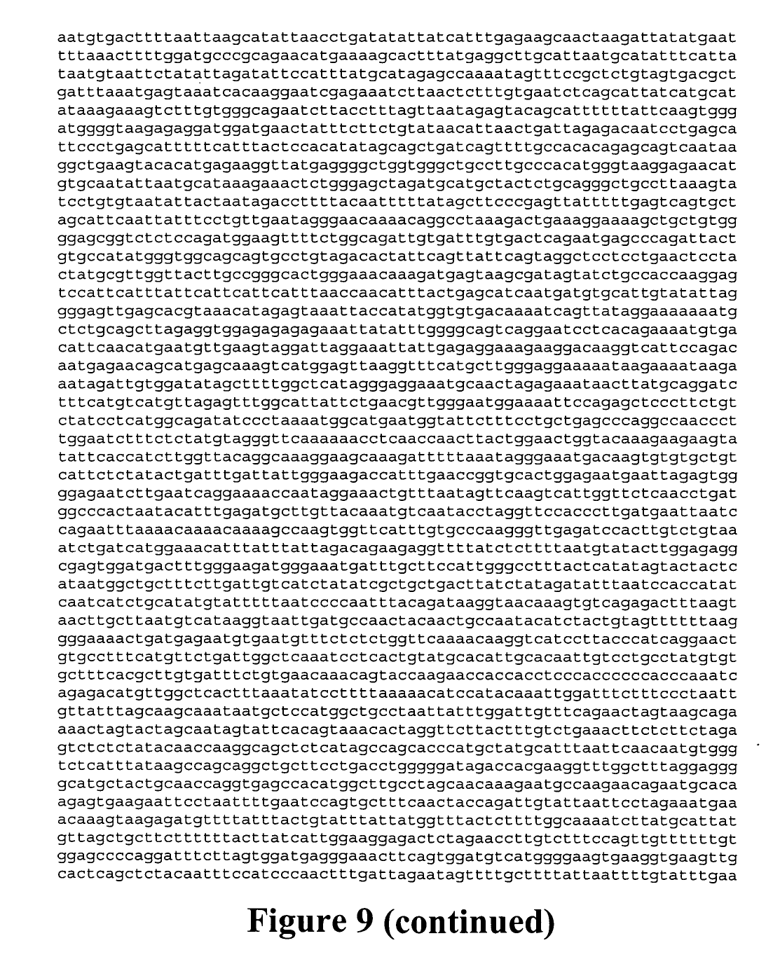

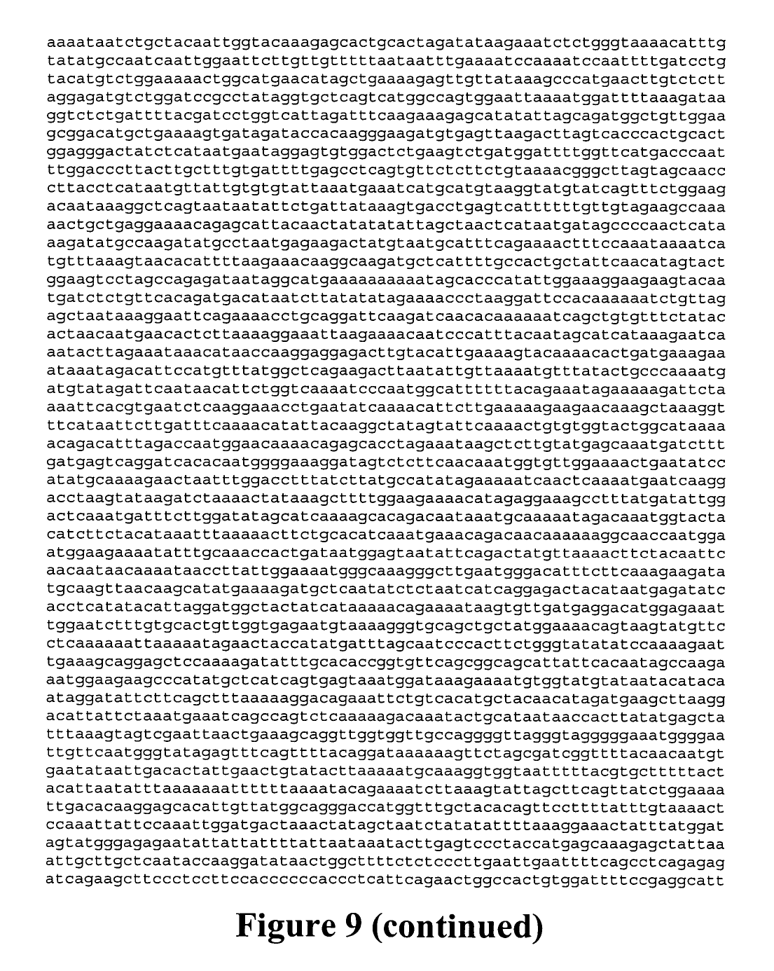

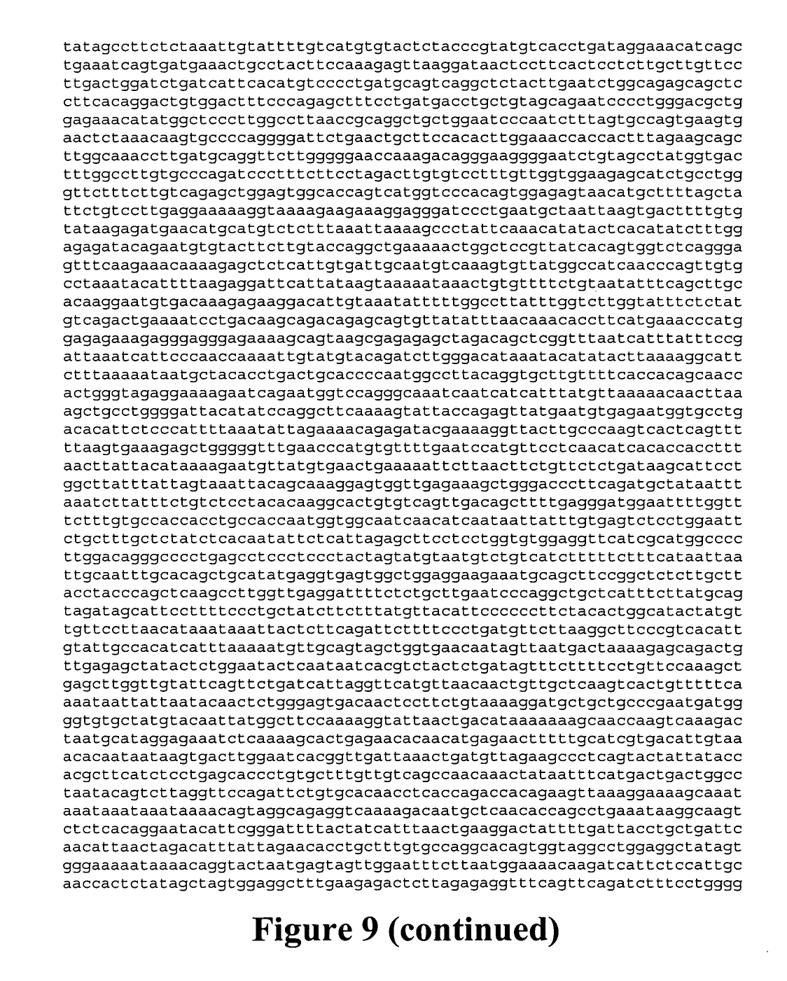

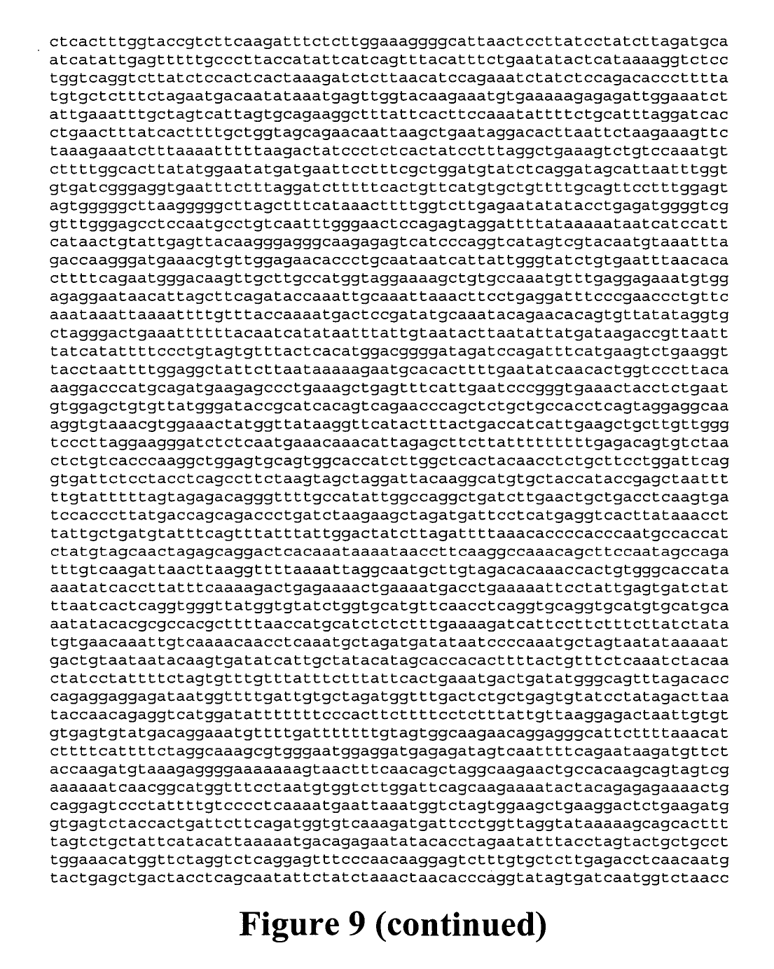

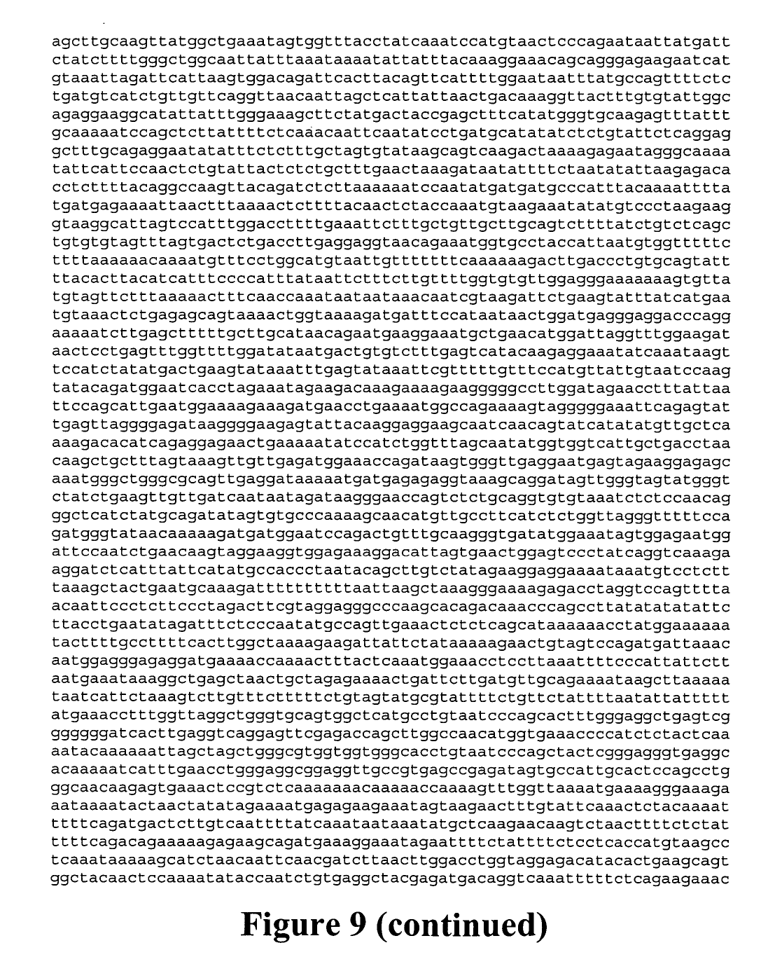

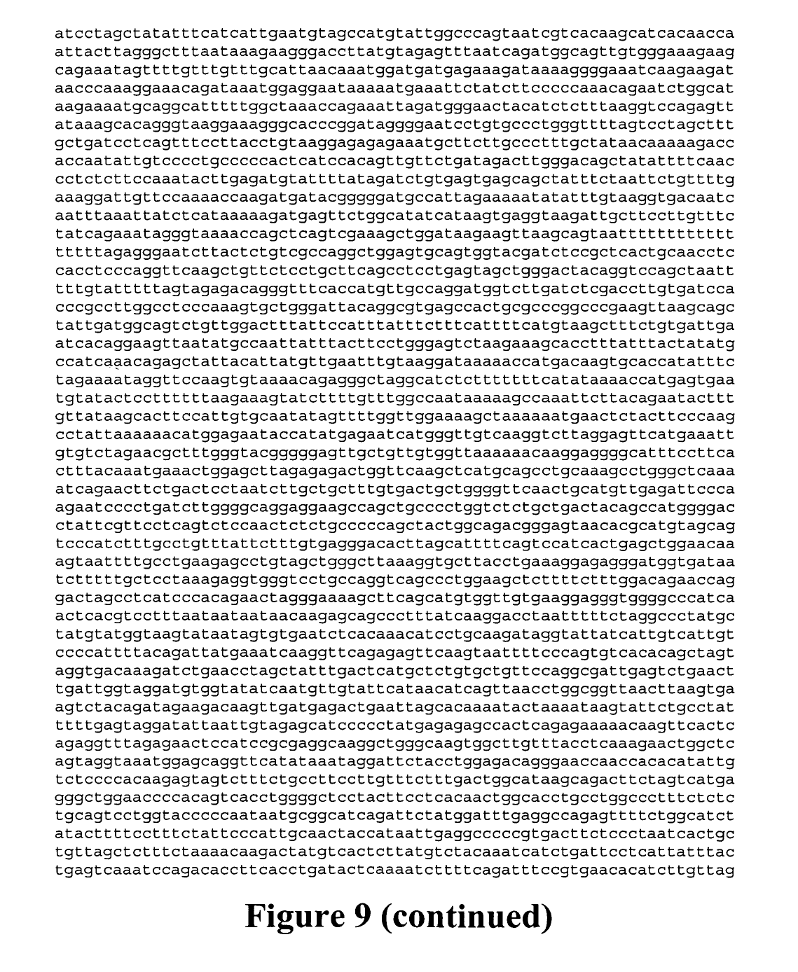

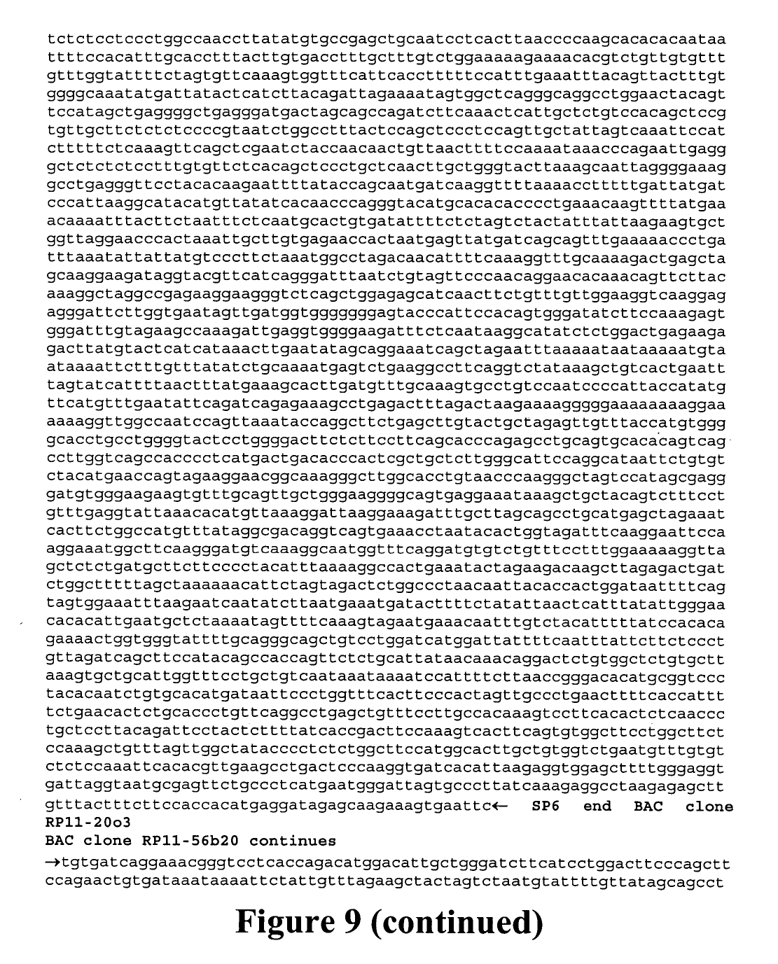

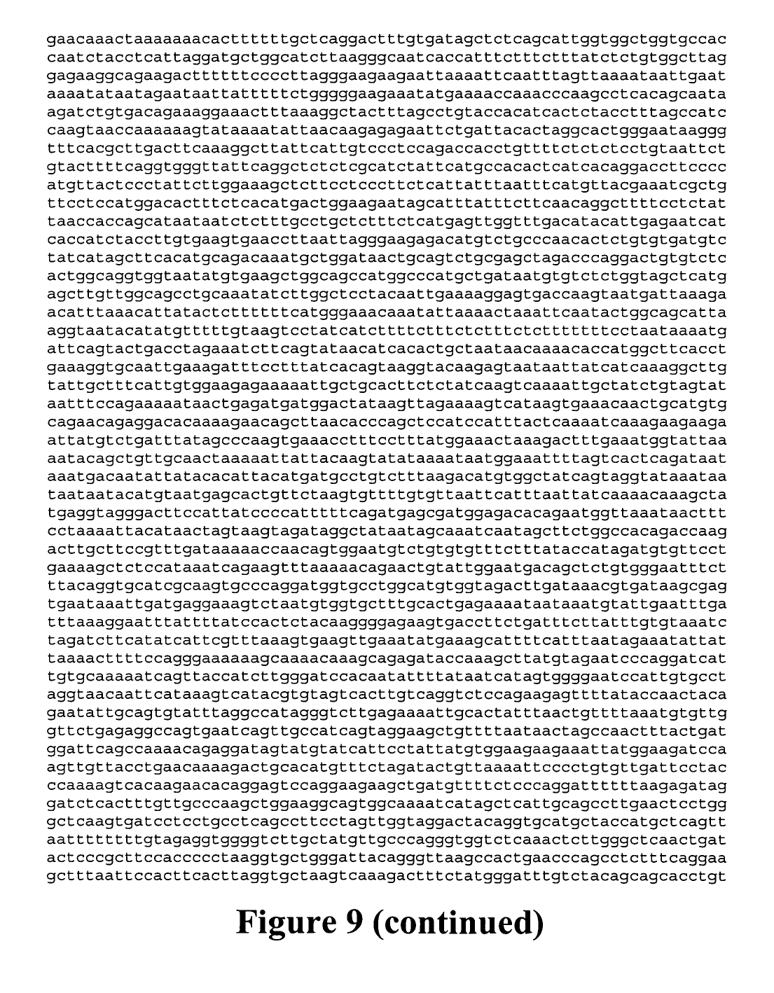

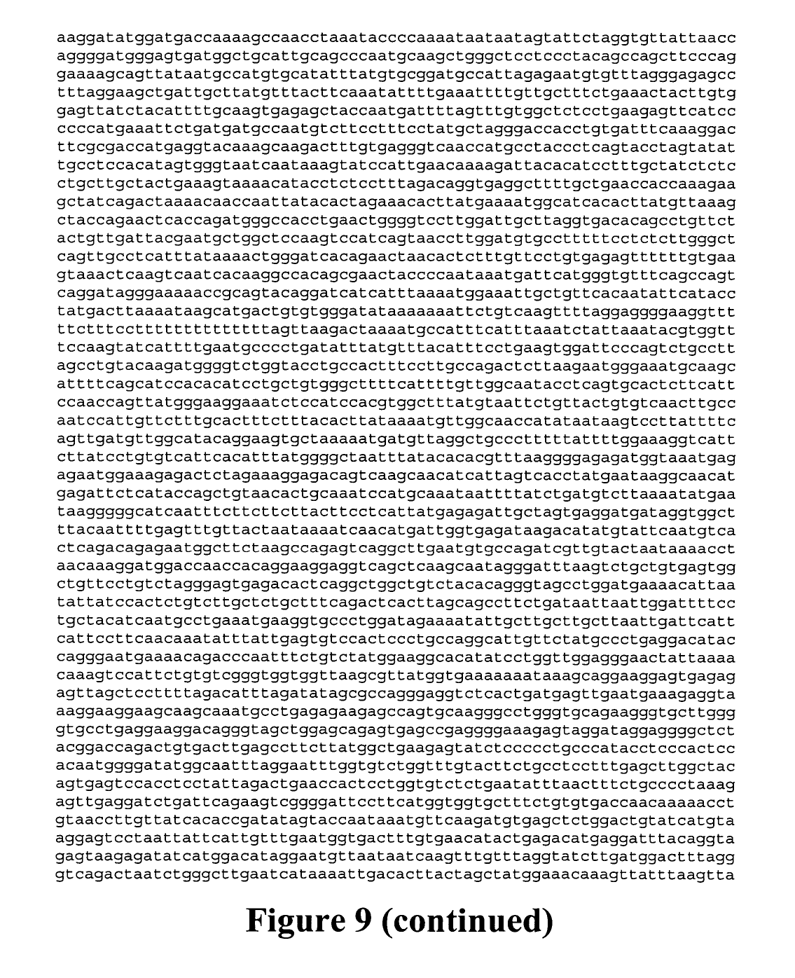

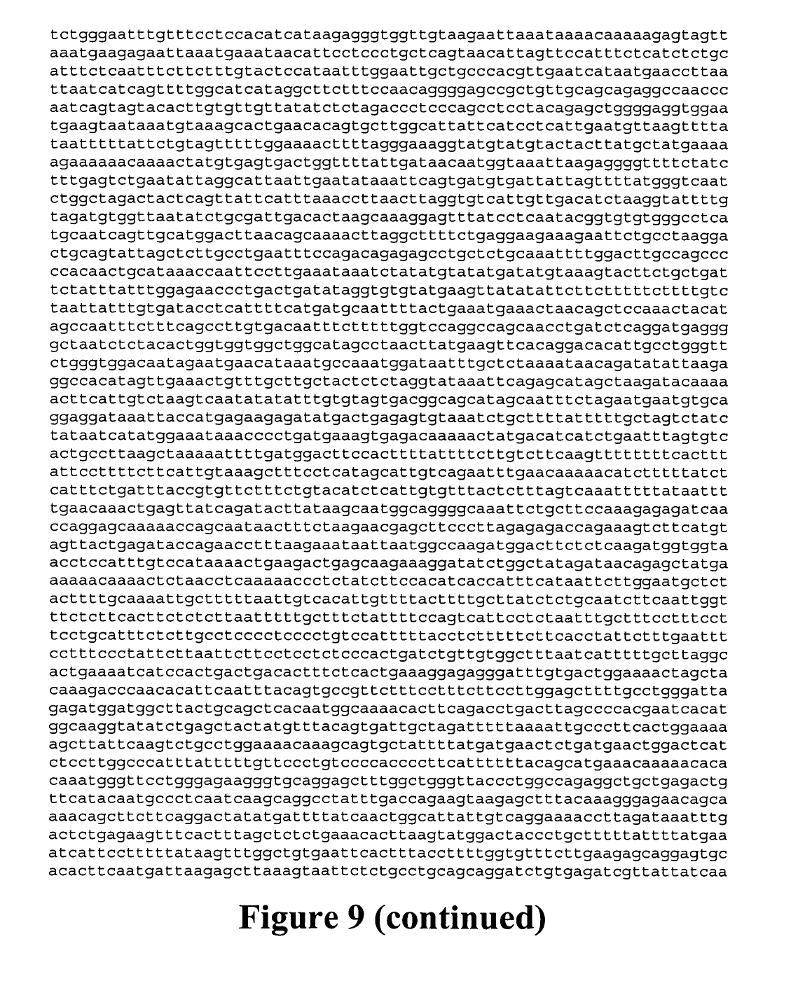

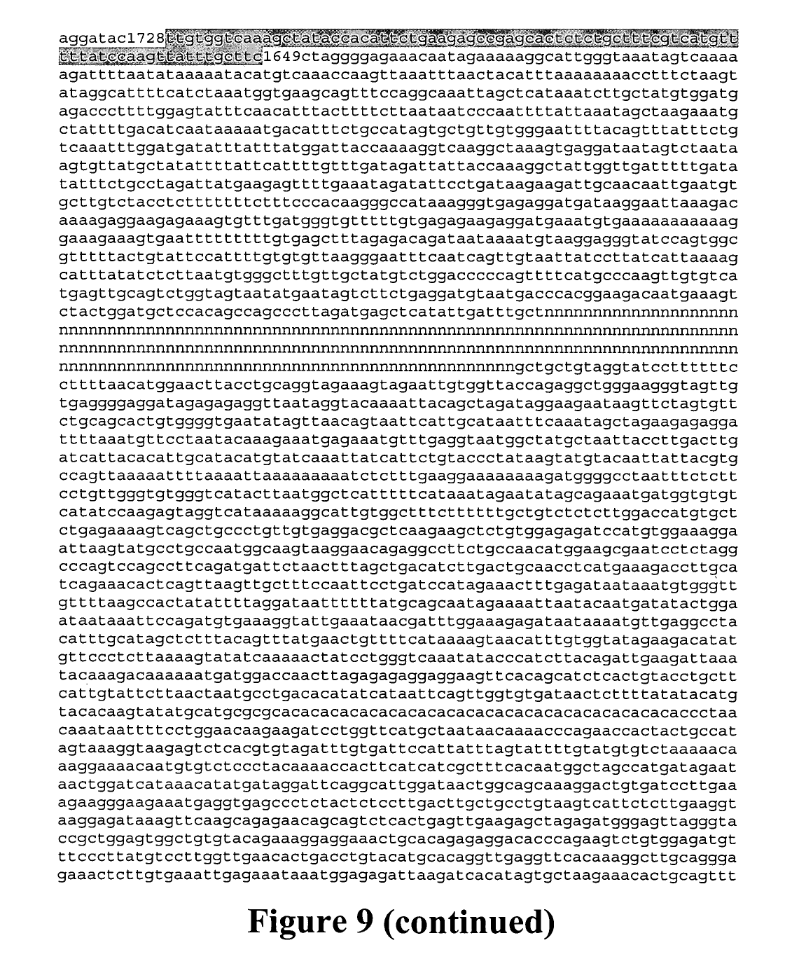

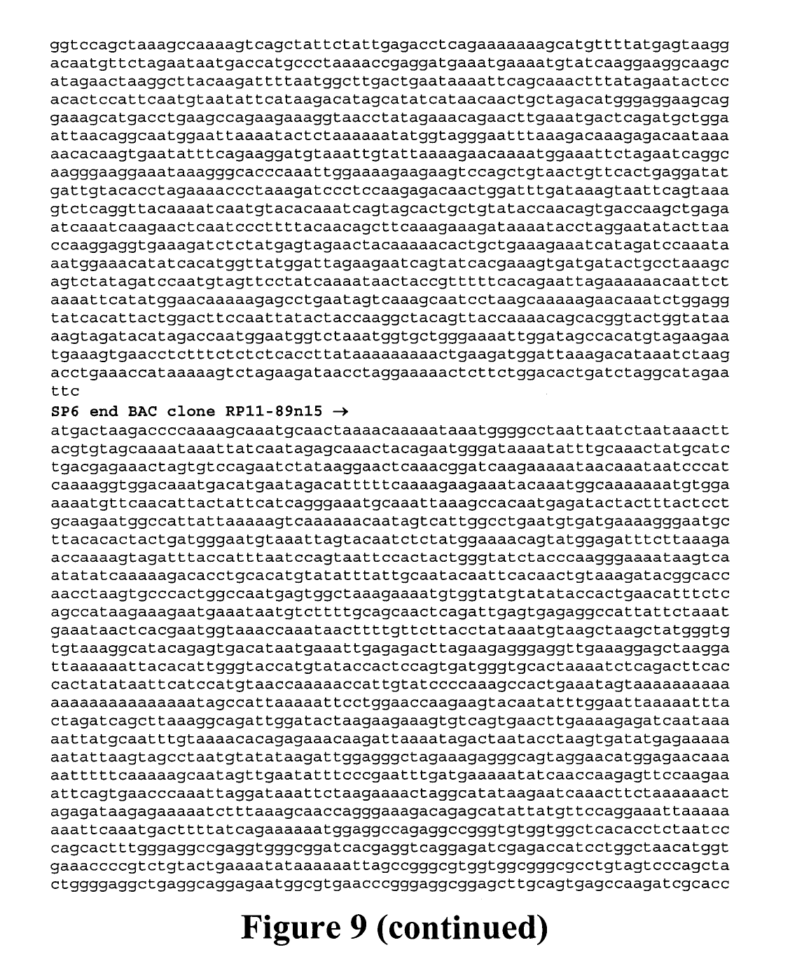

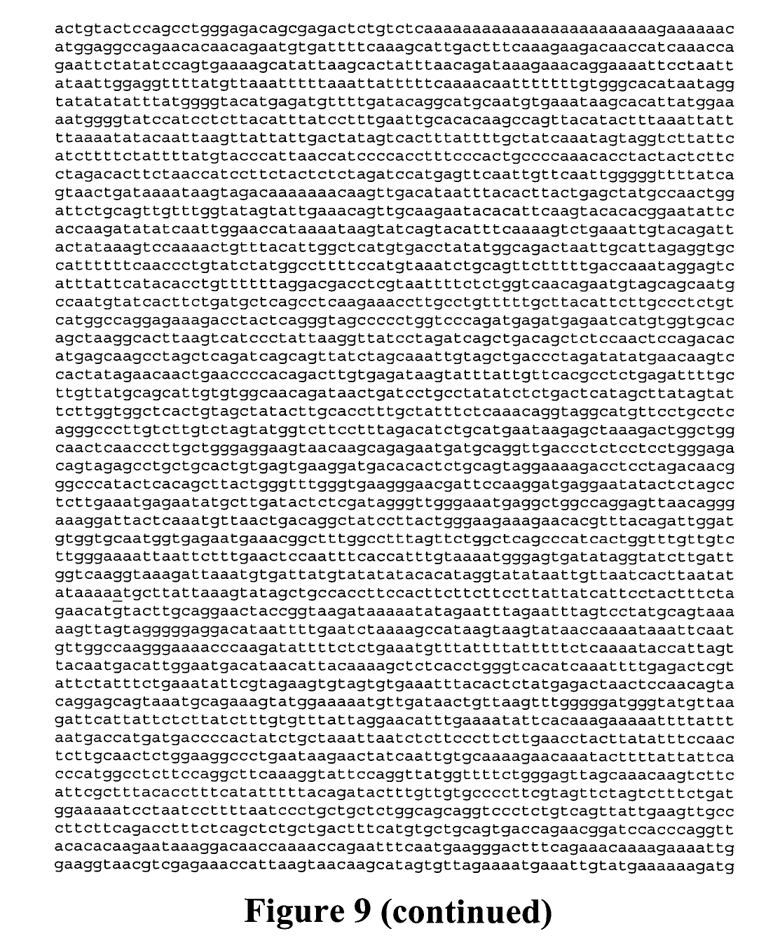

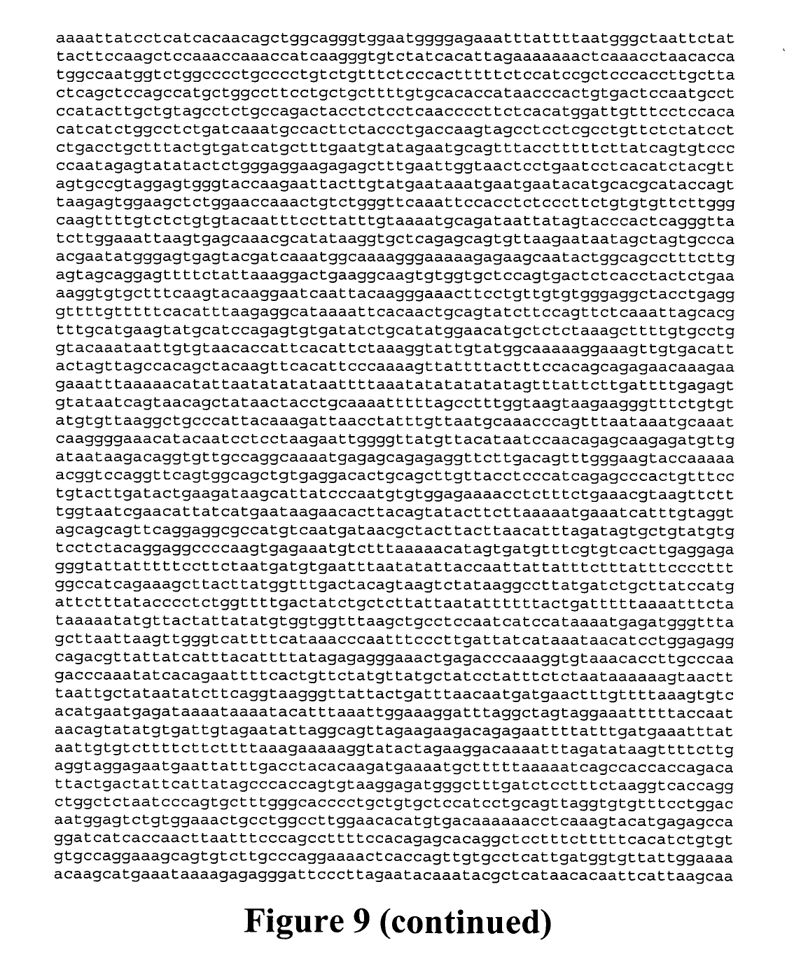

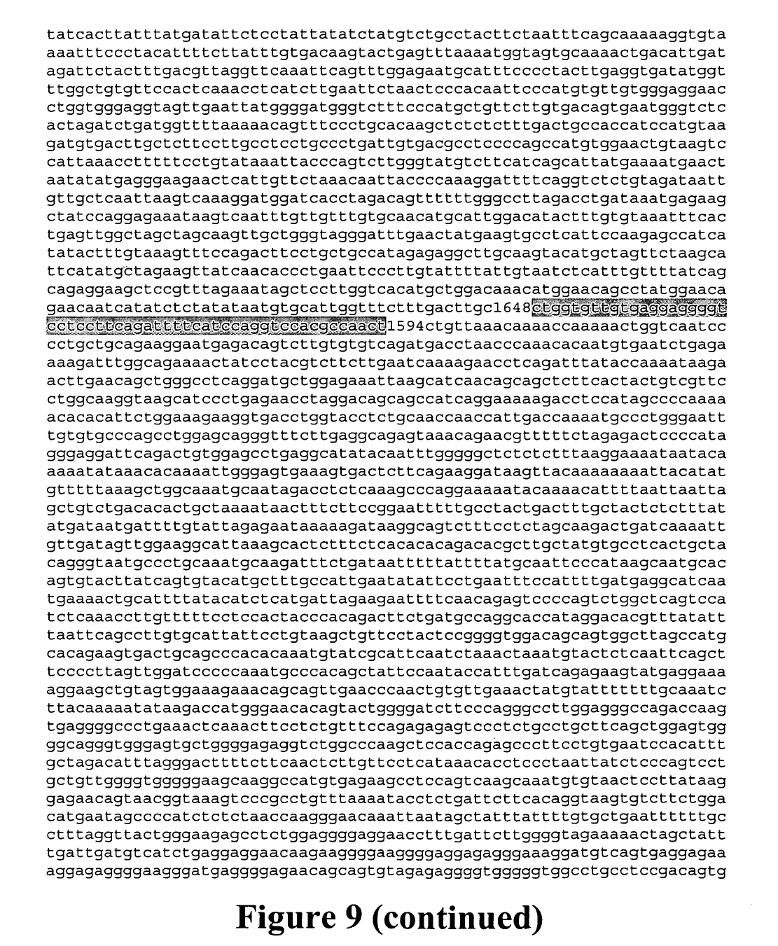

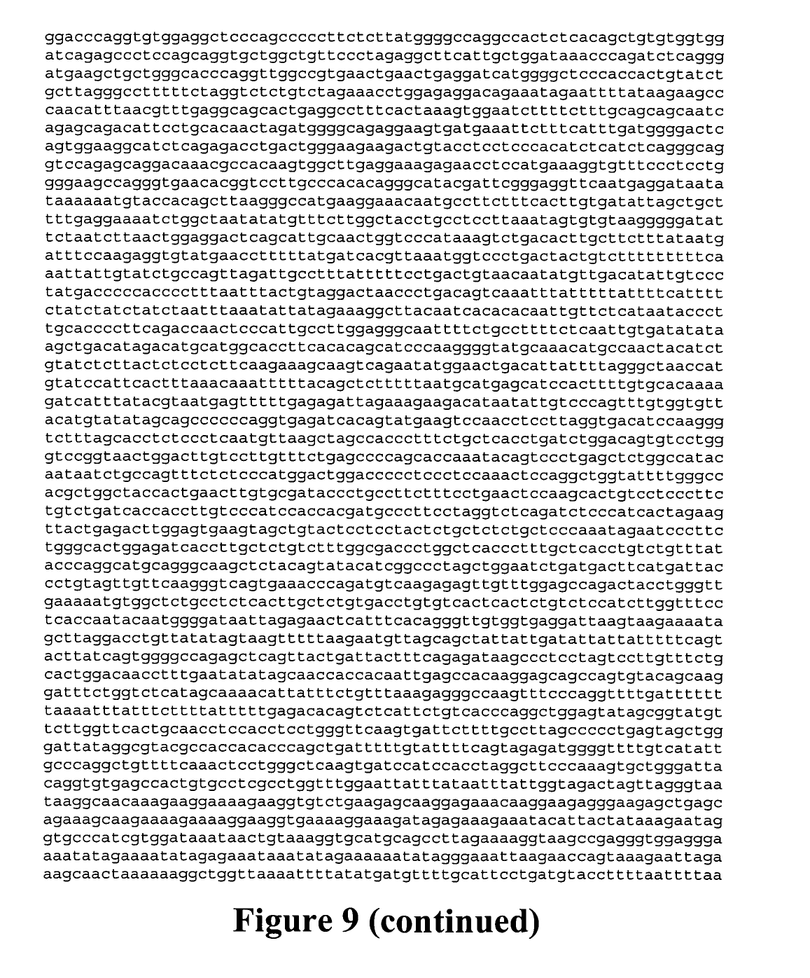

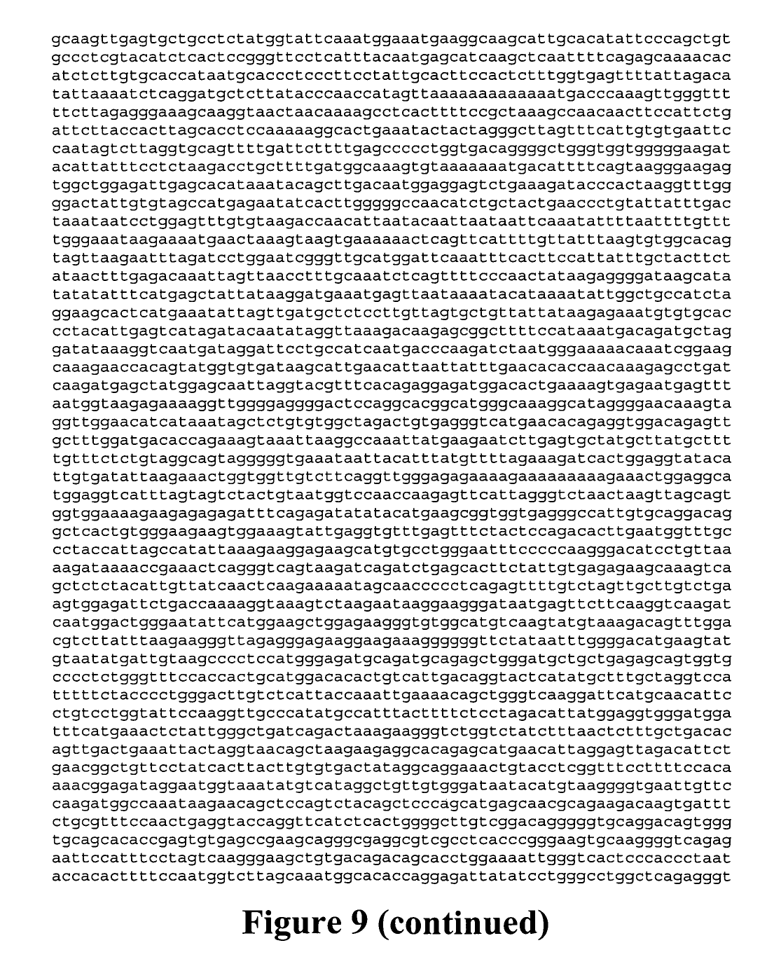

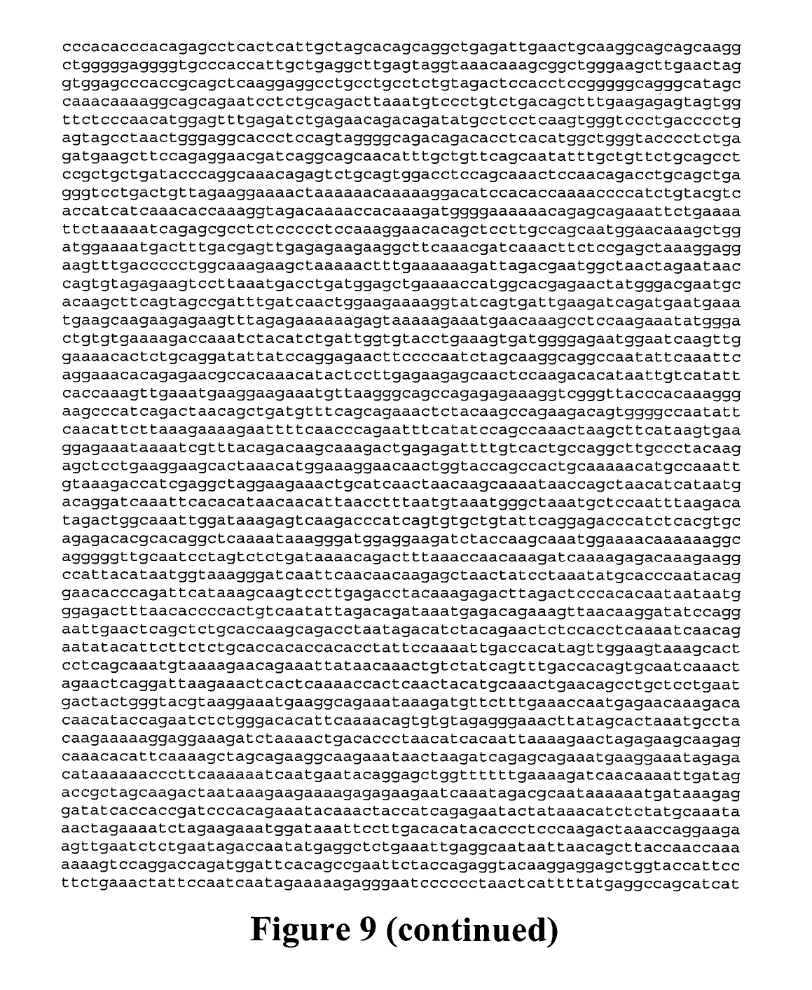

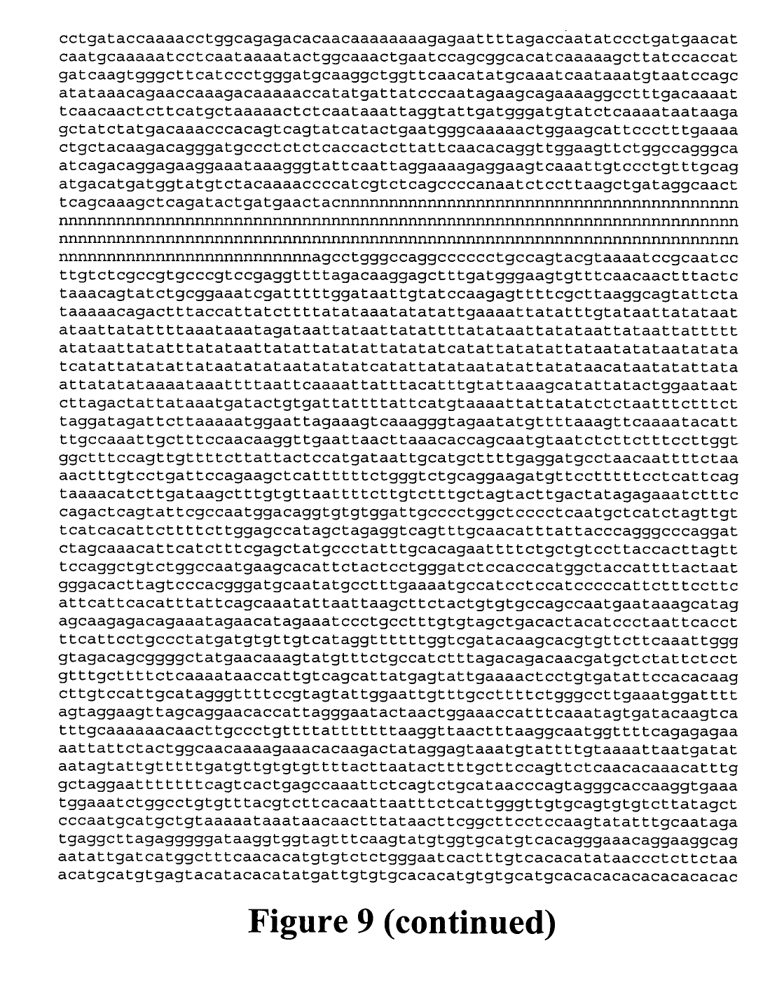

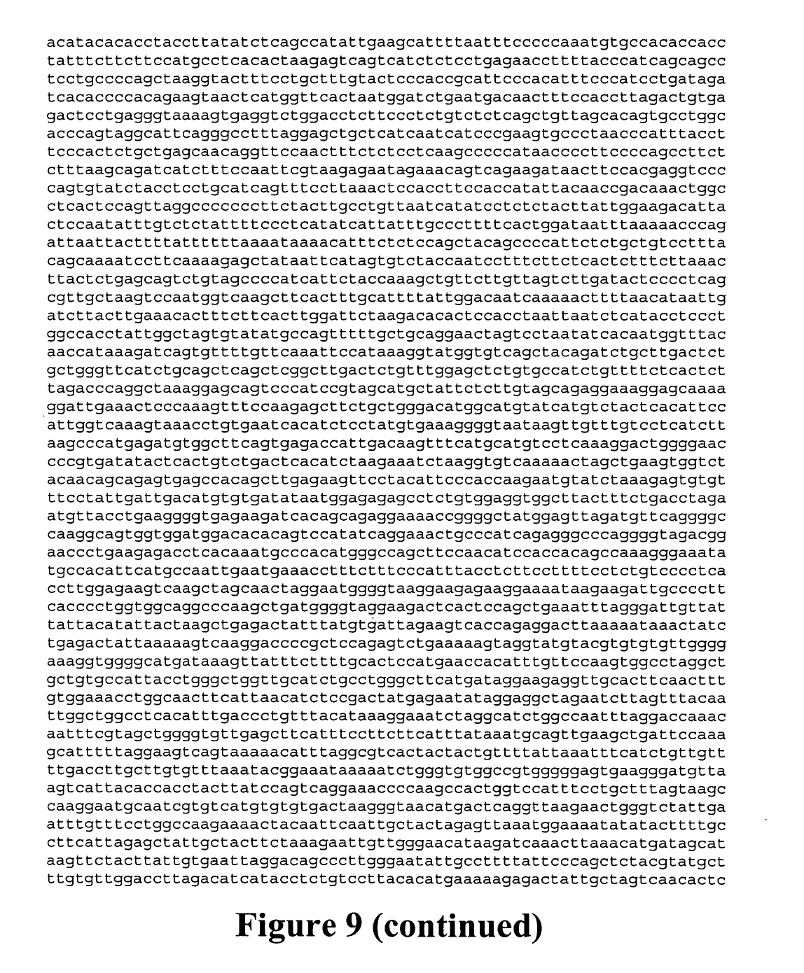

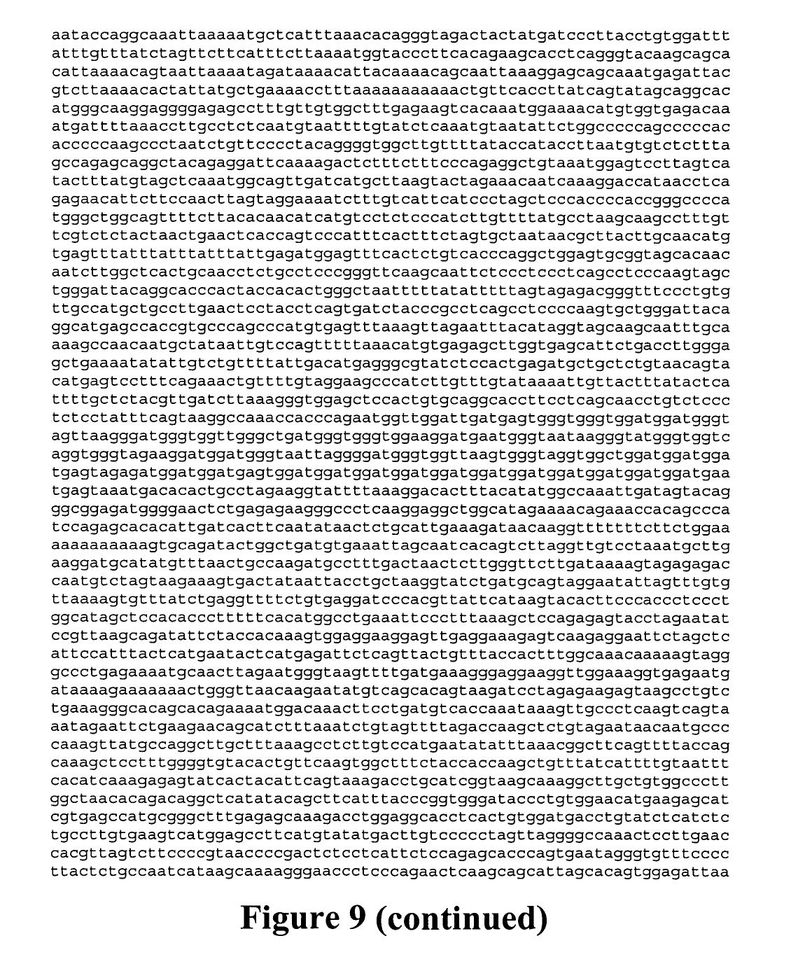

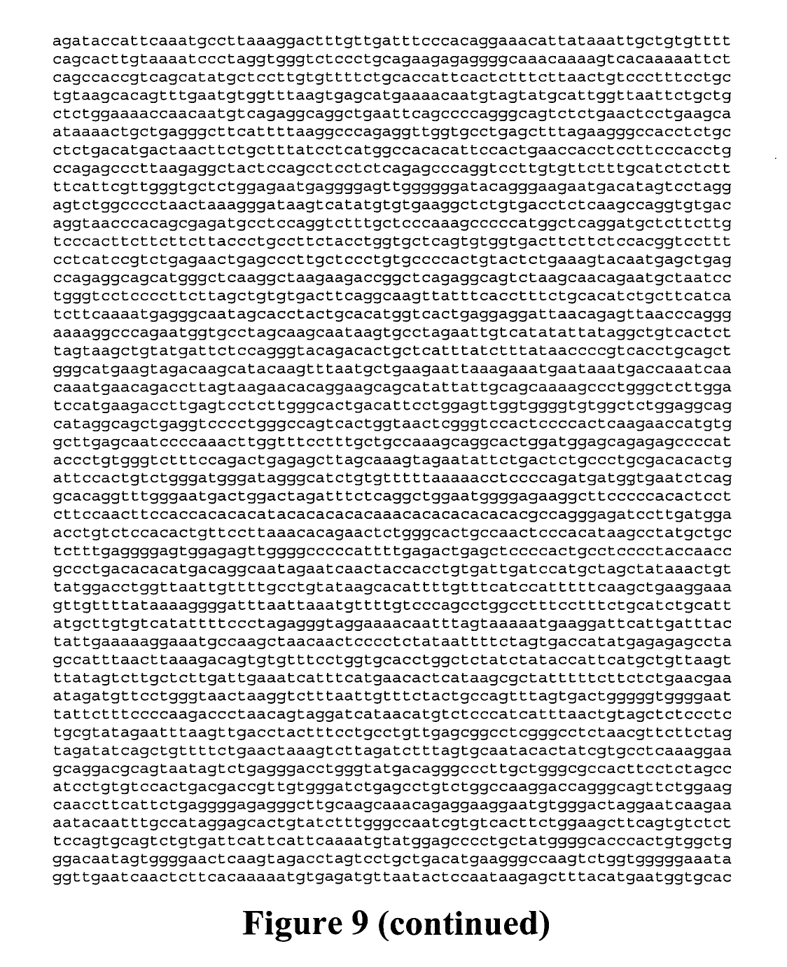

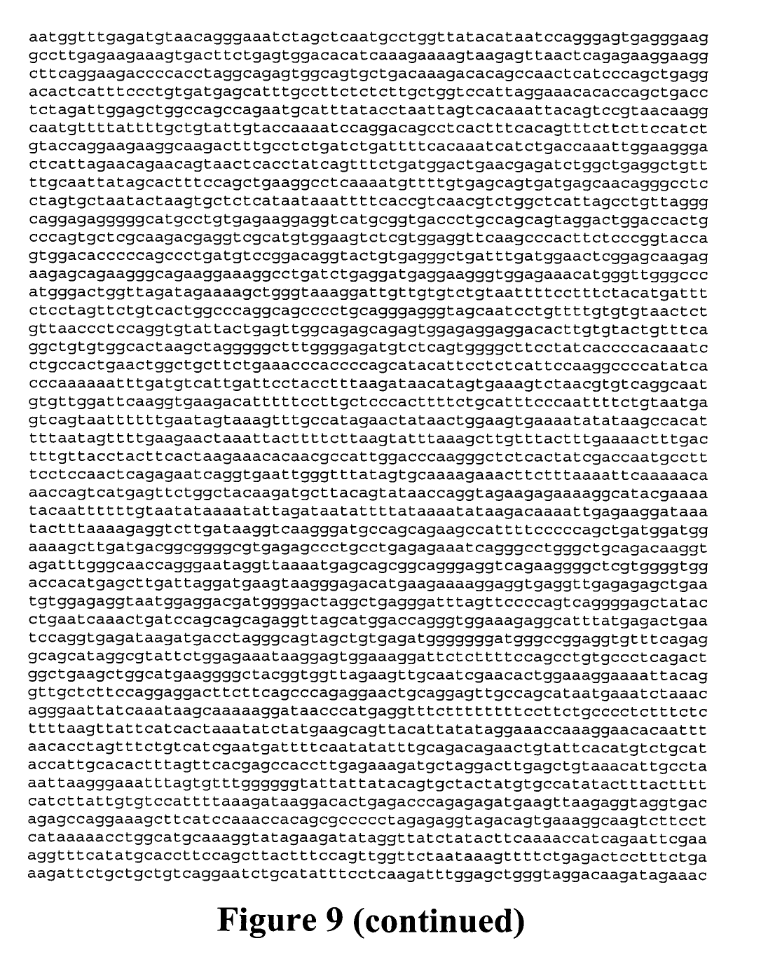

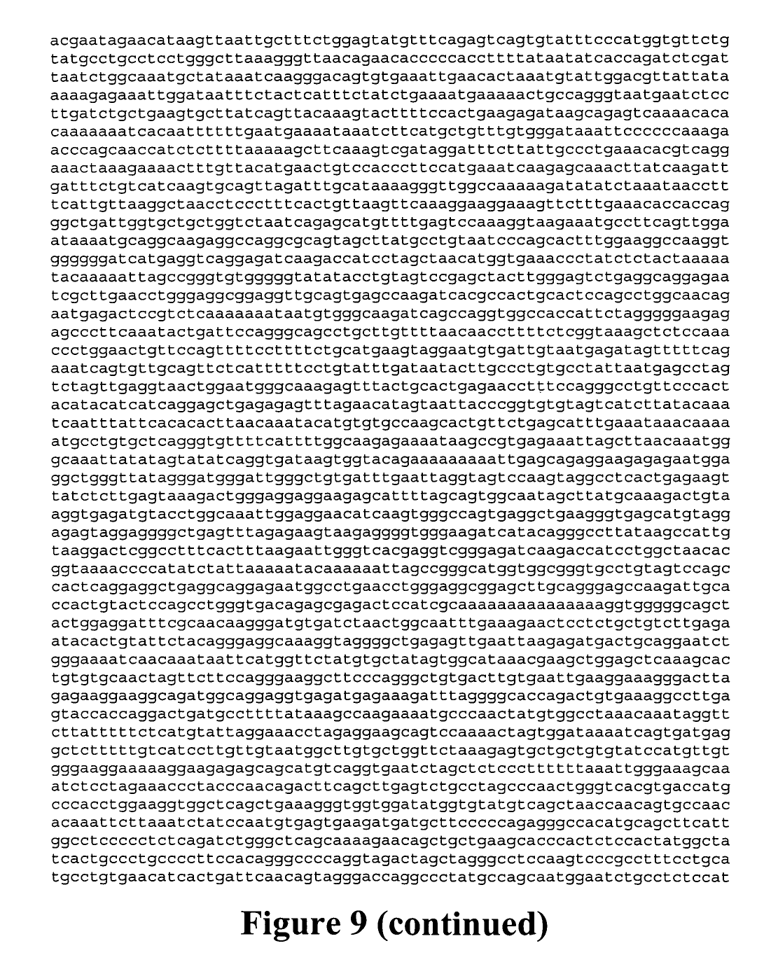

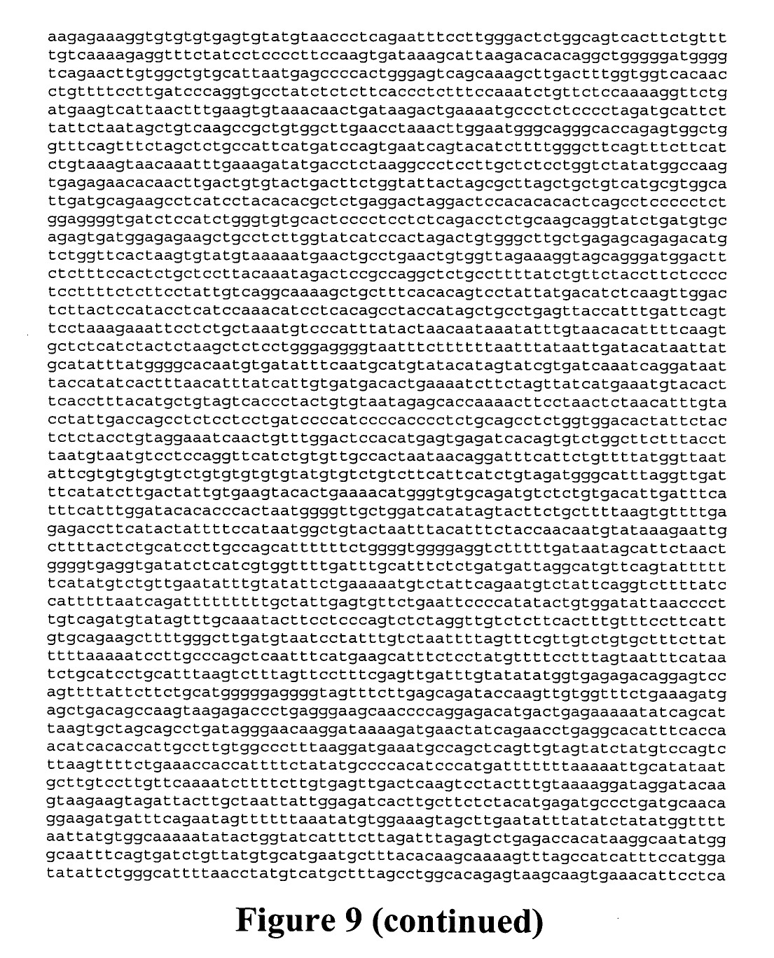

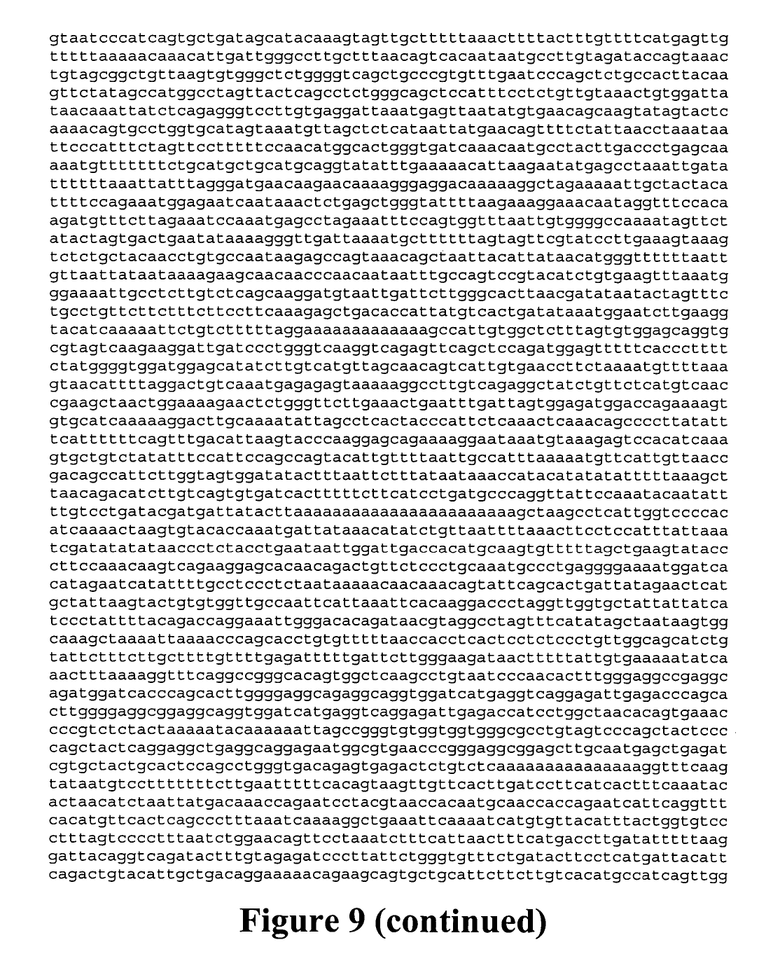

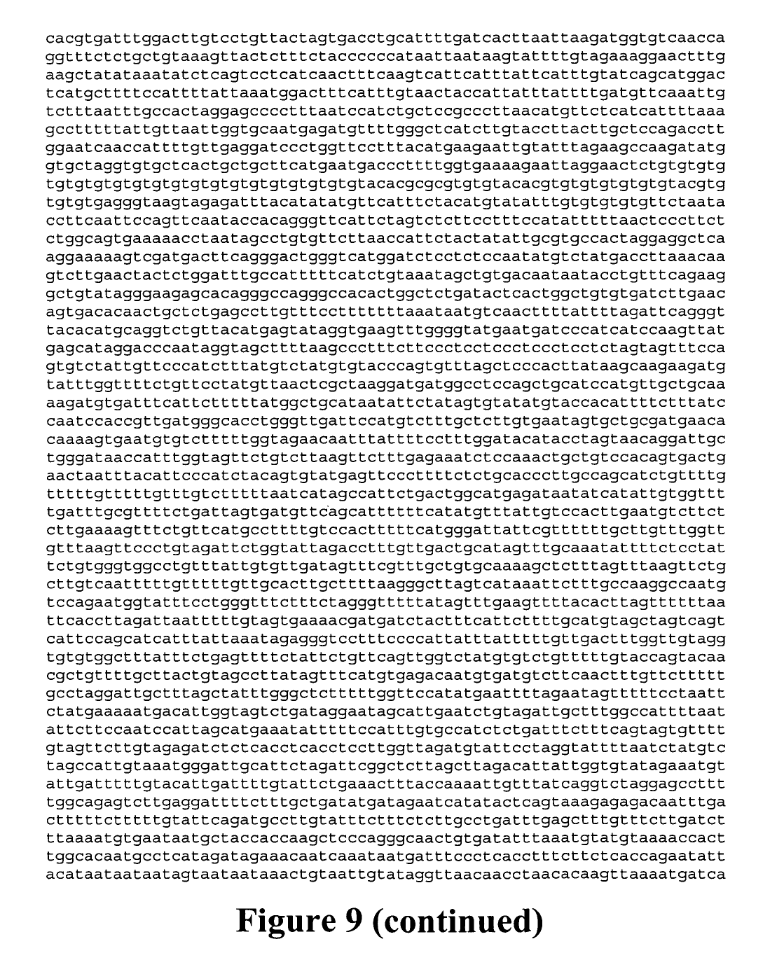

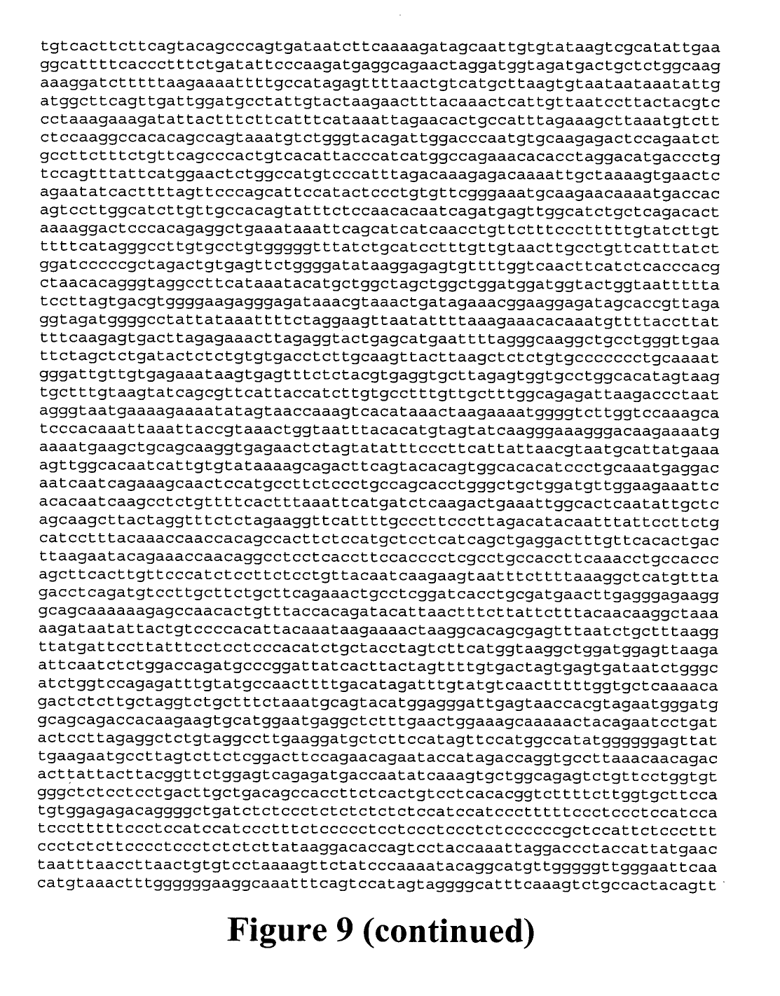

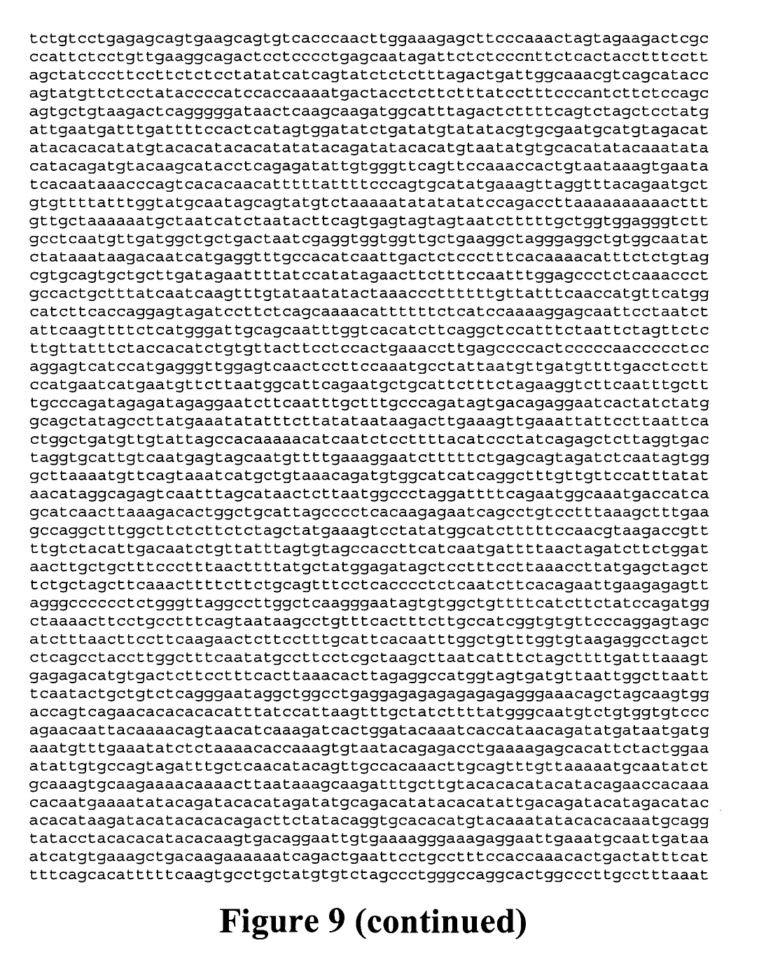

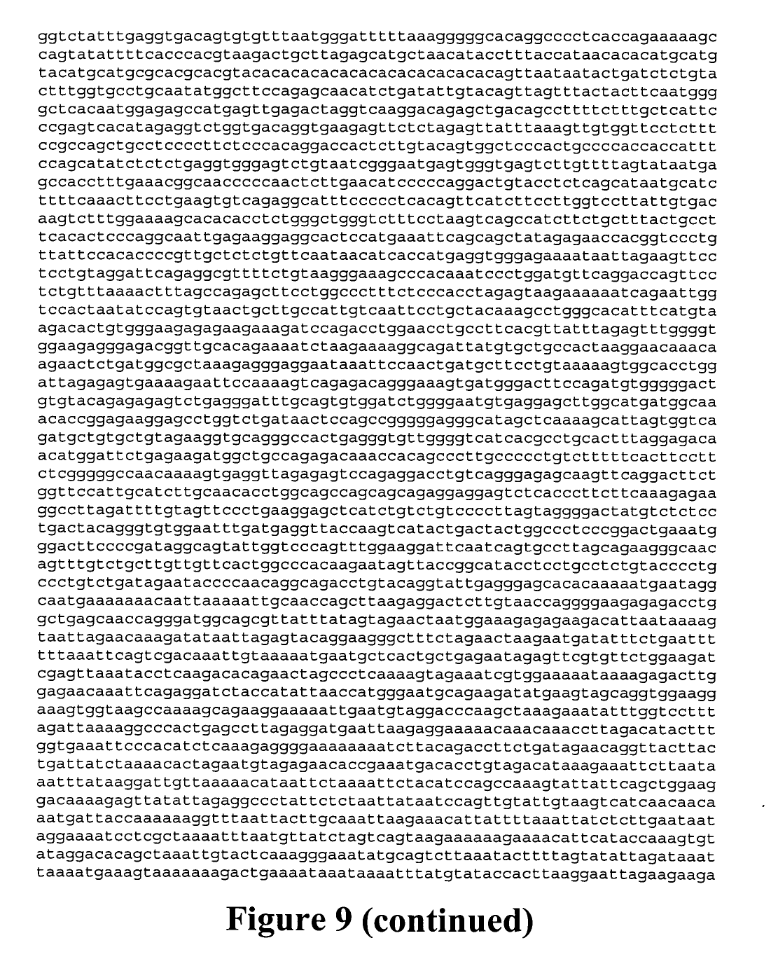

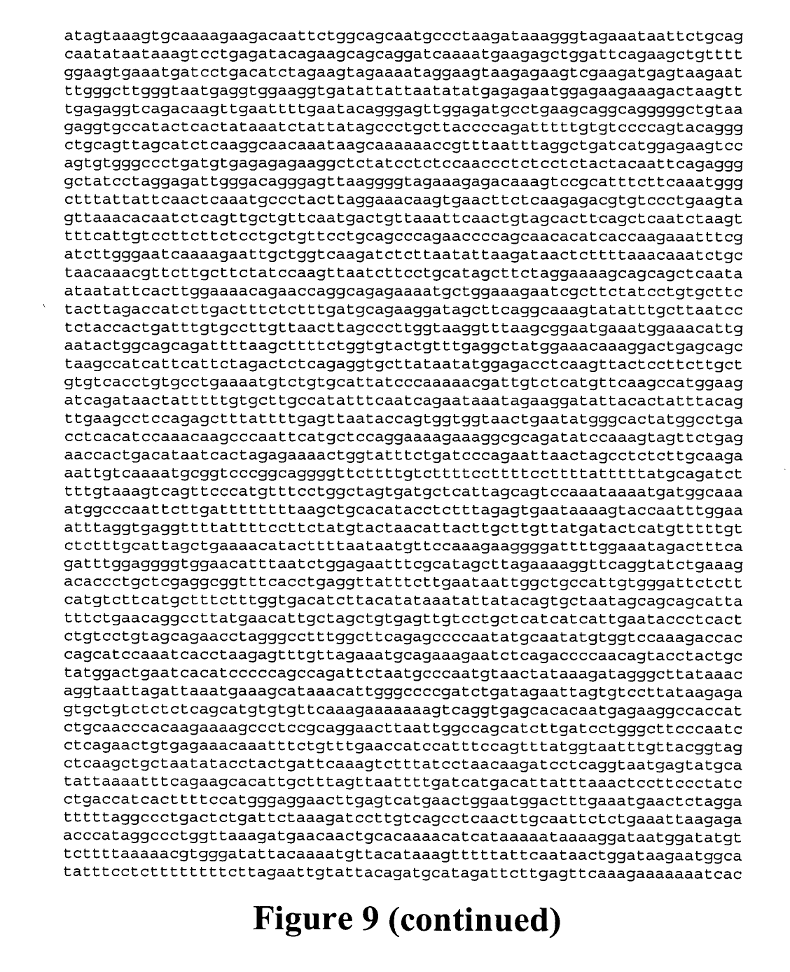

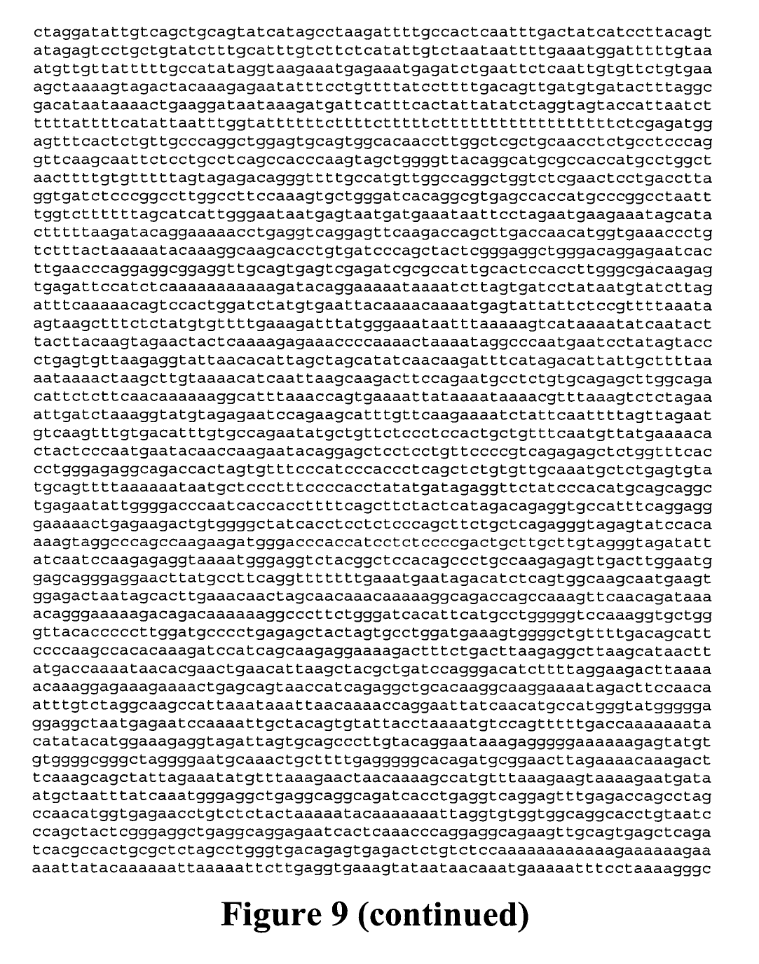

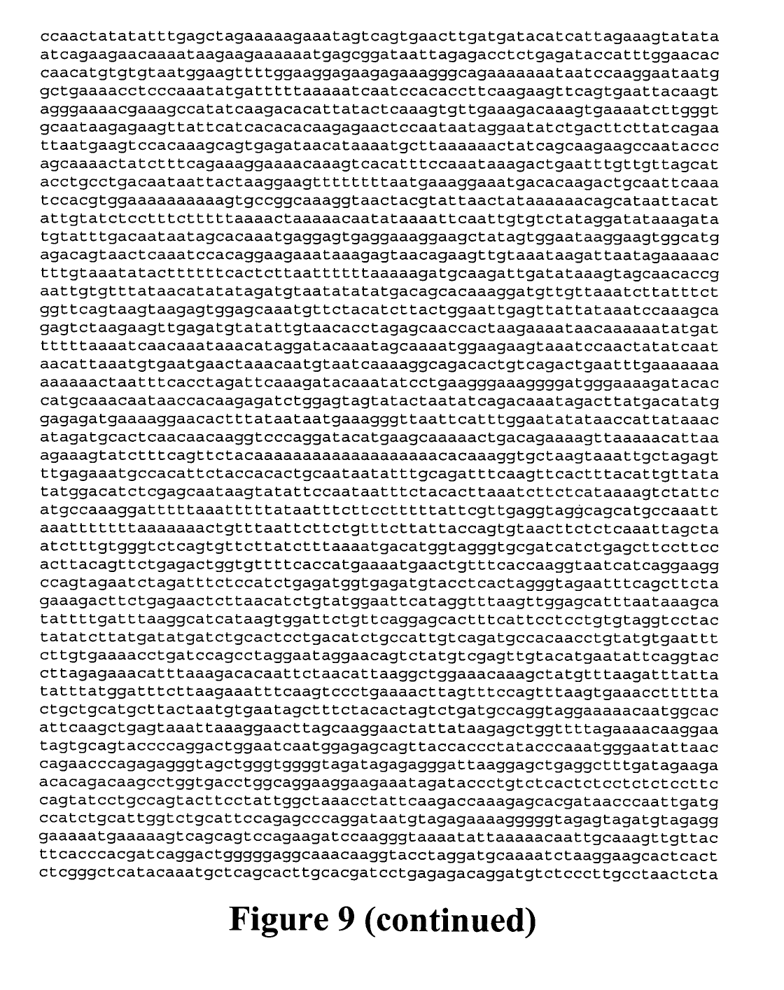

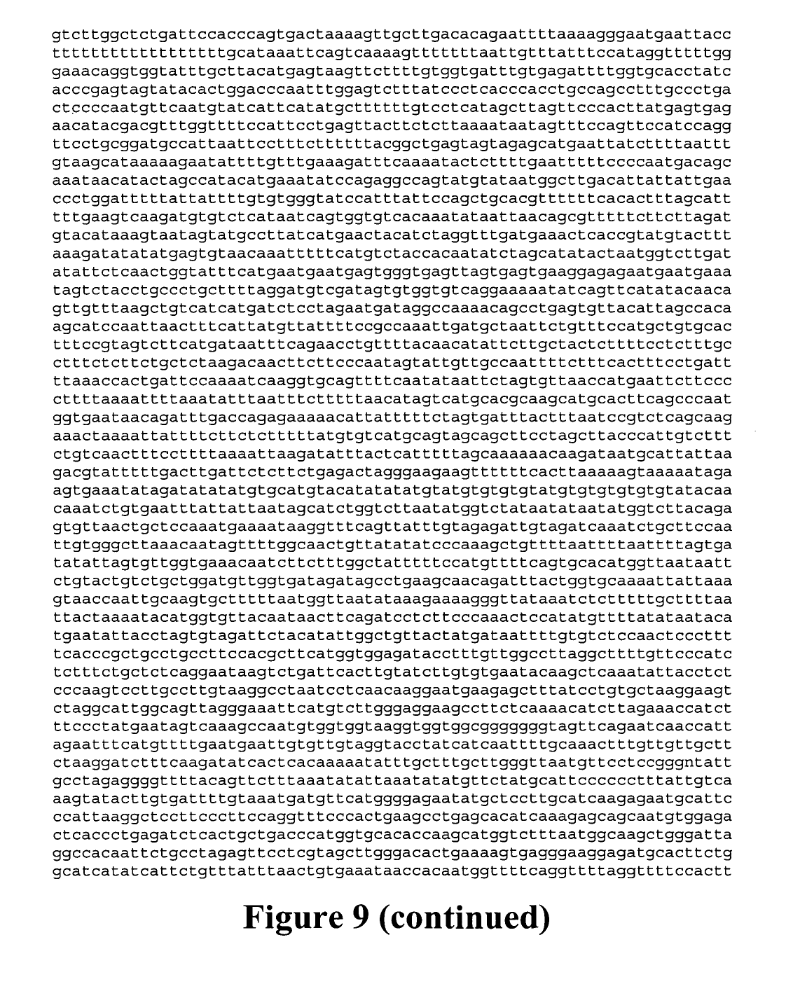

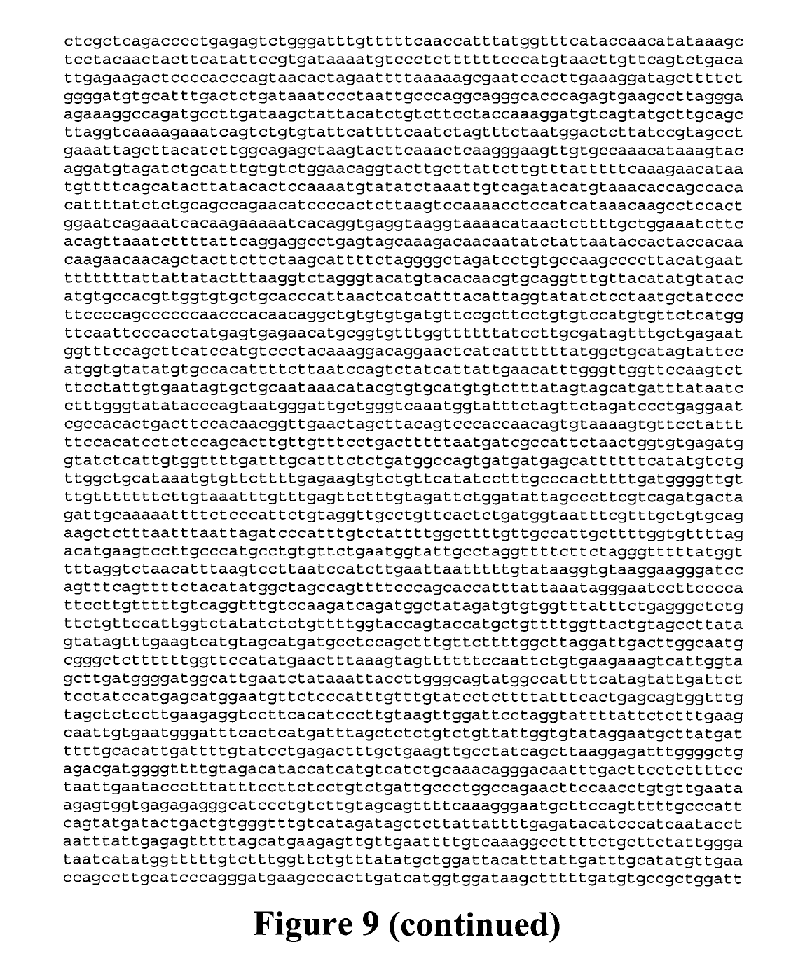

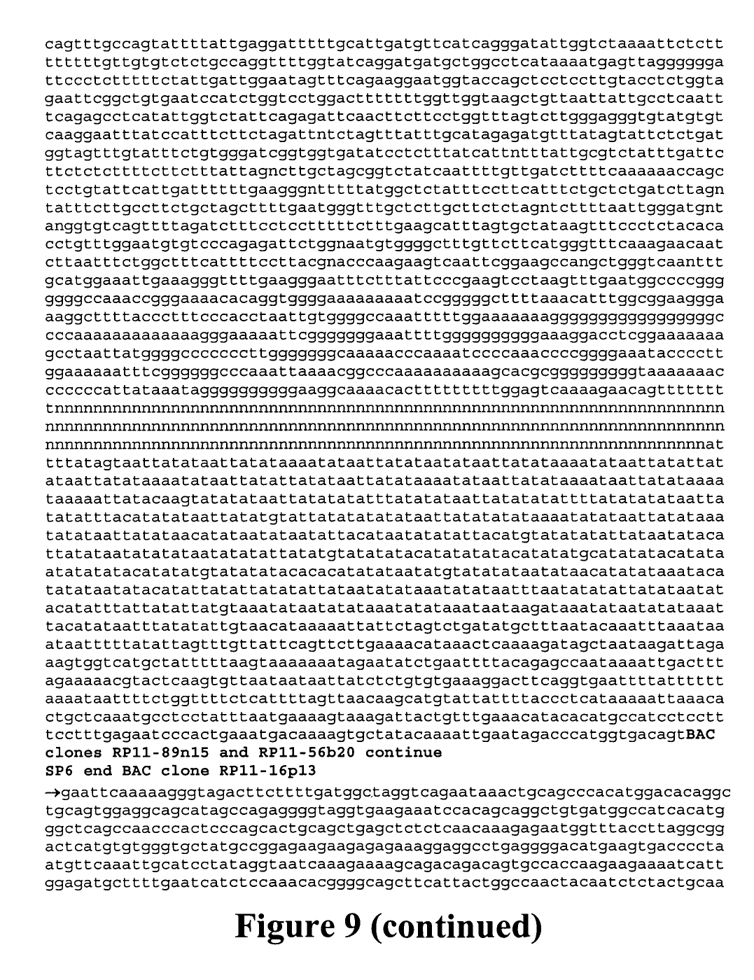

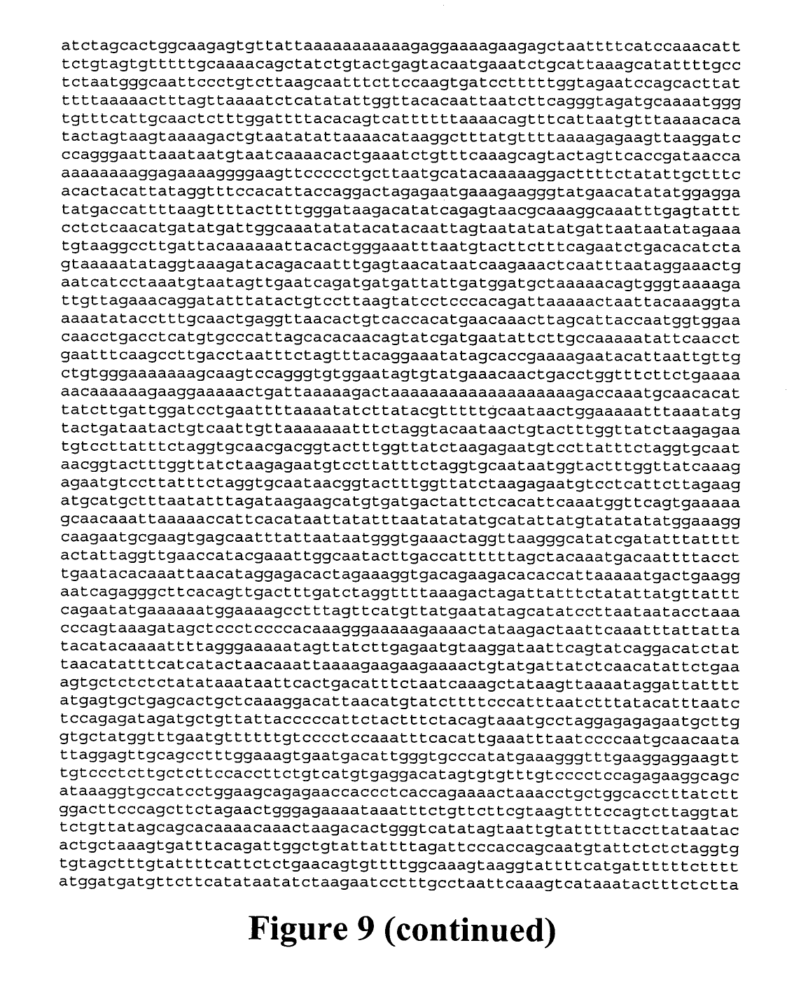

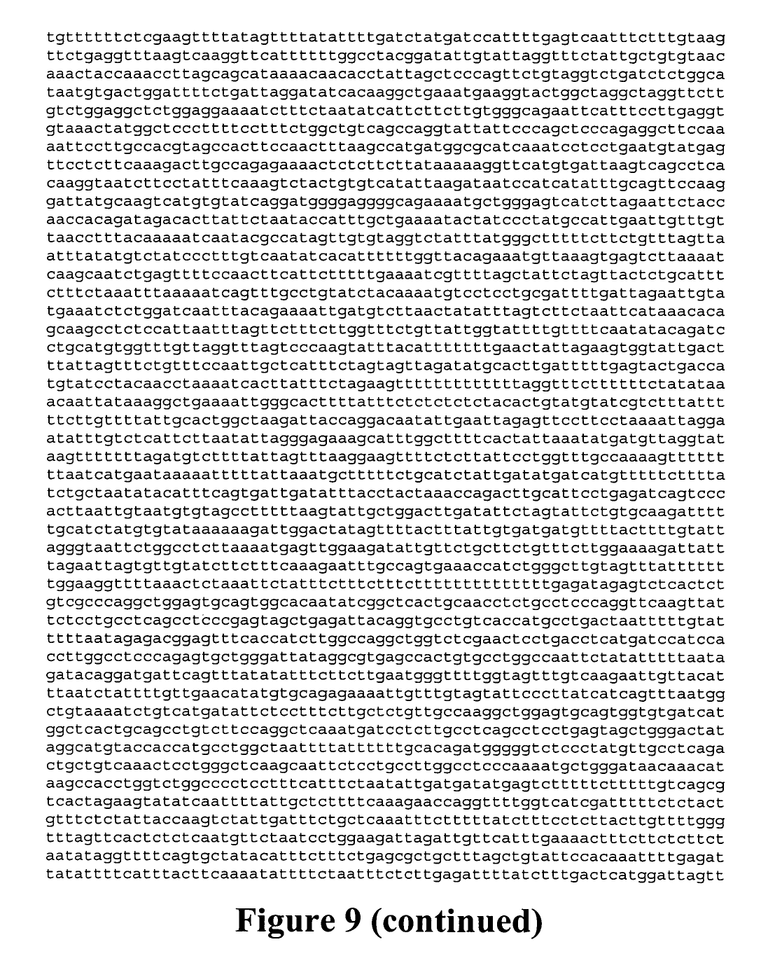

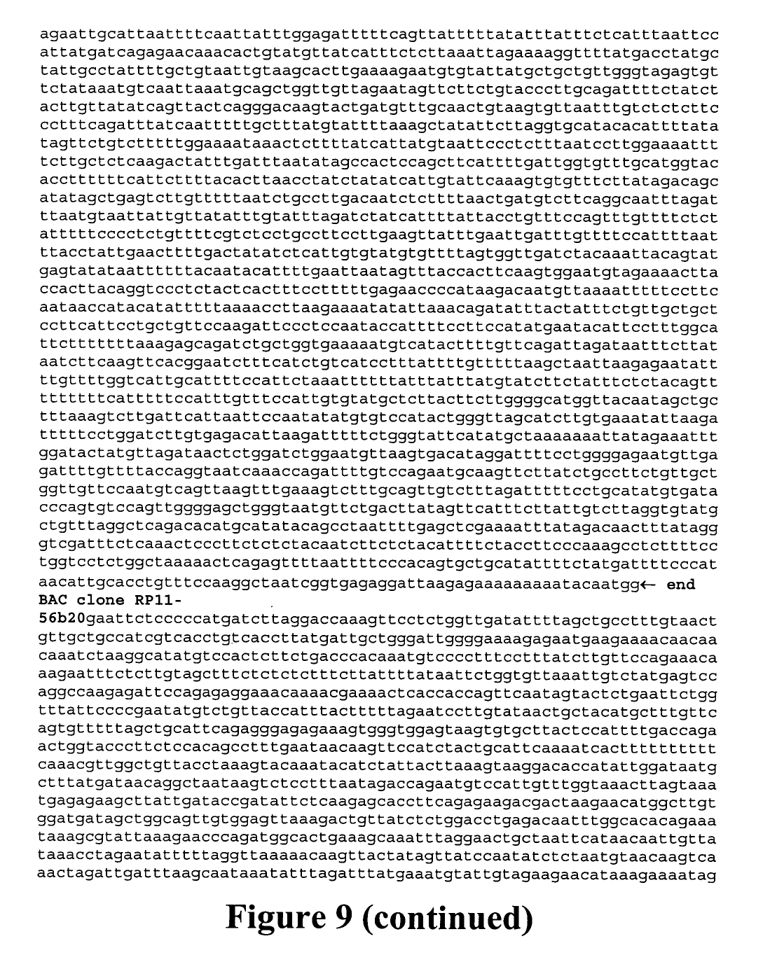

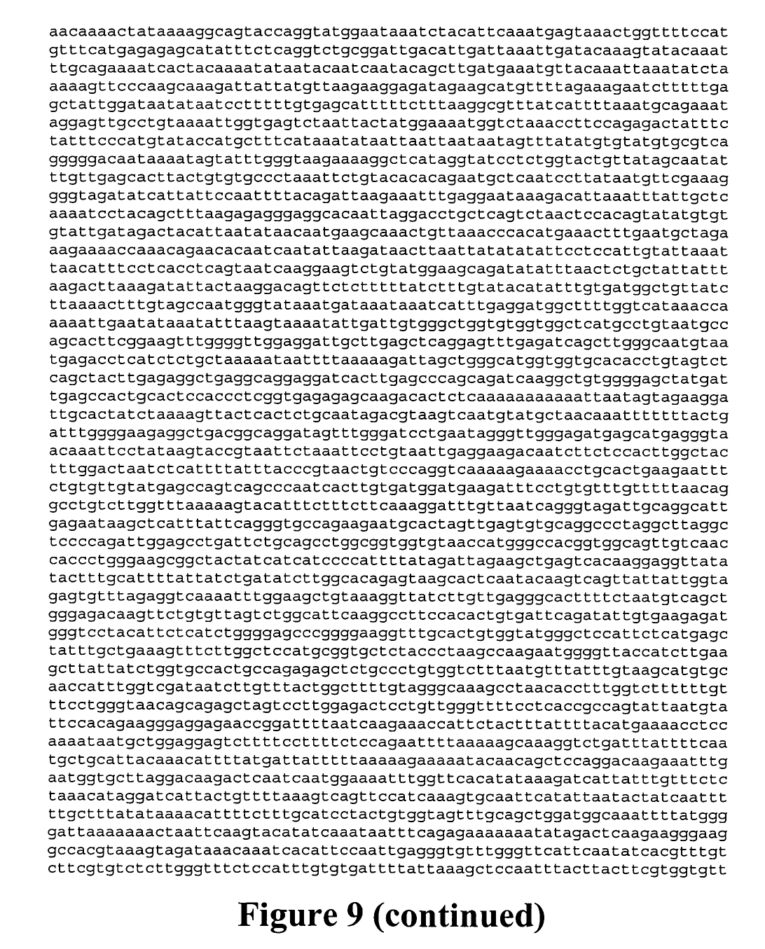

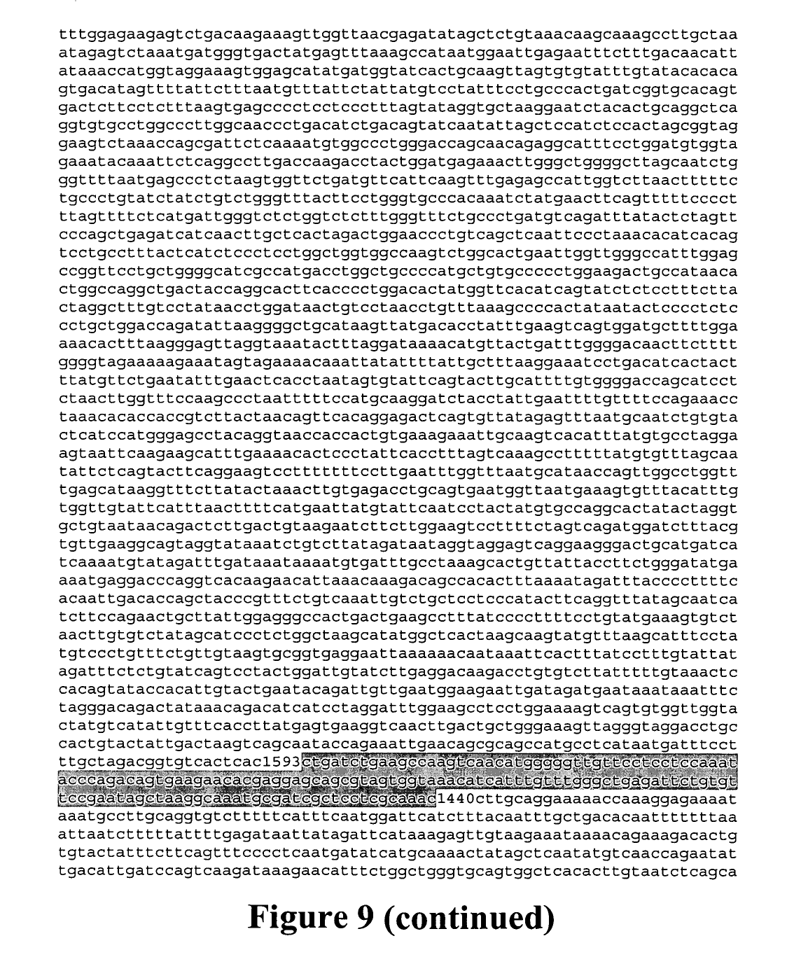

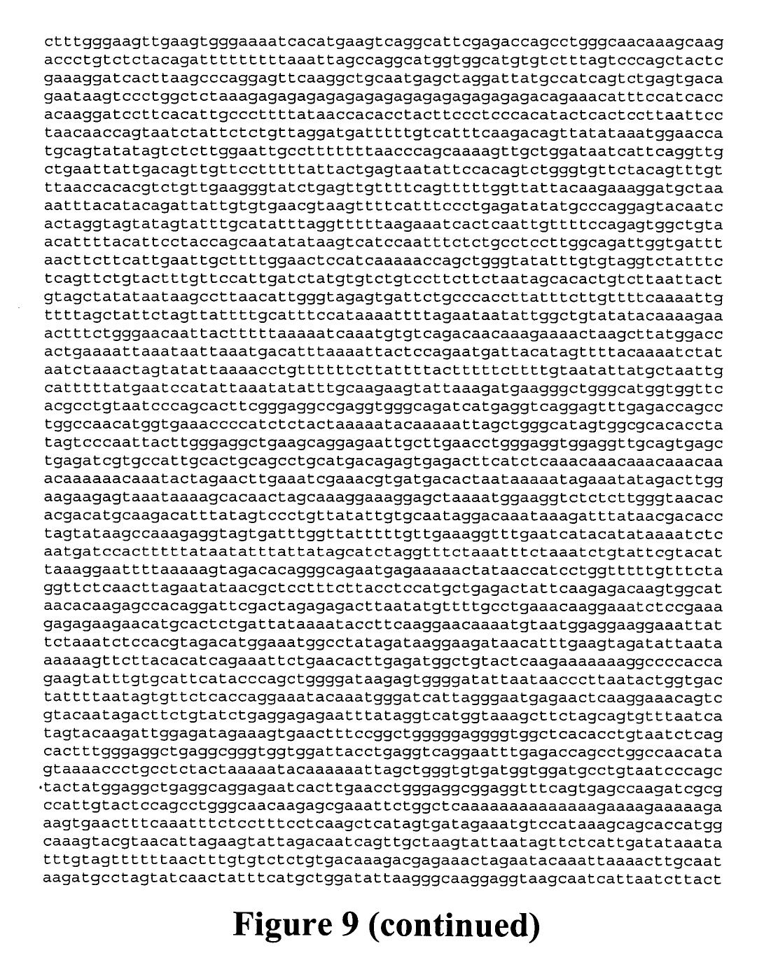

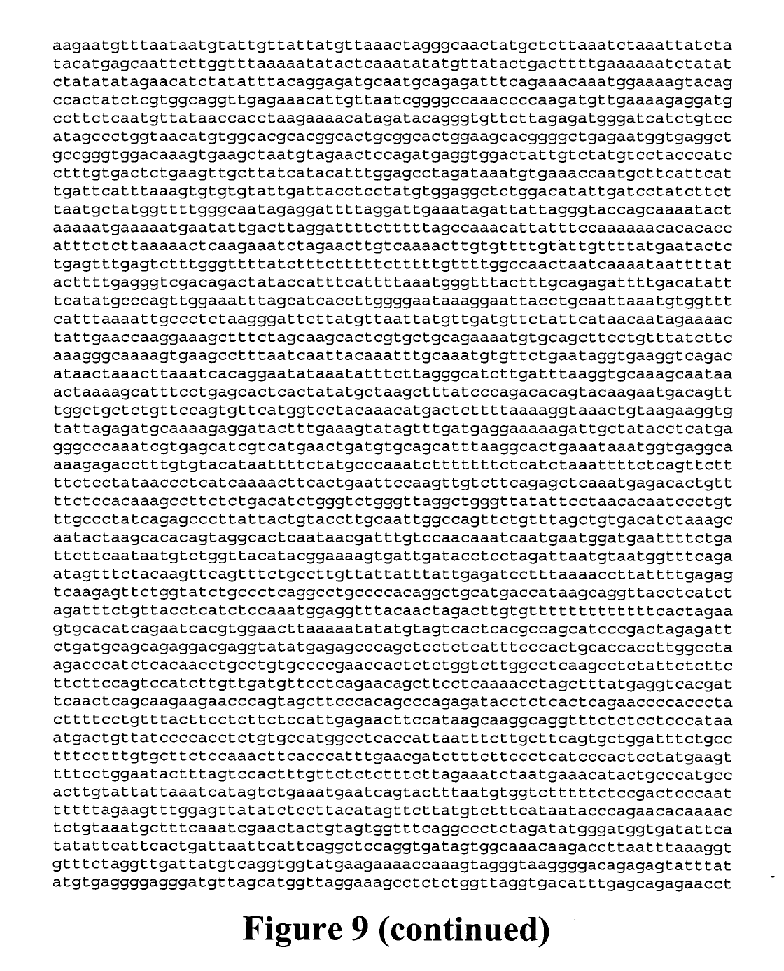

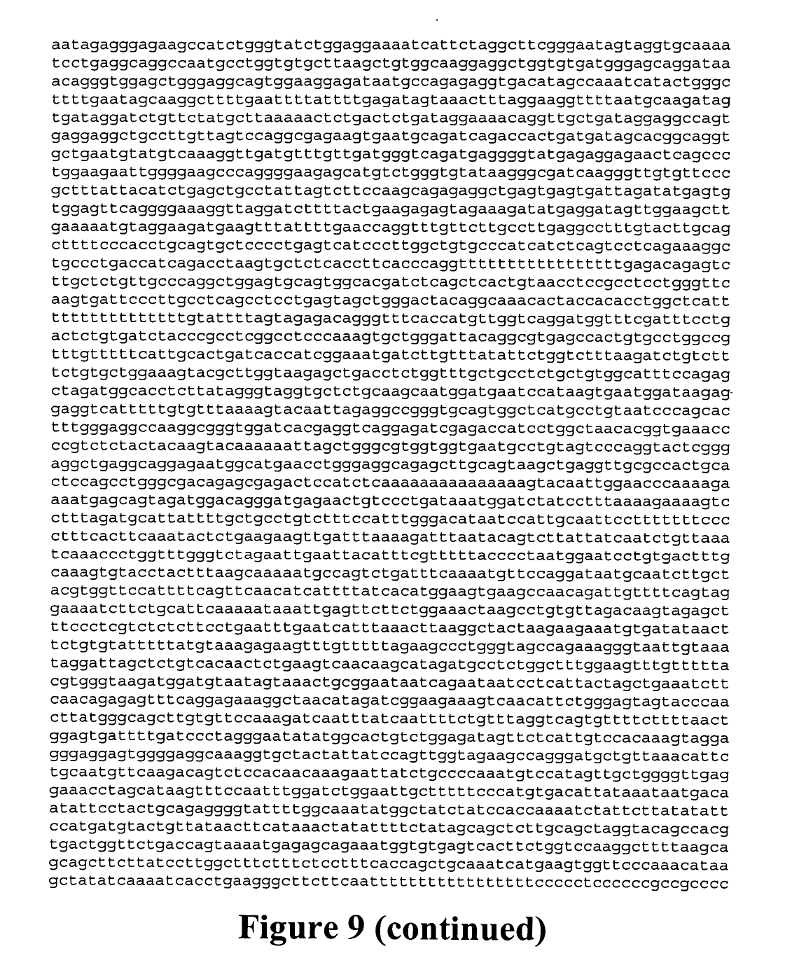

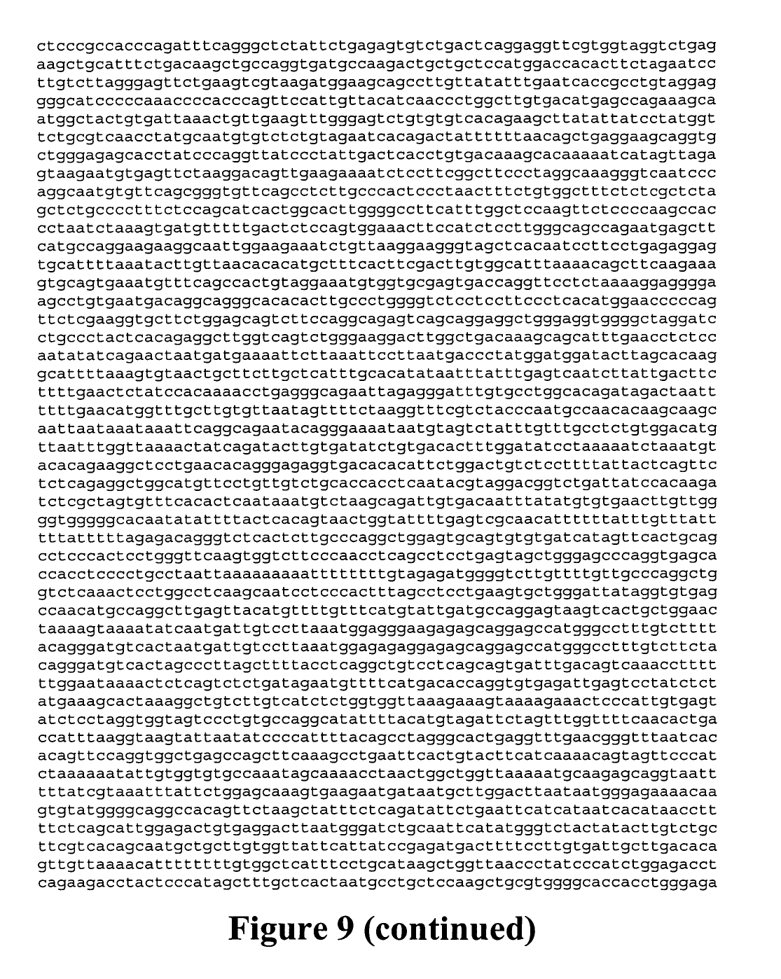

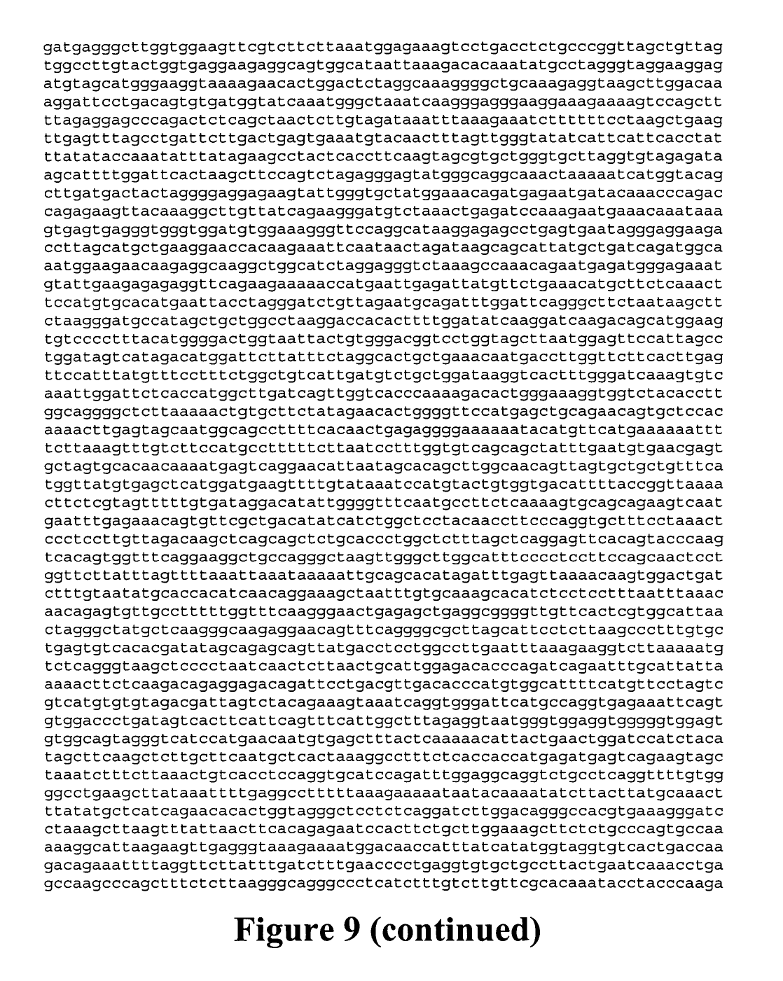

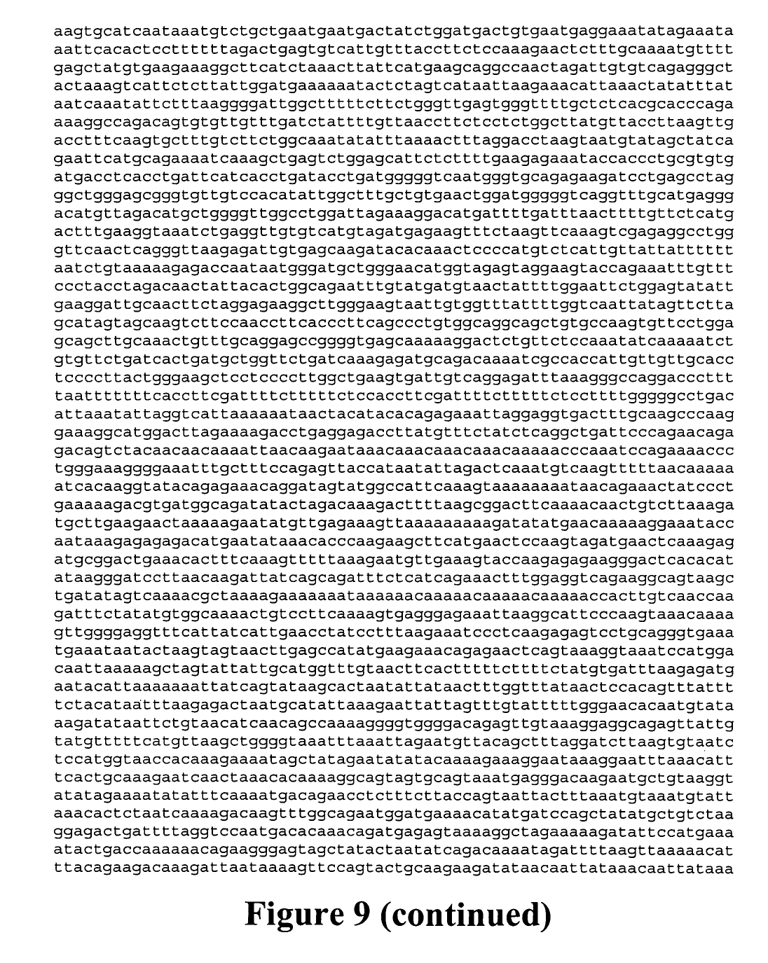

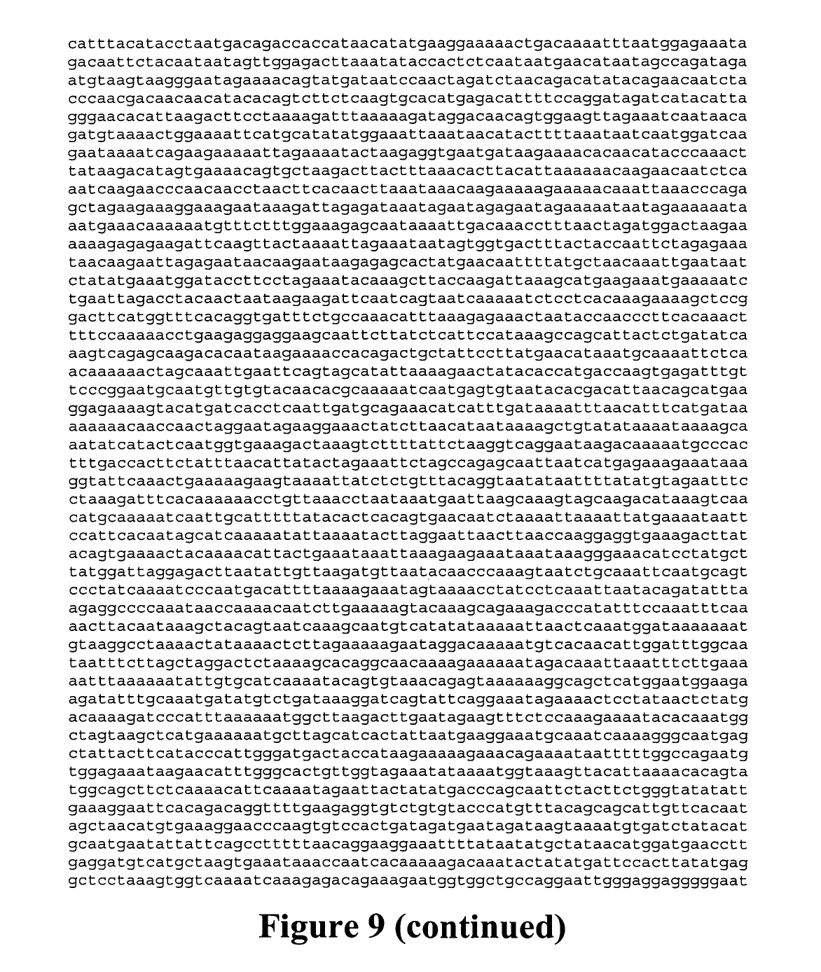

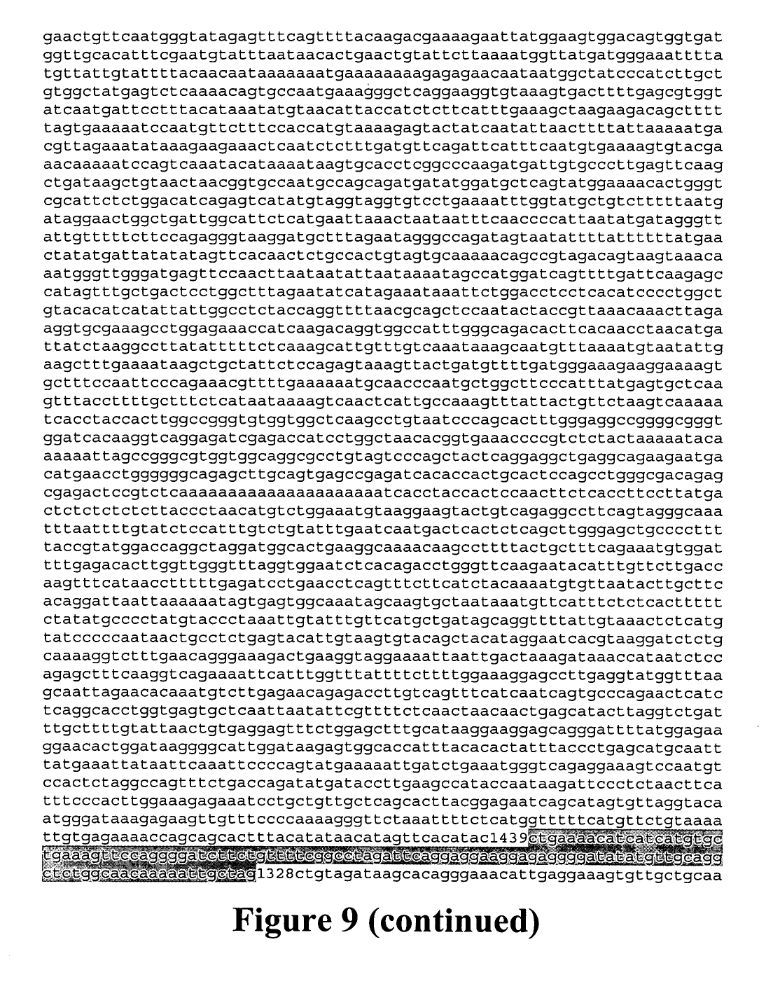

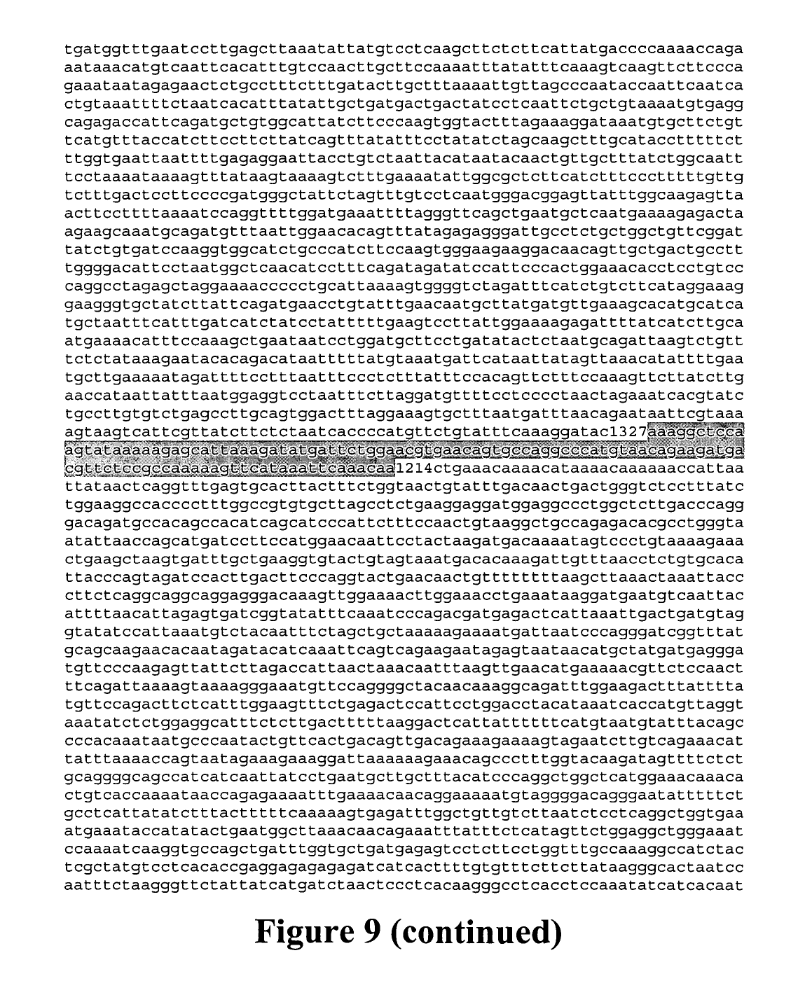

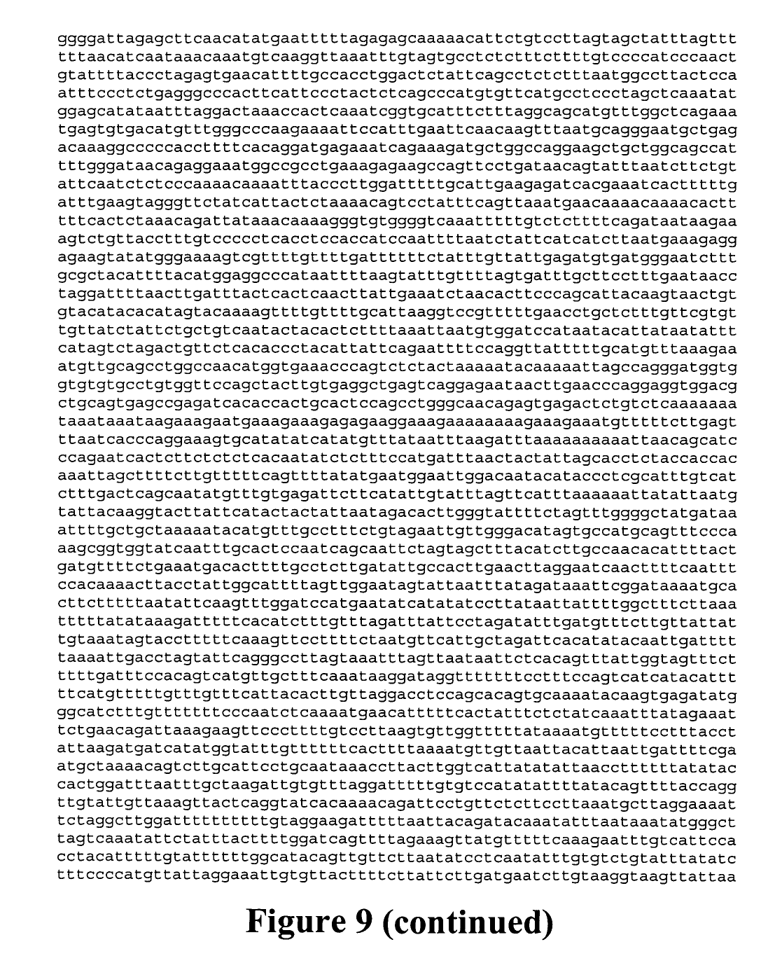

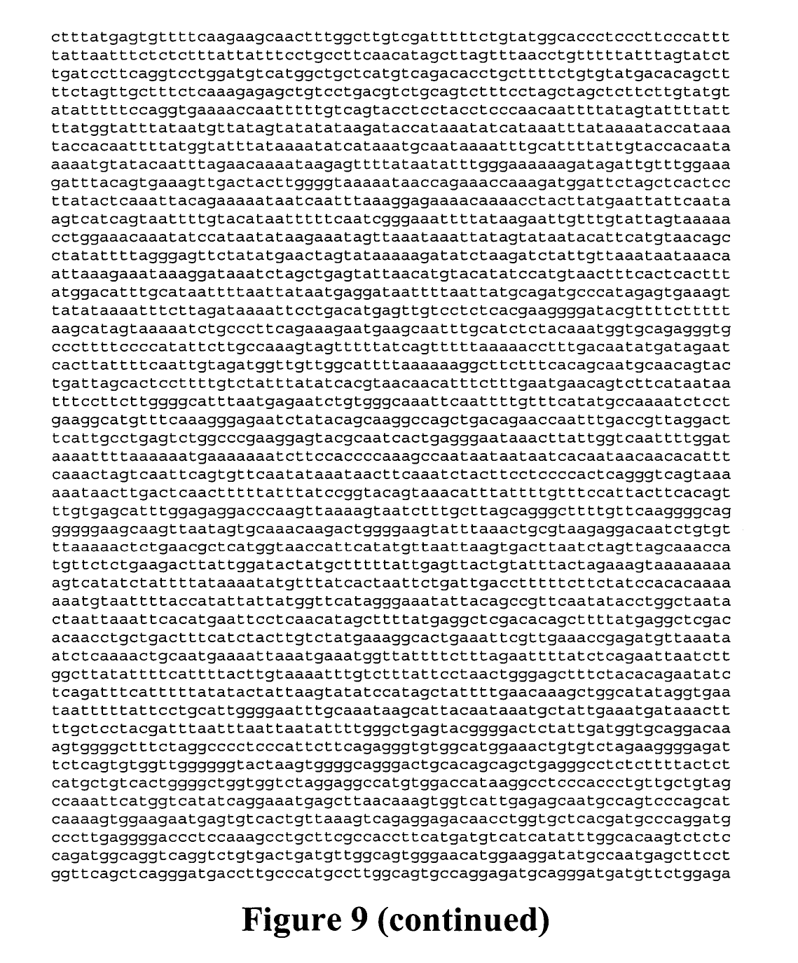

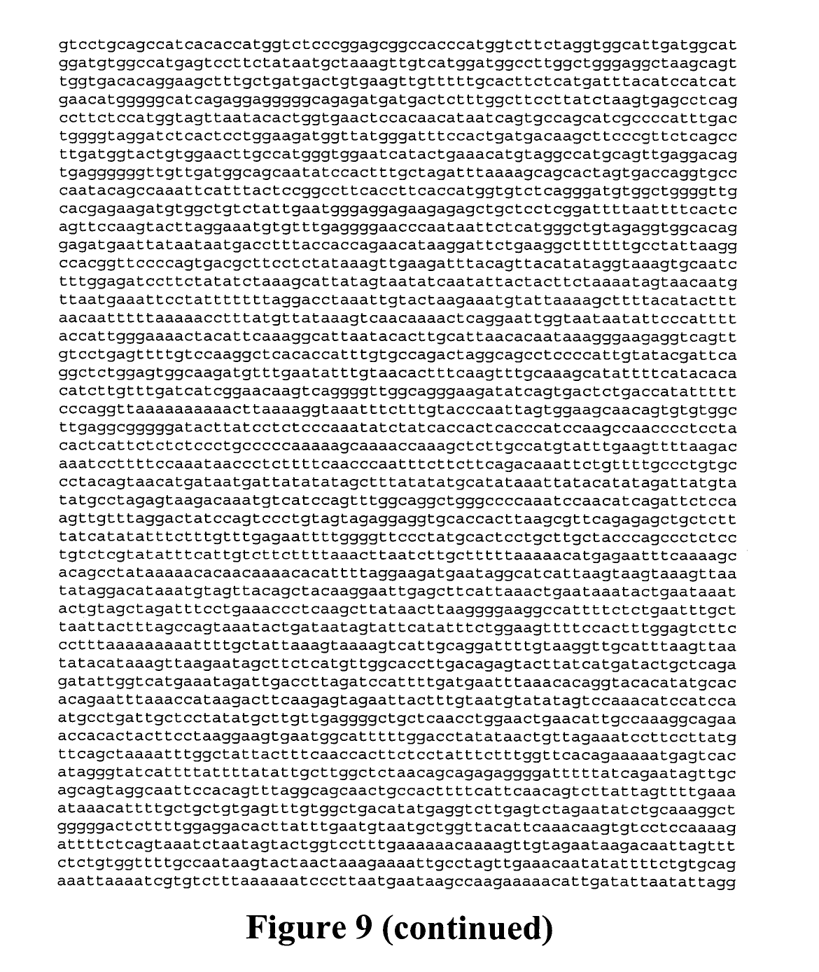

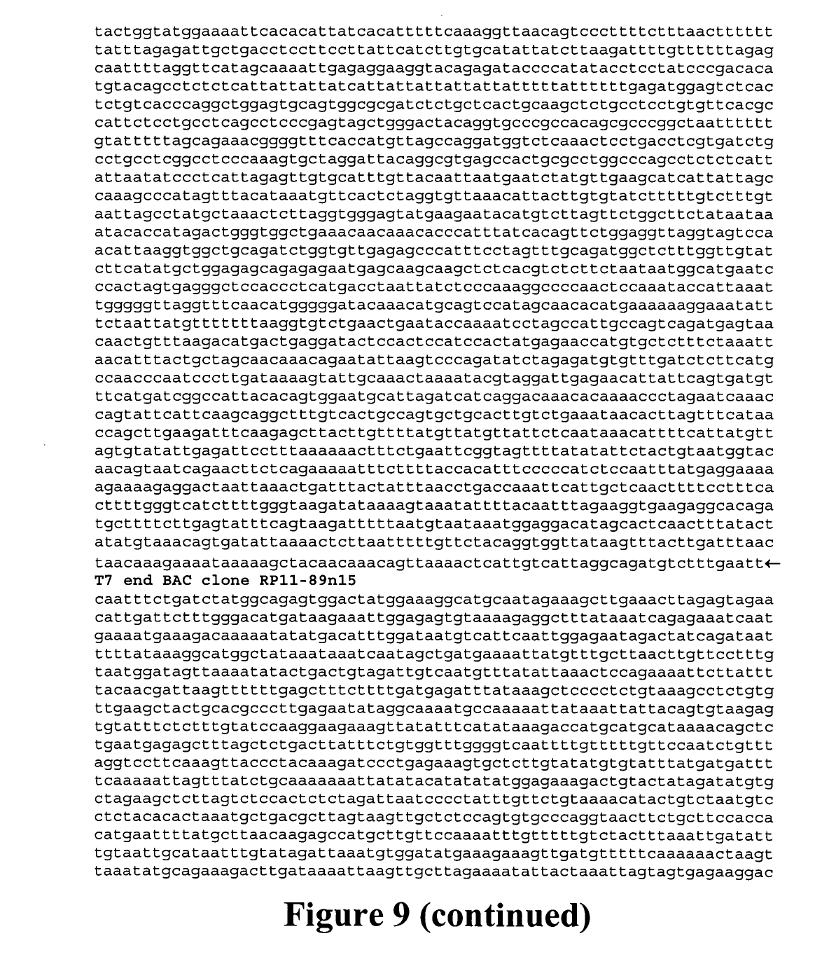

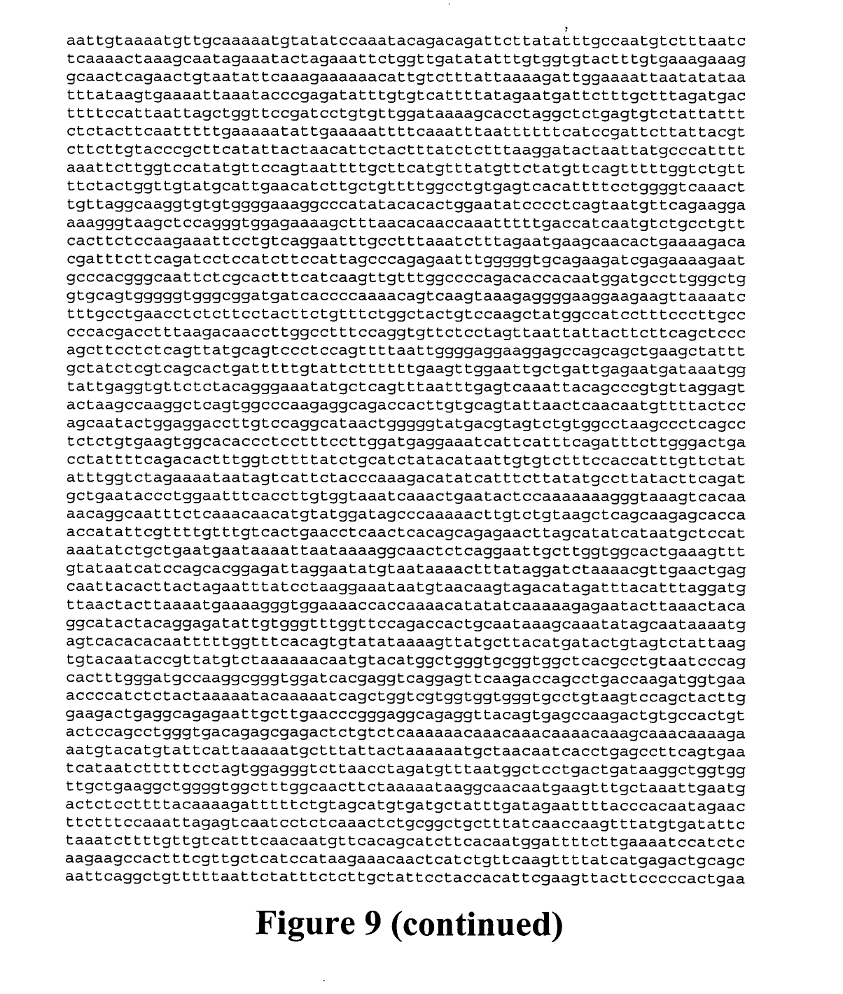

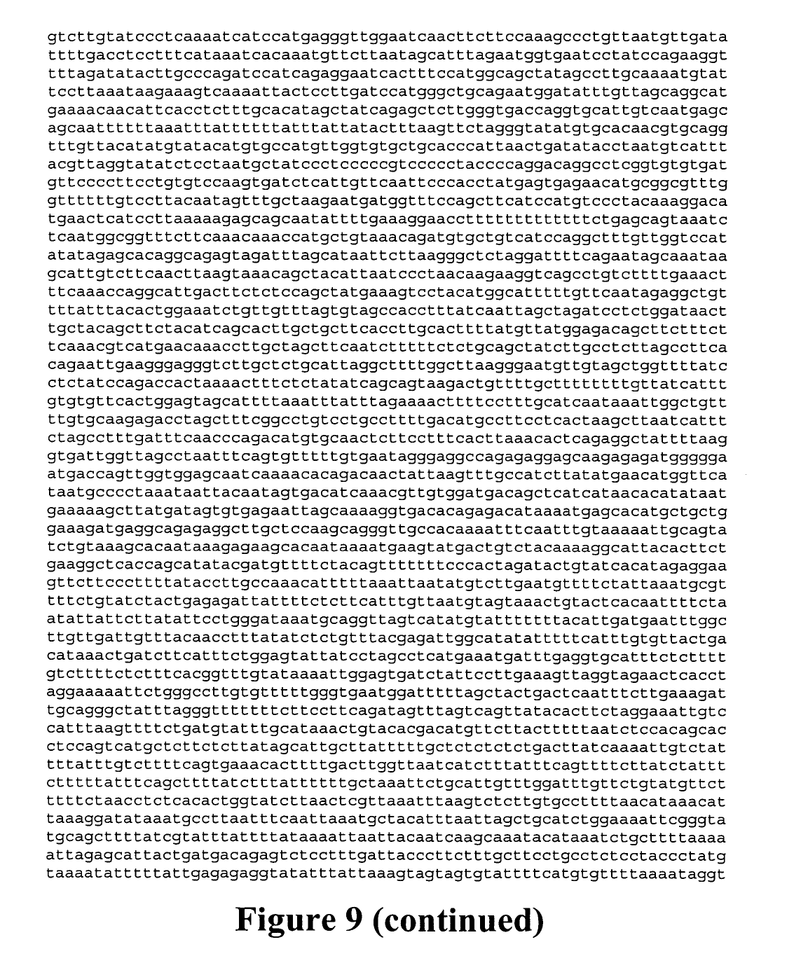

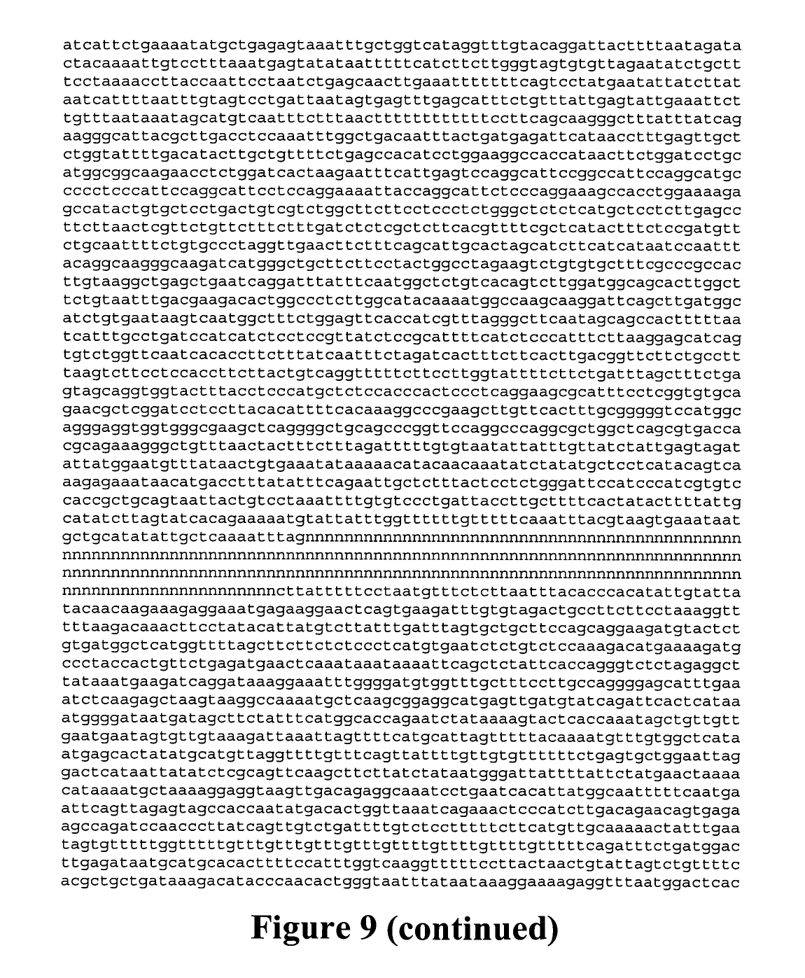

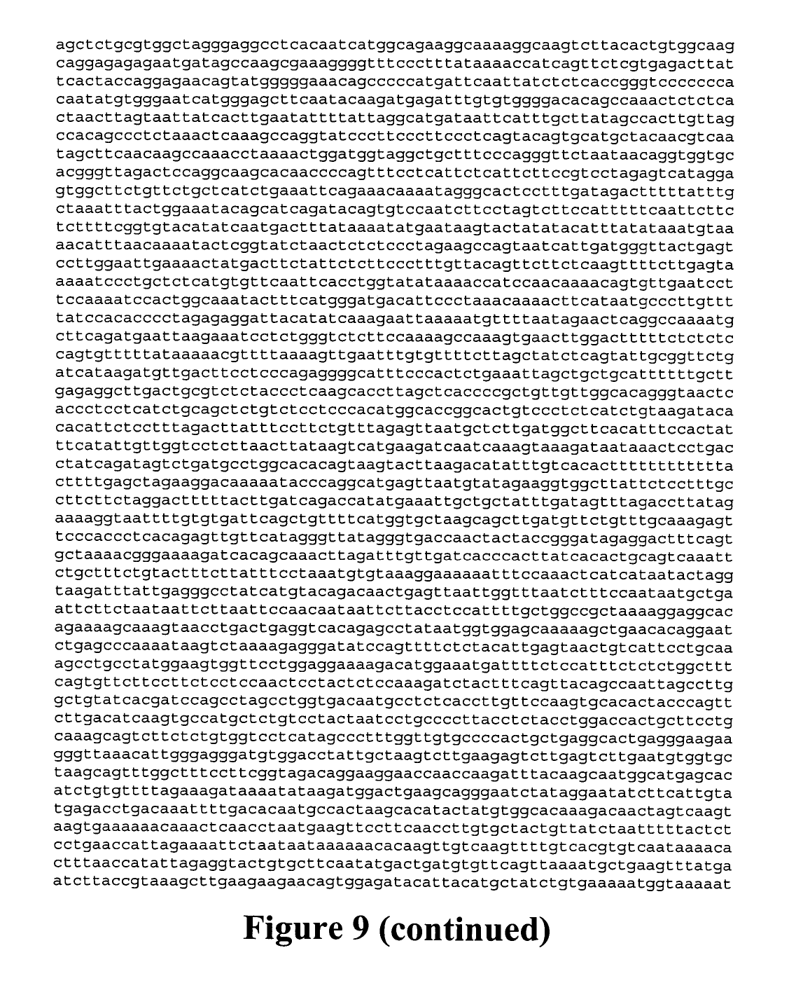

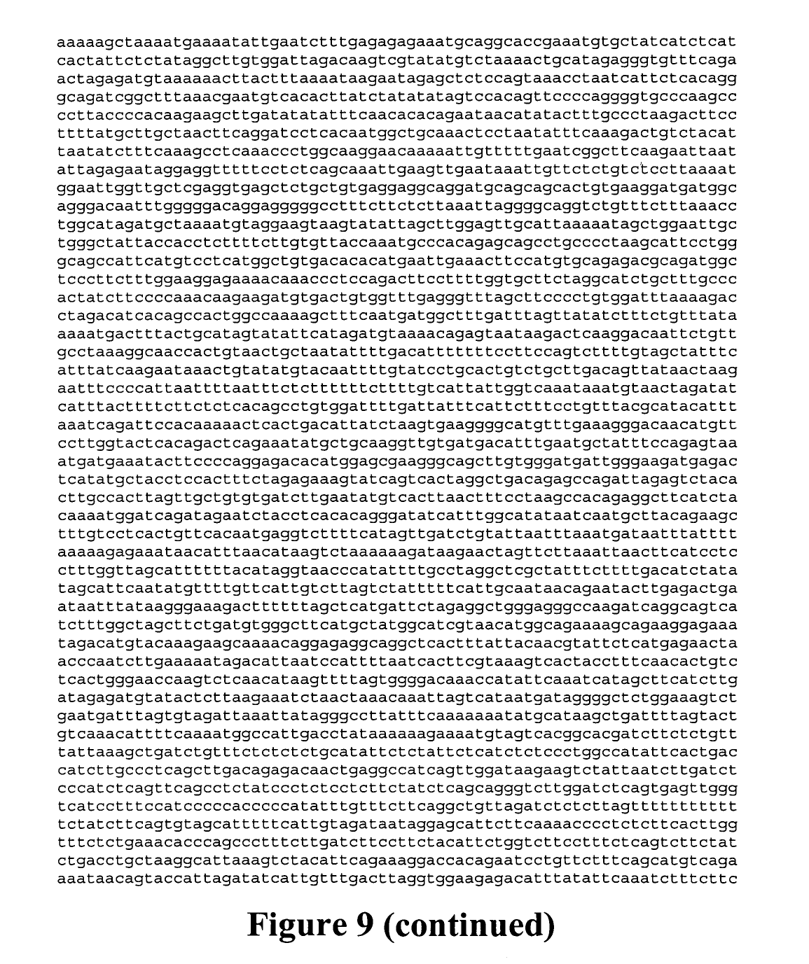

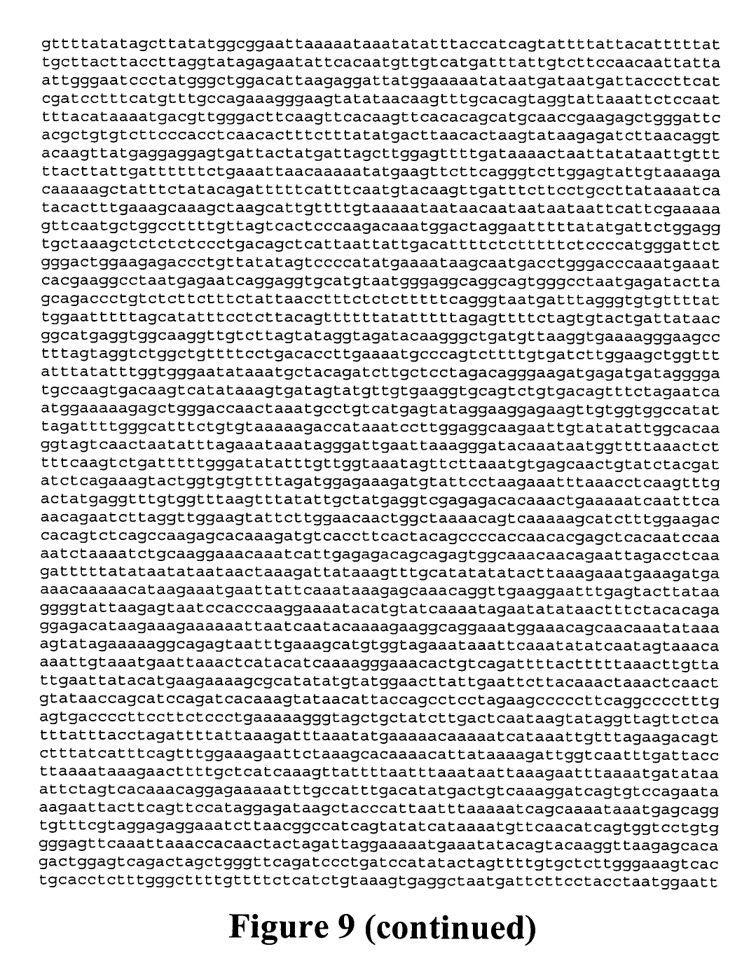

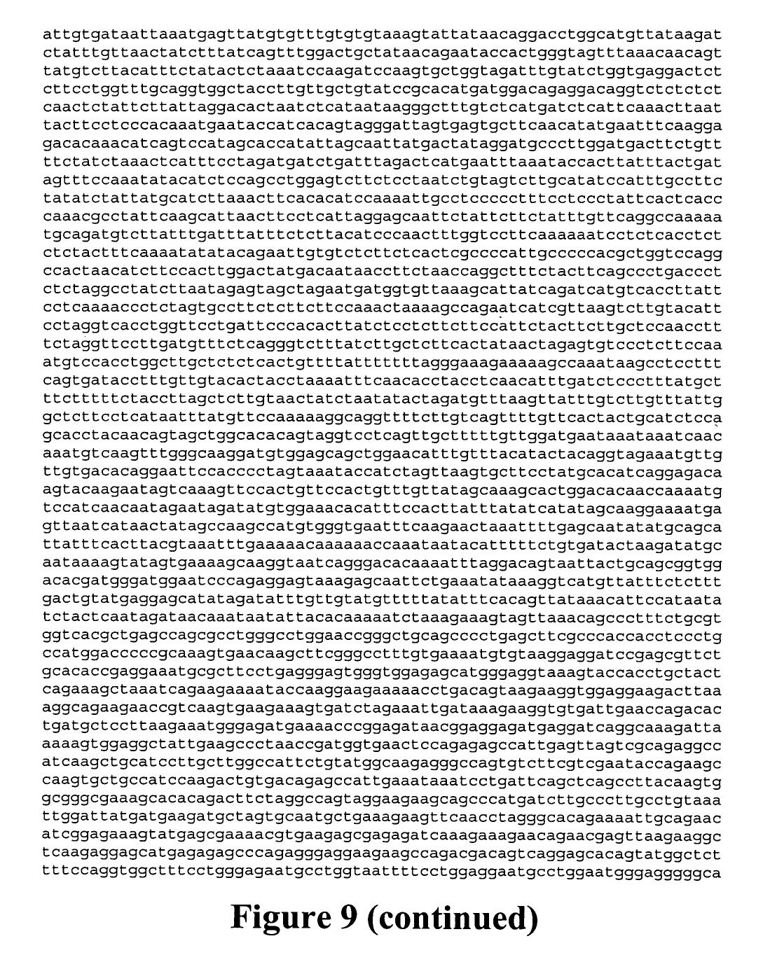

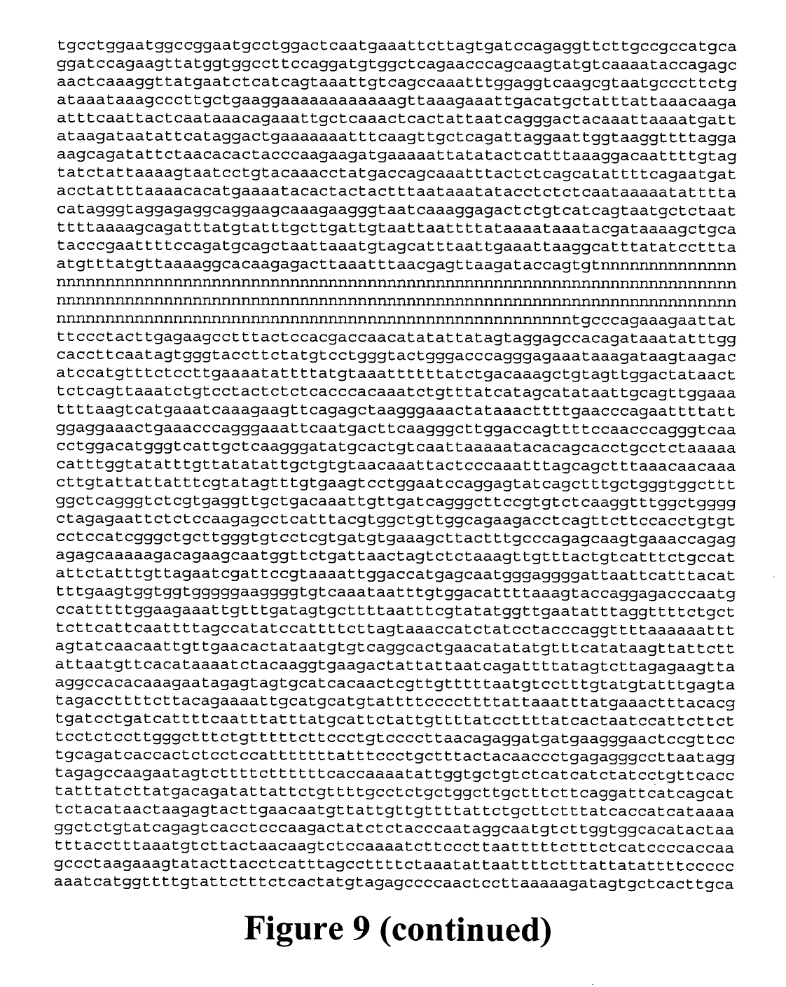

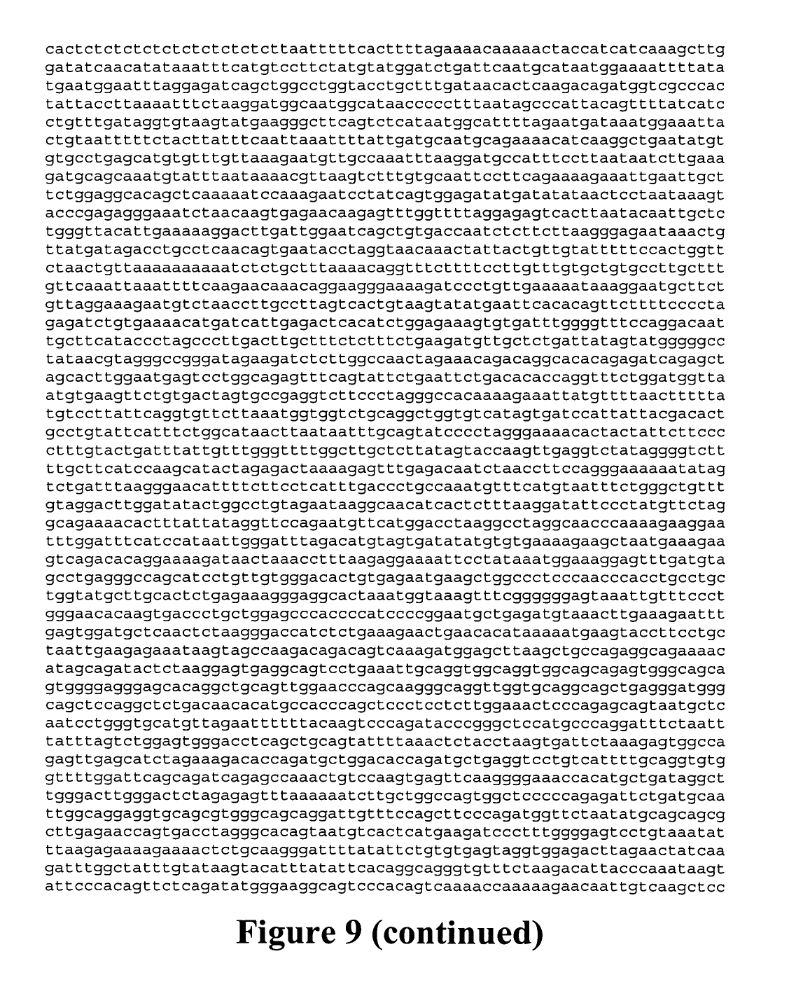

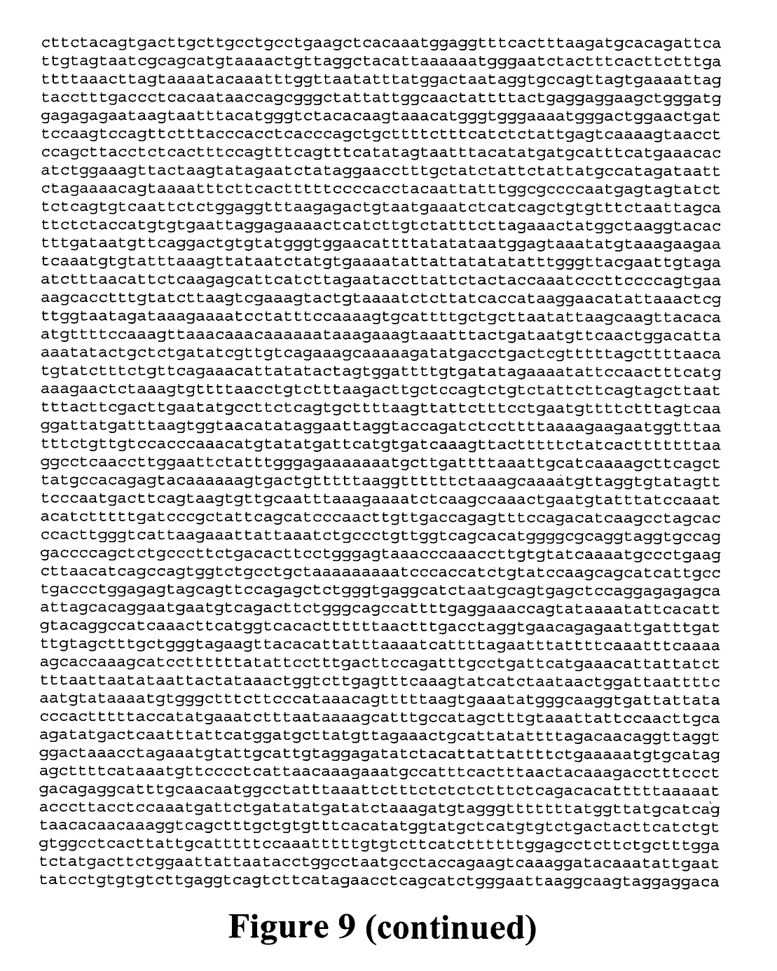

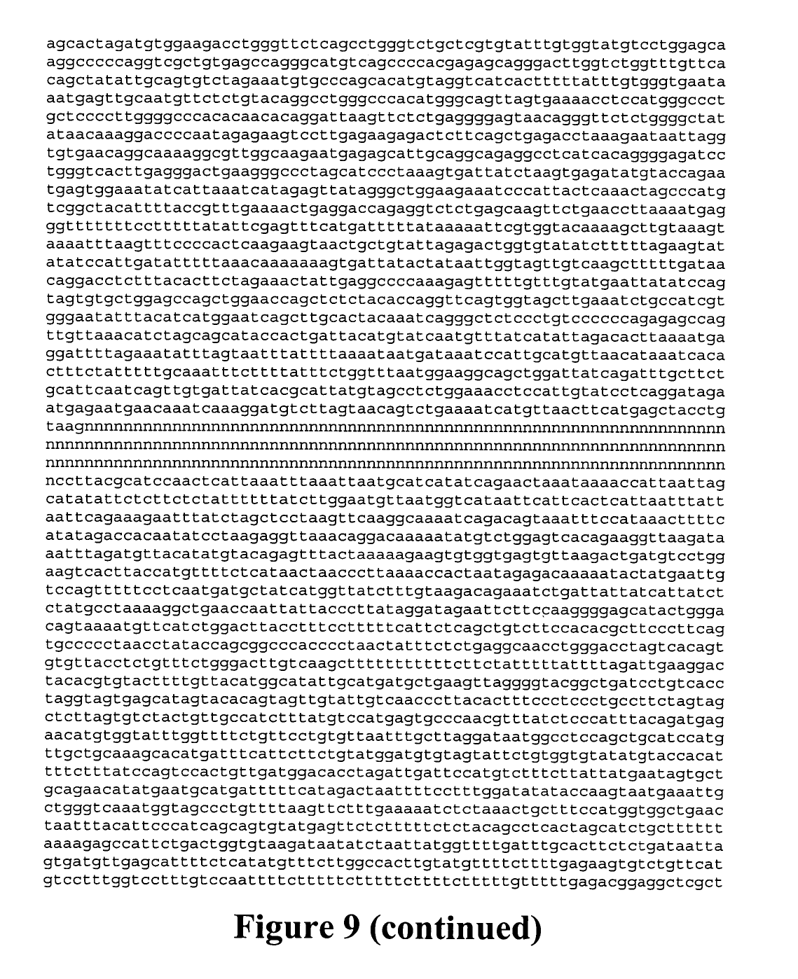

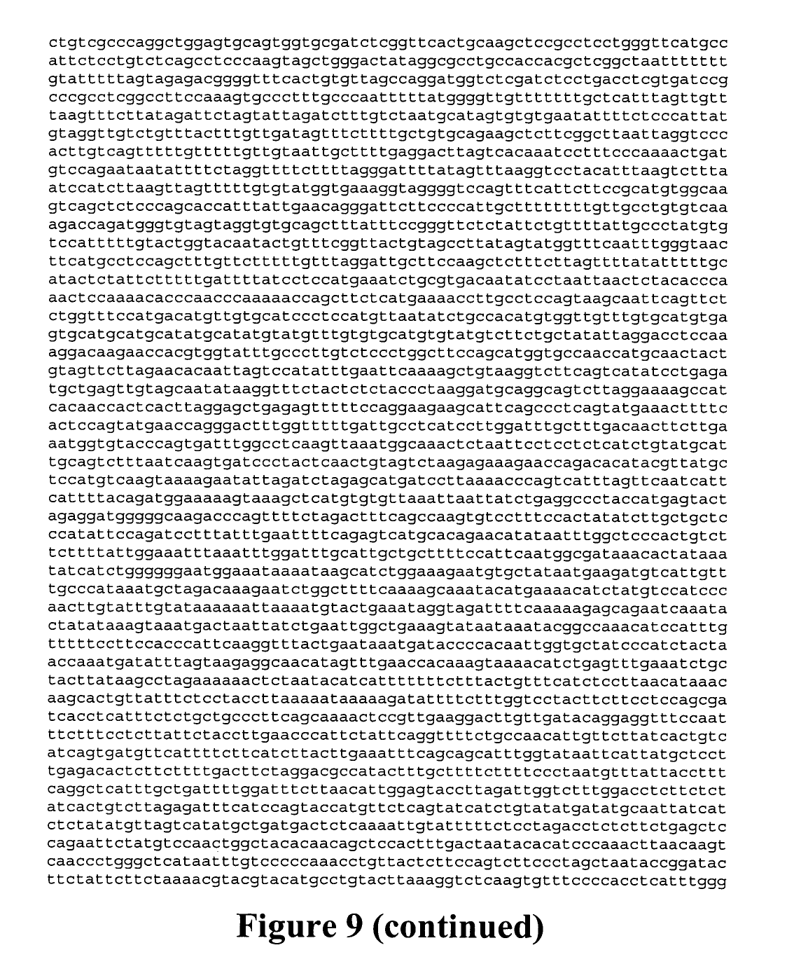

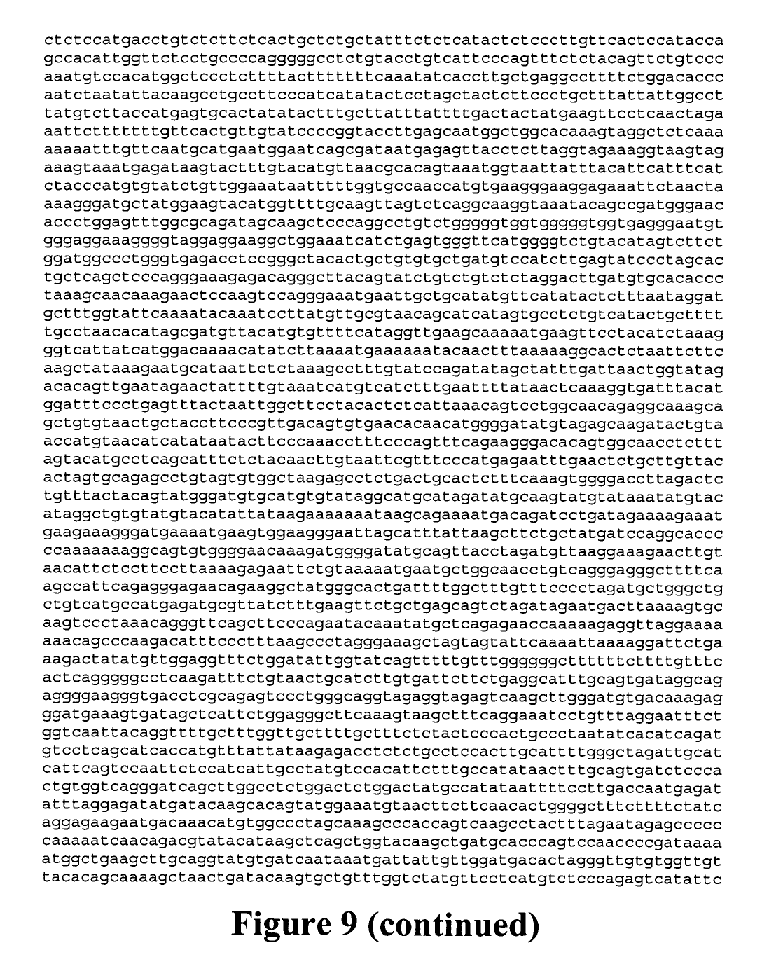

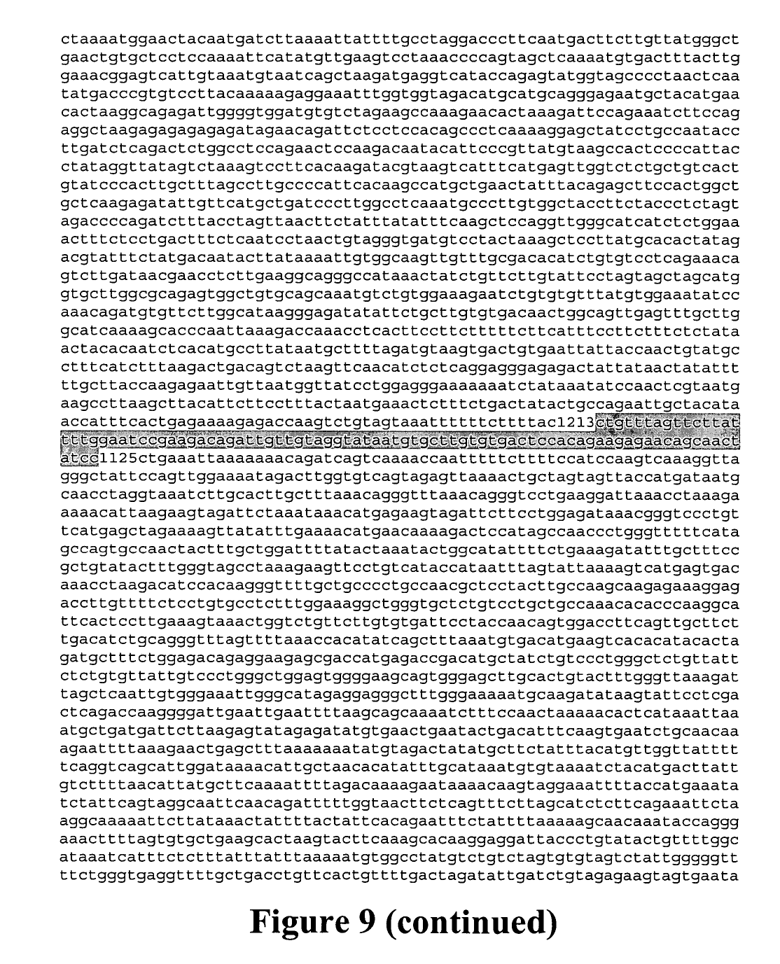

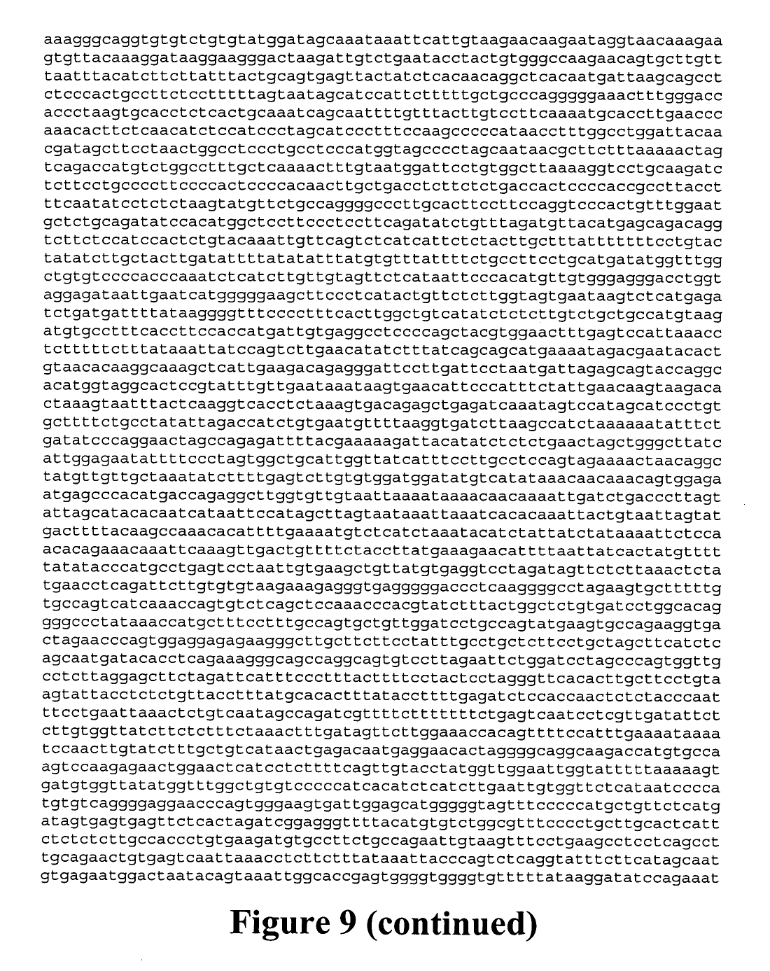

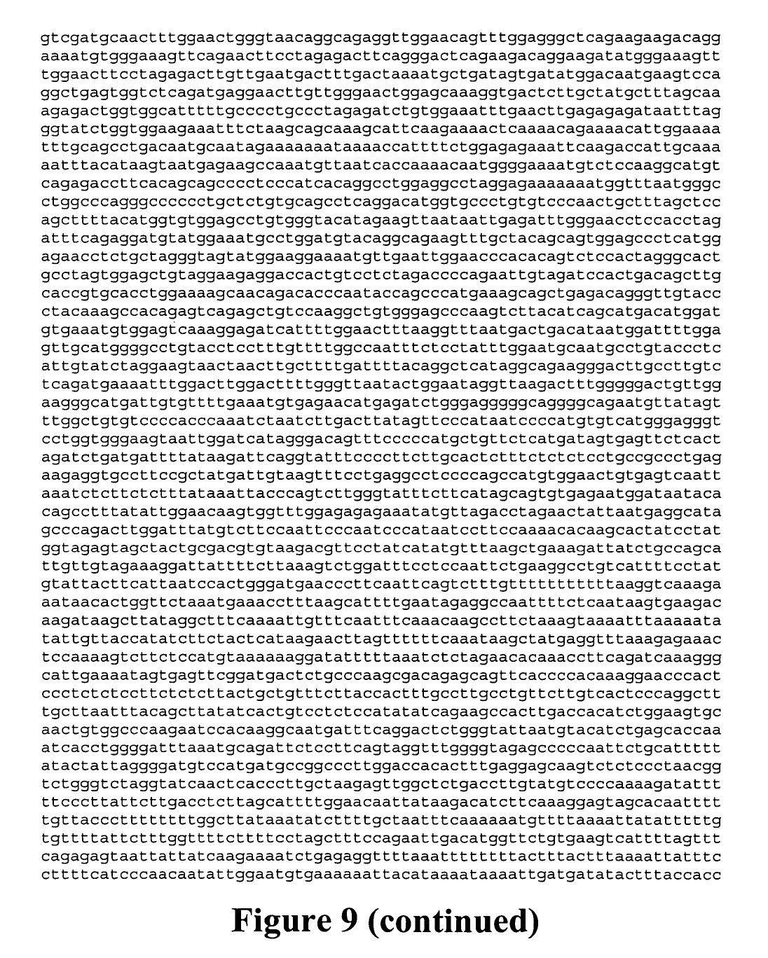

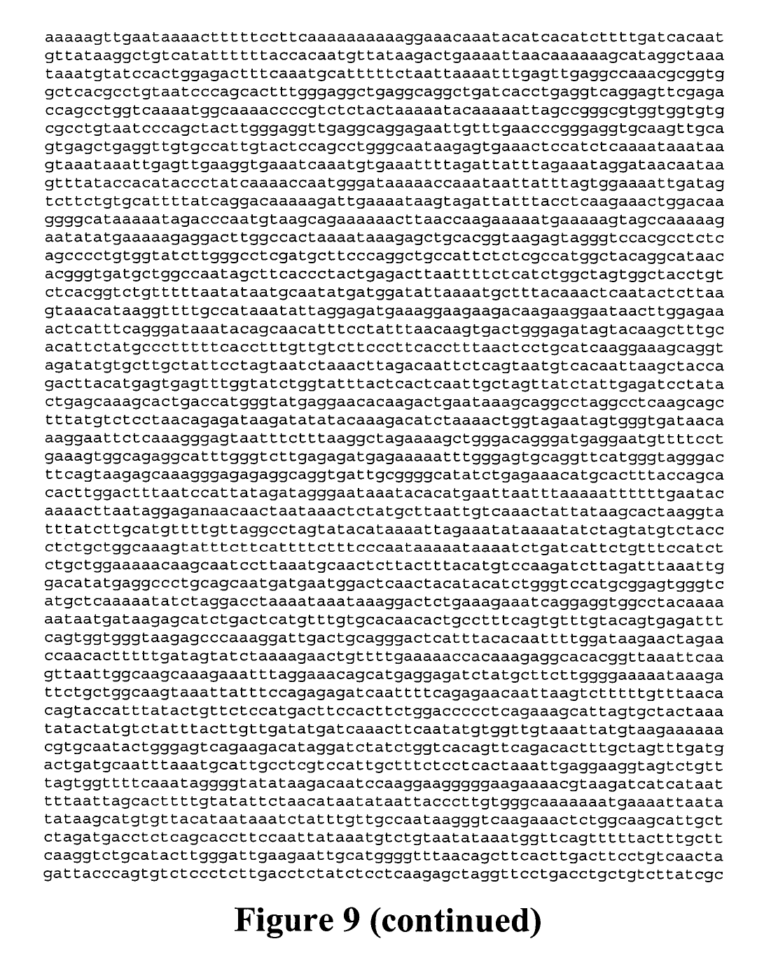

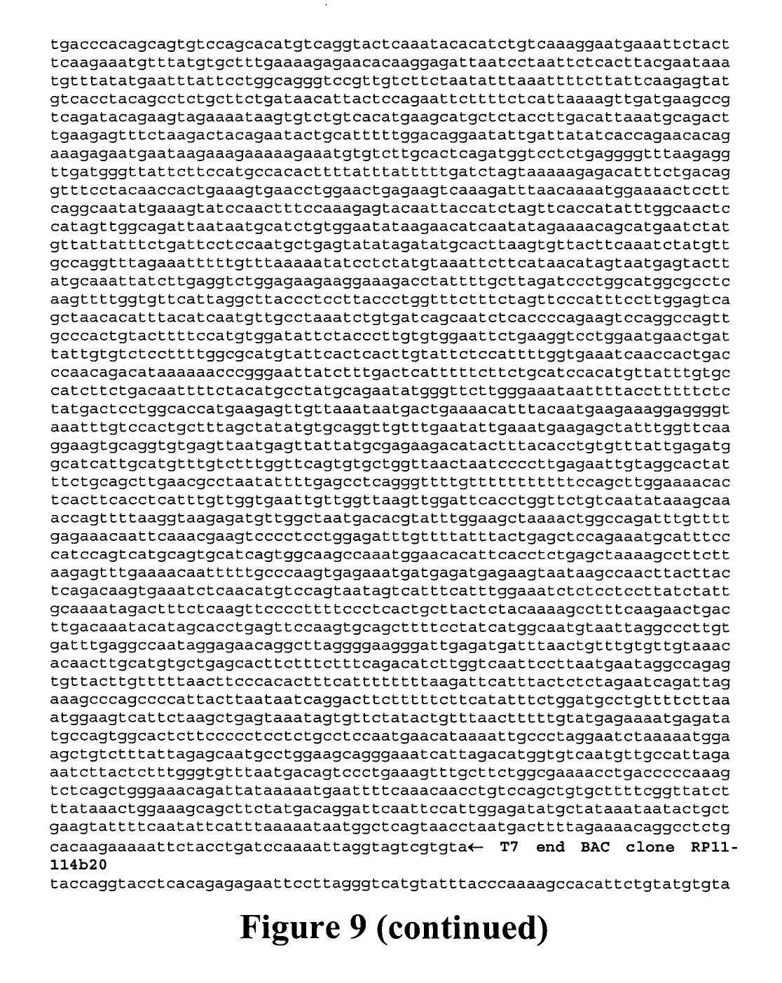

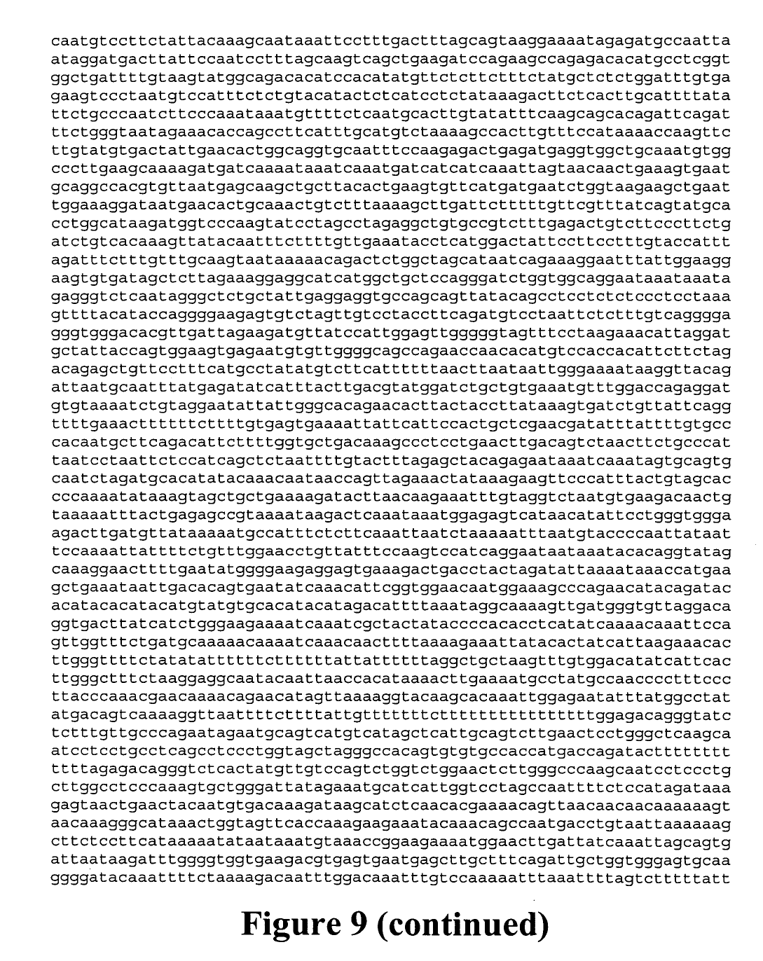

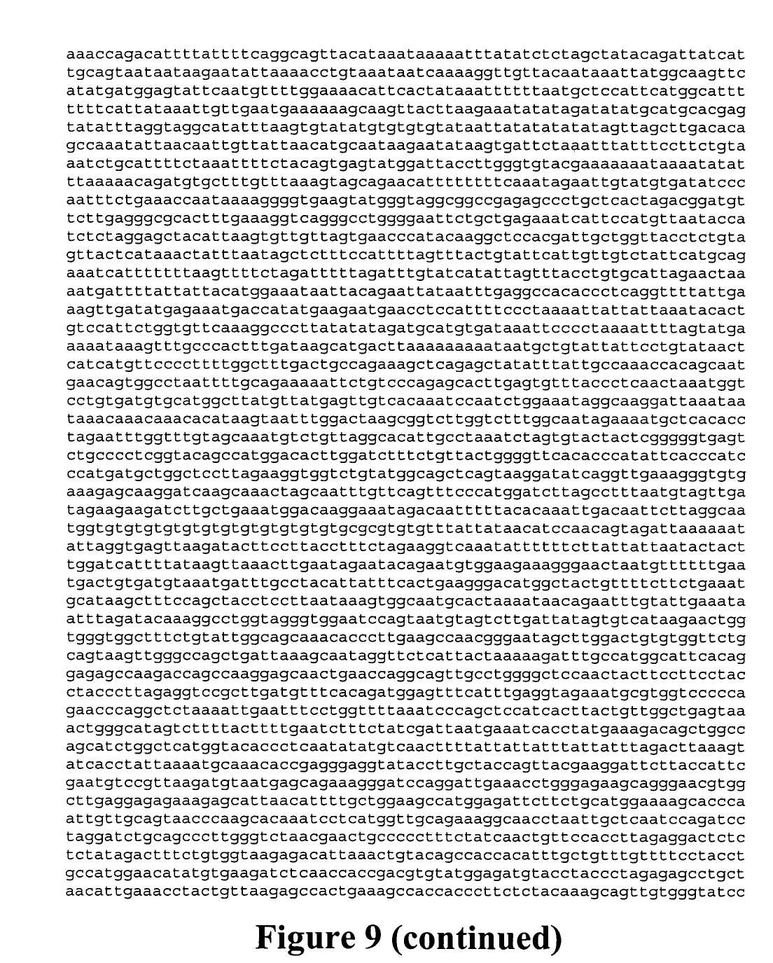

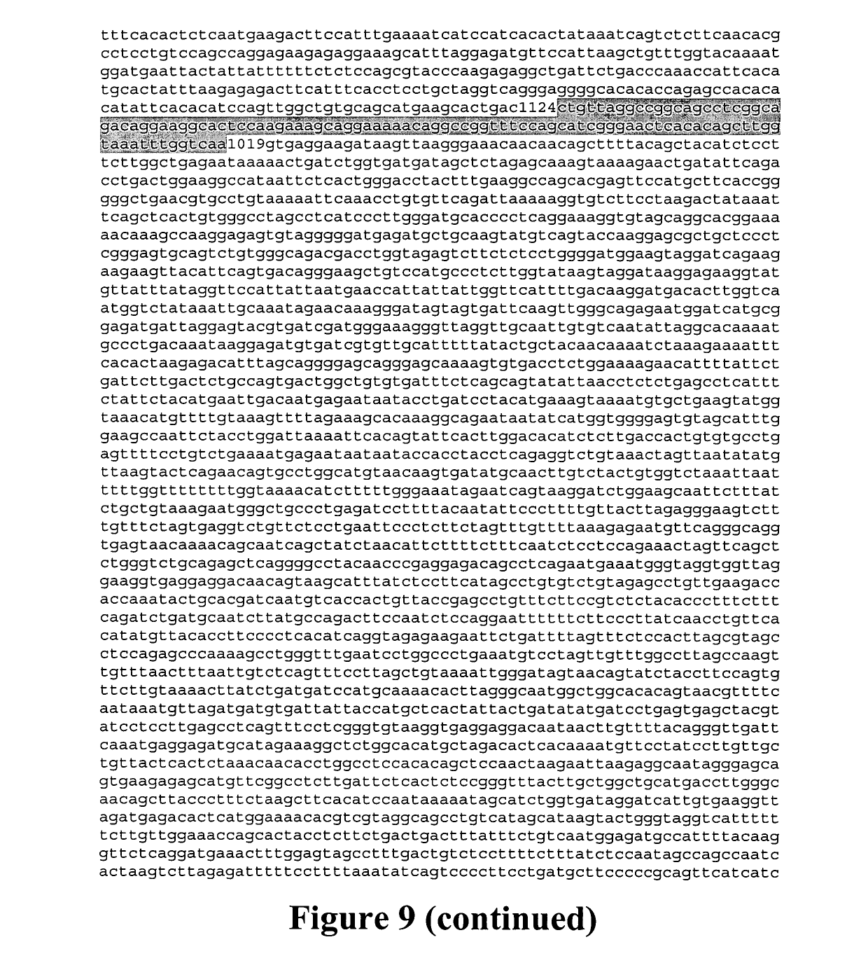

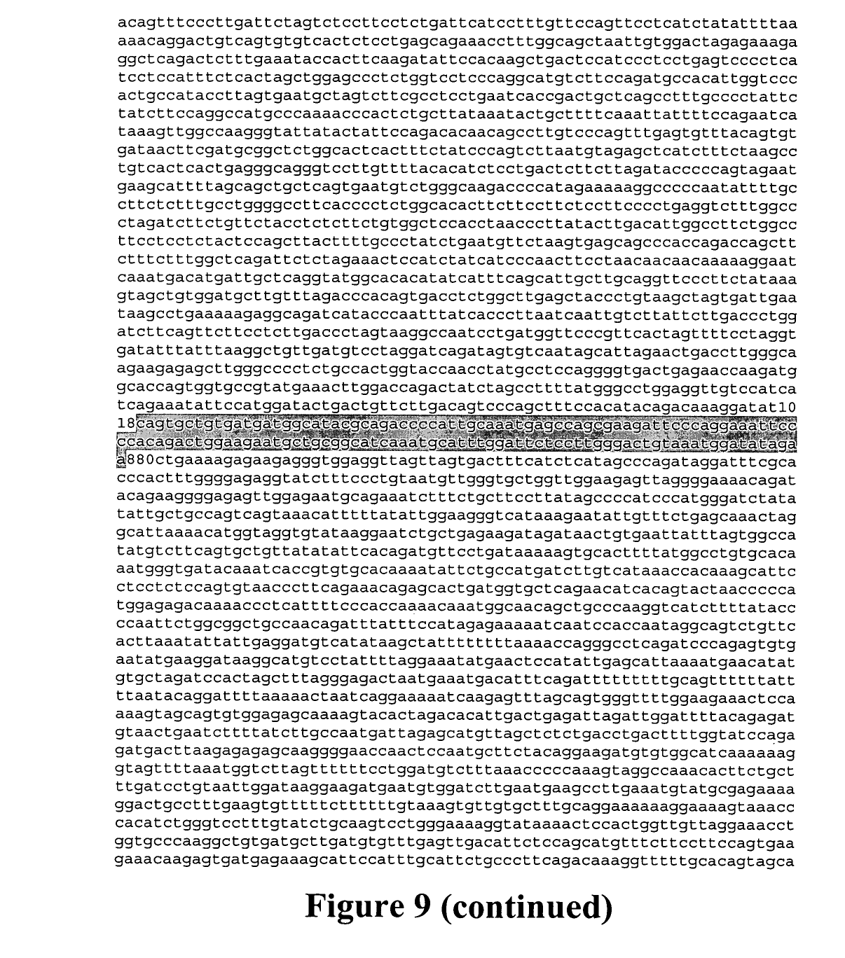

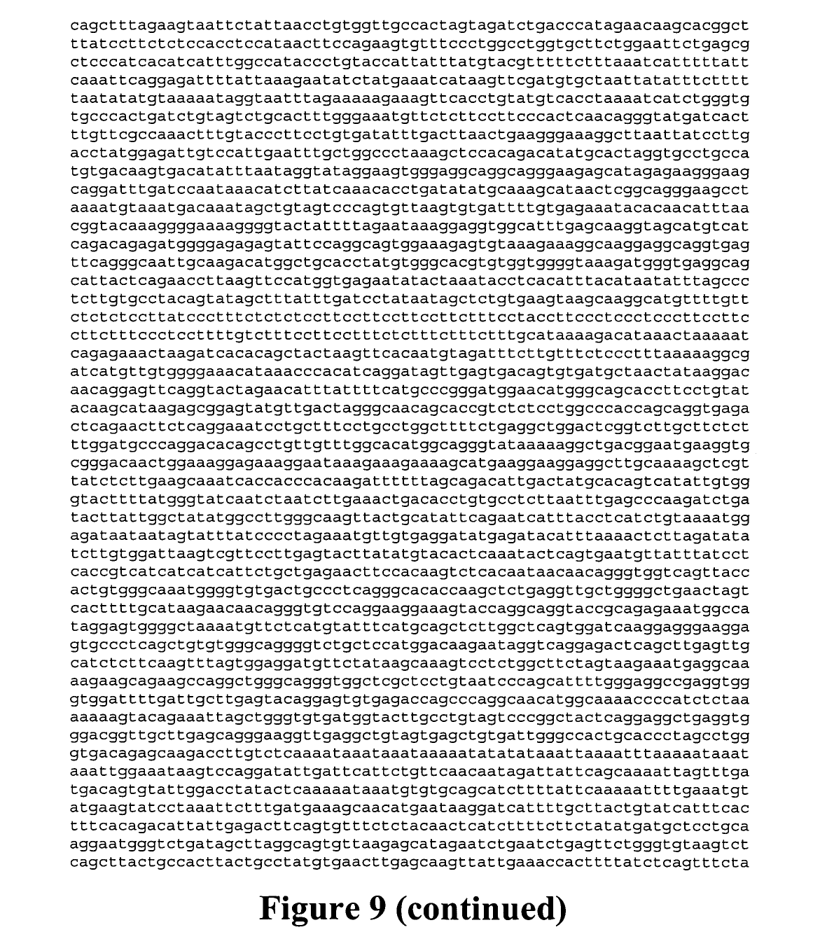

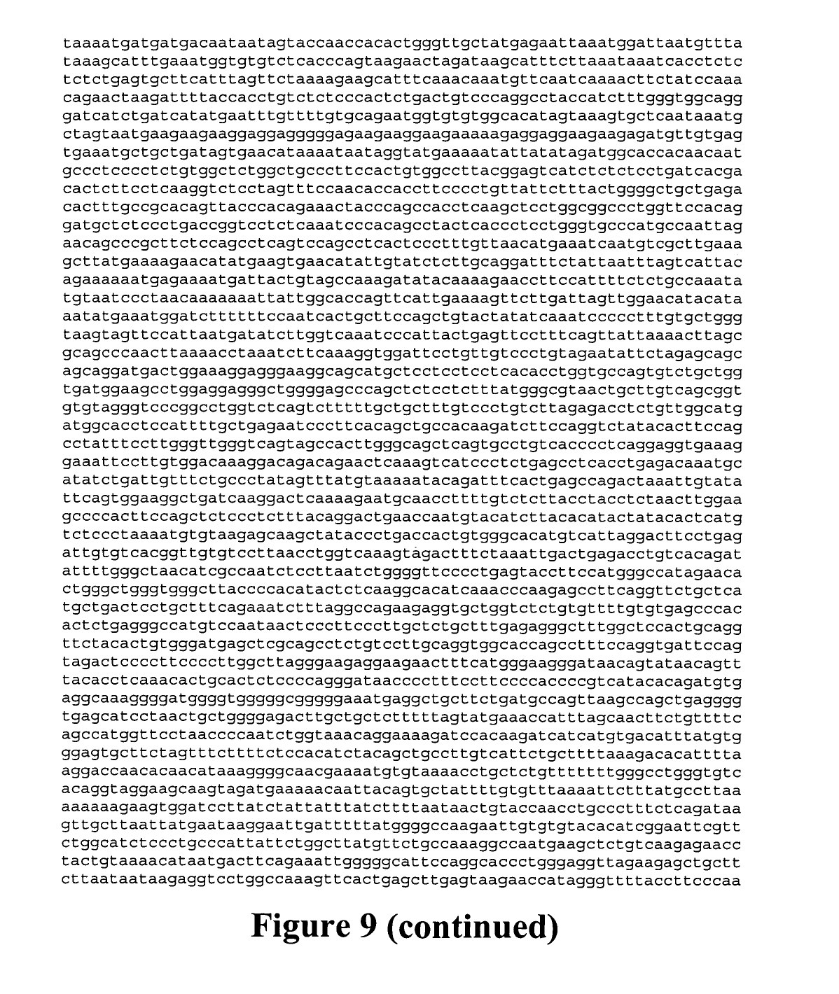

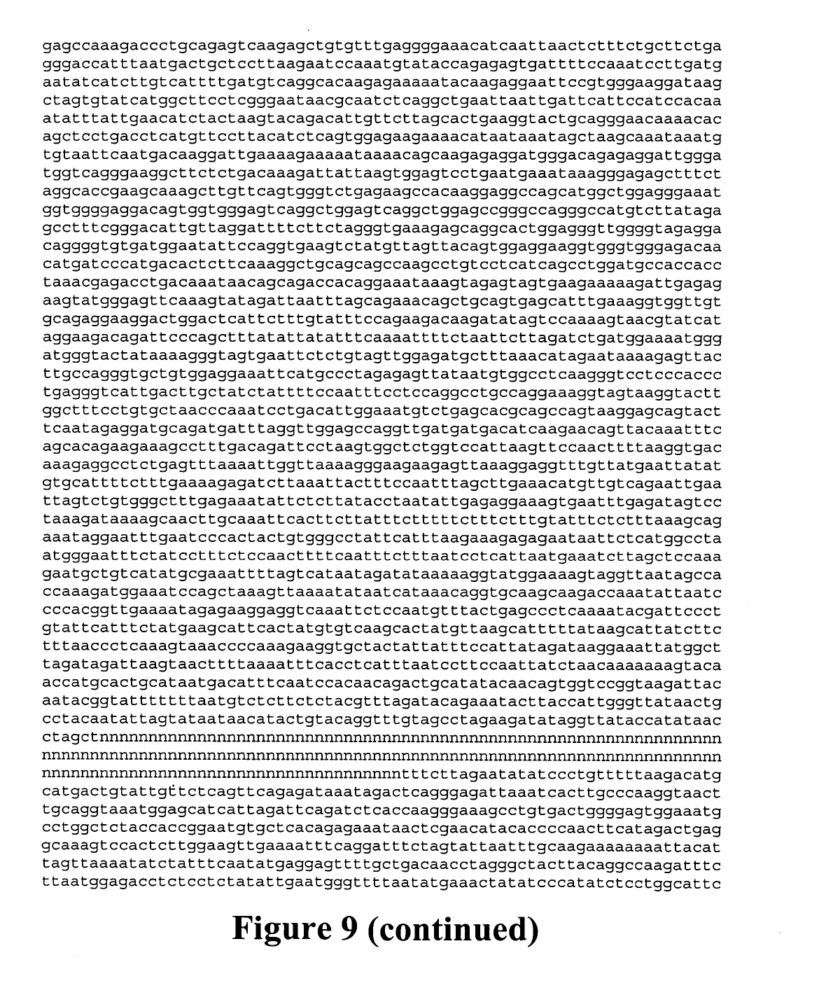

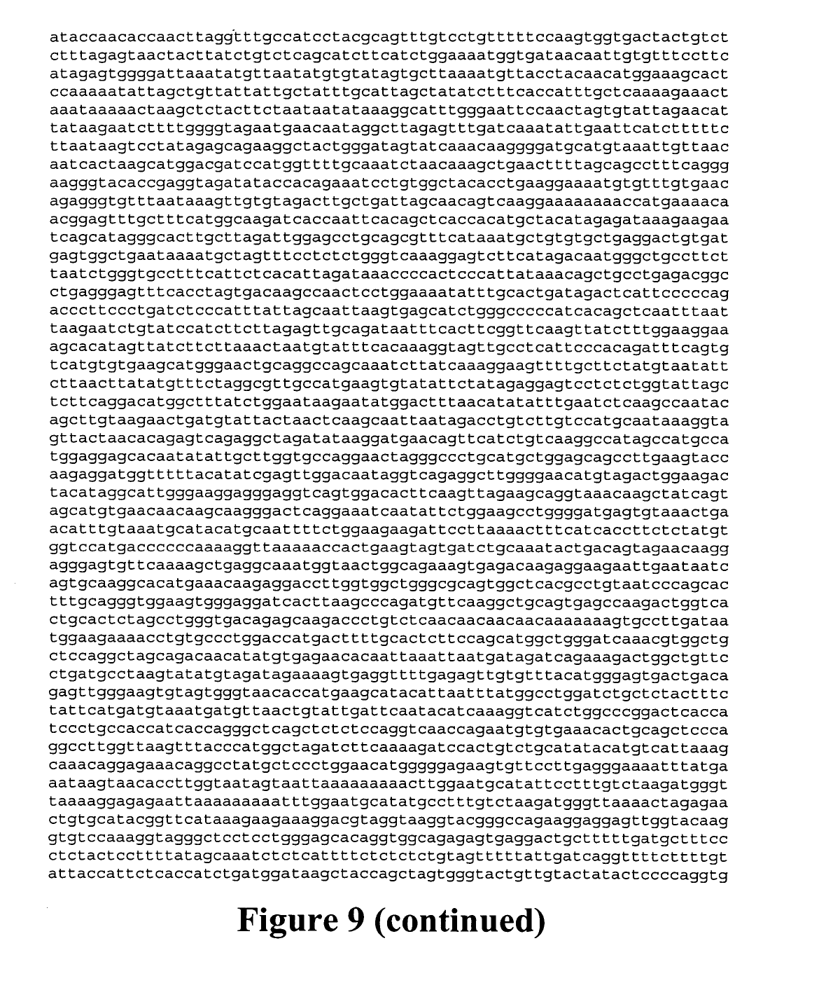

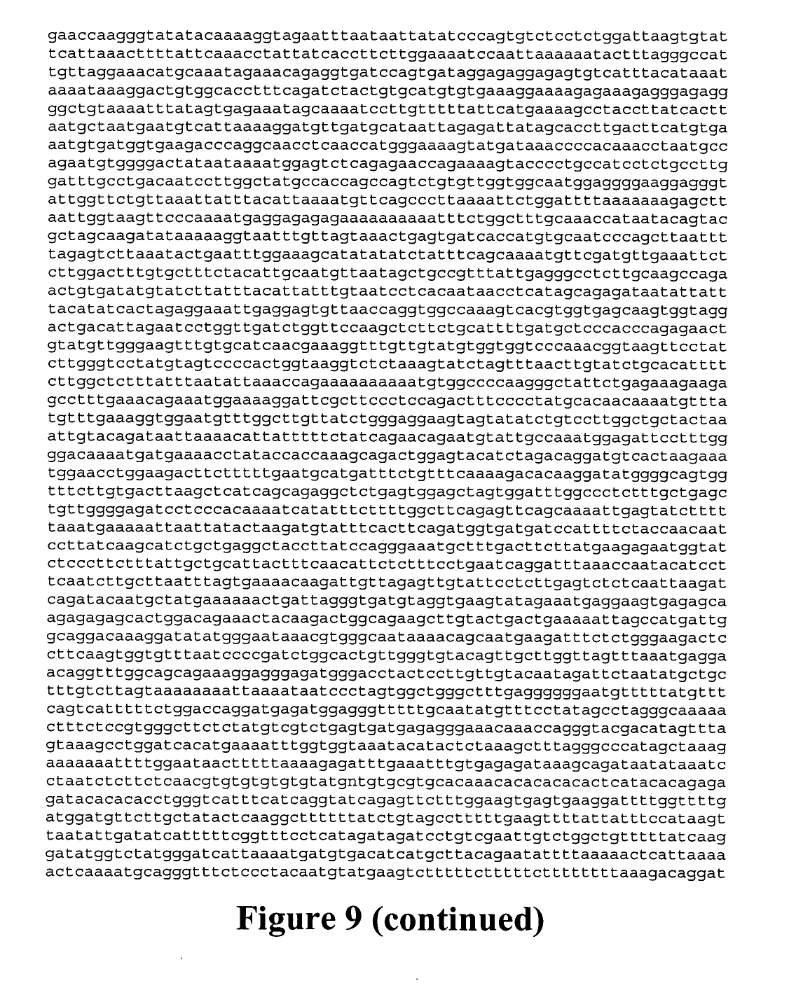

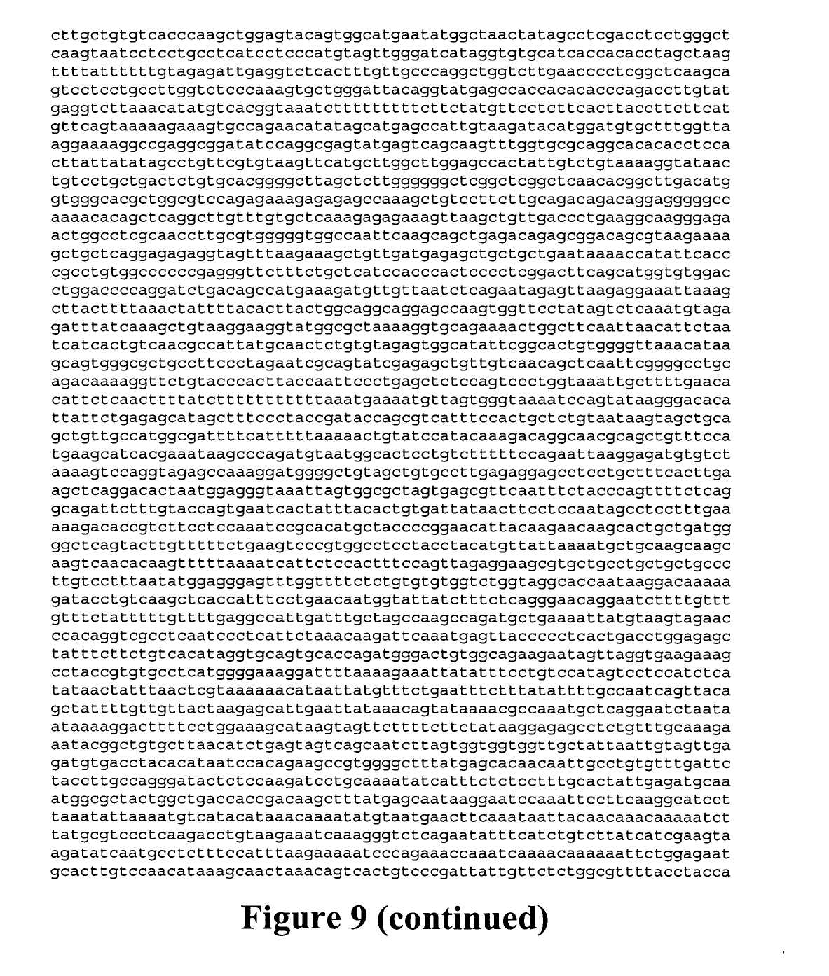

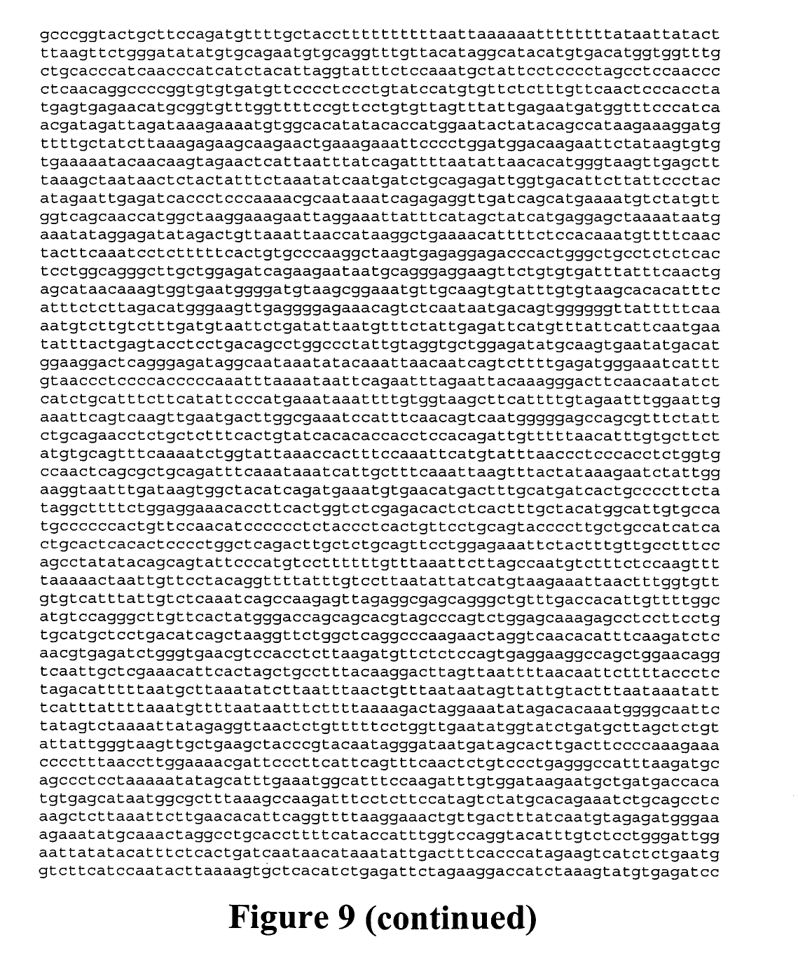

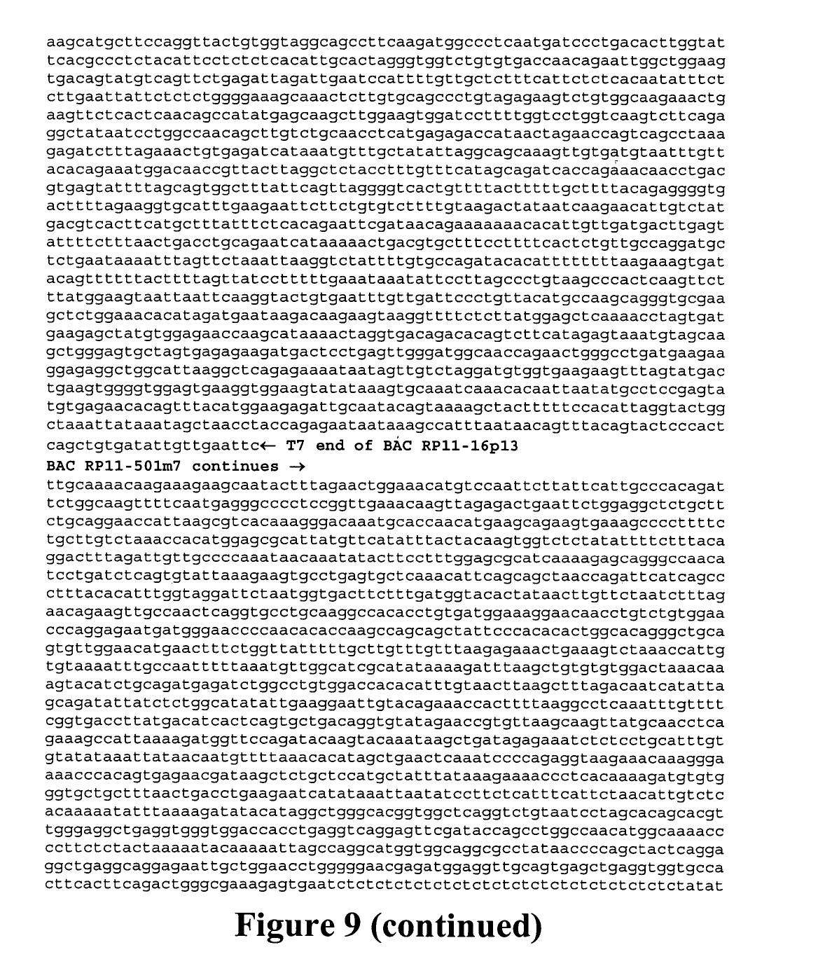

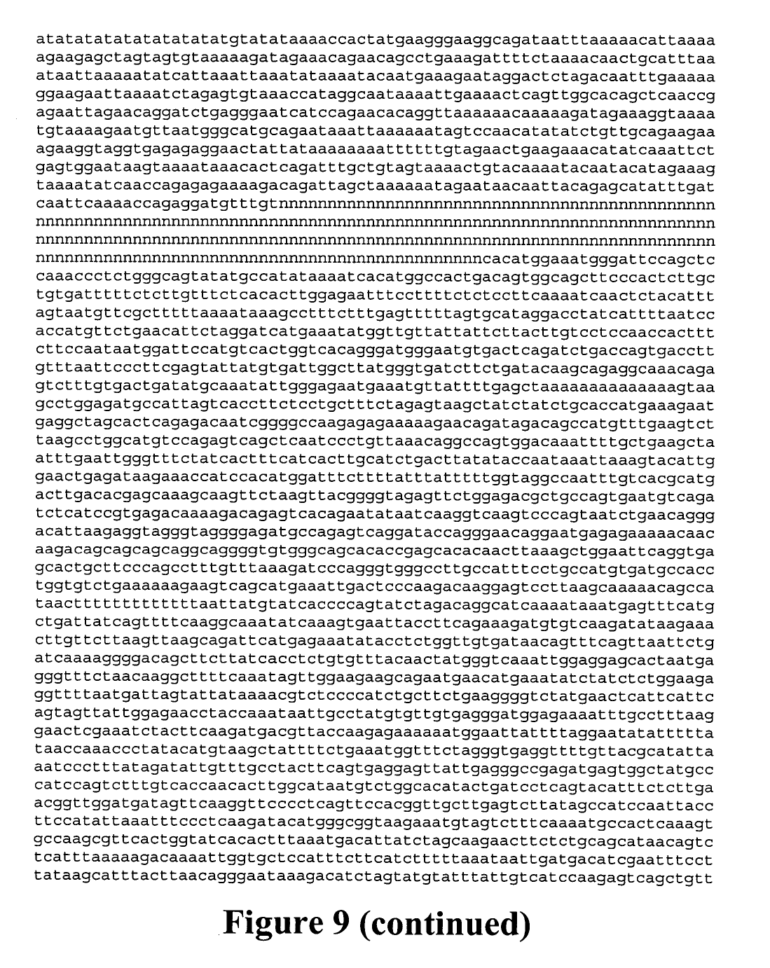

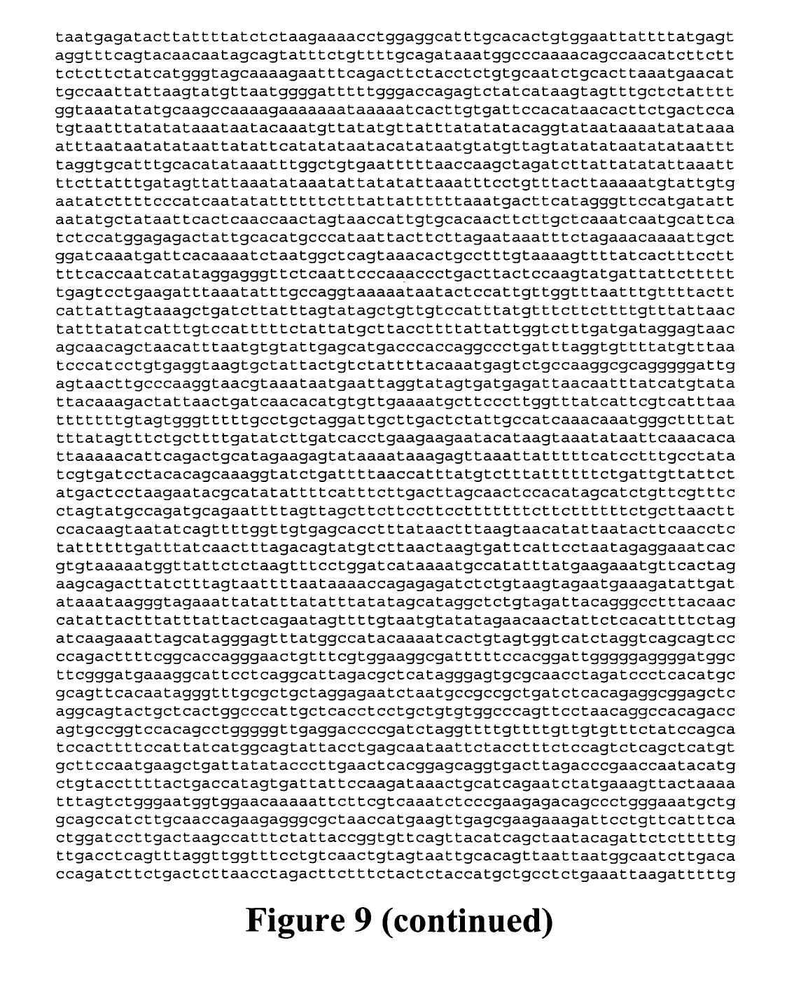

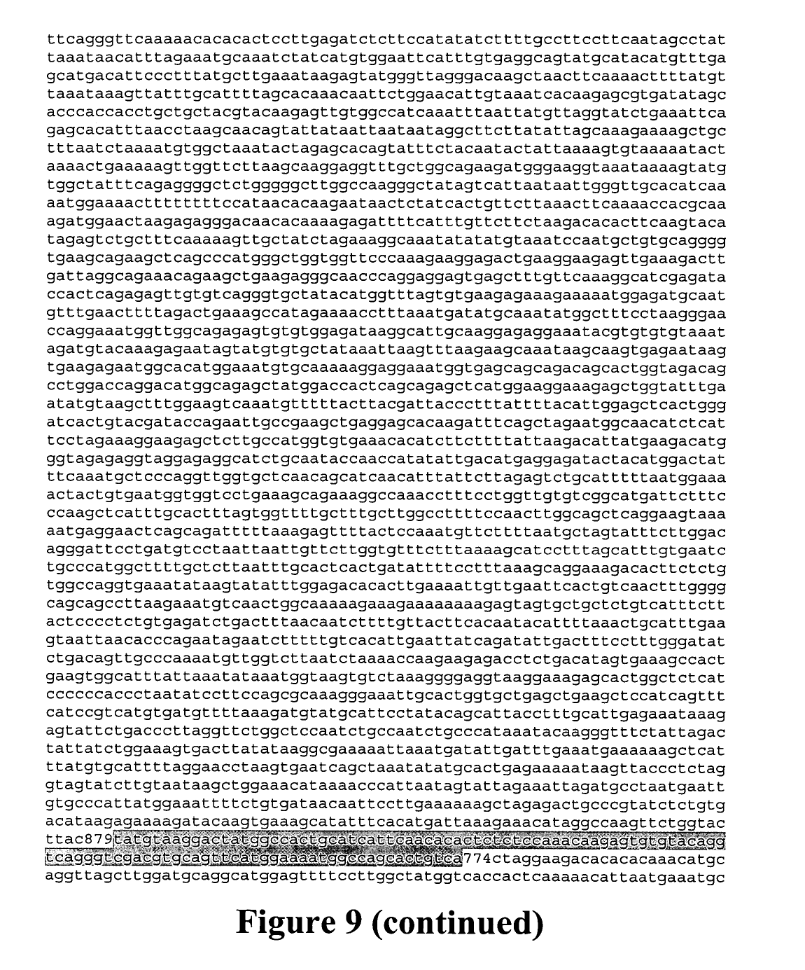

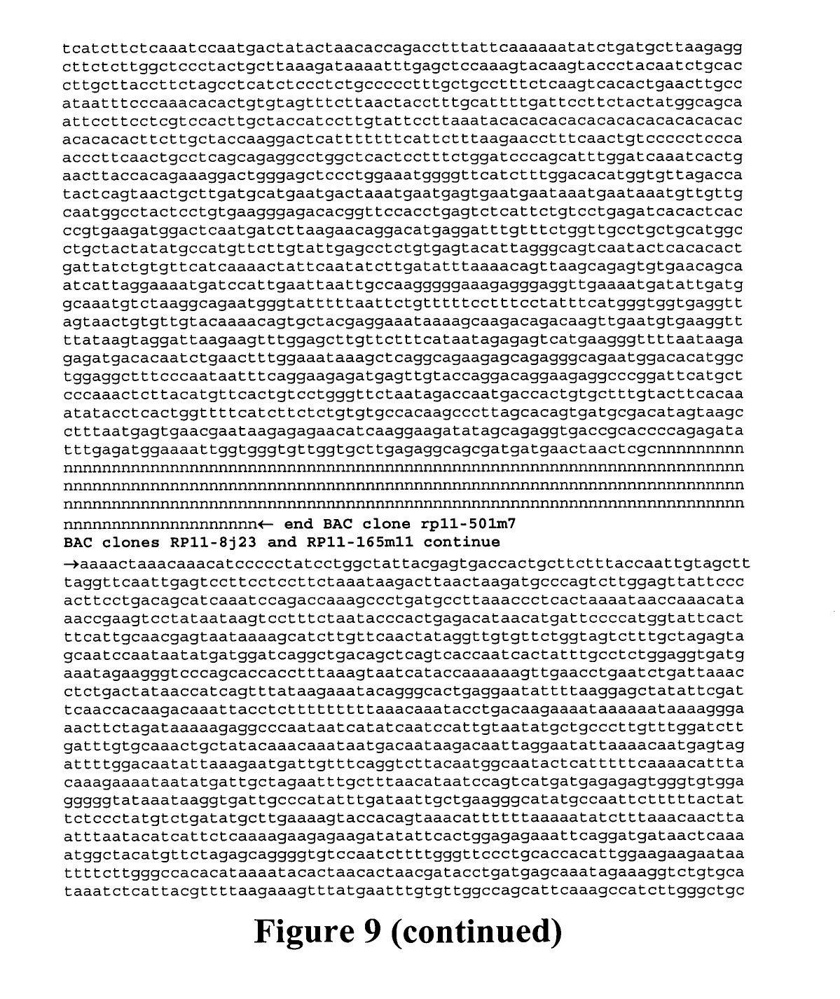

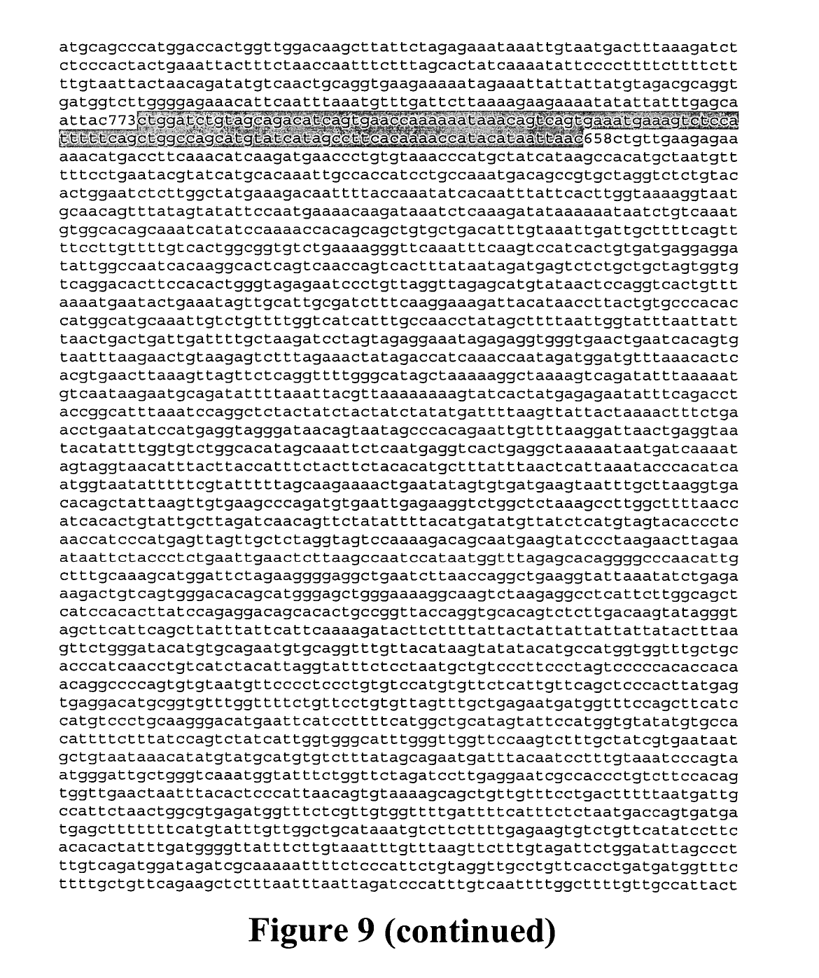

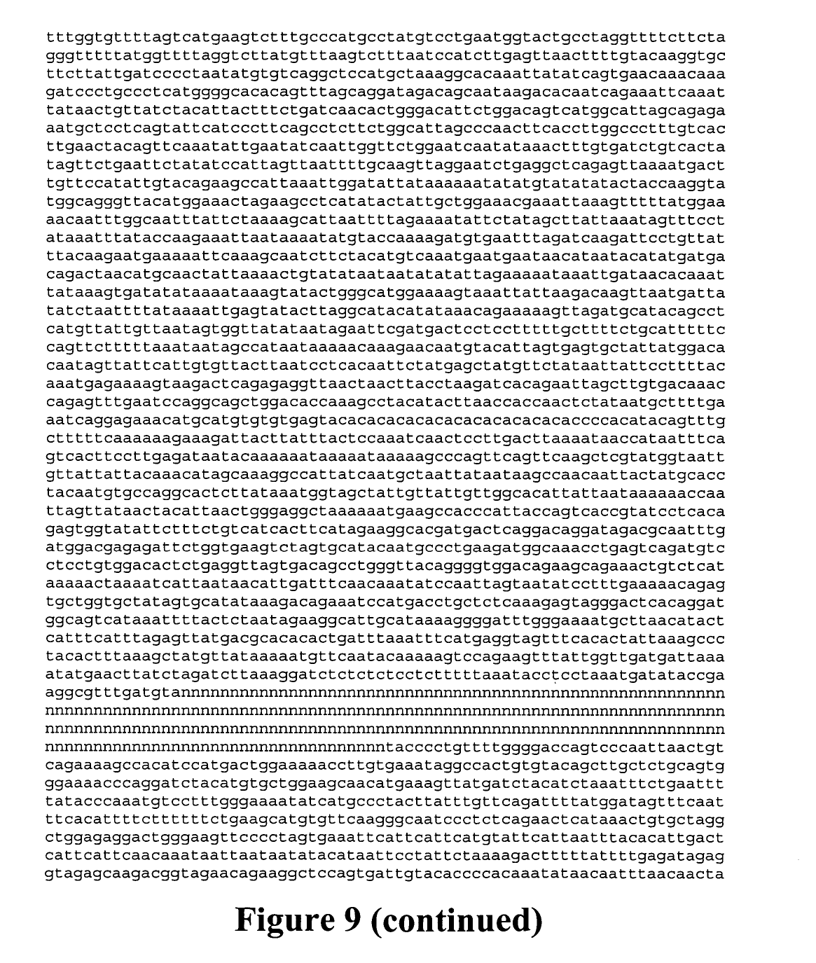

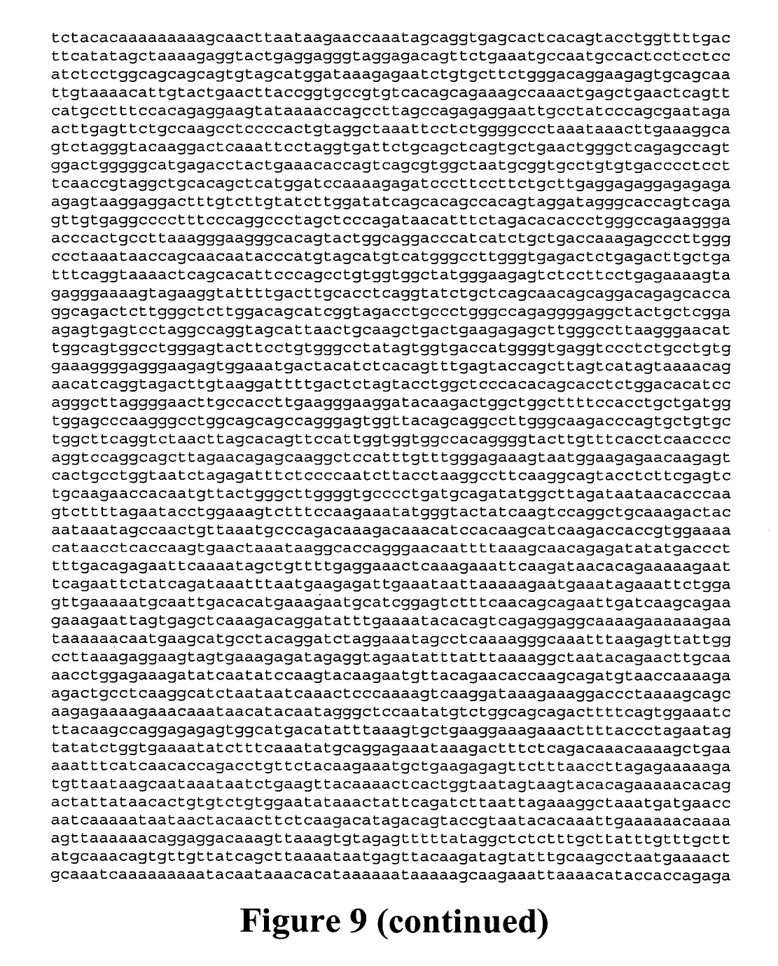

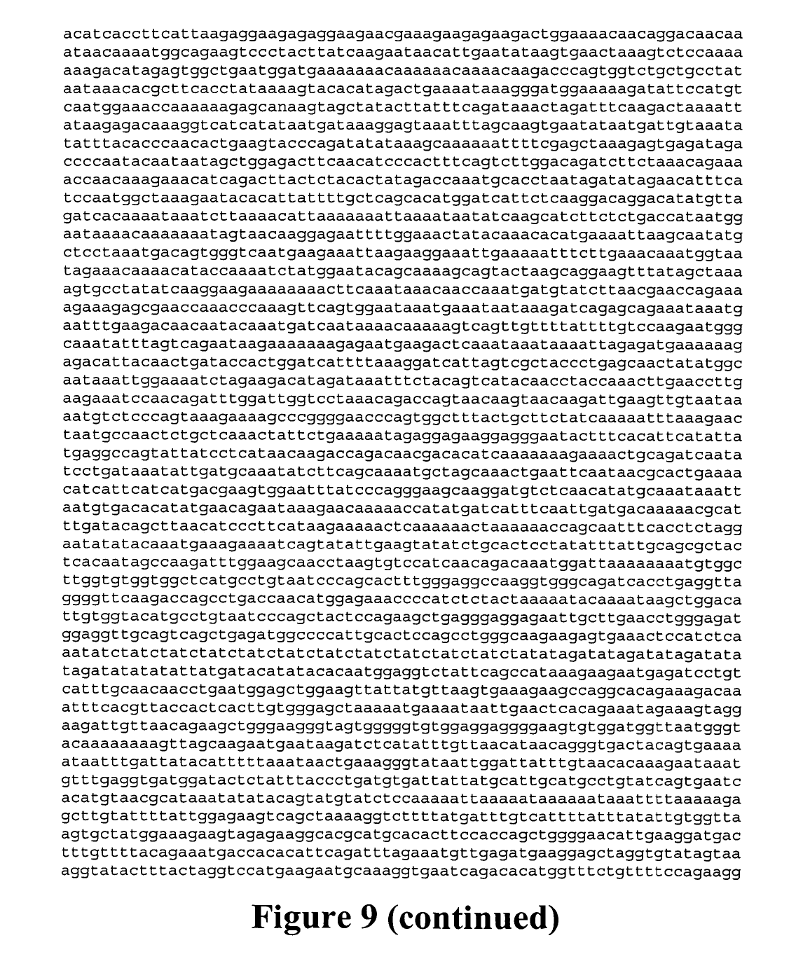

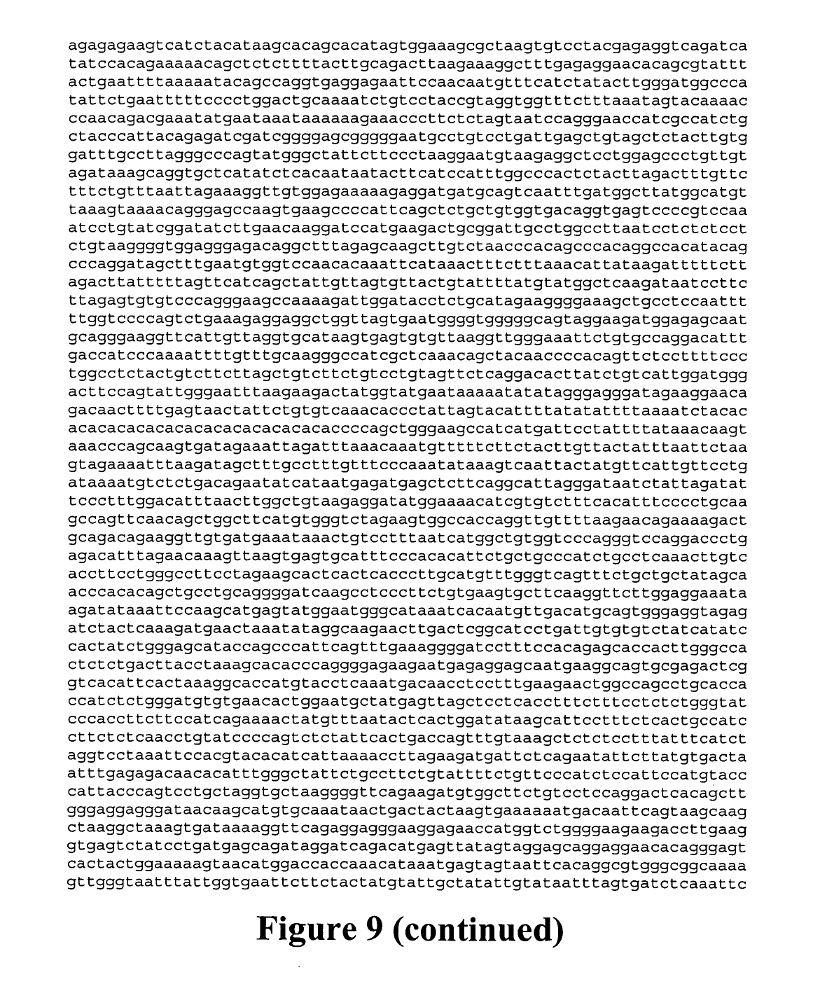

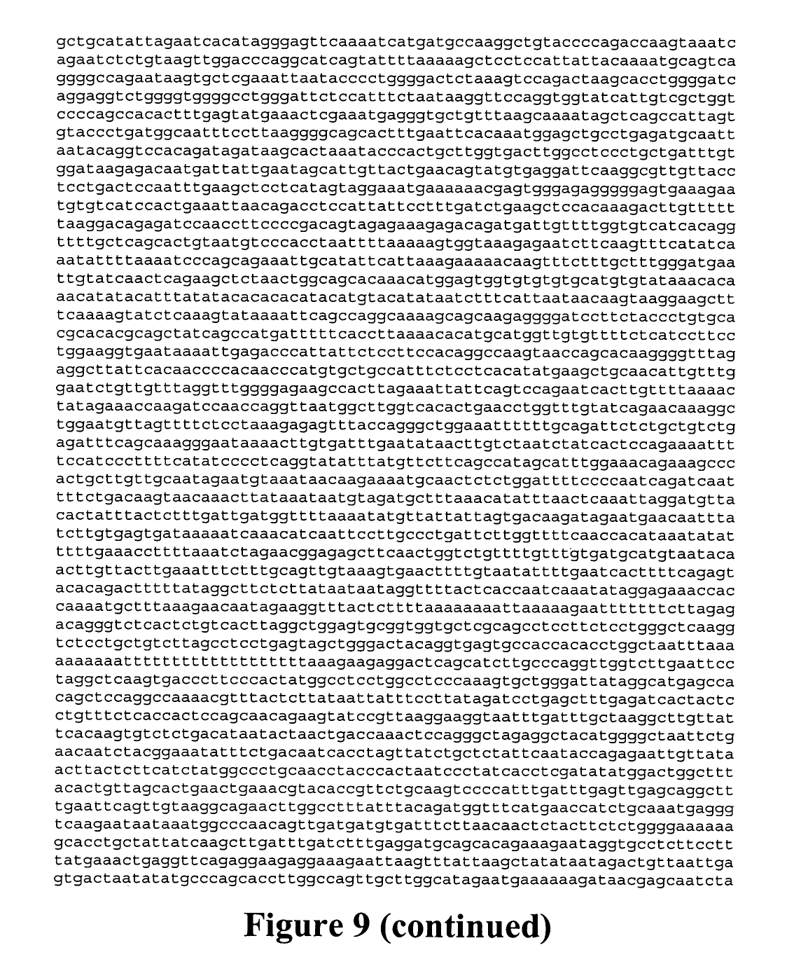

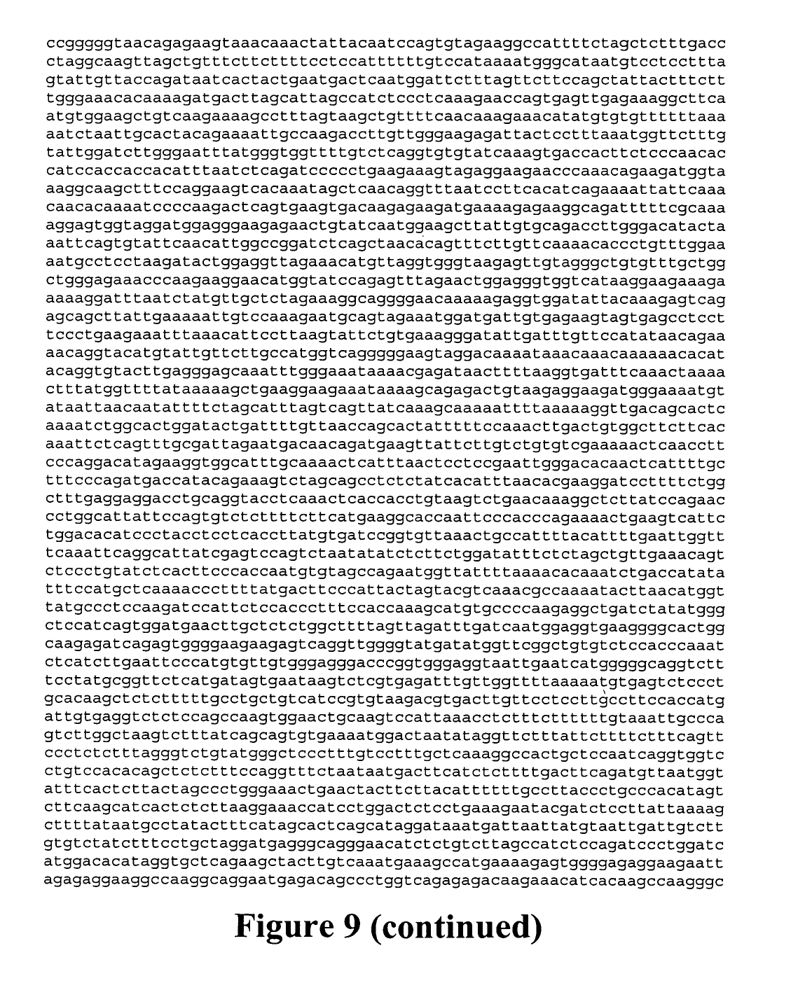

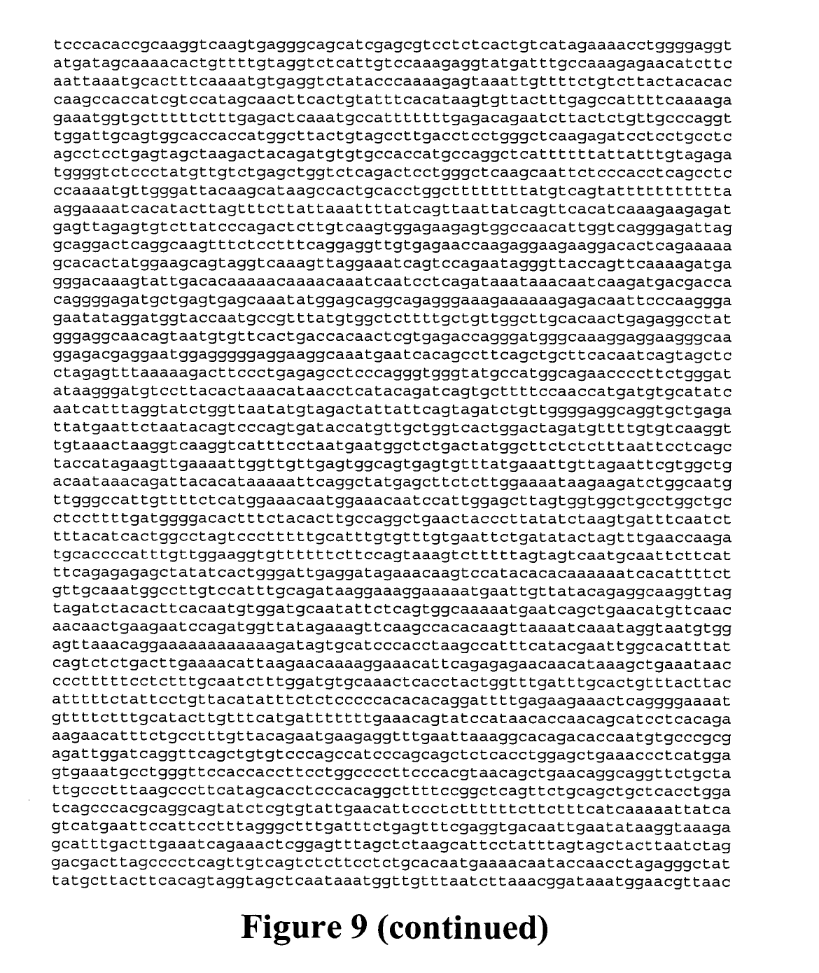

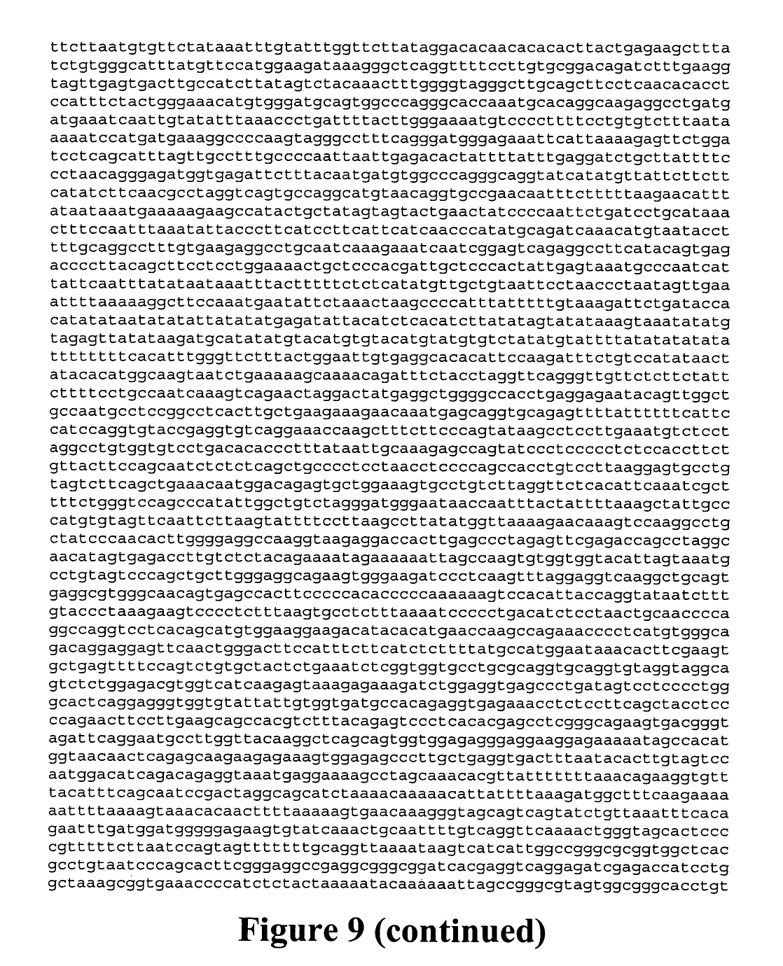

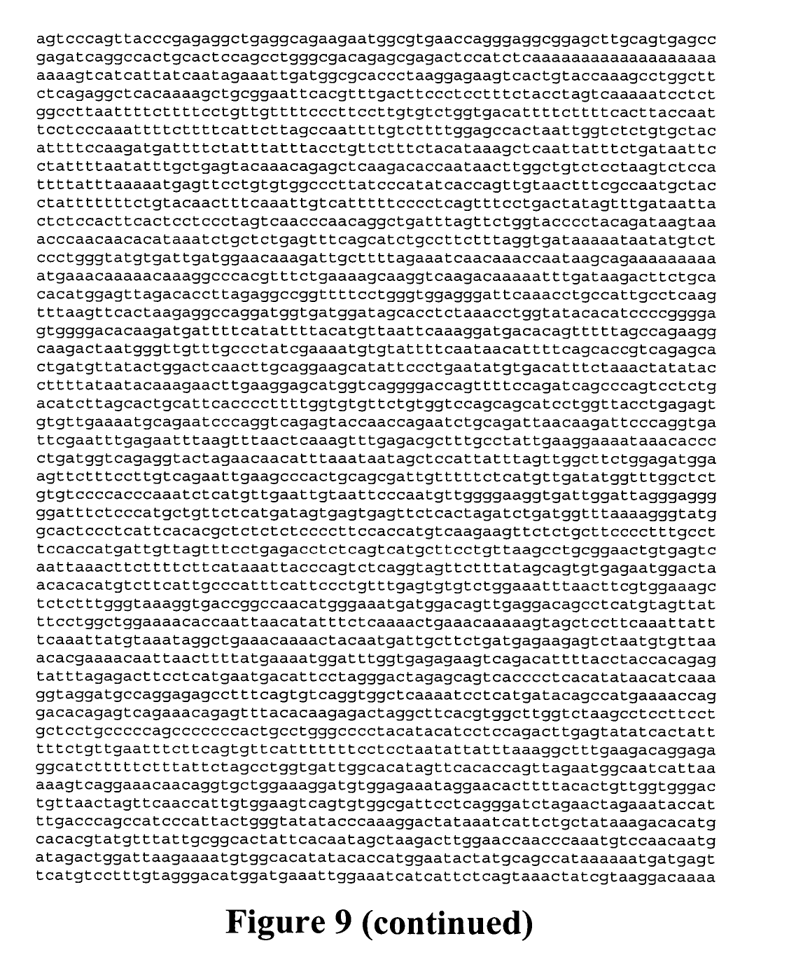

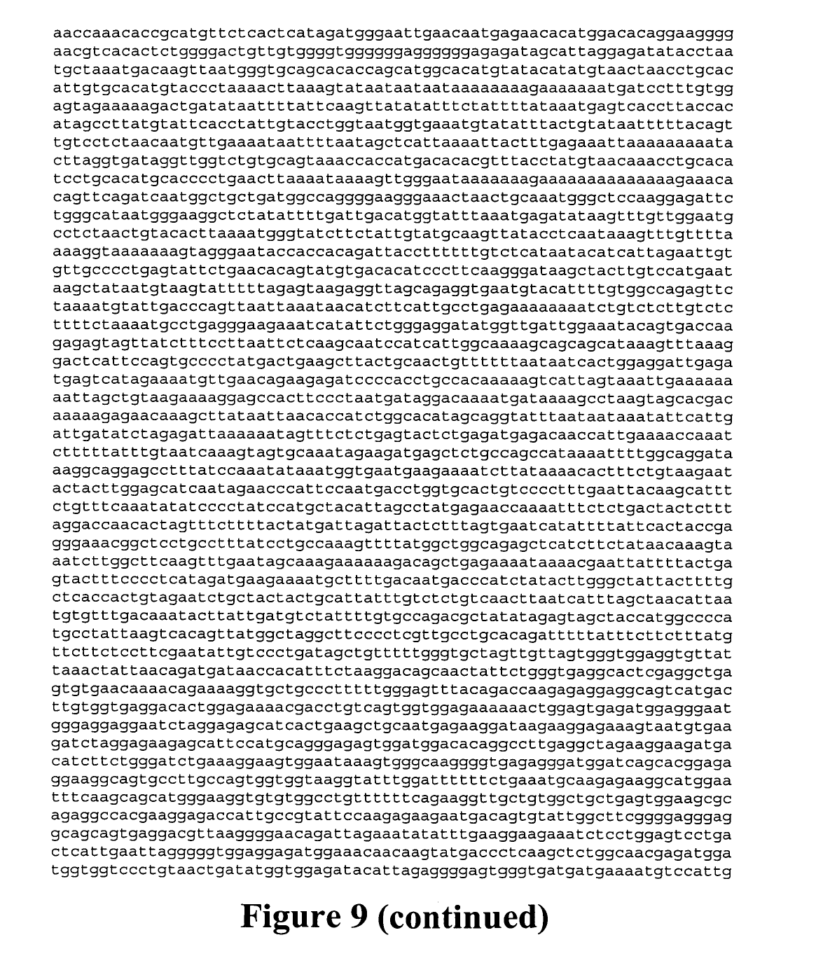

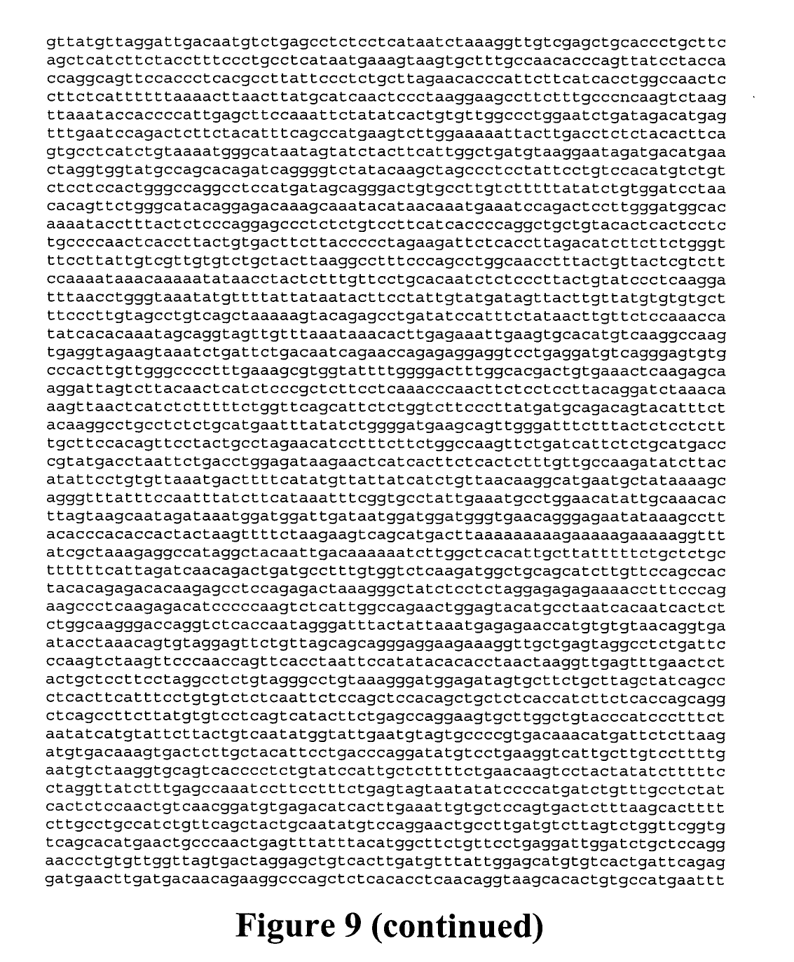

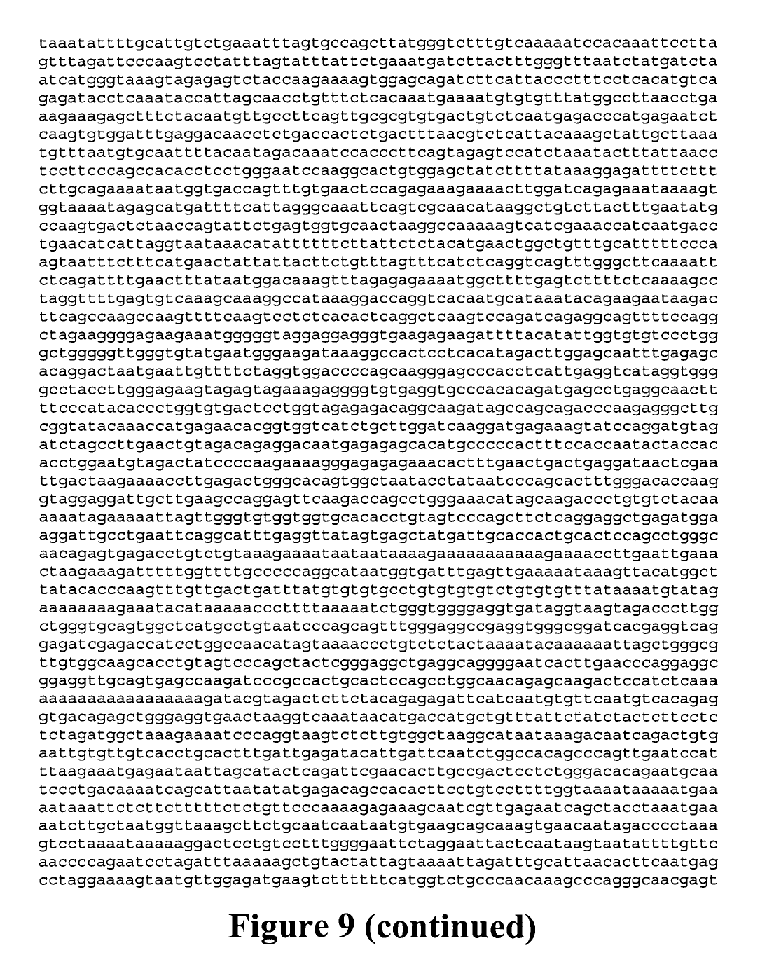

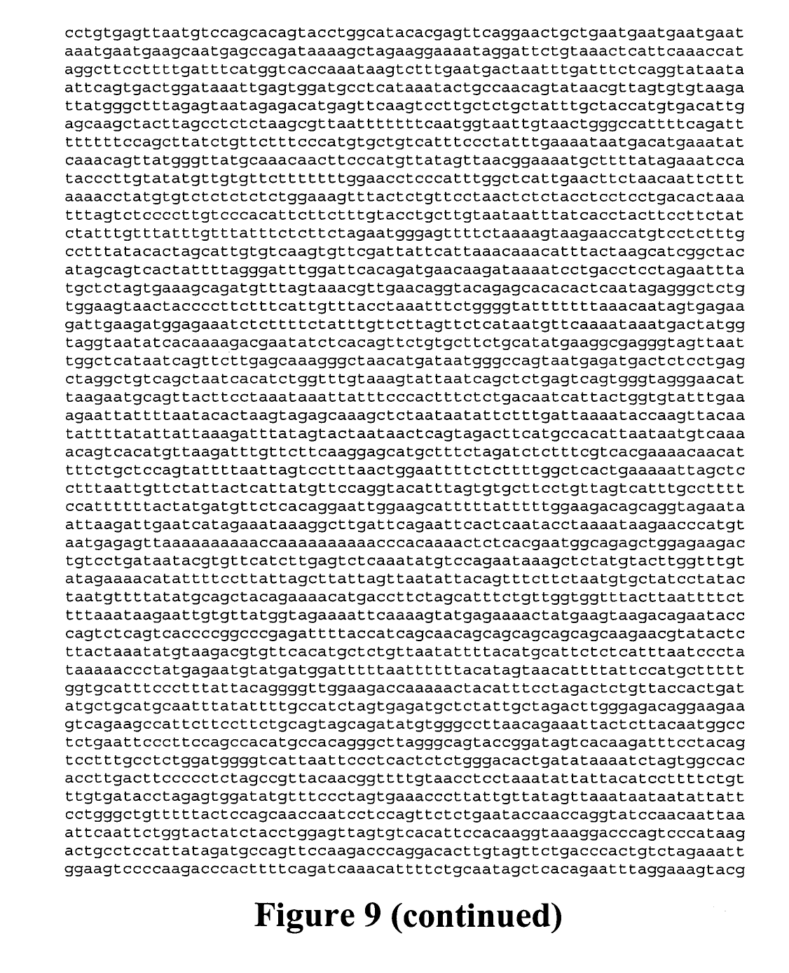

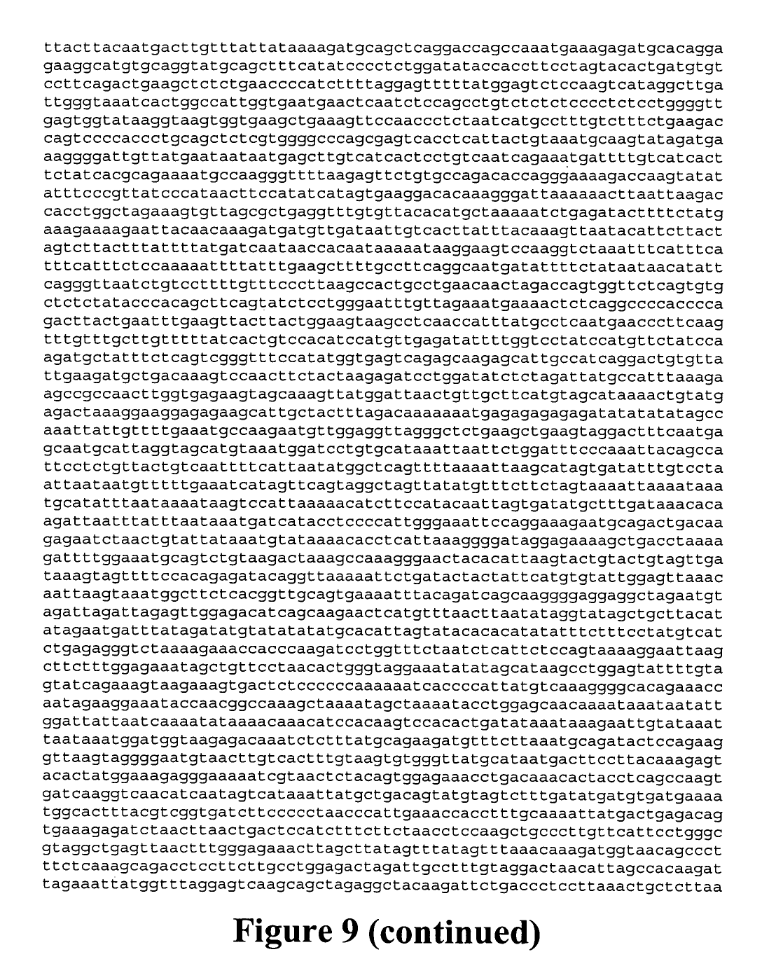

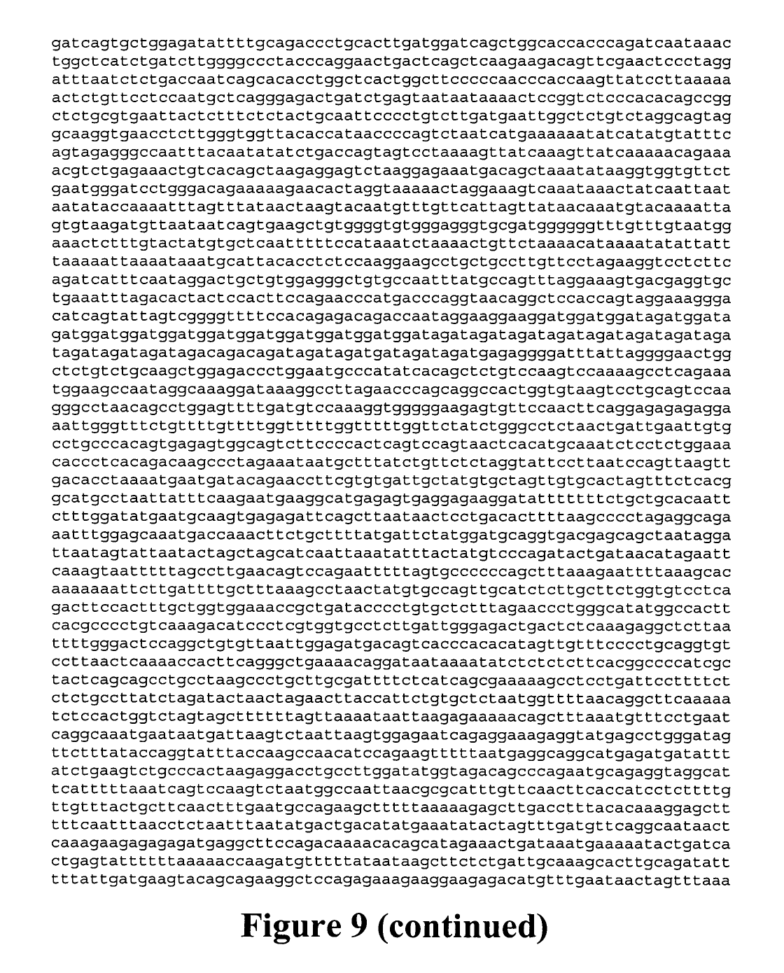

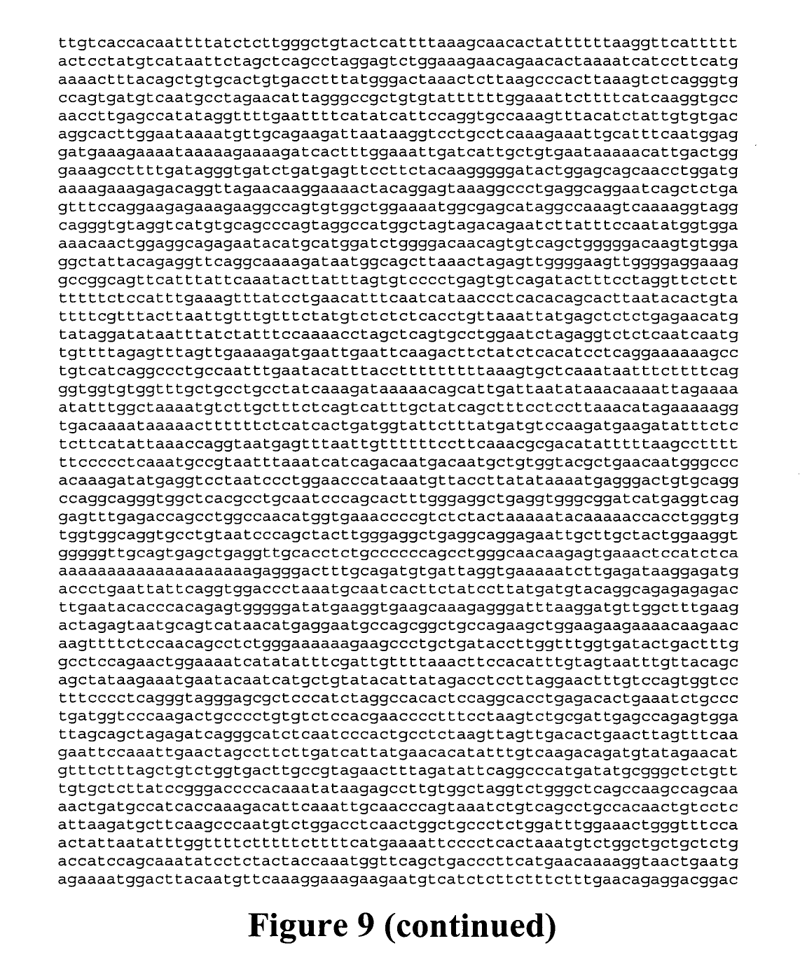

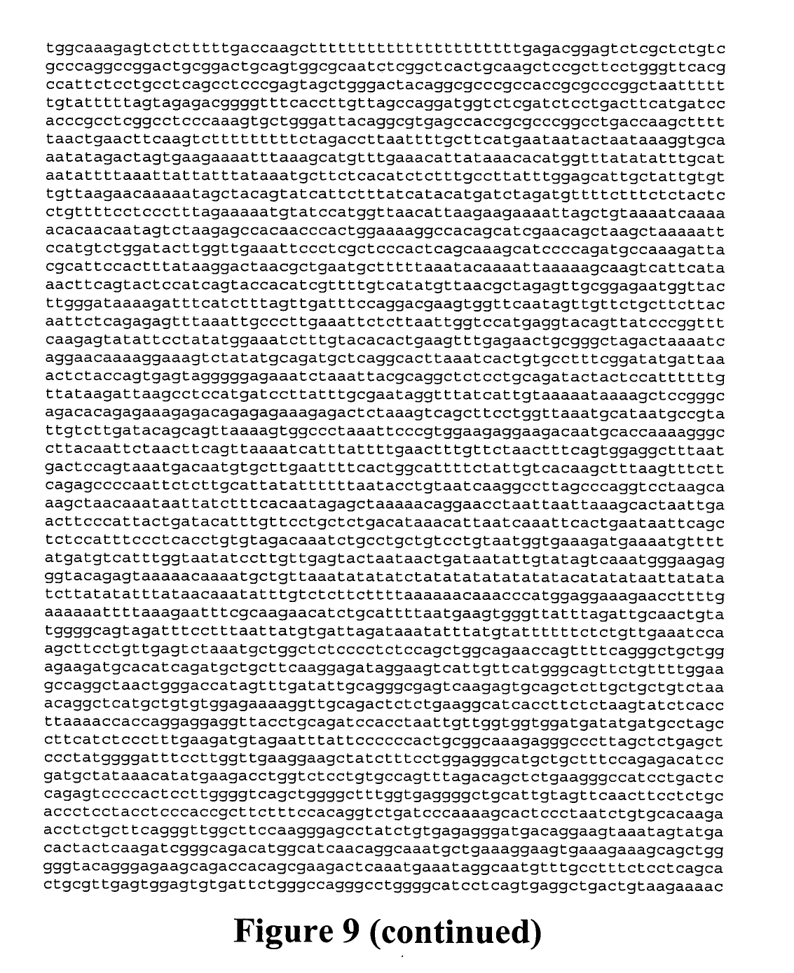

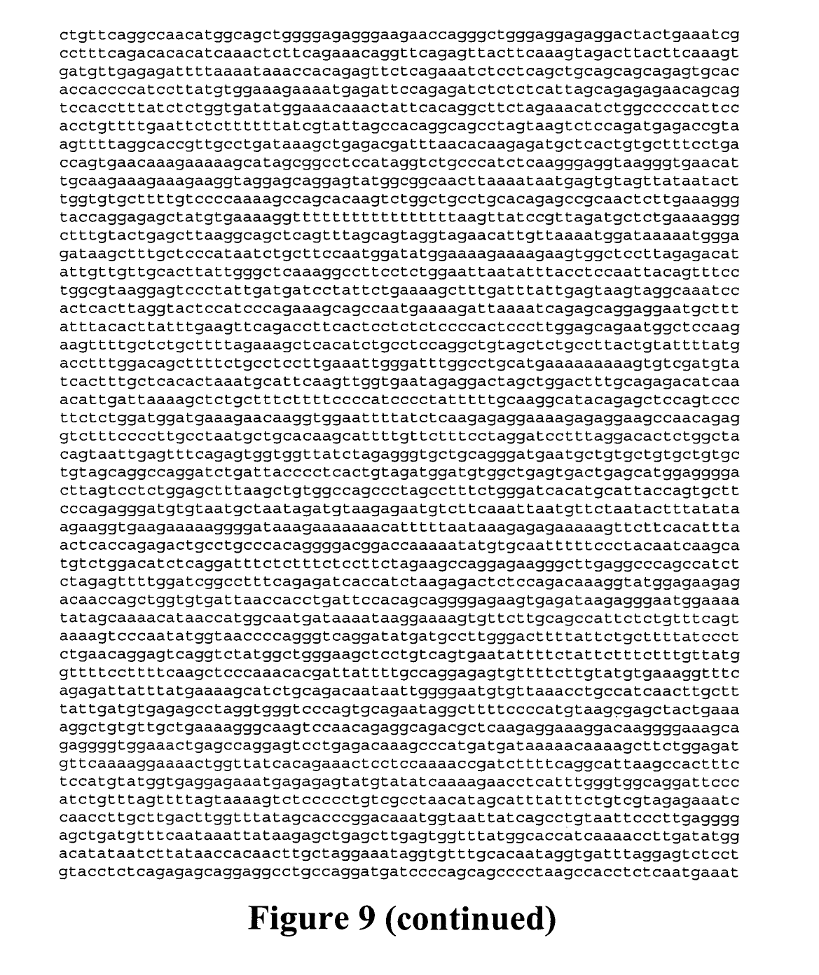

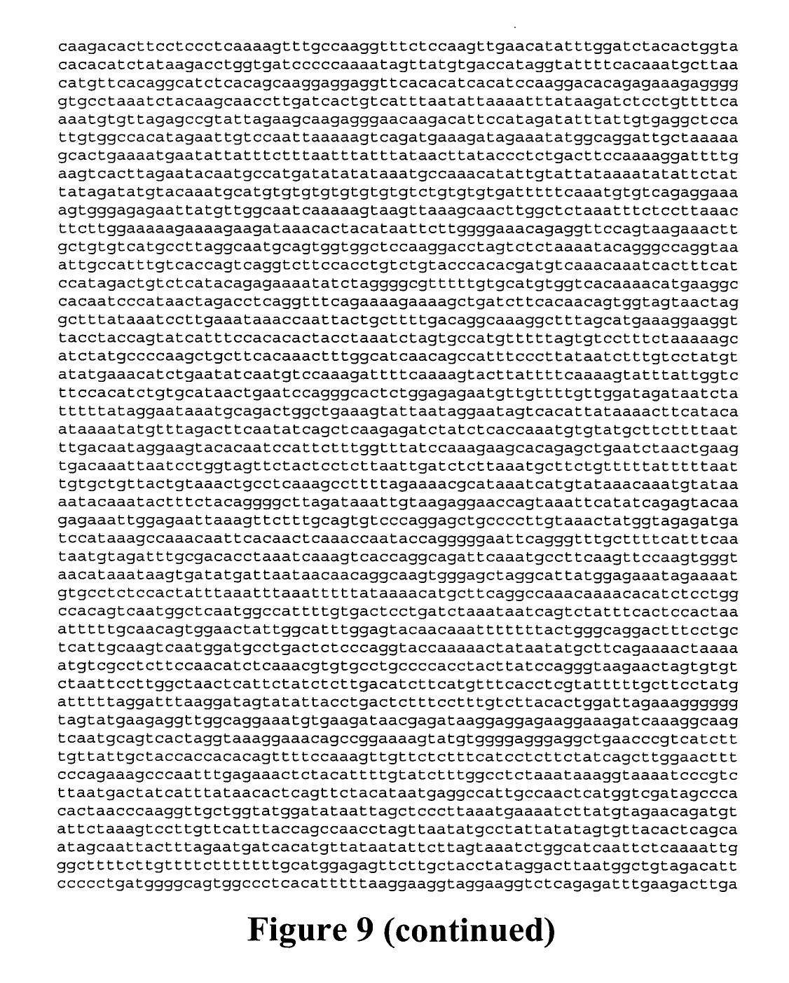

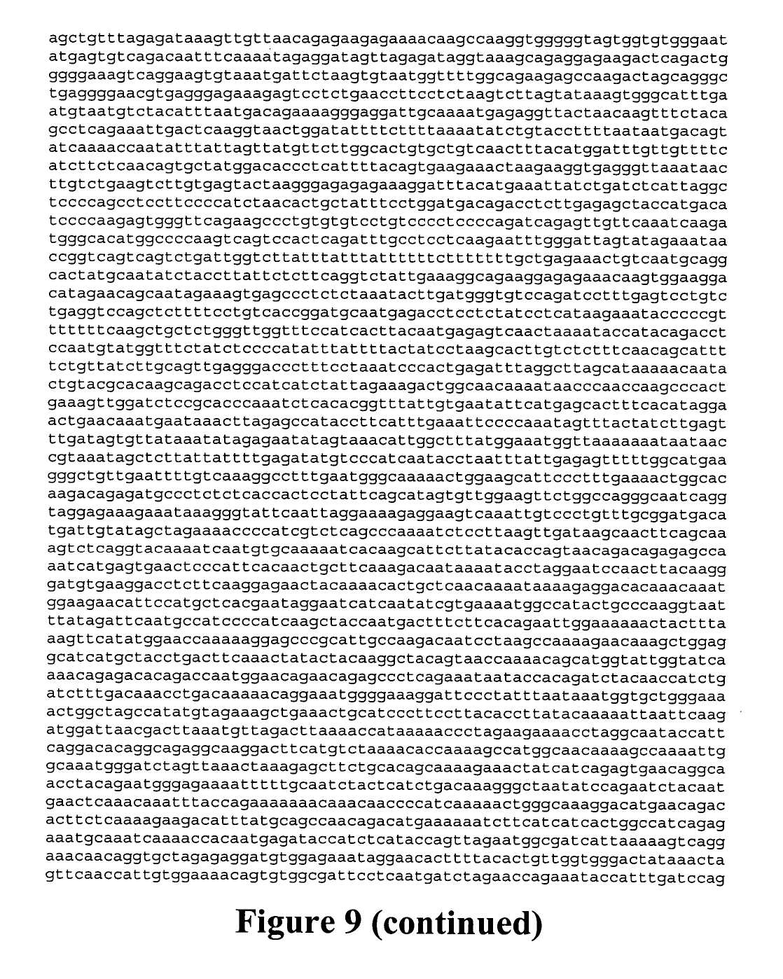

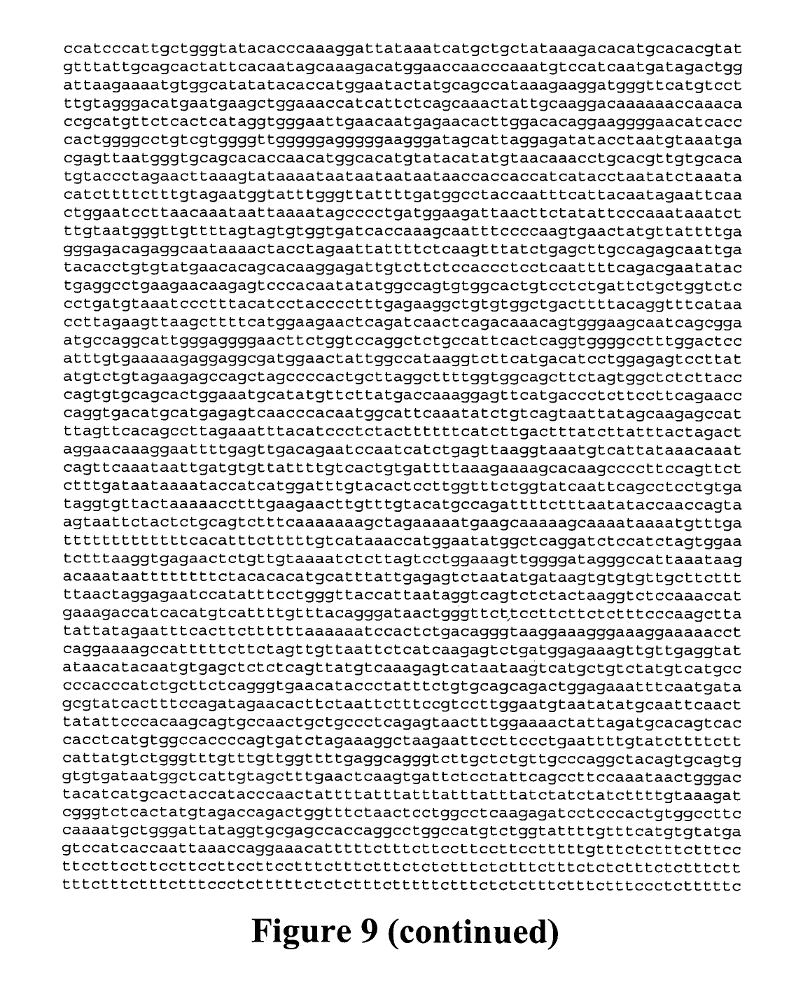

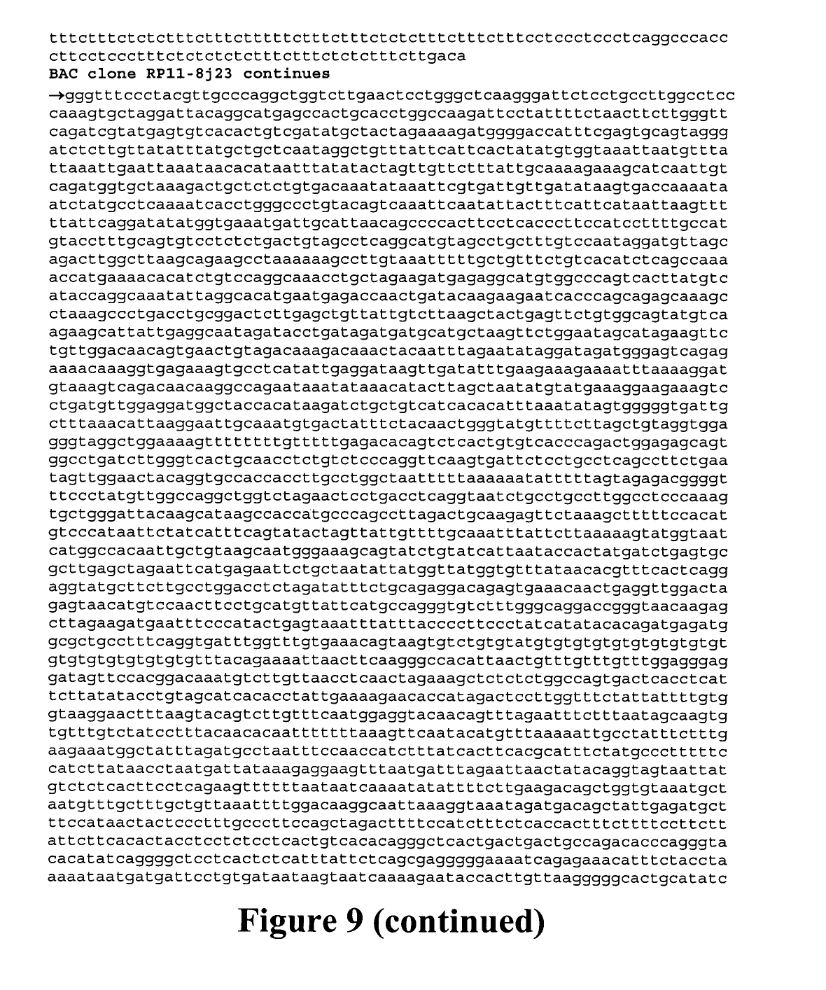

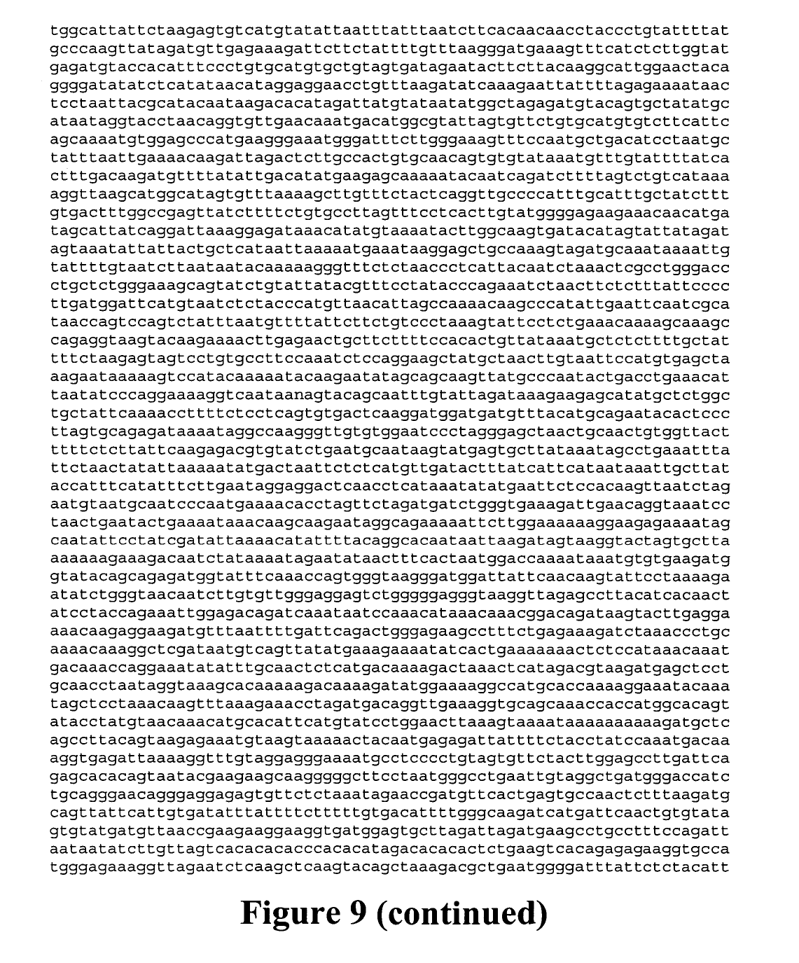

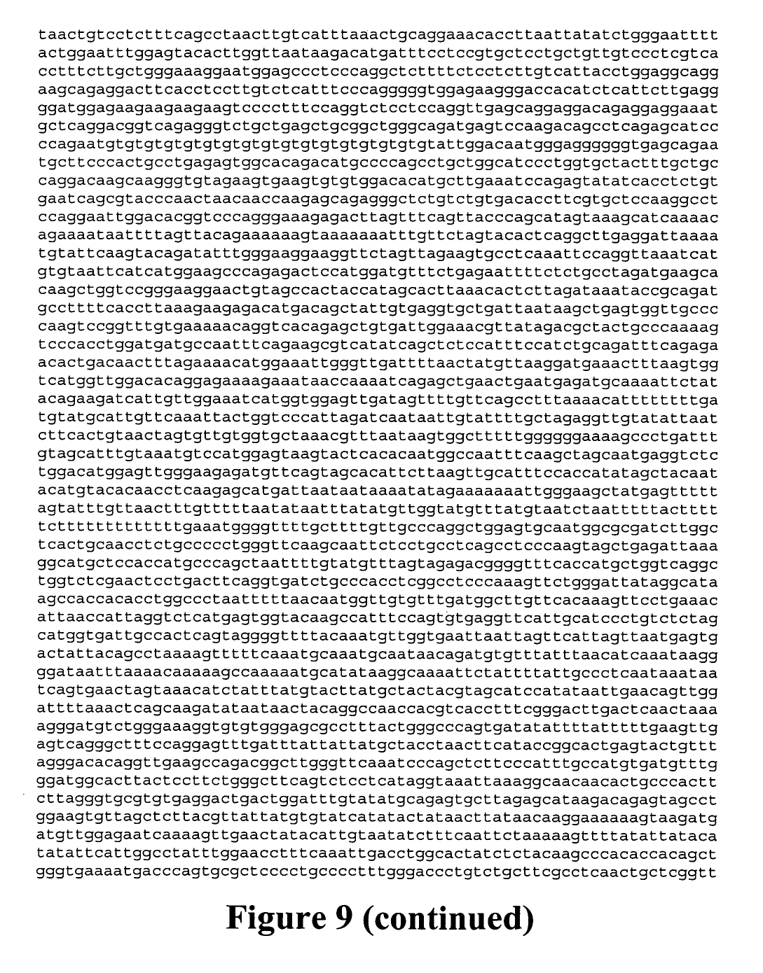

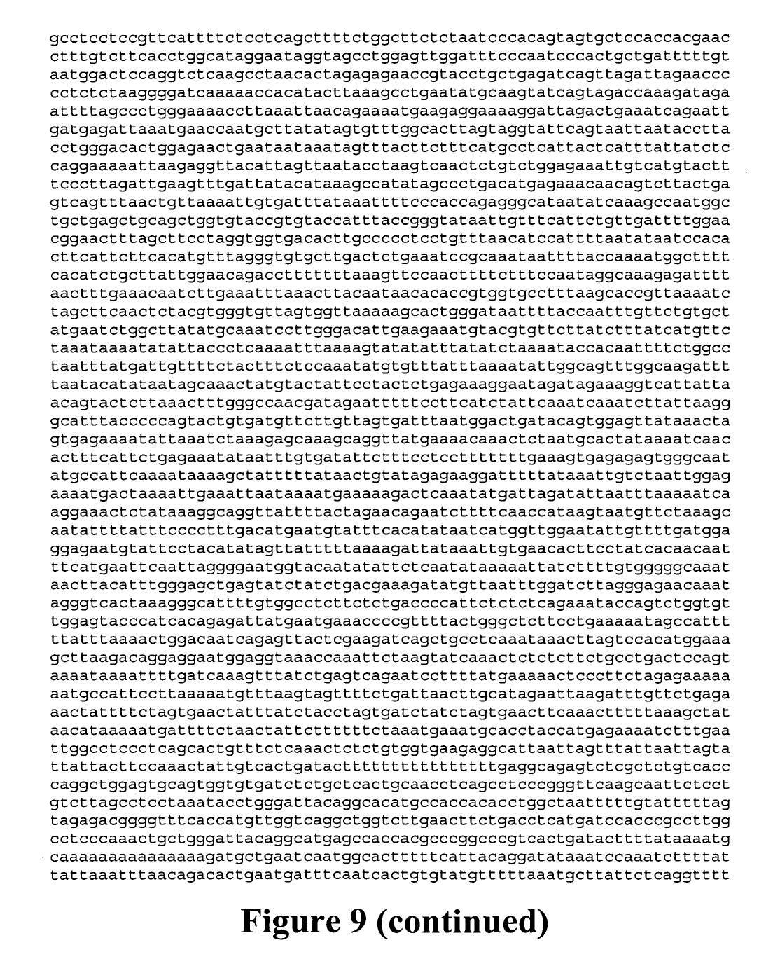

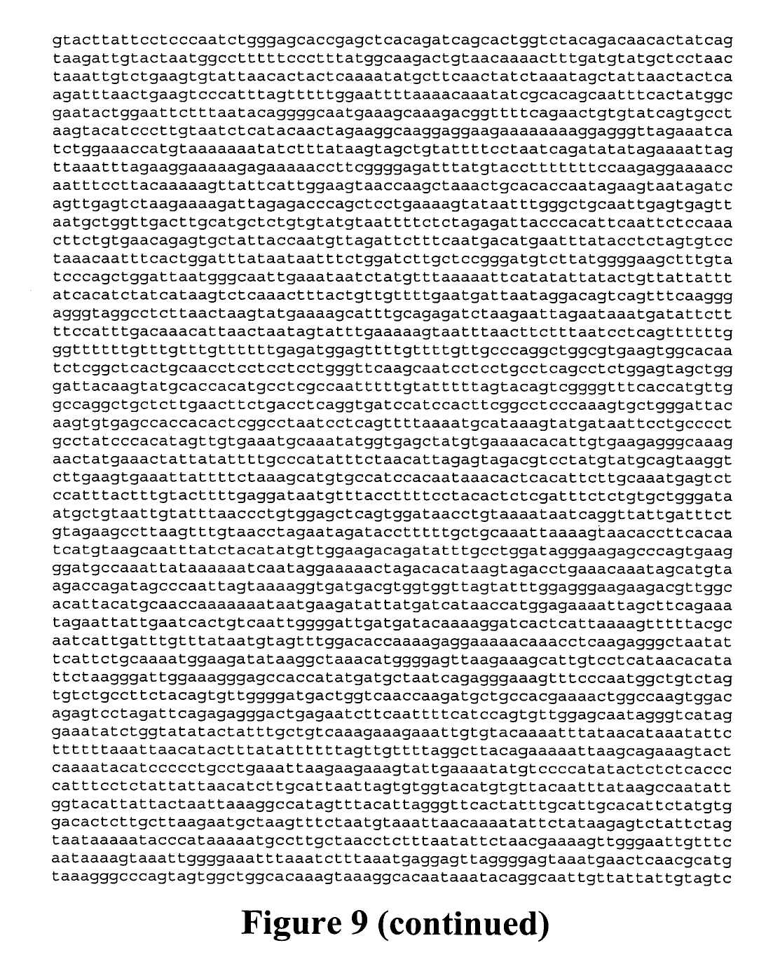

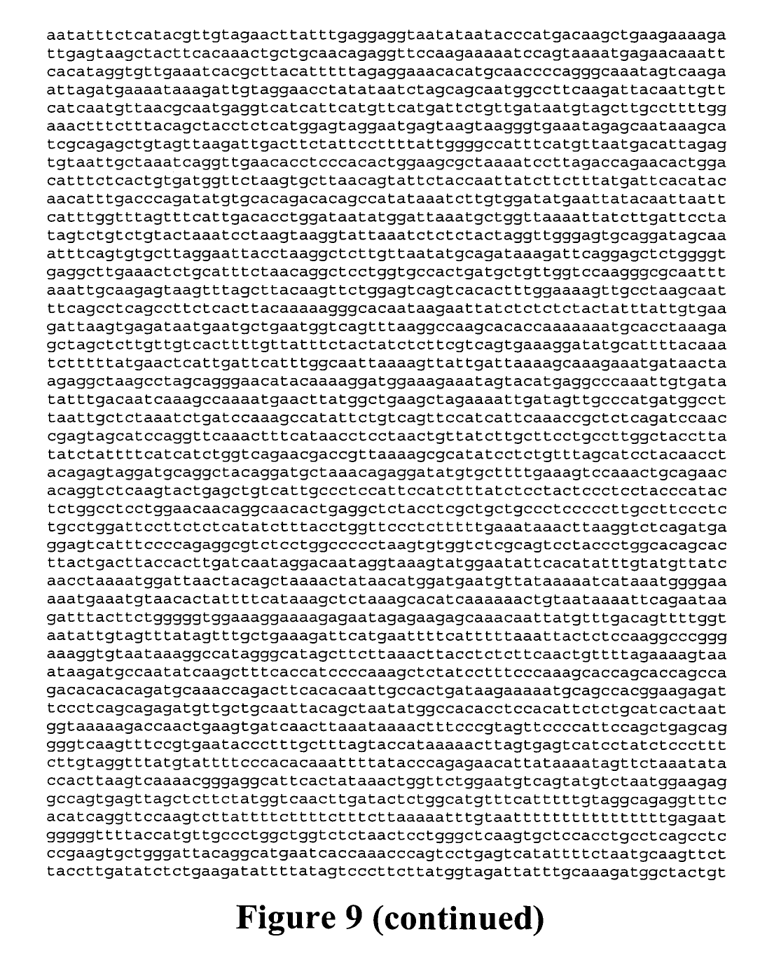

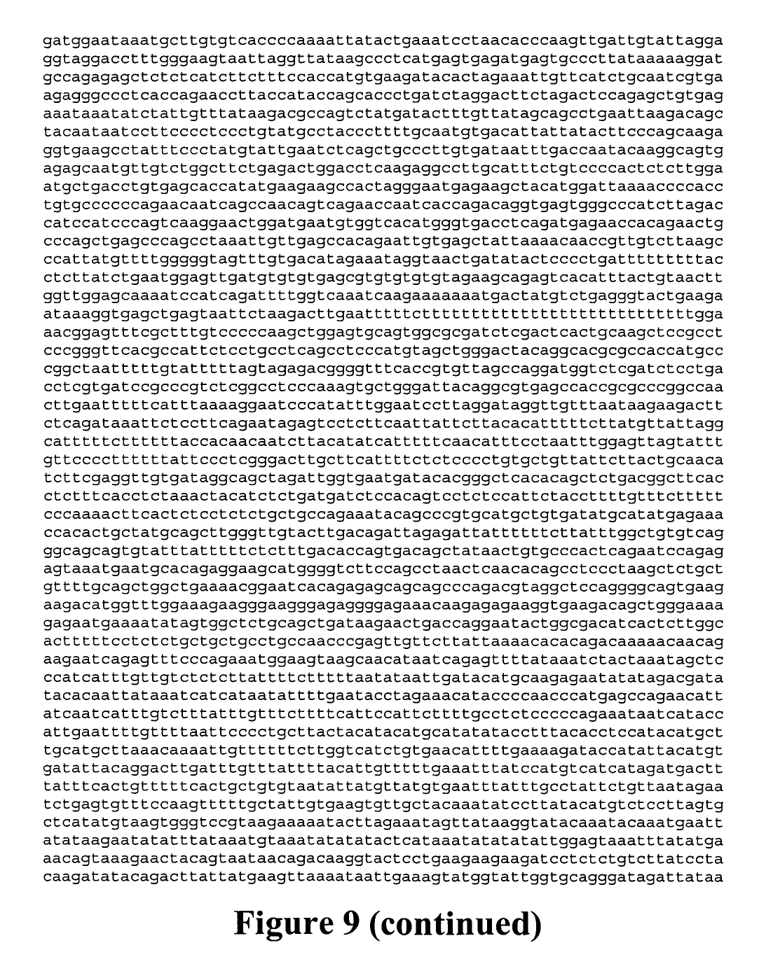

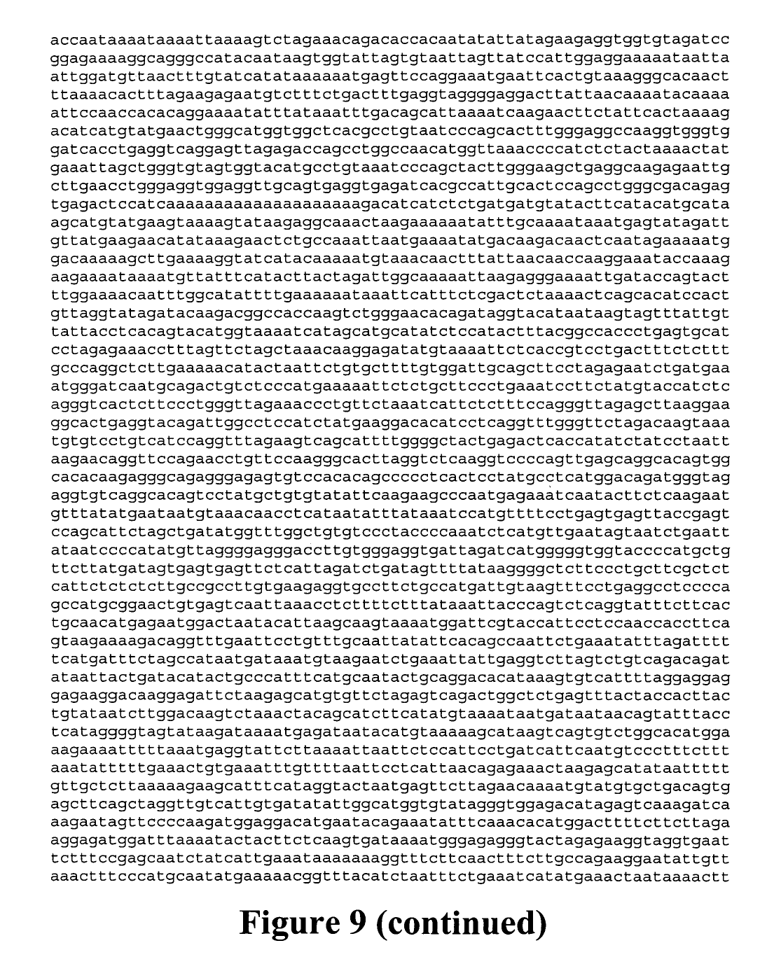

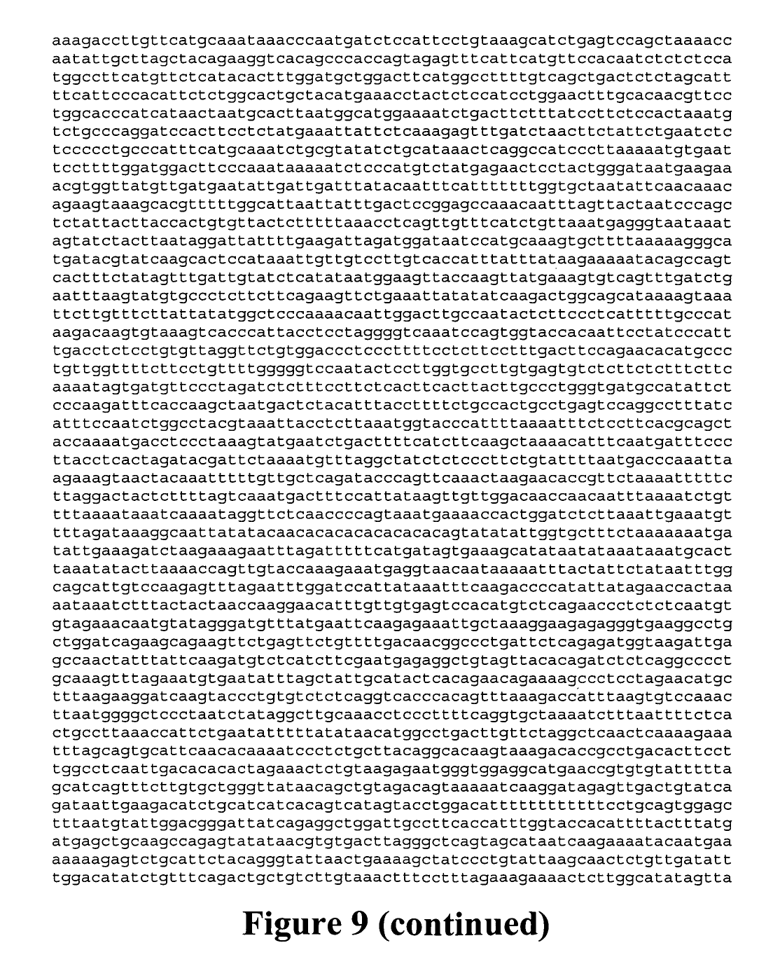

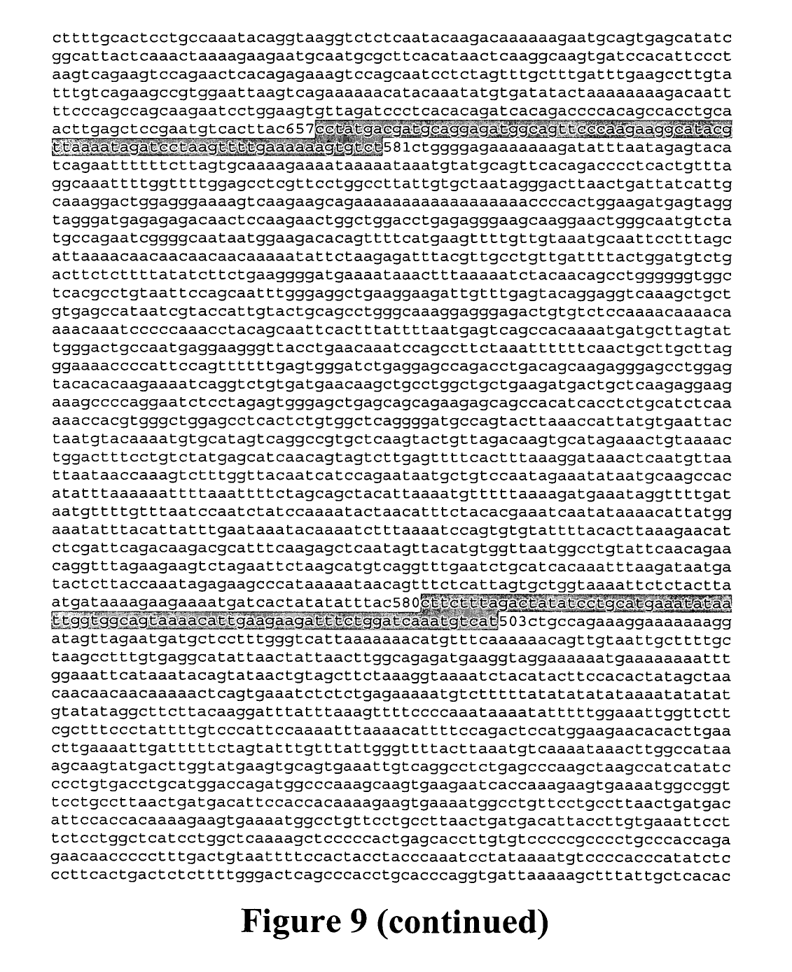

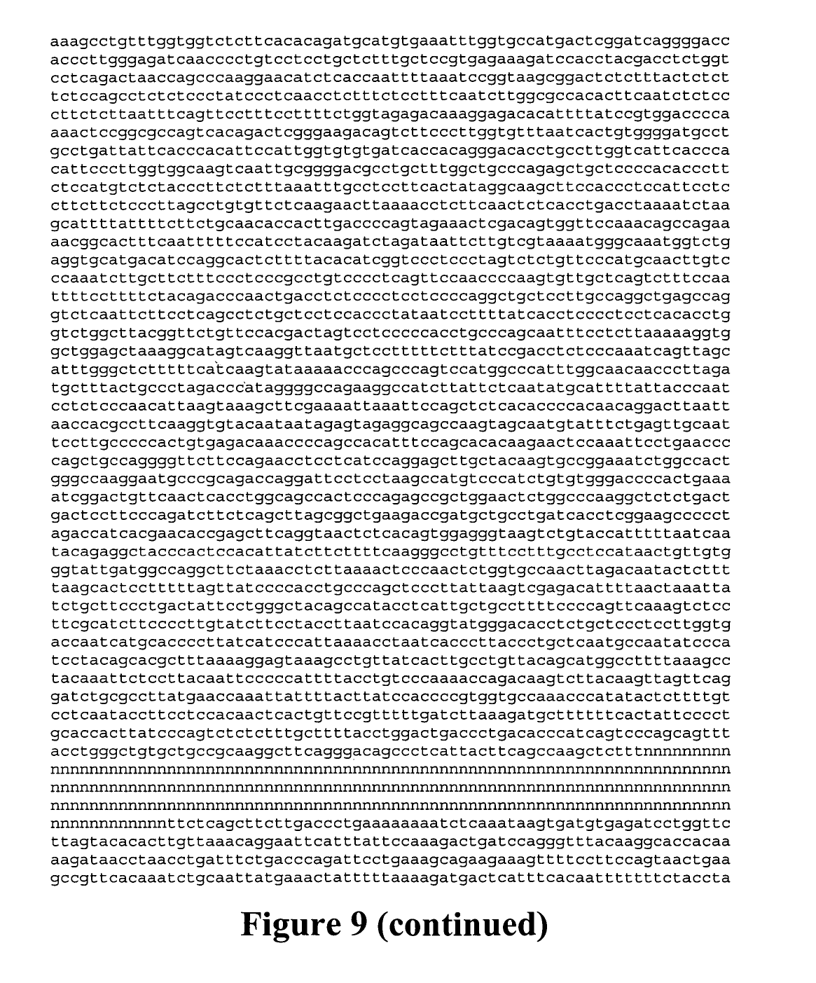

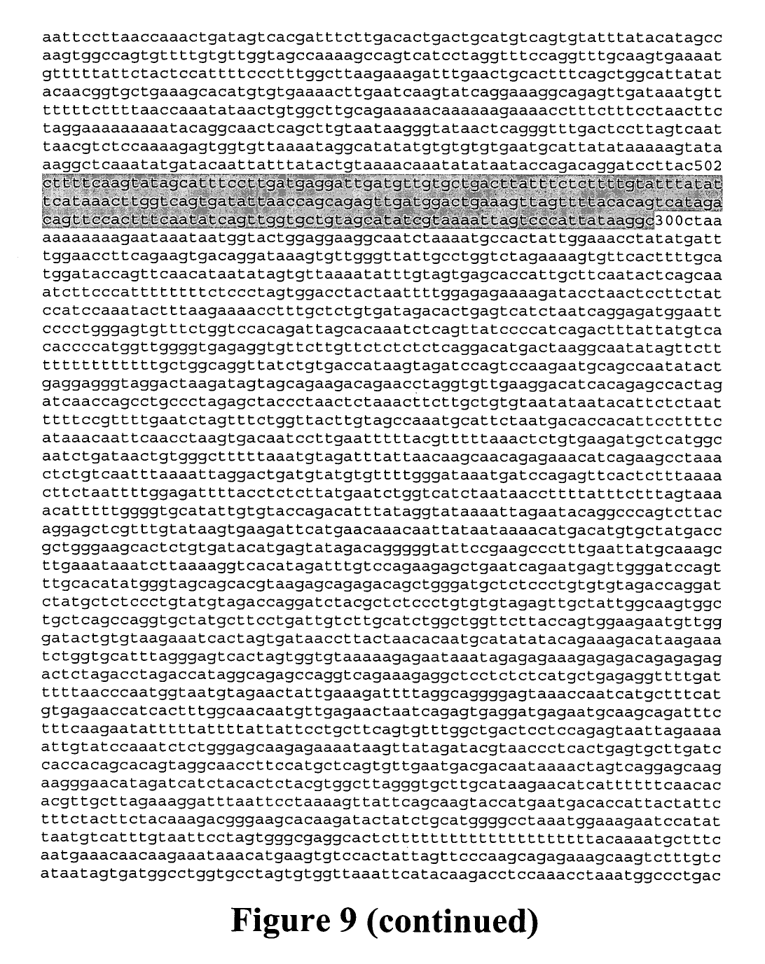

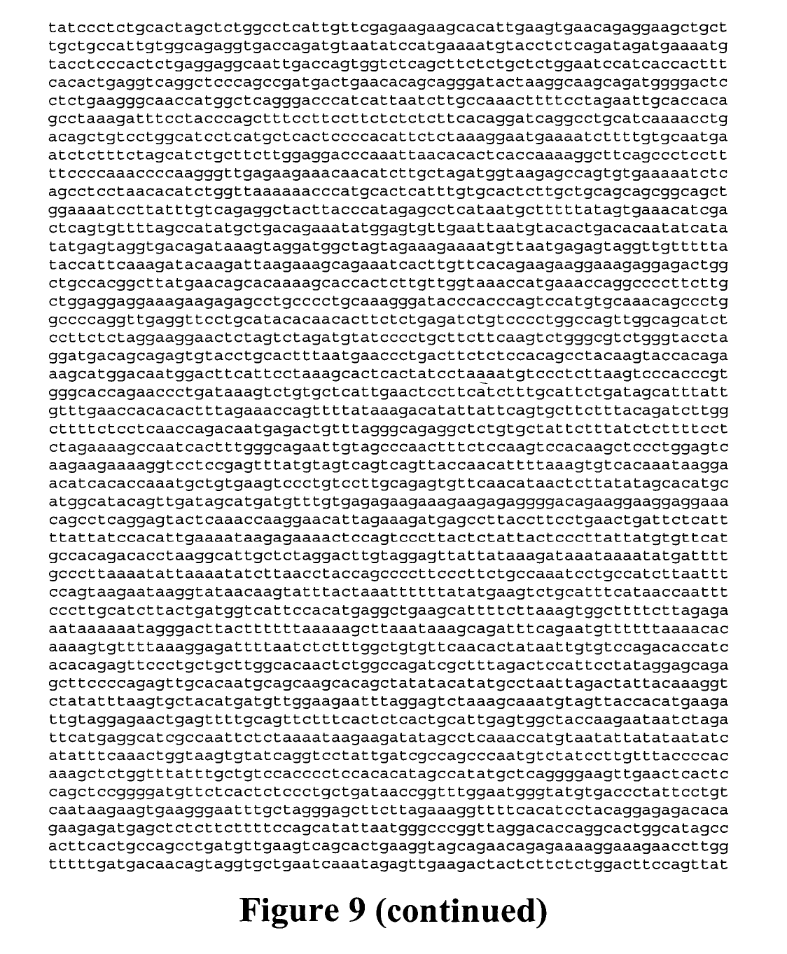

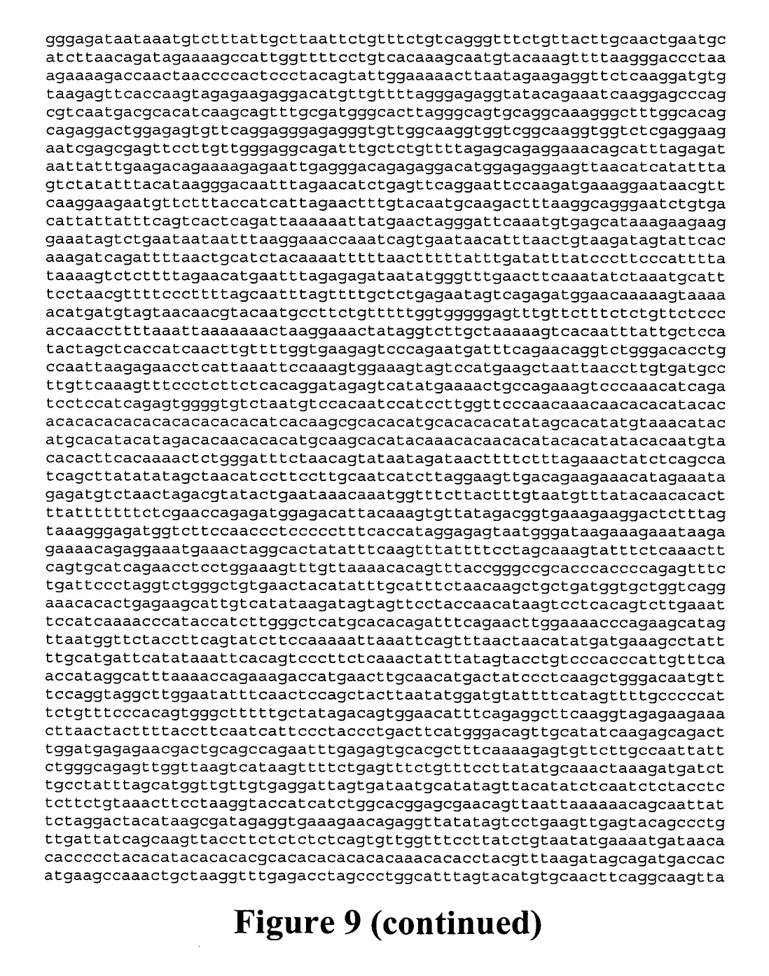

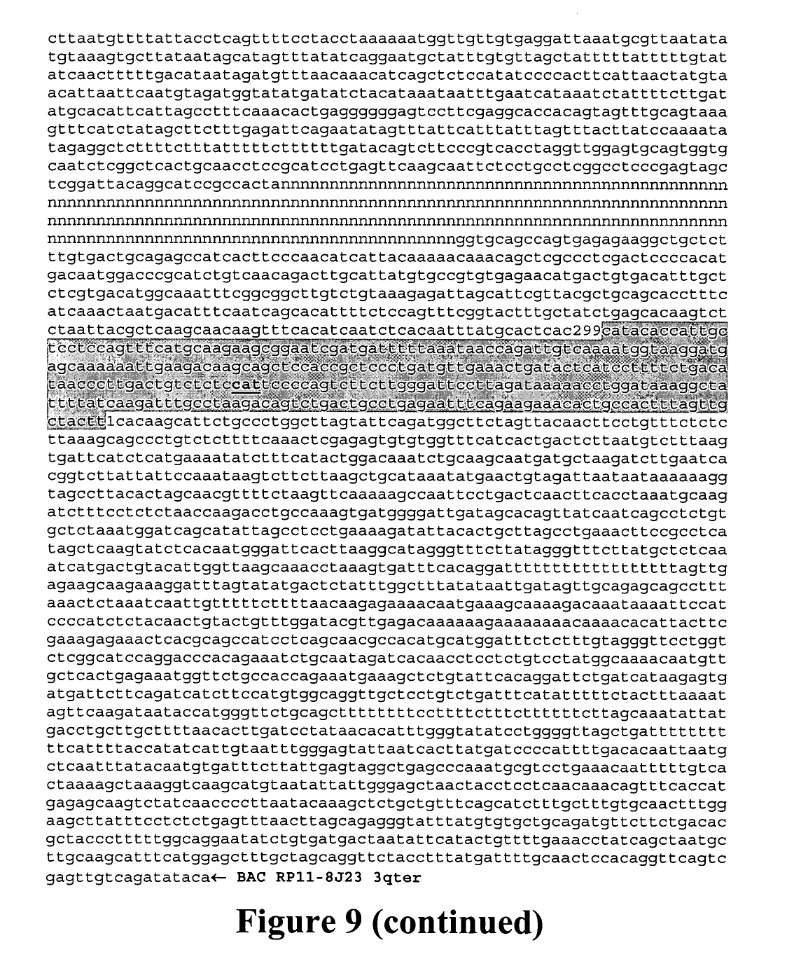

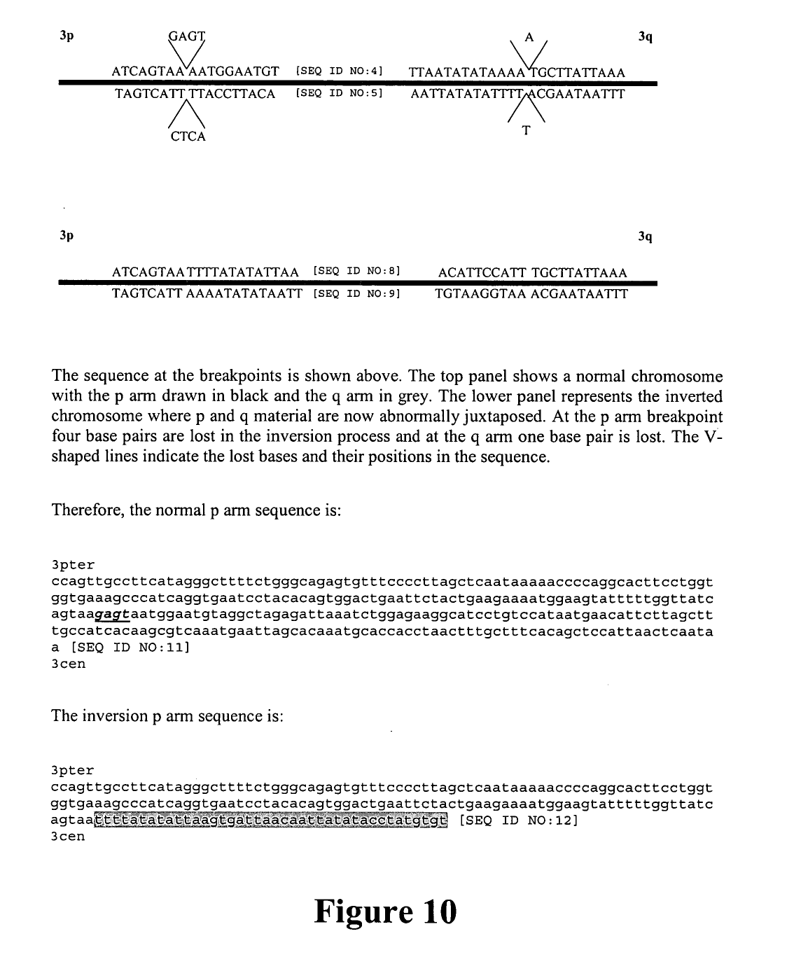

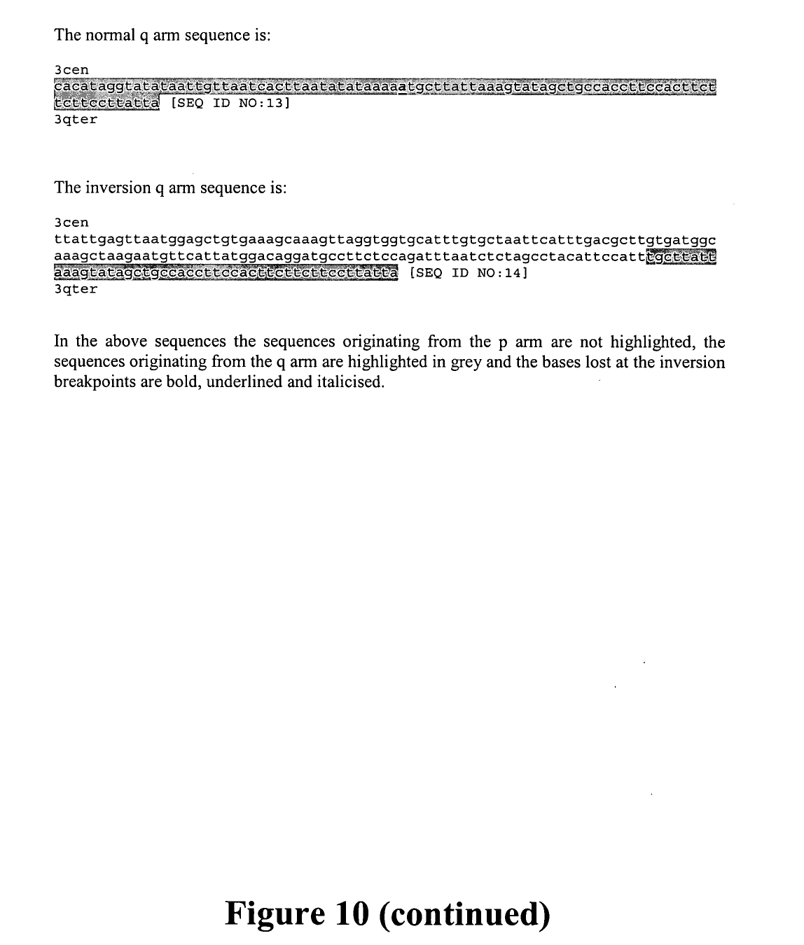

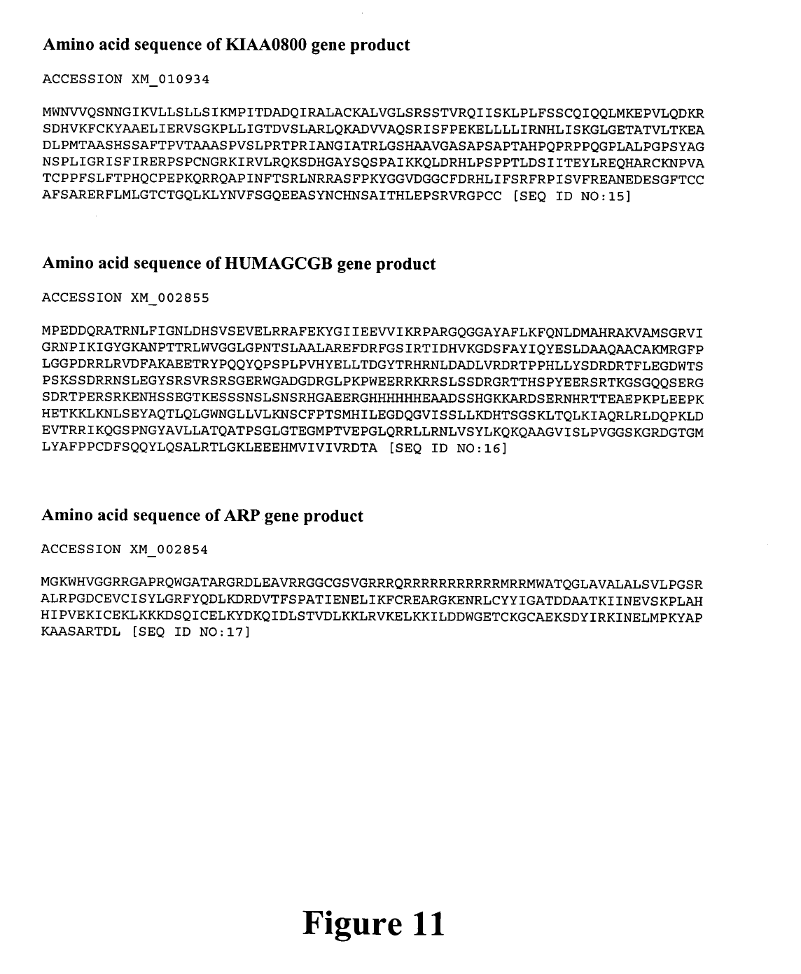

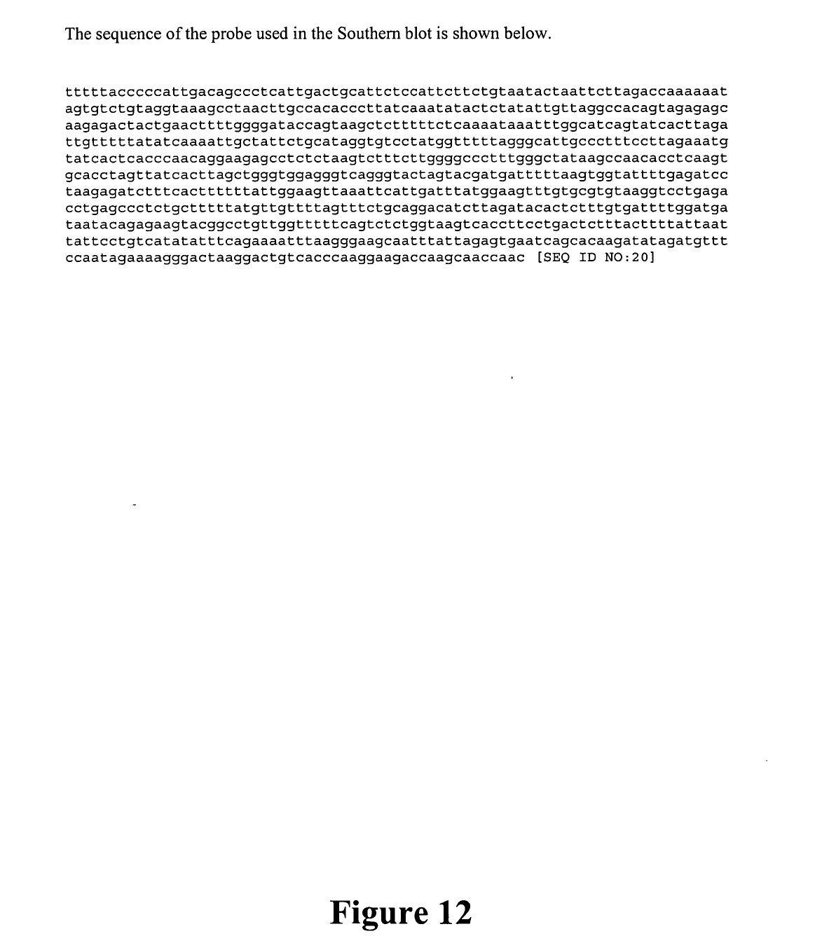

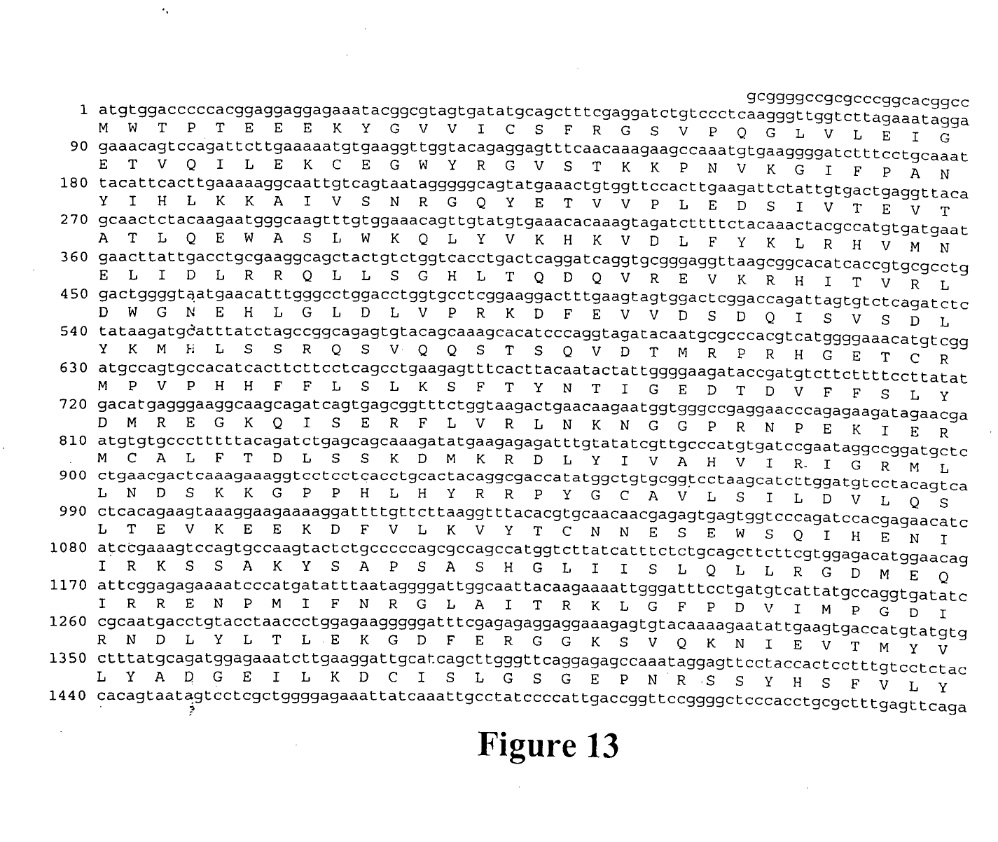

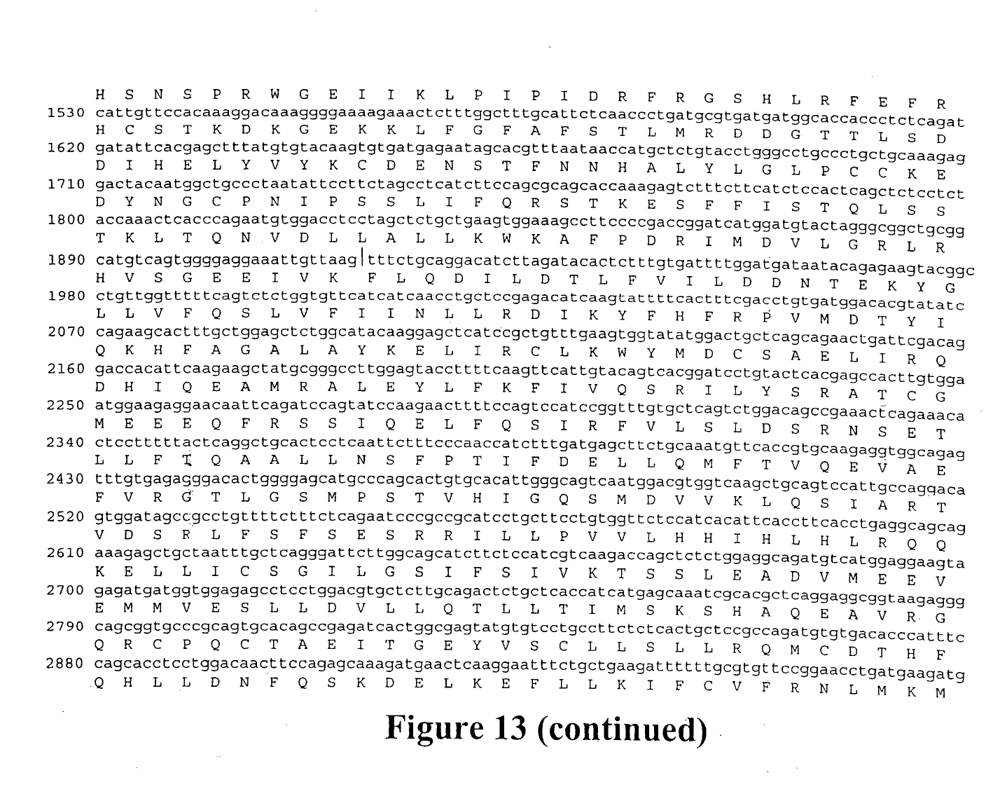

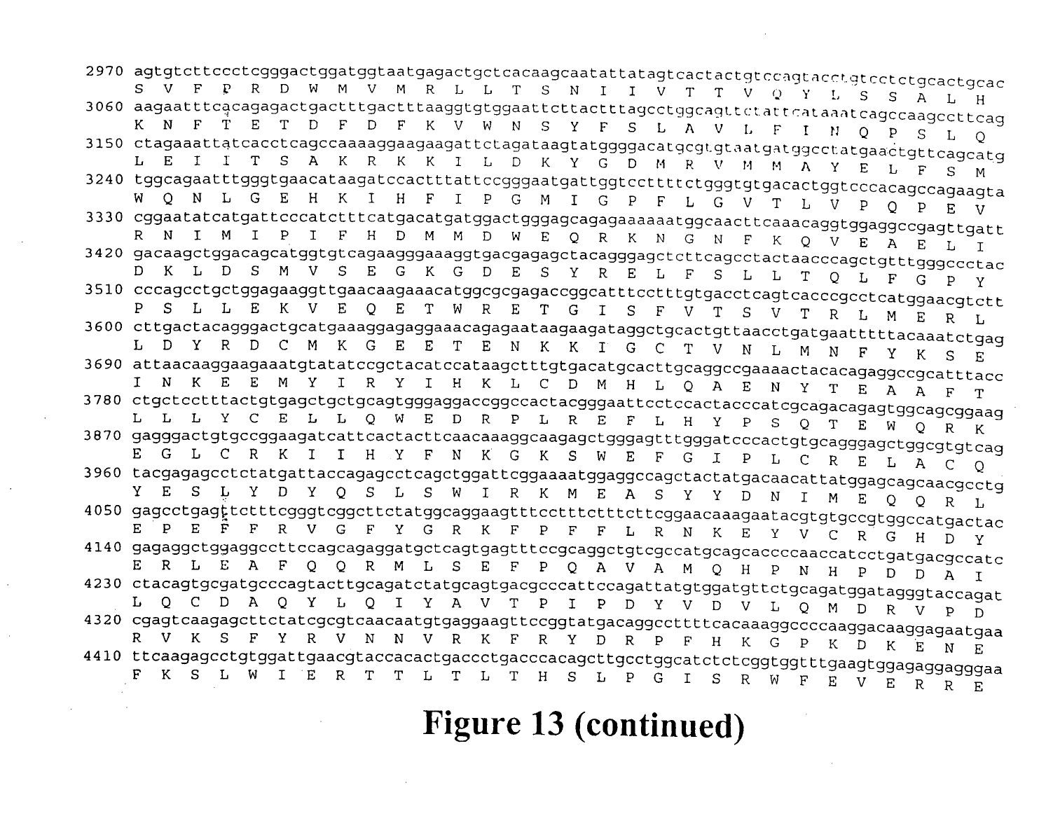

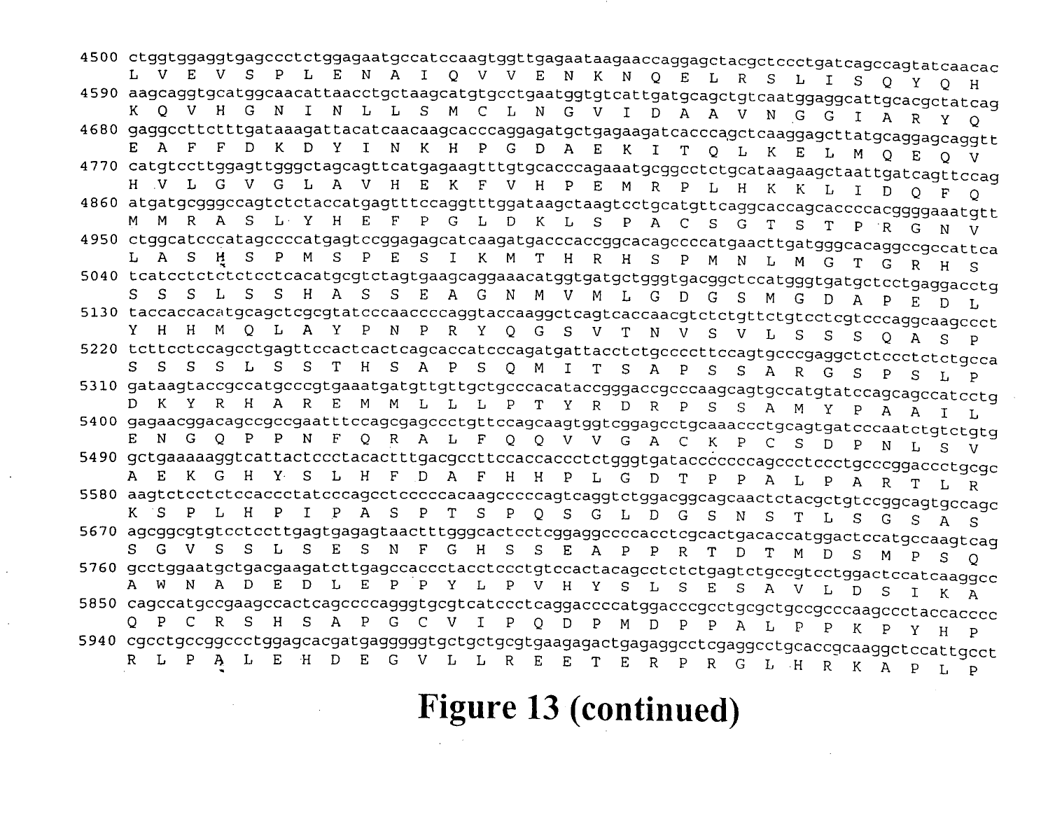

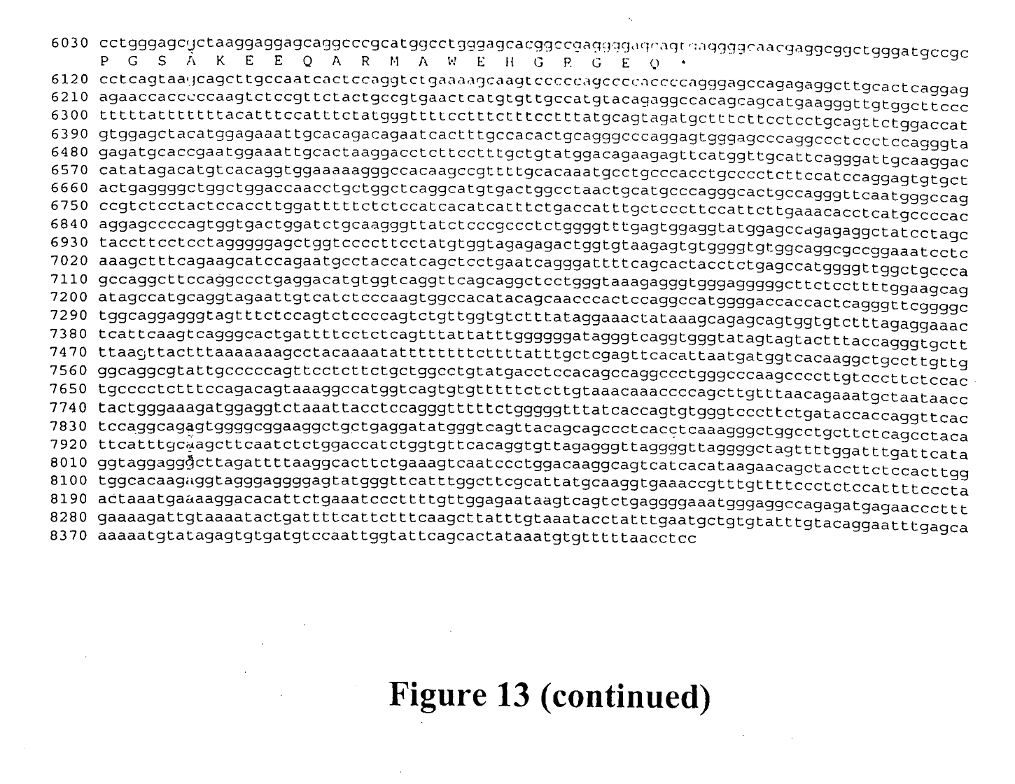

In one embodiment, the marker comprising the nucleotide and amino acid sequence of DOCK 3 which is shown in FIG. 13 and SEQ ID NOs:20 and 21, respectively. FIG. 8 also provides the intron:exon boundaries and genomic sequence for DOCK 3.

Accordingly, yet another related aspect of the present invention provides a molecular marker of a behavioral phenotype, said molecular marker comprising, in a genetic form, a nucleotide sequence or modified form thereof, or an expression product encoded thereby, said nucleotide sequence derived from the p-arm of chromosome 3 and comprising or contained within a sequence of nucleotides which hybridizes under conditions of low stringency to a nucleic acid molecule encoding all or part of the gene product whose amino acid sequence is set forth in SEQ ID NO:21 and wherein the presence of a modified form of said molecular marker is indicative of a behavioral disorder or the likelihood that a subject may develop a behavioral disorder.

In a related embodiment, there is provided a molecular marker of a behavioral phenotype, said molecular marker comprising, in a genetic form, a nucleotide sequence or modified form thereof, or an expression product encoded thereby, said nucleotide sequence derived from the p-arm of chromosome 3 and comprising or contained within a sequence of nucleotides which hybridizes under conditions of low stringency to a nucleic acid molecule comprising the nucleotide sequence set forth in SEQ ID NO:20 or its complementary form wherein said molecular marker is indicative of a behavioral disorder or the likelihood that a subject may develop a behavioral disorder.

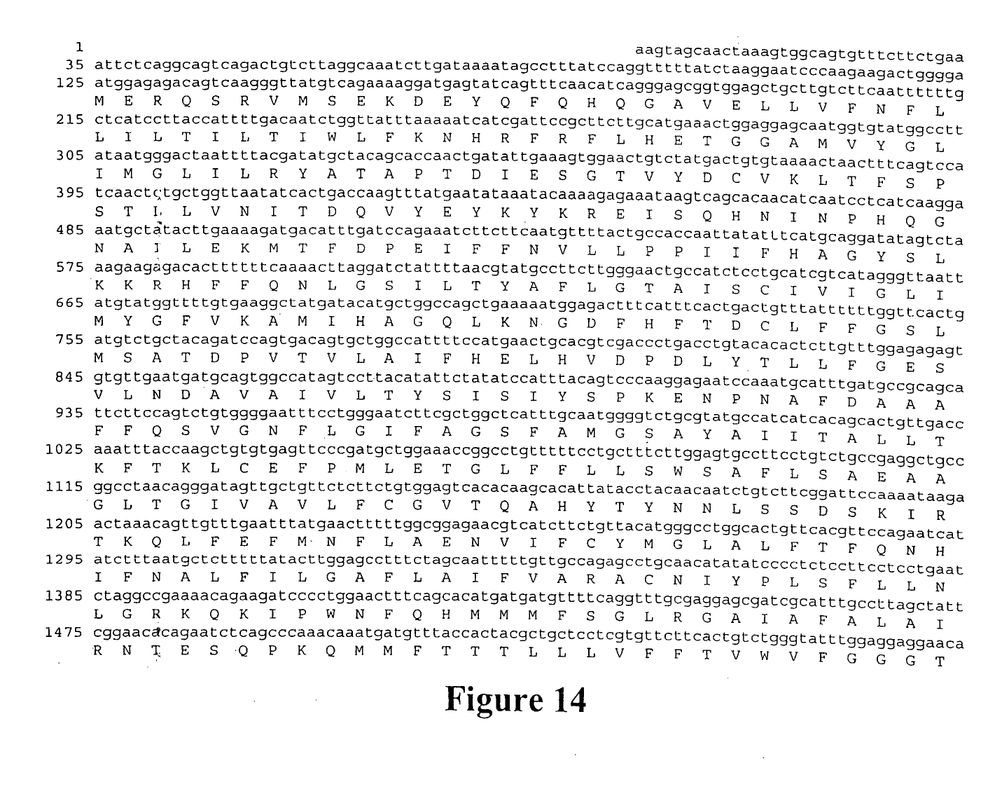

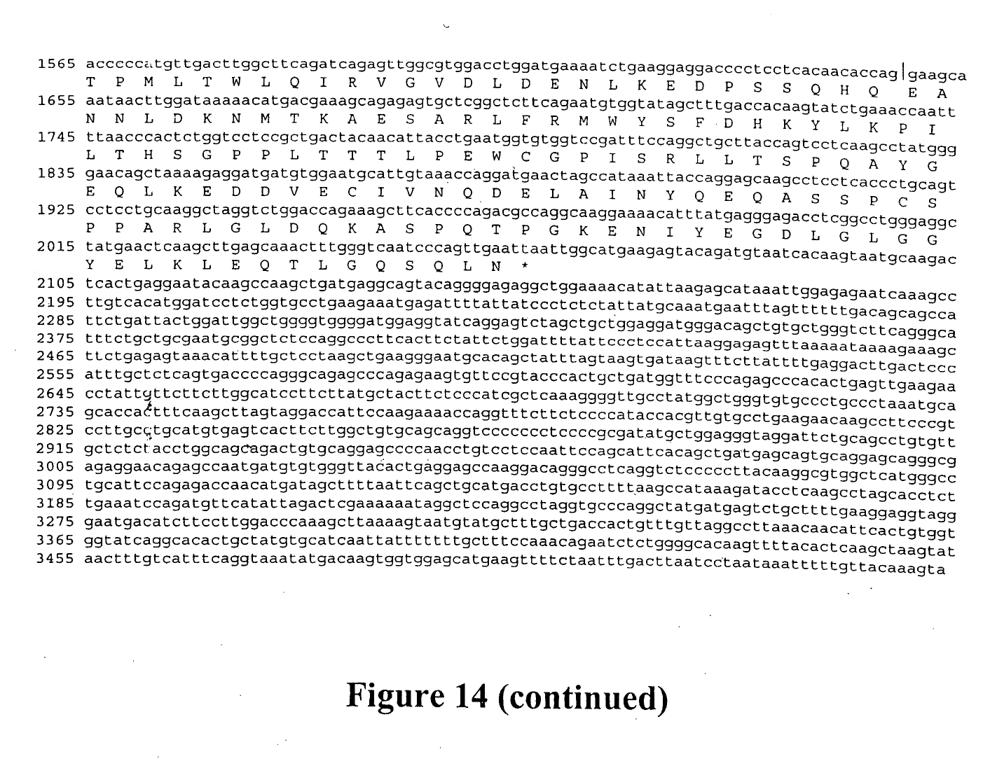

In an alternative embodiment, the marker comprises the nucleotide sequence and corresponding amino acid sequence of NHE as shown in FIG. 14 and SEQ ID NOs:22 and 23, respectively.

Accordingly, another related aspect of the present invention provides a molecular marker of a behavioral phenotype, said molecular marker comprising, in a genetic form, a nucleotide sequence or modified form thereof, or an expression product encoded thereby, said nucleotide sequence derived from the p-arm of chromosome 3 and comprising or contained within a sequence of nucleotides which hybridize under conditions of low stringency to a nucleic acid molecule encoding all or part of the gene product whose amino acid sequence is set forth in SEQ ID NO:22 and wherein the presence of a modified form of said molecular marker is indicative of a behavioral disorder or the likelihood that a subject may develop a behavioral disorder.

In a related embodiment, there is provided a molecular marker of a behavioral phenotype, said molecular marker comprising, in a genetic form, a nucleotide sequence or modified form thereof, or an expression product encoded thereby, said nucleotide sequence derived from the p-arm of chromosome 3 and comprising or contained within a sequence of nucleotides which hybridize under conditions of low stringency to a nucleic acid molecule comprising the nucleotide sequence set forth in SEQ ID NO:23 or its complementary form, said molecular marker is indicative of a behavioral disorder or the likelihood that a subject may develop a behavioral disorder.

Genes associated with the molecular marker of the present invention include KIAA0800, HUMAGCGB and ARP. A further aspect of the present invention provides for determining whether any of these genes have been disrupted in individuals with a behavioral disorder.

Accordingly, a further aspect of the present invention provides a molecular marker of a behavioral phenotype, said molecular marker comprising, in a genetic form, a nucleotide sequence or modified form thereof, or an expression product encoded thereby, said genetic sequence derived from the chromosome 3 and comprising or contained within a sequence of nucleotides which hybridize under conditions of low stringency to a nucleic acid molecule encoding all or part of the gene product whose amino acid sequence is set forth in one or more of SEQ ID NO:16 (KIAA0800), SEQ ID NO:17 (HUMAGCGB) and/or SEQ ID NO:18 (ARP) and wherein the presence of a modified form of said molecular marker is indicative of a behavioral disorder or the likelihood that a subject may develop a behavioral disorder. This is particularly the case when there is a modification to DOCK 3 and/or NHE.

The proteinaceous form of the molecular marker of the present invention includes normal polypeptides or proteins and mutant polypeptides or proteins. By “normal polypeptides” is meant forms of the polypeptide which are not associated with a behavioral disorder while “mutant polypeptides” refers broadly to one or more forms of the polypeptide which are associated with the development or progression of a behavioral disorder. A mutant polypeptide includes polypeptides or peptides encoded by the mutant polynucleotides of the preset invention. A mutant polypeptide includes the case where no polypeptide is made as well as when a non-sense polypeptide is produced due to non-contiguous nucleotide sequences being fused together. Mutation detection methods such as the protein truncation test (PTT) or methods relying on altered electrophoretic ability may conveniently be used in the assessment or screening for mutant polypeptides.

Homologues and derivatives of the isolated or recombinant proteinaceous molecular markers described herein are contemplated. The term derivatives, in this context includes fragments, parts, portions, mutants, homologues and analogues. Derivatives also include single or multiple amino acid substitutions, deletions and/or additions. “Additions” to amino acids include fusions with other peptides, polypeptides or proteins or fusions to nucleotide sequences. Derivatives may be from about 10 amino acids to about 10,000 amino acids in length, from about 30 amino acids to about 300 amino acids, from about 70 to about 5000 amino acids or from about 100 amino acids to about 700 amino acids. Reference herein to the proteinaceous molecular markers includes reference to all derivatives thereof including functional and non-functional derivatives, peptides and polypeptides. Preferably, the derivative has enhanced function for diagnosis, prophylaxis or therapy including immunotherapy of a behavioral disorder such as ADHD.

The amino acid sequence of proteinaceous forms of the present molecular marker are exemplified in SEQ ID NO:21 (DOCK 3 gene product) and SEQ ID NO:23 (NHE gene product) SEQ ID NO:17 (HUMAGCGB gene product); SEQ ID NO:18 (ARP gene product) and SEQ ID NO:16 (KIA0800 gene product).

The present invention provides, therefore, genetic probes such as oligonucleotides to screen for the molecular markers on chromosome 3 or its equivalent.

In another embodiment, the present invention contemplates antibody reagents for use in the diagnosis and/or treatment of a behavioral phenotype such as but not limited to ADHD which specifically recognize an epitope determined by a proteinaceous form of the subject molecular markers.

Antibodies to a proteinaceous form of a molecular marker of the present invention may be monoclonal or polyclonal. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. Antibodies may also be used to distinguish between “normal” polypeptides and “mutant” polypeptides.

In one embodiment, specific antibodies can be used to screen for a proteinaceous form of a molecular marker. The latter would be important, for example, as a means for screening for levels of a molecular marker in a cell extract or other biological fluid or purifying a proteinaceous form of a molecular marker made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies or synthetic antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of a proteinaceous form of a molecular marker.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of a proteinaceous form of a molecular marker, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Another aspect of the present invention contemplates a method for detecting a proteinaceous form of a molecular marker in a biological sample from a subject said method comprising contacting said biological sample with an antibody specific for a proteinaceous form of a molecular marker or its derivatives or homologues for a time and under conditions sufficient for an antibody-molecular marker complex to form, and then detecting said complex.

The presence of a proteinaceous form of a molecular marker may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain a proteinaceous form of a molecular marker including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, a first antibody having specificity for a proteinaceous form of a molecular marker or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking, covalently binding or physically adsorbing, the polymer-antibody complex is washed in preparation for the test sample. An aliquot of the sample to be tested is then added to the solid phase complex and incubated for a period of time sufficient (e.g. 2-40 minutes or where more convenient, overnight) and under suitable conditions (e.g. for about 20° C. to about 40° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the molecule of interest. Immunofluorescence and EIA techniques are both very well established in the art and are particularly preferred for the present method. However, other reporter molecules, such as radioisotope, chemiluminescent or bioluminescent molecules, may also be employed.

Synthetic forms of proteinaceous markers are also contemplated.

The present invention extends to a composition for the prophylaxis and/or treatment of a behavioral disorder such as ADHD comprising one or more of the present molecular markers. Gene or protein replacement compositions are especially contemplated.

Accordingly, yet another aspect of the invention pertains to a composition for the treatment and/or prophylaxis of a behavioral phenotype such as but not limited to ADHD, said composition containing a nucleic acid molecule comprising a sequence of nucleotides which hybridizes under conditions of low stringency to a sequence of nucleotides comprising or contained within SEQ ID NO:1 or SEQ IDN NO:2 or SEQ ID NO:3 or a complementary or derivative form thereof and one or more acceptable carriers and/or excipients.

A further aspect of the invention pertains to a composition for the treatment and/or prophylaxis of a behavioral phenotype such as but not limited to ADHD, said composition containing a polypeptide or protein or peptide comprising a contiguous sequence of amino acids as set forth in SEQ ID NO:21 or SEQ ID NO:22 or an amino acid sequence having at least 70% similarity thereto or a derivative thereof and one or more acceptable carriers and/or excipients.

The present invention provides, therefore, both gene-replacement and protein-replacement therapy in the treatment of a behavioral disorder. The protein-replacement therapy may involve providing gene products of DOCK 3 and/or NHE and optionally of gene products of the associated genes KIAA0800, HUMAGCGB and/or ARP. Alternatively, or in addition, the protein-replacement therapy may involve other members of a metabolic, physiological and/or neurological pathway but which DOCK 3, NHE, KIAA0800, HUMAGCGB and/or ARP are associated.

Another aspect of the present invention contemplates an isolated nucleic acid construct or vector including an expression vector or cell line comprising genetic forms of the subject molecular markers.

Yet one further aspect of the present invention extends to the use of a nucleic acid molecule comprising a sequence of nucleotides which hybridizes under conditions of low stringency to a sequence of nucleotides comprising or contained within SEQ ID NO:1 or SEQ ID NO:2, SEQ ID NO:3, SEQ ID NO:20 and/or SEQ ID NO:21 or SEQ ID NO:4 or SEQ ID NO:5 or SEQ ID NO:12 or SEQ ID NO:14 or a complementary or derivative from thereof or having at least 60% similarity thereto in the detection of a mutant genotype wherein the presence of said mutant genotype is indicative of a behavioral disorder or the likelihood that a subject may develop a behavioral disorder.

A further aspect of the present invention is directed to the use of a nucleic acid molecule in the manufacture of a medicament suitable for the treatment of a behavioral disorder wherein said nucleic acid molecule comprises a sequence of nucleotides which hybridizes under conditions of low stringency to a sequence of nucleotides comprising or contained within SEQ ID NO:1 or SEQ ID NO:2, SEQ ID NO:3, SEQ ID NO:20 and/or SEQ ID NO:21 or SEQ ID NO:4 or SEQ ID NO:5 or SEQ ID NO:12 or SEQ ID NO:14 or a complementary or derivative from thereof or having at least 60% similarity thereto.

Yet another related aspect of the invention contemplates a genetically modified animal model of a behavioral phenotype such as but not limited to ADHD. Preferably, the animal model is a mouse. Preferably the mouse is modified by the inclusion and/or exclusion of more of the present molecular markers.

A still yet further aspect of the present invention contemplates the use of a genetically modified animal model of a behavioral phenotype such as but not limited to ADHD for screening for modifying mutations which affect said behavioral phenotype.

Still another aspect of the present invention provides an isolated molecular marker for diagnosing a behavioral phenotype such as but not limited to ADHD.

Even yet another aspect of this invention contemplates a method for diagnosing a behavioral phenotype or assessing the likelihood that a subject may develop a behavioral phenotype such as but not limited to ADHD, said method comprising contacting a sample derived from said subject with a probe capable of detecting any aberrations in DOCK 3 and/or NHE or in any of SEQ ID NO:1 or SEQ ID NO:2, SEQ ID NO:3, SEQ ID NO:4 to SEQ ID NO:8 or SEQ ID NO:12 or SEQ ID NO:14 or derivatives thereof under conditions suitable for selective binding to occur wherein the absence of binding is indicative of a disrupted genetic region.

A further aspect of the present invention contemplates a method for diagnosing a behavioral phenotype or assessing the likelihood that a subject may develop a behavioral phenotype such as but not limited to ADHD is contemplated wherein said method comprises contacting a biological sample of a subject a ligand such as antibody to a proteinaceous form of the subject molecule marker wherein the absence of the molecular marker is indicative of a potential for development or presence of a behavioral disorder with a proteinaceous form of a molecular marker according to the present invention or antibodies thereto under conditions suitable for selective binding to occur.

Another aspect of the present invention is directed to a method for screening a compound for an ability to ameliorate one or more symptoms of a behavioral phenotype in a human subject comprising administering one or more of said compounds to a genetically modified animal model of said behavioral phenotype and assessing the animal for changes consistent with the amelioration of one or more symptoms of said disorder.

The present invention is further described by the following non-limiting Examples.

### EXAMPLE 1

### Physical Mapping of the Mutation

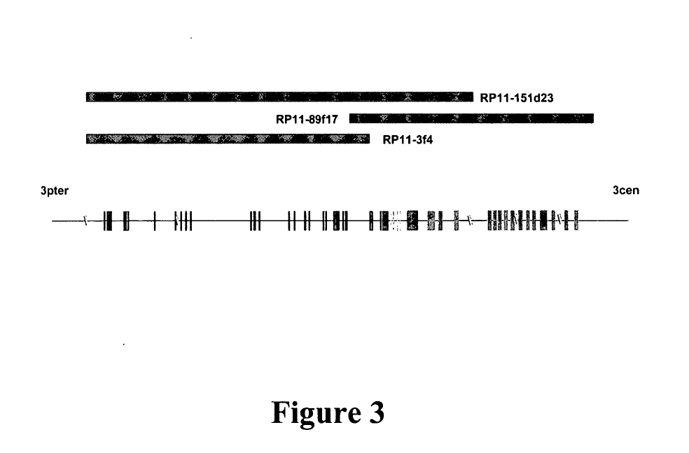

At the p arm a phage library in the lambda ZAP Express vector (Stratagene, La Jolla, Calif.) was constructed from one of the YAC clones that spanned the p arm breakpoint. EST and STS markers in the vicinity of the breakpoint were used as probes to screen this library by hybridization. This screen initially yielded two clones positive for EST markers in the area of interest. Sequence analysis of these and another three clones isolated on subsequent screens of the lambda library revealed the presence of four closely mapping genes, ARP, HUMAGCGB, KIAA0800 and KIAA0299. Three overlapping human BAC clones containing these genes were found via the high throughput genome sequence database at the National Centre for Biotechnology Information (NCBI, Bethesda, Md.; http://www.ncbi.nlm.nih.gov/). The BAC clones were analyzed by fluorescence in situ hybridization (FISH) with one localizing within the breakpoint and two clones crossing the p arm breakpoint (FIG. 3).

### EXAMPLE 2

### Fine Mapping the p arm Breakpoint

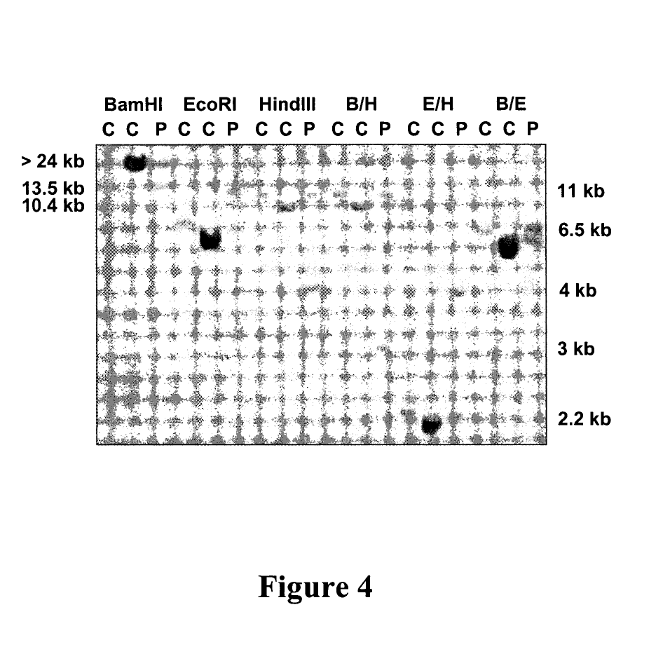

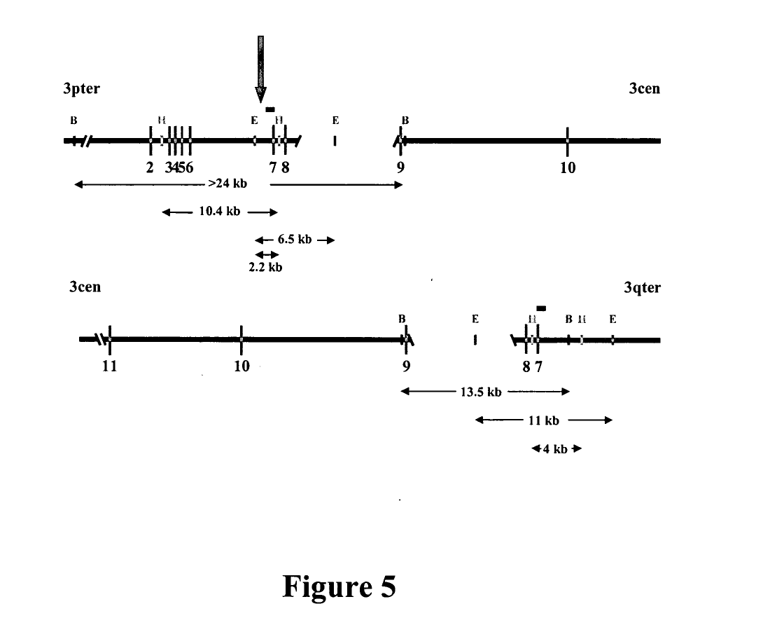

Southern blots of genomic DNA digests from an affected family member and male and female control subjects were probed with PCR products generated from unique genomic sequence in the introns of the KIAA0299 gene at the telomeric ends of the breakpoint-spanning BAC clones. A probe located 5′ and encompassing exon “7” yielded fragments of altered size in the patient compared to the control DNA (FIGS. 4 and 5). From the banding pattern seen on Southern analysis, a model of the breakpoint could be postulated. This was later confirmed by sequencing.

### EXAMPLE 3

### Identification of Unknown 3q Breakpoint Sequences

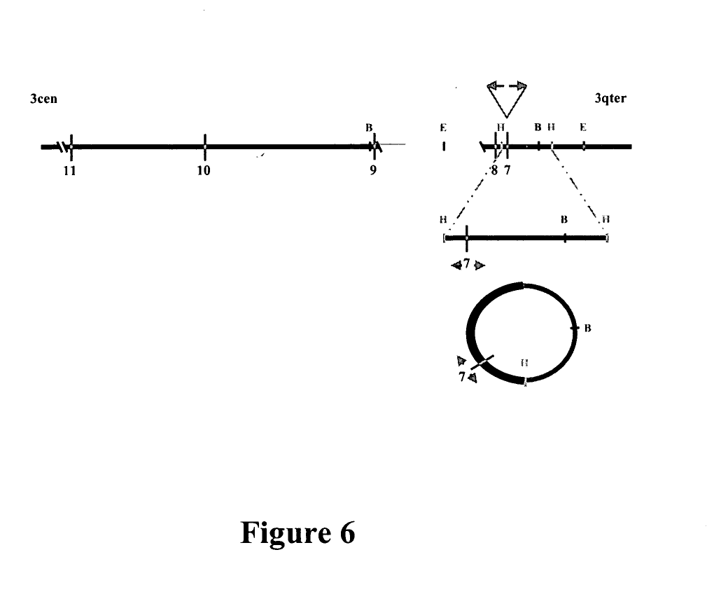

The known genomic sequence around the 3p breakpoint were used in inverse PCR experiments to identify the unknown sequences of q arm origin. Oligomers were designed that primed outwards from either side of “exon 7” (see FIG. 6). The genomic DNA from the patient and the two control individuals was digested with HindIII to excise a fragment surrounding the primers. This fragment was then re-circularized. The circular template was then used in long-range PCR experiments to amplify the mutant fragment. In the case of a HindIII digest, an approximately 10 kb band was expected from the normal chromosome and the inverted chromosome was expected to yield a 4 kb fusion fragment (see FIG. 5). The resulting 4 kb mutant fragment was excised from an agarose gel, purified and sequenced using big dye chemistry with primers from several sites within the known 3p sequence.

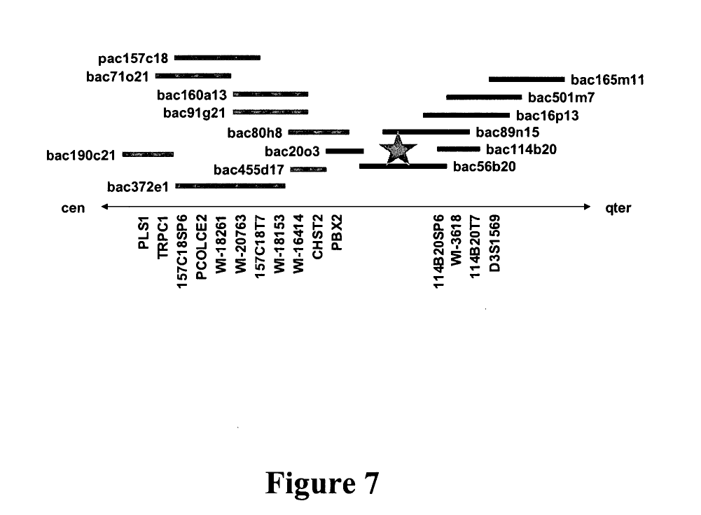

A BAC and PAC clone contig was assembled across the critical region of 3q using both laboratory-based chromosome walking and in silico methods (FIG. 7). By analyzing the resultant sequences using the BLAST programme at NCBI, it was found that the 3q breakpoint sequence originated from the overlap between BAC clones RP11-89n15 and RP11-56b20. Analysis of the overlapping sequence by database searching revealed a small expressed sequence tag site (EST) with homology to a sodium/hydrogen ion-exchanger family. This gene, termed NHE, appears to have ubiquitous expression throughout human tissues. Its function is as yet uncharacterized.

### EXAMPLE 4

### Assessment Procedure

Behavior Rating Scales

Behavior rating scales are valuable instruments, capable of providing a great deal of data quickly and efficiently (Conners et al., Psychopharmacology Bulletin 21: 809-843, 1985; Barkley et al., J. Child Psychol. Psychiat 18: 137-165, 1977). The rater (or informant) is asked whether a range of specified maladaptive behaviors are present in the child. Normative data allow comparison with a reference population so that the severity of a child's symptomatology can be meaningfully quantified.

Several standardized behavior rating scales were posted to the families for completion before the clinical assessment. These included the DSM-IV ADHD Parent and Teacher Rating Scales, Conners' Parent Rating Scale and Achenbach Child Behavior Checklist discussed below.

1. DSM-IVADHD Parent and Teacher Rating Scales (DuPaul and Barkley, ADHD Rating Scale, 1995)

This rating scale consists of the 18 items in The American Psychiatric Association's Diagnostic and Statistical Manual of Mental Disorders, 4th edition (DSM-IV) diagnostic criteria for ADHD (see appendix), listed such that the Inattention items alternate with the Hyperactive/Impulsive items. The respondent is required to mark each item on a four-point scale: “never or rarely” (0), “sometimes” (1), “often” (2) or “very often” (3). The scores are then dichotomized, such that 0's and 1's equate with the absence of the behavior, and 2's and 3's indicate its presence. This scale enables greater precision than simply rating each item as present or absent (as the criteria specify).

The DSM-IV ADHD Parent and Teacher Rating Scales was used in the present study to determine whether subjects met DSM-IV diagnostic criteria for ADHD and to classify subjects into types (Predominantly-Inattentive, Predominantly-Hyperactive/Impulsive, or Combined).

2. Conners' Parent Rating Scale—Revised, Conners' Teacher Rating Scale—Revised

(Goyette et al., J. Abnormal Child Psychology 6: 221-236,1978; Conners et al., 1985, supra)

The Conners' rating scales have been the most frequently used behavior rating scales in ADHD research (Barkley et al., 1977, supra). The revised 48-item version of the Conners' Parent Rating Scale—revised (CPRS—R) yields five factors—Conduct Problems, Learning Problems, Psychosomatic, Impulsive-Hyperactive, and Anxiety. In addition a Hyperactivity Index has been derived from the ten items with the highest loading from the factor scales. Each item is rated on a four point scale—not at all=0, just a little=1, pretty much=2, very much=3. The informant is asked to rate the items according to observed behavior over the preceding month. Scores for each factor are obtained by summing the scores on all items contained within the scale, and dividing by the number of items in the scale. Normative data have been published for the CPRS—R by age and sex categories for children from 3 to 17 years (Goyette et al., 1978, supra; Conners et al., 1985, supra). Raw scores for each factor are transformed by age and sex into T scores, with a mean of 50 and a standard deviation of 10.

The Conners' Teacher Rating Scale—revised (CTRS—R) complements the CPRS—R and scoring is identical. The revised 28-item version has three factors—Conduct Problem, Hyperactivity, and Inattentive-Passive. A Hyperactivity Index has again been derived from the ten highest loading items.

The CPRS—R and CTRS—R were used in this study to complement and expand on the DSM-IV ADHD Parent and Teacher Rating Scale data.

3. Achenbach Child Behavior Checklist, Teacher's Report Form

The Child Behavior Checklist (CBCL) (Achenbach, Manual for the Child Behavior Checklist/4-18 and 1991, Profile, 1991) and complimentary Teacher's Report Form (TRF) (Achenbach, Manual for the Teacher's Report Form and 1991, Profile, 1991) are broad band standardized behavior rating scales which are widely used in pediatric and child psychiatry clinical research and practice in many countries. These 113-item checklists provide a multidimensional profile of empirically derived problem behavior syndromes.

Each symptom is rated on a 3-point scale (0=not true, 1=somewhat or sometimes true, 2=very true or often true). The informant is asked to rate the items according to observed behavior over the preceding six months. The items are grouped into eight separate narrow band syndrome scales: Withdrawn, Somatic Complaints, Anxious/Depressed, Social Problems, Thought Problems, Attention Problems, Delinquent Behavior and Aggressive Behavior. These subscales are grouped into two broad-band factors or global dimensions—Internalizing (Withdrawn, Somatic Complaints, Anxious/Depressed) and Externalising (Aggressive, Delinquent Behaviors). In addition a Total Problems score is computed. Norms are provided in the form of T scores by age and sex category. T scores were derived using the CBCL publisher's computerized scoring program.

In addition to these rating scales, results of formal psychometric testing were sought where these had been conducted.

### EXAMPLE 5

### Clinical Assessment

All subjects then underwent a clinical evaluation. This consisted of a semi-structured interview (pregnancy/perinatal period, early development, temperament/behavior, learning, general health) and a neurodevelopmental assessment.

### EXAMPLE 6

### Northern Blot Analysis of Expression of DOCK 3 and NHE

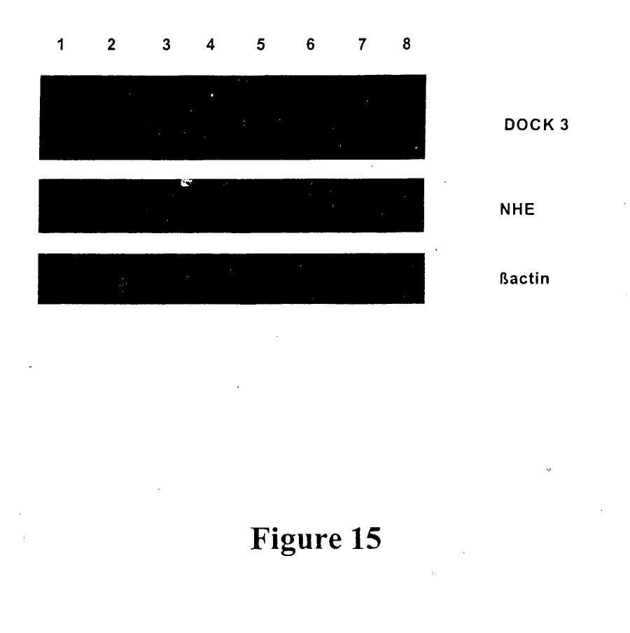

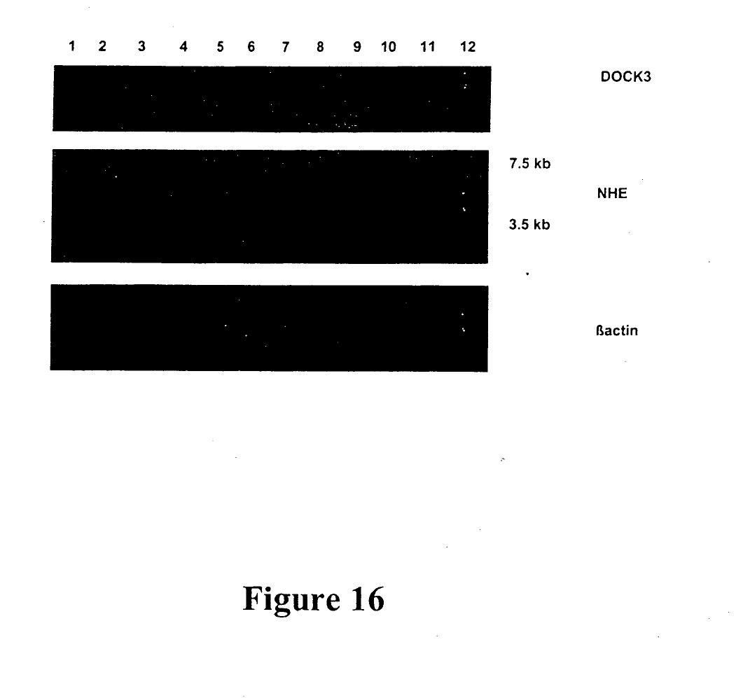

Northern blot analysis of a panel of human adult tissues confirmed that the expression of DOCK 3 is restricted to the brain. Specifically the gene is expressed in the cerebellum, cerebral cortex, medulla, occipital pole, frontal lobe, temporal lobe and putamen (FIG. 15). There was no expression in the spinal cord or in heart, skeletal muscle, colon, thymus, spleen, kidney, liver, small intestine, placenta, lung and peripheral blood leukocyte (FIG. 16). Evidence from the HUGE database (http://www.kazusa.or.jp/huge/index.html) suggests that this gene is also expressed in the testis. The NHE gene is more widely expressed. Specifically the gene is expressed in the cerebellum, cerebral cortex, medulla, occipital pole, frontal lobe, temporal lobe, putamen and spinal cord (FIG. 15) as well as the heart, skeletal muscle, thymus, spleen, kidney, liver, small intestine, placenta, lung and peripheral blood leukocyte (FIG. 16). The gene is not expressed in the colon. The NHE probe hybridized to a larger, 7.5 kb band in skeletal muscle.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

We claim:

1. A method of diagnosing a behavioral disorder in a subject comprising
obtaining a sample from said subject; and
analyzing said sample for the presence of a molecular marker of the behavioral disorder,
wherein said molecular marker comprises a genetic location on chromosome 3 or an equivalent location on another chromosome,
wherein a mutation at said location alone or in combination with environmental or other genetic factors is associated with, facilitates the development of, or facilitates the progression of said behavioral disorder.

2. The method of claim 1, wherein the behavioral disorder is Attention Deficit Hyperactivity Disorder (ADHD).

3. The method of claim 2, wherein the absence of the mutation is indicative of a low risk of developing ADHD.

4. The method of claim 2, wherein the genetic location of the molecular marker is associated with the DOCK 3 and/or NHE gene.

5. The method of claim 1 or 2, wherein the other genetic factors include a mutation in one or more of the HUMAGCGB, KIAA0800 and/or ARP gene.

6. The method of claim 1, wherein the mutation is selected from the group consisting of a nucleotide substitution, a deletion, an addition and an inversion.

7. The method of claim 6, wherein the mutation is a chromosome 3 inversion.

8. The method of claim 7, wherein chromosome 3 comprises p-arm and q-arm breakpoints and the inversion is between the p-arm and q-arm breakpoints.

9. The method of claim 8, wherein the inversion breakpoints are between band p21.3 and band q21.

10. The method of claim 9, wherein the molecular marker comprises a nucleotide sequence selected from the group consisting of SEQ ID NO:1, SEQ ID NO:2, a nucleotide sequence having at least about 60% similarity to SEQ ID NO:1 or SEQ ID NO:2, a nucleotide sequence capable of hybridizing to SEQ ID NO:1 and/or SEQ ID NO:2 under low stringency conditions, and complementary forms of a nucleotide sequence capable of hybridizing to SEQ ID NO:1 and/or SEQ ID NO:2 under low stringency conditions.

11. The method of claim 9, the molecular marker comprises a nucleotide sequence selected from the group consisting of SEQ ID NO:3, a nucleotide sequence having at least about 60% similarity to SEQ ID NO:3, a nucleotide sequence capable of hybridizing to SEQ ID NO:3 under low stringency conditions, and complementary forms of a nucleotide sequence capable of hybridizing to SEQ ID NO:3 under low stringency conditions.

12. The method of claim 3, wherein the molecular marker comprises a nucleotide sequence selected from the group consisting of SEQ ID NO:12, SEQ ID NO:14, a nucleotide sequence having at least about 60% similarity to SEQ ID NO:12 or SEQ ID NO:14, a nucleotide sequence capable of hybridizing to SEQ ID NO:12 and/or SEQ ID NO:14 under low stringency conditions, and complementary forms of nucleotide sequence capable of hybridizing to SEQ ID NO:12 and/or SEQ ID NO:14 under low stringency conditions, wherein the presence of said molecular marker is indicative of a low risk of developing ADHD.

13. The method of claim 2, wherein said molecular marker comprises a nucleotide sequence selected from the group consisting of SEQ ID NO:13, SEQ ID NO:15, a nucleotide sequence having at least about 60% similarity to SEQ ID NO:13 or SEQ ID NO:15, a nucleotide sequence capable of hybridizing to SEQ ID NO:13 or SEQ ID NO:15 under low stringency conditions, and complementary forms of a nucleotide sequence capable of hybridizing to SEQ ID NO:13 or SEQ ID NO:15 under low stringency conditions.

14. A method of diagnosing a behavioral disorder in a subject comprising
obtaining a sample from said subject; and
analyzing said sample for the presence of a molecular marker of the behavioral disorder,
wherein said molecular marker comprises a nucleotide sequence or a modified form thereof, whose amino acid sequence is selected from the group consisting of SEQ ID NO:21, SEQ ID NO:23, an amino acid sequence having at least about 60% similarity to SEQ ID NO:21 or SEQ ID NO:23,
wherein the presence of a modified form of said molecular marker is indicative of a behavioral disorder or the likelihood that a subject may develop a behavioral disorder.

15. The method of claim 14, wherein the molecular marker further comprises a nucleotide sequence or a modified form thereof, whose amino acid sequence is selected from the group consisting of SEQ ID NO:16, SEQ ID NO:17, SEQ ID NO:18, and an amino acid sequence having at least about 60% similarity to SEQ ID NO:16, SEQ ID NO:17 or SEQ ID NO:18.

16. The method of claim 14, wherein the amino acid sequence is SEQ ID NO:21.

17. The method of claim 14, wherein the amino acid sequence is SEQ ID NO:23.

18. The method of claim 14, wherein the modified form produces an absence of the gene product, an amino acid substitution in the gene product, an amino acid addition in the gene product, or amino acid deletion in the gene product.

19. The method of claim 14, wherein the behavioral disorder is ADHD.

20. An isolated nucleic acid molecule comprising a nucleic acid sequence selected from the group consisting of SEQ ID NOs:1 to 20, SEQ ID NO:22, a nucleotide sequence having at least about 60% similarity to SEQ ID NOs:1 to 20 or SEQ ID NO:22, a nucleotide sequence capable of hybridizing to SEQ ID NOs:1 to 22 or SEQ ID NO:22 under low stringency conditions, and complementary forms of a nucleotide sequence capable of hybridizing to SEQ ID NOs:1 to 22 or SEQ ID NO:22 under low stringency conditions.

21. An isolated protein comprising an amino acid sequence selected from the group consisting of SEQ ID NO:21, SEQ ID NO:23, SEQ ID NO:16, SEQ ID NO:17, SEQ ID NO:18, and an amino acid sequence having at least about 60% similarity to SEQ ID NO:21, SEQ ID NO:23, SEQ ID NO:16, SEQ ID NO:17, SEQ ID NO:18.

22. An isolated antibody to the isolated protein of claim 21.

23. The isolated antibody of claim 22, wherein the antibody is a monoclonal antibody.

24. A method for determining the likelihood of a subject having a behavioral disorder comprising
obtaining a sample from said subject;
determining the presence of a mutation in a nucleotide sequence on chromosome 3 in said sample, wherein the nucleotide sequence is selected from the group consisting of SEQ ID NO:1, SEQ ID NO:2, SEQ ID NO:3, SEQ ID NO:4 and SEQ ID NO:14.

25. The method of claim 24, wherein the nucleotide sequence comprises more than one member of the group.

26. The method of claim 24, wherein the behavioral disorder is ADHD.

27. The method of claim 26, wherein a mutated nucleotide sequence is selected from SEQ ID NO:13 and/or SEQ ID NO:15.

28. A kit for diagnosing a behavioral disorder, said kit in compartmental form comprising a genetic probe capable of detecting the presence of or a mutation in any one of SEQ ID NOs:1 to 20 and/or SEQ ID NO:22.
